(19) United States
(12) Patent Application Publication
(10) Pub. No.: US 2025/0223307 A1
(43) Pub. Date: Jul. 10, 2025

(54) MDM2/MDMX DOUBLE-TARGET INHIBITOR COMPOUND, PRODRUG, PHARMACEUTICAL COMPOSITION, AND PREPARATION METHOD THEREFOR AND USE THEREOF

(71) Applicant: GLOBAL HEALTH DRUG DISCOVERY INSTITUTE, Beijing (CN)

(72) Inventors: Zizhong LI, Beijing (CN); Ting WANG, Beijing (CN); Lei CHANG, Beijing (CN); Hui GAO, Beijing (CN)

(21) Appl. No.: 19/092,000

(22) Filed: Mar. 27, 2025

Related U.S. Application Data
(63) Continuation of application No. PCT/CN2023/131362, filed on Nov. 13, 2023.

(30) Foreign Application Priority Data
Nov. 14, 2022 (CN) 202211419976.3

(51) Int. Cl. C07F 9/6558; A61K 31/4166; A61K 31/4178; A61K 31/675; C07D 233/96; C07D 403/06

(52) U.S. Cl. CPC C07F 9/65583; A61K 31/4166; A61K 31/4178; A61K 31/675; C07D 233/96; C07D 403/06

(57) ABSTRACT

The disclosure provides a compound shown in formula (I) and a prodrug thereof, which have a good MDM2 and/or MDMX inhibiting effect and may be used for treating MDM2 and/or MDMX-mediated symptoms and/or diseases such as neoplastic diseases and/or idiopathic pulmonary fibrosis and preparing drugs for such symptoms or diseases. In particular, a compound containing a phosphoric acid (phosphate) structure has higher solubility. As the prodrug, the compound can release an MDM2 and/or MDMX double-target inhibitor compound with high activity in an animal body, which solves the problem that such double-target inhibitors are hardly prepared into drugs.

The disclosure claims priority of a priori application filed with China National Intellectual Property Administration on 14 Nov. 2022 and titled “MDM2/MDMX double-target inhibitor compound, prodrug, pharmaceutical composition, and preparation method therefor and use thereof” with application No. 202211419976.3. The entire content of the priori application is incorporated into the disclosure by reference.

### TECHNICAL FIELD

The disclosure belongs to the field of medicines, and specifically relates to an MDM2/MDMX double-target inhibitor compound, a prodrug, a pharmaceutical composition, and a preparation method therefor and use thereof.

### BACKGROUND

Members of murine double minute (MDM) family, MDM2 and MDMX, are genes closely related to occurrence and development of malignant tumors and primary negative regulators for p53. MDM2, the member of MDM family, is one of the strongest apoptosis inhibitory factors discovered so far. It is a ubiquitin-protein ligase taking part in identification of a target protein by ubiquitin, which results in that the target protein is degraded by proteasome. Its encoded protein may bind with p53 protein to degrade the p53 protein in a ubiquitylated manner and reduce its activity. The p53 protein, in return, binds with its gene promoter sequence to inhibit its transcriptional activity and reduce expression of MDM2, so as to form a negative feedback loop, so that the ability of the cell which is transformed, proliferated, and malignantly transformed is enhanced. The homologous protein MDMX of MDM2 is another important binding factor for p53, which is similar to MDM2 in inhibition function on p53 protein. The difference is that MDMX does not have E3 ligase activity.

p53 may be divided into two types: wild p53 and mutant p53. The wild p53 is a tumor suppressor gene which is studies mostly deeply at present, which may induce cell apoptosis rapidly and prevent generation of cells with a potential canceration probability, so as to effectively exert the cancer inhibition effect. The wild p53 accounts for about 50% of all cancers. In these cancers, overexpression of MDM2/X proteins may be observed in most cases. Based on 28 different types of tumors, analysis of about 4000 tumor samples shows that the MDM2 gene is amplified in about 7% of tumor samples averagely, with the amplitude of amplification of 2-10 times. Over 80% of liposarcoma has amplification of the MDM2 gene and overexpression of the protein. In addition, overexpression of MDM2 is also common in osteosarcoma, esophagus cancer, and breast cancer. In many human cancers, MDMX is overexpressed, including glioma, soft tissue sarcoma, melanoma, retinoblastoma, breast cancer, and hematological malignancies. Through comparative study of data published in the cancer genome atlas (TCGA), scientists may recognize the expression of MDMX in patients with different cancer types. It is worth noting that in AML patients, the expression of MDMX is considerably higher than the expression of MDMX in other most tumor types. It is worth noting that compared with other tumor cells, malignant tumors originated from the blood system also have highly expressed MDMX.

Studies show that in fibrotic lesion of human idiopathic pulmonary fibrosis (IPF) and a pulmonary fibrosis animal model of an aged mouse, the MDMX is overexpressed. The MDMX is a matrix stiffness regulated p53 endogenous inhibitor. Reduction of matrix stiffness will down-regulate expression of the MDMX, resulting in that p53 in a primary lung myofibroblast separated from an IPF patient is activated. Increase of p53 function activates a gene program which can sensitize the lung myofibroblast to a process of cell apoptosis and promote clearance of apoptotic lung myofibroblast by macrophages. Clearance of the MDMX in a fibrotic lung matrix by a targeted non-enzymatic cross-linking or gene knockout method may activate an MDMX-p53 channel, so as to eliminate pulmonary fibrosis of the mouse induced by agedness. These discoveries show that mechanically sensitive MDMX is a molecular target which has a therapeutic potential and fights against senescence-associated persistent pulmonary fibrosis.

At present, MDM2-p53 interacting small-molecule inhibitors under clinical studies are as follows: AMG232 (KRT-232) from Kartos, APG-115 from Ascentage Pharma, and DS-3032b from Daiichi Sankyo Company Limited are under clinical studies in different stages. These compounds have low affinity to the MDMX and only inhibit MDM2-p53 protein interaction. Since the MDMX has a compensation effect on the MDM2, when p53 is activated by using the MDM2 selective inhibitor, MDMX-p53 interaction will reduce the p53 activating effect generated due to MDM2 inhibition. This phenomenon may be a reason that the clinical efficacy of the MDM2 selective inhibitor is non-significant. So far, there have been no MDMX selective inhibitors with high activity. ALRN-6924 is a unique p53-MDM2/MDMX double-inhibitor entering clinical practice, which is a “binding peptide” capable of blocking the interaction between p53 and MDM2 and MDMX. Clinical study results show that its safety index is superior to that of the MDM2 selective inhibitor, but its micromole level cell activity limits clinical widespread application.

### SUMMARY

To improve the above technical problem, the disclosure provides a compound shown in formula (I), and a raceme, a stereoisomer, a tautomer, an isotopic label, a solvate, a polymorph, a pharmaceutically acceptable salt or a prodrug compound thereof:

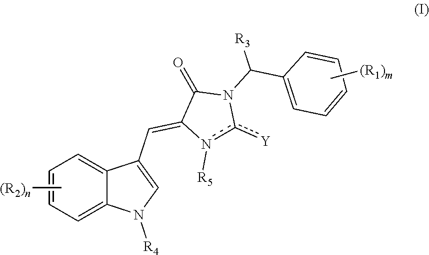

where
R₁ is selected from H, halogen, CN, C₁₋₆ alkyl, C₂₋₆ alkenyl, C₂₋₆ alkynyl, C₁₋₆ alkoxyl, halogenated C₁₋₆ alkyl, and halogenated C₁₋₆ alkoxyl;
R₂ is selected from H, C₁₋₆ alkyl, halogen, C₁₋₆alkoxyl, C₁₋₆ alkylthio, halogenated C₁₋₆ alkyl, and halogenated C₁₋₆ alkoxyl;
R₃ is selected from H, R₃ₐ—O—C₁₋₆ alkyl, —CH(═O), and —C(═O)—NH—R_{3b}; R₃ₐ is selected from H or —P(═O)(OR₃₁)(OR₃₂); R_{3b} is selected from the following radicals which are unsubstituted or optionally substituted with one, two, three or more R_{3c}: C₁₋₆ alkyl, C₃₋₈ cycloalkyl, and C₃₋₈ cycloalkyl-C₁₋₆ alkyl; R_{3c} is selected from OH, C₁₋₆ alkyl-NH—, (C₁₋₆ alkyl)₂-N—, and —O—P(═O)(OR₃₁)(OR₃₂); and R₃₁ and R₃₂ are same or different, and are independently selected from H and C₁₋₆ alkyl;
R₄ is selected from H and —P(═O)(OR₄₁)(OR₄₂); and R₄₁ and R₄₂ are same or different, and are independently selected from H and C₁₋₆ alkyl;
R₅ is selected from H and —P(═O)(OR₅₁)(OR₅₂); and R₅₁ and R₅₂ are same or different, and are independently selected from H and C₁₋₆ alkyl;
Y is O or OR₆; R₆ is selected from H and —P(═O)(OR₆₁)(OR₆₂); and R₆₁ and R₆₂ are same or different, and are independently selected from H and C₁₋₆ alkyl;
[symbol] is selected from a single bond or a double bond;
m is selected from 0, 1, 2, 3, 4 or 5; and
n is selected from 0, 1, 2, 3 or 4.

According to an embodiment of the disclosure, R₁ is selected from H, halogen, CN, C₁₋₃ alkyl, C₂₋₃ alkenyl, C₁₋₃ alkoxyl, halogenated C₁₋₃ alkyl, and halogenated C₁₋₃ alkoxyl;
according to an embodiment of the disclosure, R₁ is selected from H, F, Cl, CN, acetenyl, methyl, ethyl, and propyl.
according to an embodiment of the disclosure, R₂ is selected from H, C₁₋₃ alkyl, halogen, C₁₋₃ alkoxyl, C₁₋₃ alkylthio, and halogenated C₁₋₃ alkoxyl;
according to an embodiment of the disclosure, R₂ is selected from H, F, Cl, Br, methyl, methoxyl, fluoromethoxyl, and methylthio.

According to an embodiment of the disclosure, R₃ is selected from H, R₃ₐ—O—C₁₋₃ alkyl, —CH(═O), and —C(═O)—NH—R_{3b}; R₃ₐ is selected from H or —P(═O)(OH)(OH); R_{3b} is selected from the following radicals which are unsubstituted or optionally substituted with one, two, three or more R_{3c}: C₁₋₃ alkyl, C₃₋₈ cycloalkyl, and C₃₋₈ cycloalkyl-C₁₋₃ alkyl; R_{3c} is selected from OH, C₁₋₃ alkyl-NH—, (C₁₋₃ alkyl)₂-N—, and —O—P(═O)(OH)(OH).

According to an embodiment of the disclosure, R₃ is selected from H, methyl,

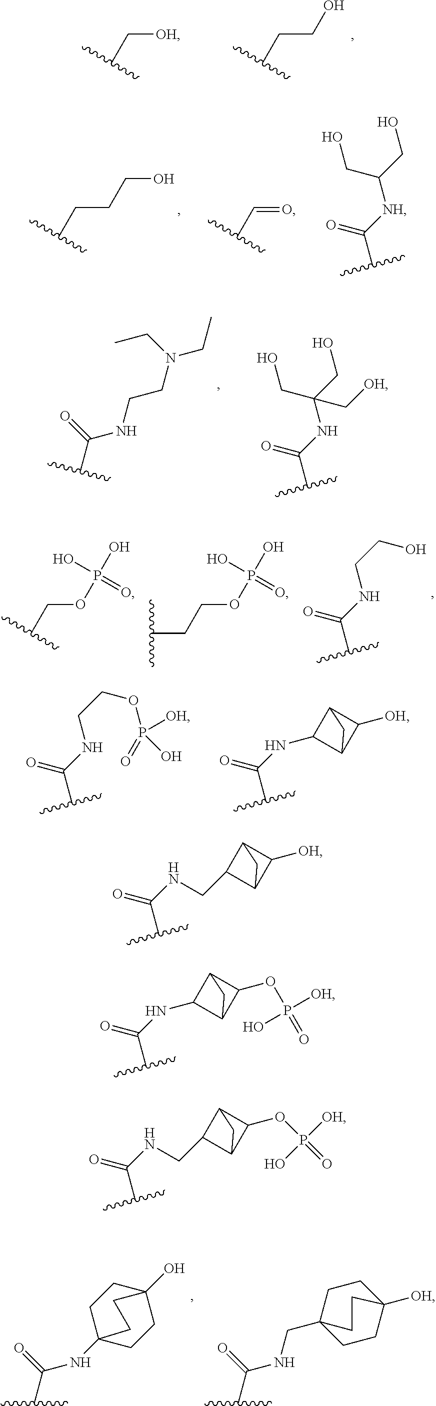

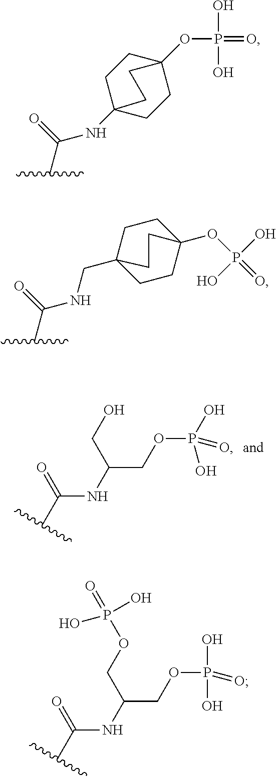 [SMILES: *C(=O)NC12CCC(OP(=O)(O)O)(CC1)CC2 ; *C(=O)NCC12CCC(OP(=O)(O)O)(CC1)CC2 ; *C(=O)NC(CO)COP(=O)(O)O ; *C(=O)NC(COP(=O)(O)O)COP(=O)(O)O]

and for example, selected from

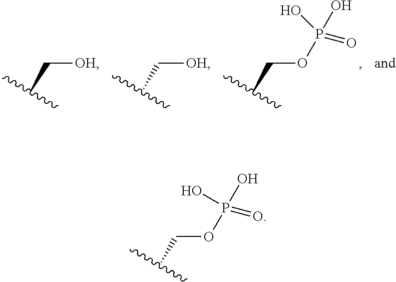

According to an embodiment of the disclosure, R₄ is selected from H and —P(═O)(OR₄₁)(OR₄₂); and R₄₁ and R₄₂ are same or different, and are independently selected from H and C₁₋₃ alkyl;
according to an embodiment of the disclosure, R₄ selected from H and —P(═O)(OH)(OH);
according to an embodiment of the disclosure, R₅ is selected from H and —P(═O)(OR₅₁)(OR₅₂); and R₅₁ and R₅₂ are same or different, and are independently selected from H and C₁₋₃ alkyl;
according to an embodiment of the disclosure, R₅ selected from H and —P(═O)(OH)(OH);
according to an embodiment of the disclosure, Y is O or OR₆; R₆ is selected from H and —P(═O)(OR₆₁)(OR₆₂); and R₆₁ and R₆₂ are same or different, and are independently selected from H and C₁₋₃ alkyl;
according to an embodiment of the disclosure, R₆ selected from H and —P(═O)(OH)(OH);
according to an embodiment of the disclosure, m is selected from 1, 2 or 3.

According to an embodiment of the disclosure, n is selected from 1 or 2.

According to an embodiment of the disclosure, the prodrug of the compound shown in formula (I) has a structure shown in formula II:

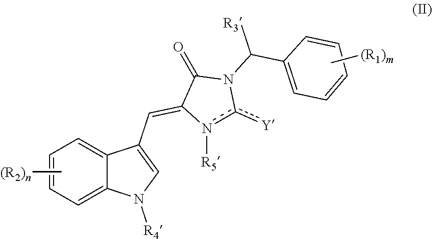

where R₁, R₂, m, and n independently have definitions described above;
R₃′ has a definition of R₃;
R₄′ is selected from H and —P(═O)(OR₄₁′)(OR₄₂′); and R₄₁′ and R₄₂′ are same or different, and are independently selected from H and C₁₋₆ alkyl;
R₅′ is selected from H and —P(═O)(OR₅₁′)(OR₅₂′); and R₅₁′ and R₅₂′ are same or different, and are independently selected from H and C₁₋₆ alkyl;
Y′ is O or OR₆′; R₆′ is selected from H and —P(═O)(OR₆₁′)(OR₆₂′); and R₆₁′ and R₆₂′ are same or different, and are independently selected from H and C₁₋₆alkyl; and
[symbol] is selected from a single bond or a double bond.

According to the technical solution of the disclosure:
R₃′ is, for example, selected from H, R₃ₐ—O—C₁₋₆ alkyl, —CH(═O), and —C(═O)—NH—R_{3b}; R₃ₐ is selected from H or —P(═O)(OH)(OH); R_{3b} is selected from the following radicals which are unsubstituted or optionally substituted with one, two, three or more R_{3c}: C₁₋₆ alkyl, C₃₋₈ cycloalkyl, and C₃₋₈ cycloalkyl-C₁₋₆ alkyl; R_{3c} is selected from OH, C₁₋₆ alkyl-NH—, (C₁₋₆ alkyl)₂-N—, and —O—P(═O)(OH)(OH);
R₄′ is selected from H and —P(═O)(OR₄₁′)(OR₄₂′); and R₄₁′ and R₄₂′ are same or different, and are independently selected from H and C₁₋₃ alkyl; preferably, R₄′ is selected from H and —P(═O)(OH)(OH);
R₅′ is selected from H and —P(═O)(OR₅₁′)(OR₅₂′); and R₅₁′ and R₅₂′ are same or different, and are independently selected from H and C₁₋₃ alkyl; preferably, R₅′ is selected from H and —P(═O)(OH)(OH);
Y′ is O or OR₆′; R₆′ is selected from H and —P(═O)(OR₆₁′)(OR₆₂′); and R₆₁′ and R₆₂′ are same or different, and are independently selected from H and C₁₋₃ alkyl; preferably, R₆′ is selected from H and —P(═O)(OH)(OH);
[symbol] is selected from a single bond or a double bond; and
at least one of R₄′, R₅′, R₆′, R₃ₐ, and R_{3c} is —P(═O)(OH)(OH).

According to an embodiment of the disclosure, the compound is selected from the following structures:

| Number | Structure |
|---|---|
| 1 | 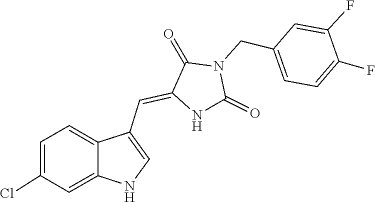 [SMILES: O=C1N/C(=C\c2c[nH]c3cc(Cl)ccc23)C(=O)N1Cc1ccc(F)c(F)c1] |
| 2 | 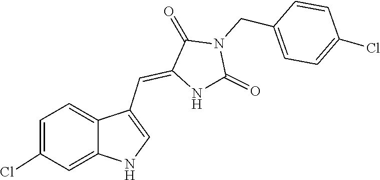 [SMILES: O=C1N/C(=C\c2c[nH]c3cc(Cl)ccc23)C(=O)N1Cc1ccc(Cl)cc1] |
| 3 | 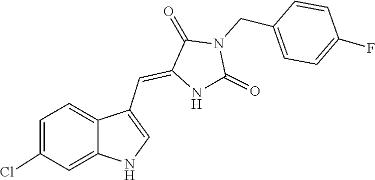 [SMILES: O=C1N/C(=C\c2c[nH]c3cc(Cl)ccc23)C(=O)N1Cc1ccc(F)cc1] |
| 4 | 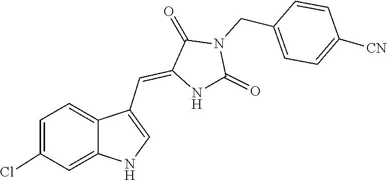 [SMILES: N#Cc1ccc(CN2C(=O)N/C(=C\c3c[nH]c4cc(Cl)ccc34)C2=O)cc1] |
| 5 | 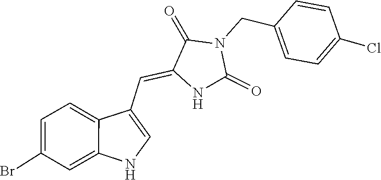 [SMILES: O=C1N/C(=C\c2c[nH]c3cc(Br)ccc23)C(=O)N1Cc1ccc(Cl)cc1] |
| 6 | 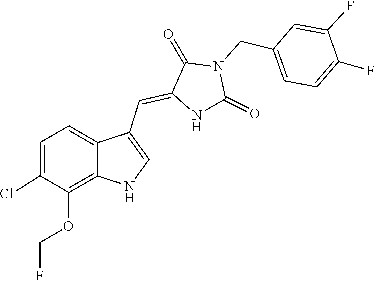 [SMILES: O=C1N/C(=C\c2c[nH]c3c(OCF)c(Cl)ccc23)C(=O)N1Cc1ccc(F)c(F)c1] |
| 7 | 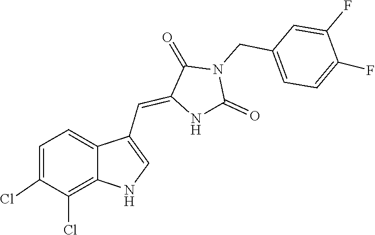 [SMILES: O=C1N/C(=C\c2c[nH]c3c(Cl)c(Cl)ccc23)C(=O)N1Cc1ccc(F)c(F)c1] |
| 8 | 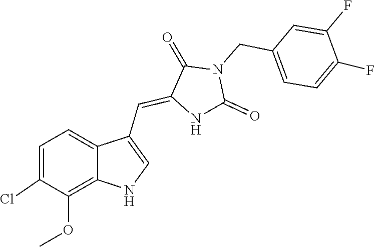 [SMILES: COc1c(Cl)ccc2c(/C=C3\NC(=O)N(Cc4ccc(F)c(F)c4)C3=O)c[nH]c12] |
| 9 | 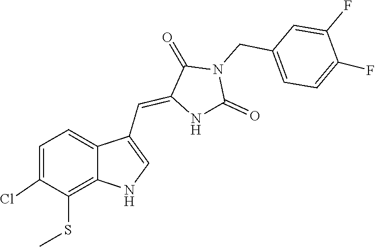 [SMILES: CSc1c(Cl)ccc2c(/C=C3\NC(=O)N(Cc4ccc(F)c(F)c4)C3=O)c[nH]c12] |
| 10 | 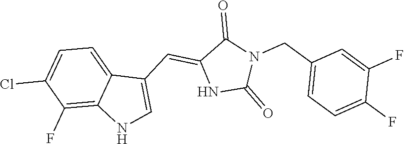 [SMILES: O=C1N/C(=C\c2c[nH]c3c(F)c(Cl)ccc23)C(=O)N1Cc1ccc(F)c(F)c1] |
| 11 | 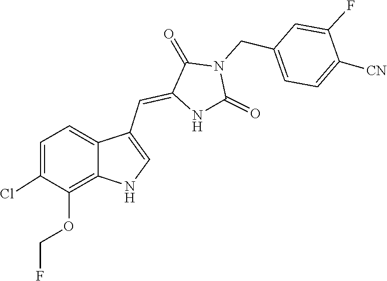 [SMILES: N#Cc1ccc(CN2C(=O)N/C(=C\c3c[nH]c4c(OCF)c(Cl)ccc34)C2=O)cc1F] |
| 12 | 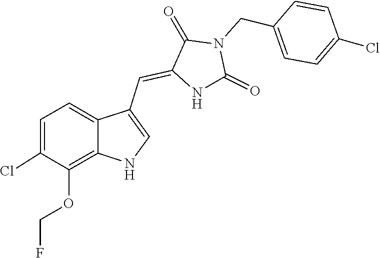 [SMILES: O=C1N/C(=C\c2c[nH]c3c(OCF)c(Cl)ccc23)C(=O)N1Cc1ccc(Cl)cc1] |
| 13 | 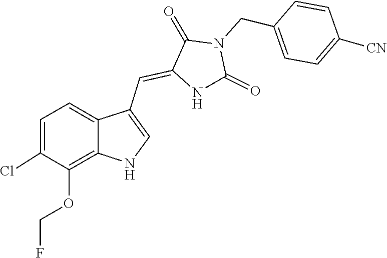 [SMILES: N#Cc1ccc(CN2C(=O)N/C(=C\c3c[nH]c4c(OCF)c(Cl)ccc34)C2=O)cc1] |
| 14 | 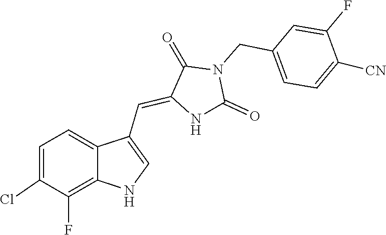 [SMILES: N#Cc1ccc(CN2C(=O)N/C(=C\c3c[nH]c4c(F)c(Cl)ccc34)C2=O)cc1F] |
| 15 | 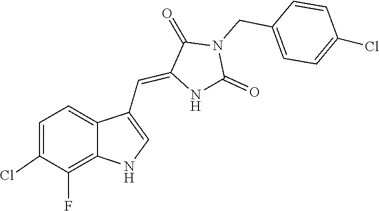 [SMILES: O=C1N/C(=C\c2c[nH]c3c(F)c(Cl)ccc23)C(=O)N1Cc1ccc(Cl)cc1] |
| 16 | 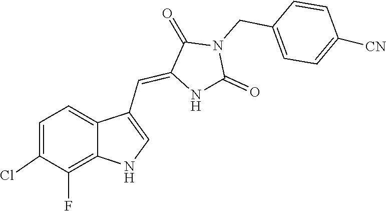 [SMILES: N#Cc1ccc(CN2C(=O)N/C(=C\c3c[nH]c4c(F)c(Cl)ccc34)C2=O)cc1] |
| 17 | 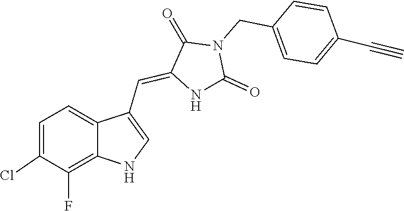 [SMILES: C#Cc1ccc(CN2C(=O)N/C(=C\c3c[nH]c4c(F)c(Cl)ccc34)C2=O)cc1] |
| 18 | 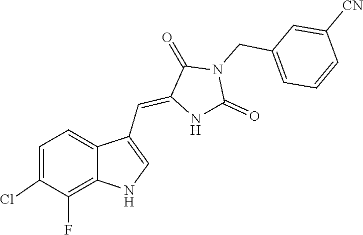 [SMILES: N#Cc1cccc(CN2C(=O)N/C(=C\c3c[nH]c4c(F)c(Cl)ccc34)C2=O)c1] |
| 19 | 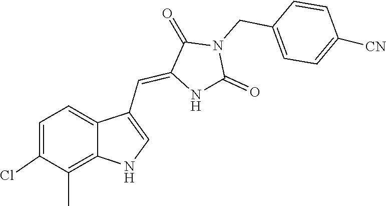 [SMILES: Cc1c(Cl)ccc2c(/C=C3\NC(=O)N(Cc4ccc(C#N)cc4)C3=O)c[nH]c12] |
| 20 | 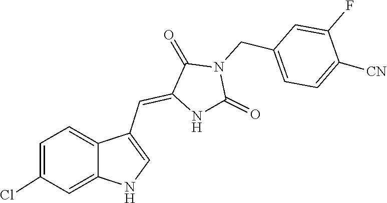 [SMILES: N#Cc1ccc(CN2C(=O)N/C(=C\c3c[nH]c4cc(Cl)ccc34)C2=O)cc1F] |
| 21 | 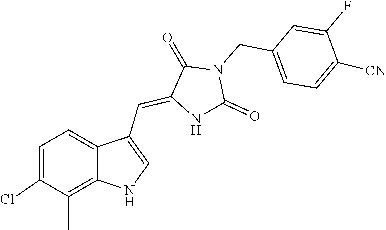 [SMILES: Cc1c(Cl)ccc2c(/C=C3\NC(=O)N(Cc4ccc(C#N)c(F)c4)C3=O)c[nH]c12] |
| 22 | 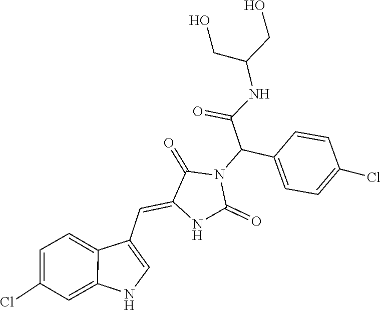 [SMILES: O=C(NC(CO)CO)C(c1ccc(Cl)cc1)N1C(=O)N/C(=C\c2c[nH]c3cc(Cl)ccc23)C1=O] |
| 23 | 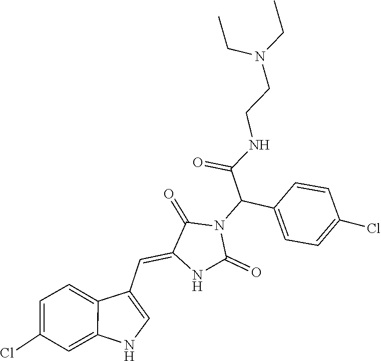 [SMILES: CCN(CC)CCNC(=O)C(c1ccc(Cl)cc1)N1C(=O)N/C(=C\c2c[nH]c3cc(Cl)ccc23)C1=O] |
| 24 | 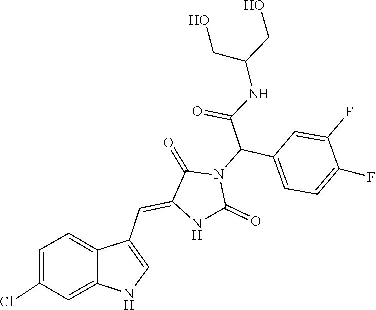 [SMILES: O=C(NC(CO)CO)C(c1ccc(F)c(F)c1)N1C(=O)N/C(=C\c2c[nH]c3cc(Cl)ccc23)C1=O] |
| 25 | 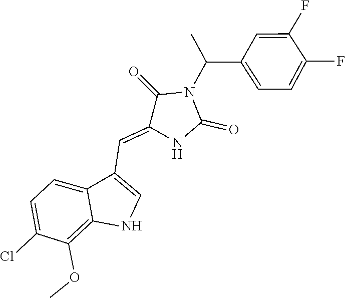 [SMILES: COc1c(Cl)ccc2c(/C=C3\NC(=O)N(C(C)c4ccc(F)c(F)c4)C3=O)c[nH]c12] |
| 26 | 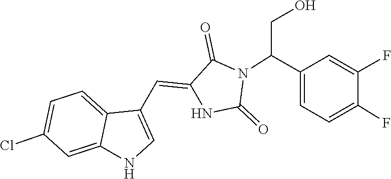 [SMILES: O=C1N/C(=C\c2c[nH]c3cc(Cl)ccc23)C(=O)N1C(CO)c1ccc(F)c(F)c1] |
| 27 | 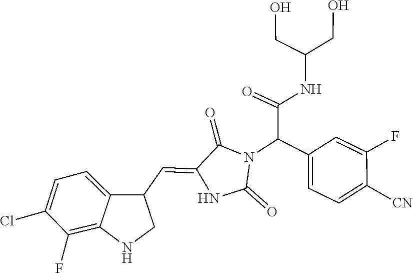 [SMILES: N#Cc1ccc(C(C(=O)NC(CO)CO)N2C(=O)N/C(=C\C3CNc4c3ccc(Cl)c4F)C2=O)cc1F] |
| 28 | 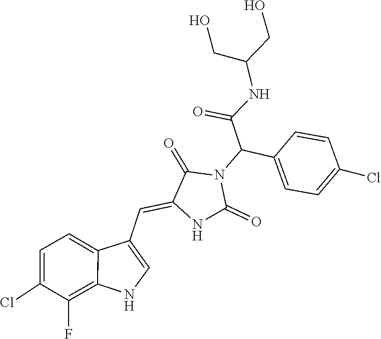 [SMILES: O=C(NC(CO)CO)C(c1ccc(Cl)cc1)N1C(=O)N/C(=C\c2c[nH]c3c(F)c(Cl)ccc23)C1=O] |
| 29 | 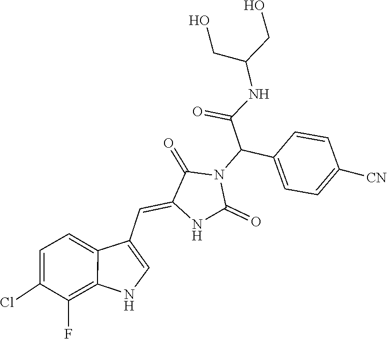 [SMILES: N#Cc1ccc(C(C(=O)NC(CO)CO)N2C(=O)N/C(=C\c3c[nH]c4c(F)c(Cl)ccc34)C2=O)cc1] |
| 30 | 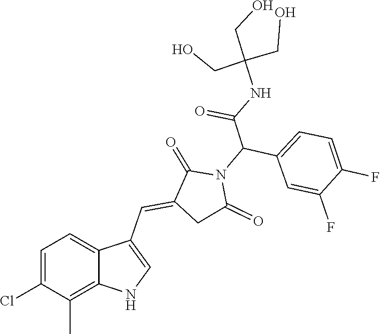 [SMILES: Cc1c(Cl)ccc2c(/C=C3\CC(=O)N(C(C(=O)NC(CO)(CO)CO)c4ccc(F)c(F)c4)C3=O)c[nH]c12] |
| 31 | 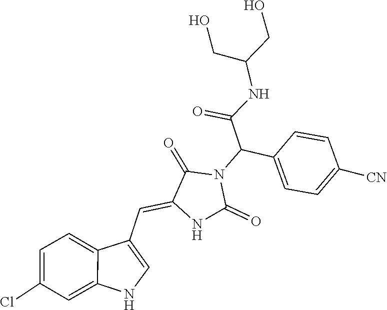 [SMILES: N#Cc1ccc(C(C(=O)NC(CO)CO)N2C(=O)N/C(=C\c3c[nH]c4cc(Cl)ccc34)C2=O)cc1] |
| 32 | 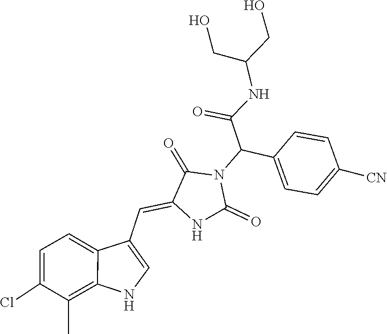 [SMILES: Cc1c(Cl)ccc2c(/C=C3\NC(=O)N(C(C(=O)NC(CO)CO)c4ccc(C#N)cc4)C3=O)c[nH]c12] |
| 33 | 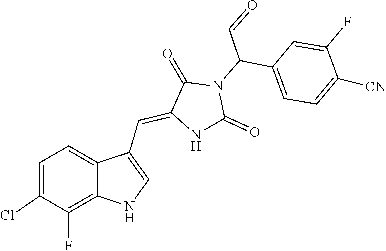 [SMILES: N#Cc1ccc(C(C=O)N2C(=O)N/C(=C\c3c[nH]c4c(F)c(Cl)ccc34)C2=O)cc1F] |
| 34 | 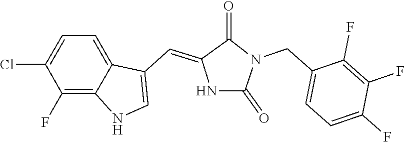 [SMILES: O=C1N/C(=C\c2c[nH]c3c(F)c(Cl)ccc23)C(=O)N1Cc1ccc(F)c(F)c1F] |
| 35 | 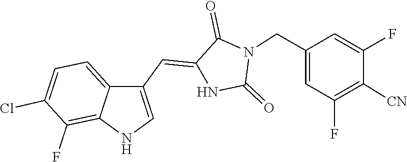 [SMILES: N#Cc1c(F)cc(CN2C(=O)N/C(=C\c3c[nH]c4c(F)c(Cl)ccc34)C2=O)cc1F] |
| 36 | 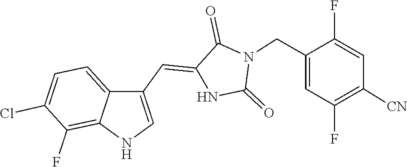 [SMILES: N#Cc1cc(F)c(CN2C(=O)N/C(=C\c3c[nH]c4c(F)c(Cl)ccc34)C2=O)cc1F] |
| 37 | 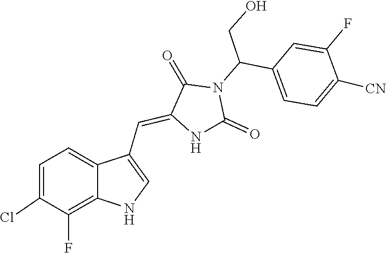 [SMILES: N#Cc1ccc(C(CO)N2C(=O)N/C(=C\c3c[nH]c4c(F)c(Cl)ccc34)C2=O)cc1F] |
| 38 | 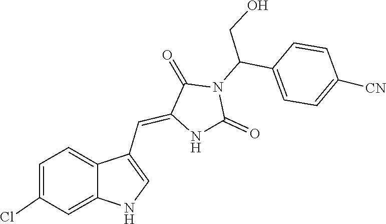 [SMILES: N#Cc1ccc(C(CO)N2C(=O)N/C(=C\c3c[nH]c4cc(Cl)ccc34)C2=O)cc1] |
| 39 | 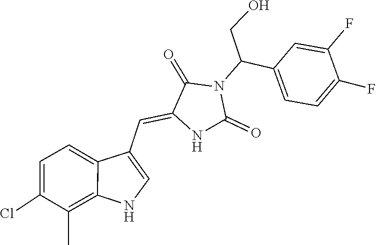 [SMILES: Cc1c(Cl)ccc2c(/C=C3\NC(=O)N(C(CO)c4ccc(F)c(F)c4)C3=O)c[nH]c12] |
| 40 | 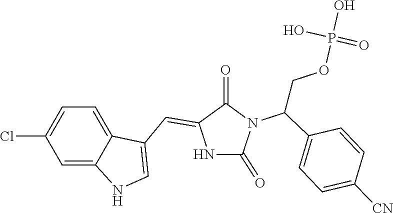 [SMILES: N#Cc1ccc(C(COP(=O)(O)O)N2C(=O)N/C(=C\c3c[nH]c4cc(Cl)ccc34)C2=O)cc1] |
| 41 | 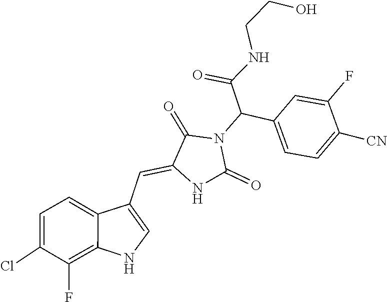 [SMILES: N#Cc1ccc(C(C(=O)NCCO)N2C(=O)N/C(=C\c3c[nH]c4c(F)c(Cl)ccc34)C2=O)cc1F] |
| 42 | 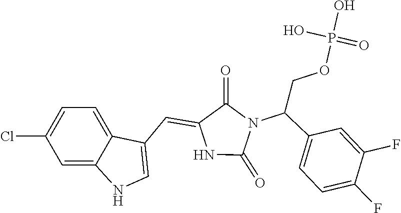 [SMILES: O=C1N/C(=C\c2c[nH]c3cc(Cl)ccc23)C(=O)N1C(COP(=O)(O)O)c1ccc(F)c(F)c1] |
| 43 | 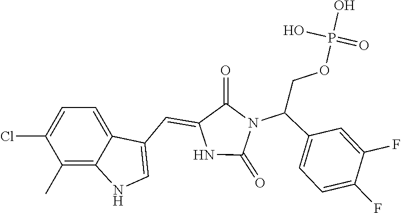 [SMILES: Cc1c(Cl)ccc2c(/C=C3\NC(=O)N(C(COP(=O)(O)O)c4ccc(F)c(F)c4)C3=O)c[nH]c12] |
| 44 | 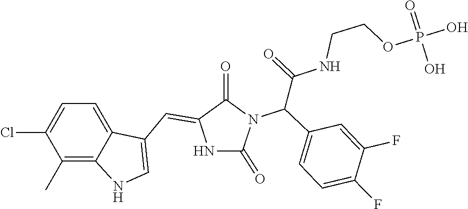 [SMILES: Cc1c(Cl)ccc2c(/C=C3\NC(=O)N(C(C(=O)NCCOP(=O)(O)O)c4ccc(F)c(F)c4)C3=O)c[nH]c12] |
| 45 | 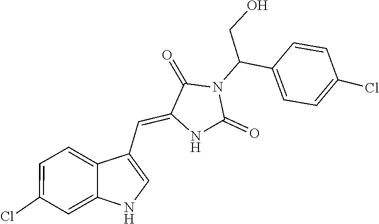 [SMILES: O=C1N/C(=C\c2c[nH]c3cc(Cl)ccc23)C(=O)N1C(CO)c1ccc(Cl)cc1] |
| 46 | 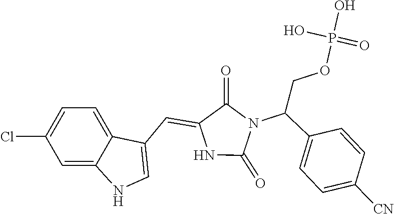 [SMILES: N#Cc1ccc(C(COP(=O)(O)O)N2C(=O)N/C(=C\c3c[nH]c4cc(Cl)ccc34)C2=O)cc1] |
| 47 | 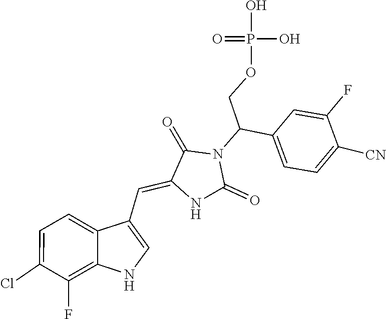 [SMILES: N#Cc1ccc(C(COP(=O)(O)O)N2C(=O)N/C(=C\c3c[nH]c4c(F)c(Cl)ccc34)C2=O)cc1F] |
| 48 | 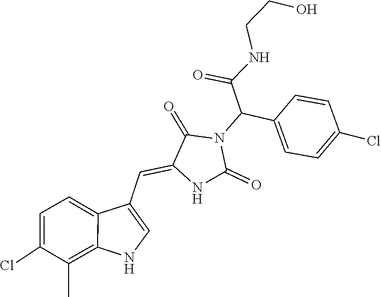 [SMILES: Cc1c(Cl)ccc2c(/C=C3\NC(=O)N(C(C(=O)NCCO)c4ccc(Cl)cc4)C3=O)c[nH]c12] |
| 49 | 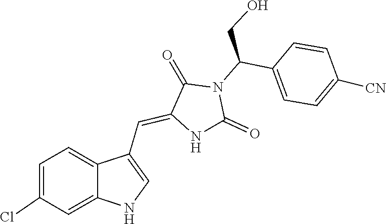 [SMILES: N#Cc1ccc([C@H](CO)N2C(=O)N/C(=C\c3c[nH]c4cc(Cl)ccc34)C2=O)cc1] |
| 50 | 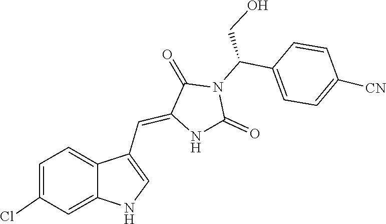 [SMILES: N#Cc1ccc([C@@H](CO)N2C(=O)N/C(=C\c3c[nH]c4cc(Cl)ccc34)C2=O)cc1] |
| 51 | 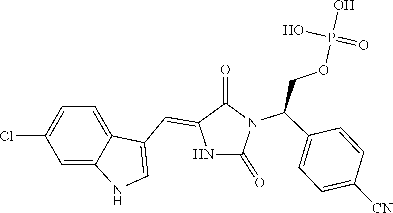 [SMILES: N#Cc1ccc([C@H](COP(=O)(O)O)N2C(=O)N/C(=C\c3c[nH]c4cc(Cl)ccc34)C2=O)cc1] |
| 52 | 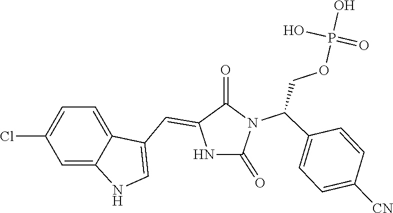 [SMILES: N#Cc1ccc([C@@H](COP(=O)(O)O)N2C(=O)N/C(=C\c3c[nH]c4cc(Cl)ccc34)C2=O)cc1] |
| 53 | 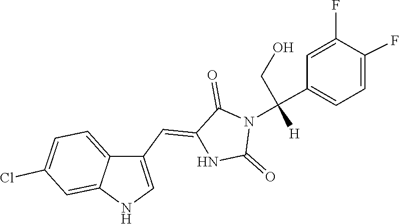 [SMILES: O=C1N/C(=C\c2c[nH]c3cc(Cl)ccc23)C(=O)N1[C@H](CO)c1ccc(F)c(F)c1] |
| 54 | 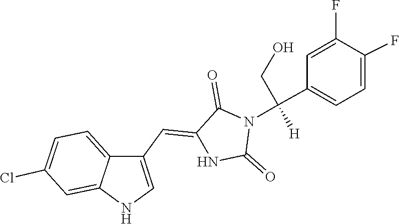 [SMILES: O=C1N/C(=C\c2c[nH]c3cc(Cl)ccc23)C(=O)N1[C@@H](CO)c1ccc(F)c(F)c1] |
| 55 | 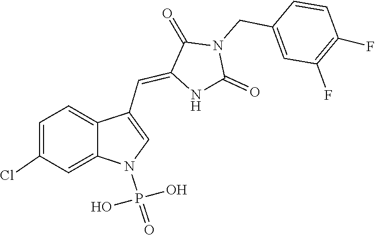 [SMILES: O=C1N/C(=C\c2cn(P(=O)(O)O)c3cc(Cl)ccc23)C(=O)N1Cc1ccc(F)c(F)c1] |
| 56 | 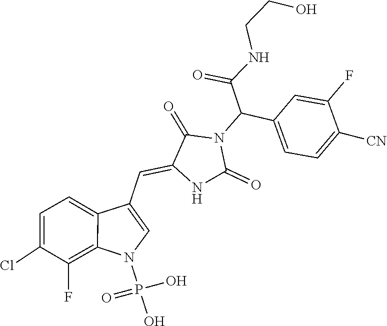 [SMILES: N#Cc1ccc(C(C(=O)NCCO)N2C(=O)N/C(=C\c3cn(P(=O)(O)O)c4c(F)c(Cl)ccc34)C2=O)cc1F] |
| 57 | 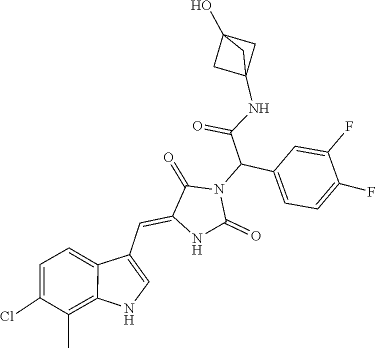 [SMILES: Cc1c(Cl)ccc2c(/C=C3\NC(=O)N(C(C(=O)NC45CC(O)(C4)C5)c4ccc(F)c(F)c4)C3=O)c[nH]c12] |
| 58 | 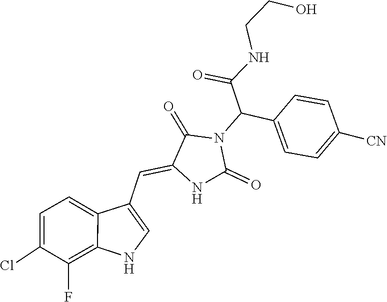 [SMILES: N#Cc1ccc(C(C(=O)NCCO)N2C(=O)N/C(=C\c3c[nH]c4c(F)c(Cl)ccc34)C2=O)cc1] |
| 59 | 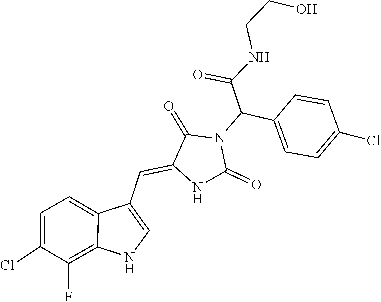 [SMILES: O=C(NCCO)C(c1ccc(Cl)cc1)N1C(=O)N/C(=C\c2c[nH]c3c(F)c(Cl)ccc23)C1=O] |
| 60 | 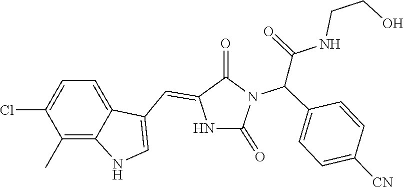 [SMILES: Cc1c(Cl)ccc2c(/C=C3\NC(=O)N(C(C(=O)NCCO)c4ccc(C#N)cc4)C3=O)c[nH]c12] |
| 61 | 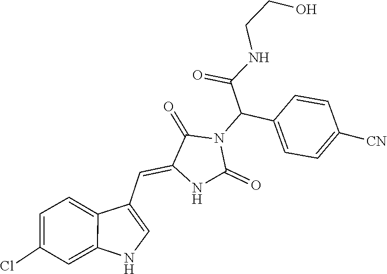 [SMILES: N#Cc1ccc(C(C(=O)NCCO)N2C(=O)N/C(=C\c3c[nH]c4cc(Cl)ccc34)C2=O)cc1] |
| 62 | 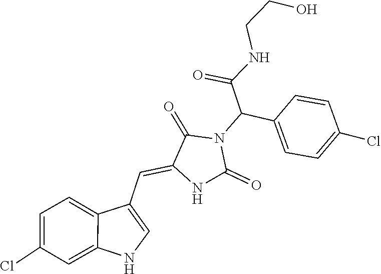 [SMILES: O=C(NCCO)C(c1ccc(Cl)cc1)N1C(=O)N/C(=C\c2c[nH]c3cc(Cl)ccc23)C1=O] |
| 63 | 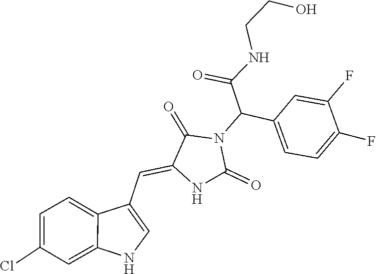 [SMILES: O=C(NCCO)C(c1ccc(F)c(F)c1)N1C(=O)N/C(=C\c2c[nH]c3cc(Cl)ccc23)C1=O] |
| 64 | 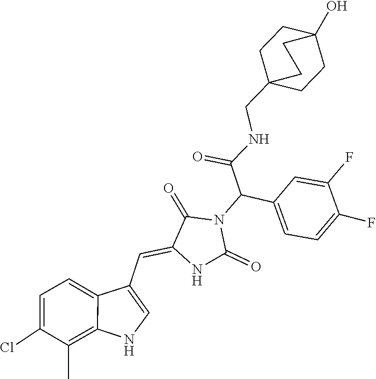 [SMILES: Cc1c(Cl)ccc2c(/C=C3\NC(=O)N(C(C(=O)NCC45CCC(O)(CC4)CC5)c4ccc(F)c(F)c4)C3=O)c[nH]c12] |
| 65 | 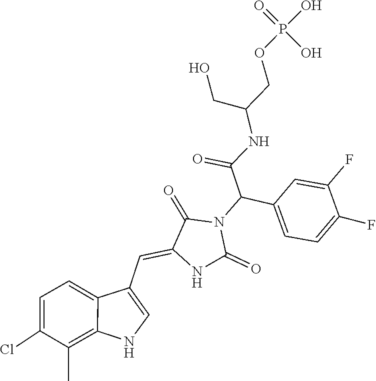 [SMILES: Cc1c(Cl)ccc2c(/C=C3\NC(=O)N(C(C(=O)NC(CO)COP(=O)(O)O)c4ccc(F)c(F)c4)C3=O)c[nH]c12] |
| 66 | 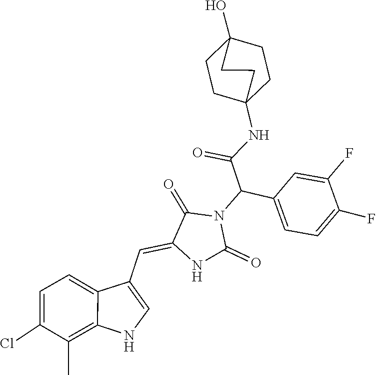 [SMILES: Cc1c(Cl)ccc2c(/C=C3\NC(=O)N(C(C(=O)NC45CCC(O)(CC4)CC5)c4ccc(F)c(F)c4)C3=O)c[nH]c12] |
| 67 | 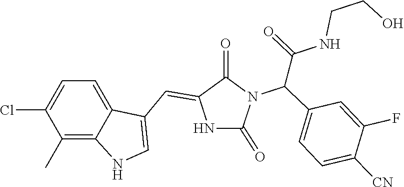 [SMILES: Cc1c(Cl)ccc2c(/C=C3\NC(=O)N(C(C(=O)NCCO)c4ccc(C#N)c(F)c4)C3=O)c[nH]c12] |
| 68 | 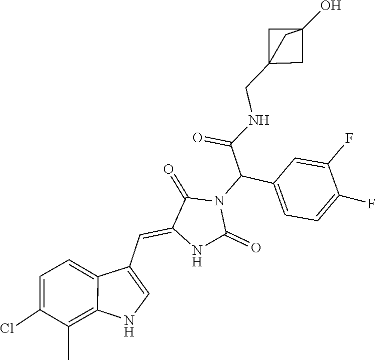 [SMILES: Cc1c(Cl)ccc2c(/C=C3\NC(=O)N(C(C(=O)NCC45CC(O)(C4)C5)c4ccc(F)c(F)c4)C3=O)c[nH]c12] |
| 69 | 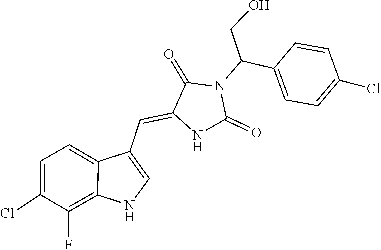 [SMILES: O=C1N/C(=C\c2c[nH]c3c(F)c(Cl)ccc23)C(=O)N1C(CO)c1ccc(Cl)cc1] |
| 70 | 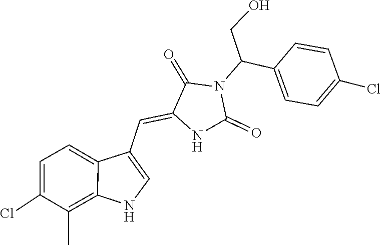 [SMILES: Cc1c(Cl)ccc2c(/C=C3\NC(=O)N(C(CO)c4ccc(Cl)cc4)C3=O)c[nH]c12] |
| 71 | 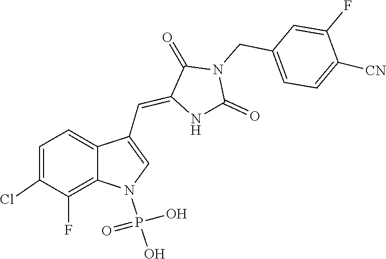 [SMILES: N#Cc1ccc(CN2C(=O)N/C(=C\c3cn(P(=O)(O)O)c4c(F)c(Cl)ccc34)C2=O)cc1F] |
| 72 | 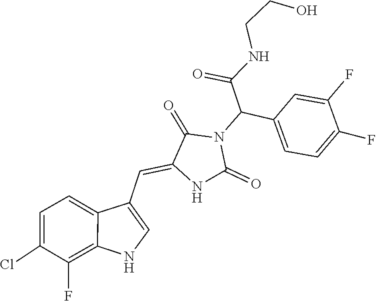 [SMILES: O=C(NCCO)C(c1ccc(F)c(F)c1)N1C(=O)N/C(=C\c2c[nH]c3c(F)c(Cl)ccc23)C1=O] |
| 73 | 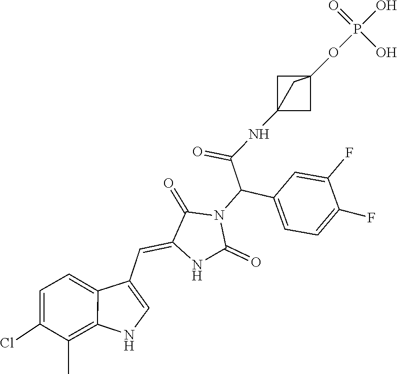 [SMILES: Cc1c(Cl)ccc2c(/C=C3\NC(=O)N(C(C(=O)NC45CC(OP(=O)(O)O)(C4)C5)c4ccc(F)c(F)c4)C3=O)c[nH]c12] |
| 74 | 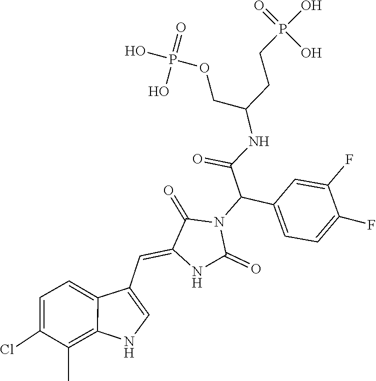 [SMILES: Cc1c(Cl)ccc2c(/C=C3\NC(=O)N(C(C(=O)NC(CCP(=O)(O)O)COP(=O)(O)O)c4ccc(F)c(F)c4)C3=O)c[nH]c12] |
| 75 | 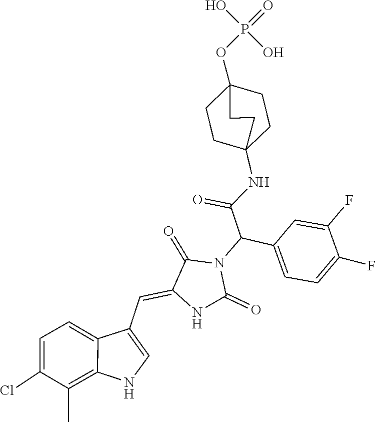 [SMILES: Cc1c(Cl)ccc2c(/C=C3\NC(=O)N(C(C(=O)NC45CCC(OP(=O)(O)O)(CC4)CC5)c4ccc(F)c(F)c4)C3=O)c[nH]c12] |
| 76 | 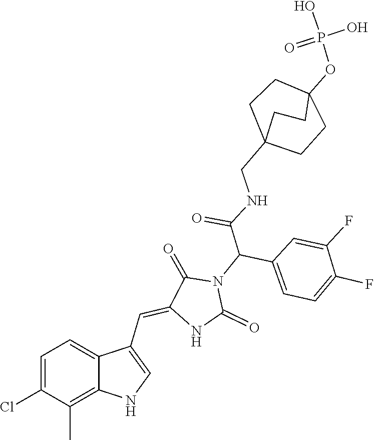 [SMILES: Cc1c(Cl)ccc2c(/C=C3\NC(=O)N(C(C(=O)NCC45CCC(OP(=O)(O)O)(CC4)CC5)c4ccc(F)c(F)c4)C3=O)c[nH]c12] |
| 77 | 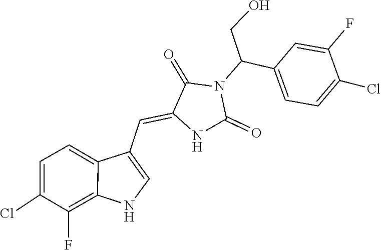 [SMILES: O=C1N/C(=C\c2c[nH]c3c(F)c(Cl)ccc23)C(=O)N1C(CO)c1ccc(Cl)c(F)c1] |
| 78 | 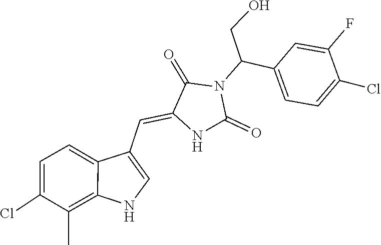 [SMILES: Cc1c(Cl)ccc2c(/C=C3\NC(=O)N(C(CO)c4ccc(Cl)c(F)c4)C3=O)c[nH]c12] |
| 79 | 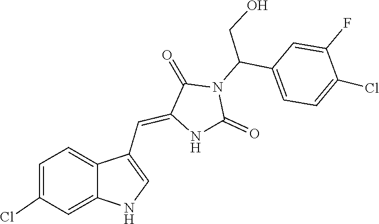 [SMILES: O=C1N/C(=C\c2c[nH]c3cc(Cl)ccc23)C(=O)N1C(CO)c1ccc(Cl)c(F)c1] |
| 80 | 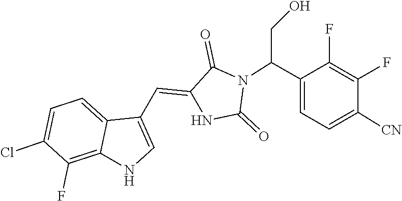 [SMILES: N#Cc1ccc(C(CO)N2C(=O)N/C(=C\c3c[nH]c4c(F)c(Cl)ccc34)C2=O)c(F)c1F] |
| 81 | 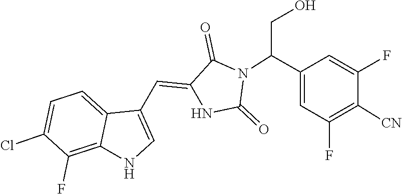 [SMILES: N#Cc1c(F)cc(C(CO)N2C(=O)N/C(=C\c3c[nH]c4c(F)c(Cl)ccc34)C2=O)cc1F] |
| 82 | 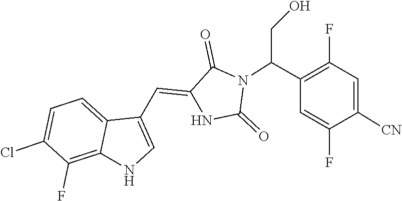 [SMILES: N#Cc1cc(F)c(C(CO)N2C(=O)N/C(=C\c3c[nH]c4c(F)c(Cl)ccc34)C2=O)cc1F] |
| 83 | 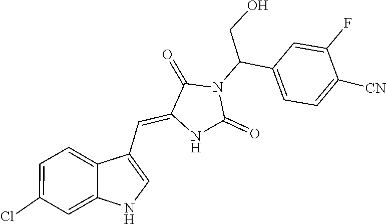 [SMILES: N#Cc1ccc(C(CO)N2C(=O)N/C(=C\c3c[nH]c4cc(Cl)ccc34)C2=O)cc1F] |
| 84 | 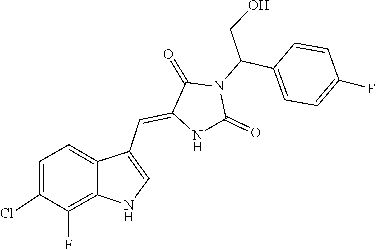 [SMILES: O=C1N/C(=C\c2c[nH]c3c(F)c(Cl)ccc23)C(=O)N1C(CO)c1ccc(F)cc1] |
| 85 | 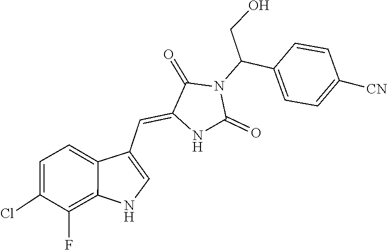 [SMILES: N#Cc1ccc(C(CO)N2C(=O)N/C(=C\c3c[nH]c4c(F)c(Cl)ccc34)C2=O)cc1] |
| 86 | 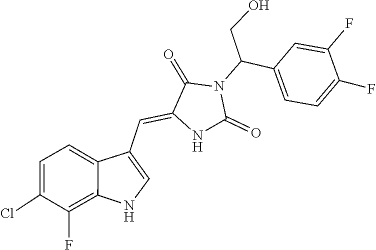 [SMILES: O=C1N/C(=C\c2c[nH]c3c(F)c(Cl)ccc23)C(=O)N1C(CO)c1ccc(F)c(F)c1] |
| 87 | 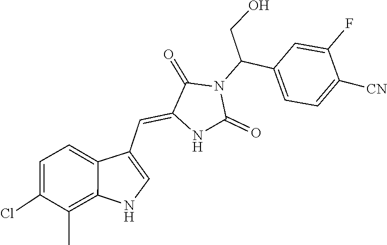 [SMILES: Cc1c(Cl)ccc2c(/C=C3\NC(=O)N(C(CO)c4ccc(C#N)c(F)c4)C3=O)c[nH]c12] |
| 88 | 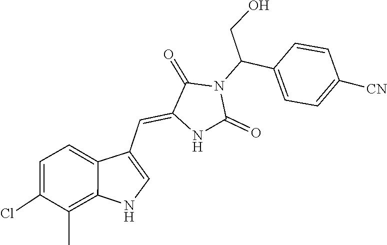 [SMILES: Cc1c(Cl)ccc2c(/C=C3\NC(=O)N(C(CO)c4ccc(C#N)cc4)C3=O)c[nH]c12] |
| 89 | 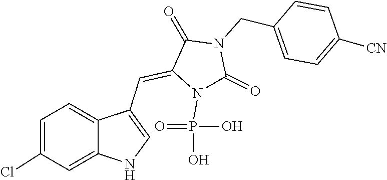 [SMILES: N#Cc1ccc(CN2C(=O)/C(=C/c3c[nH]c4cc(Cl)ccc34)N(P(=O)(O)O)C2=O)cc1] |
| 90 | 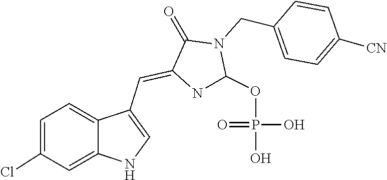 [SMILES: N#Cc1ccc(CN2C(=O)/C(=C/c3c[nH]c4cc(Cl)ccc34)NC2OP(=O)(O)O)cc1] |
| 91 | 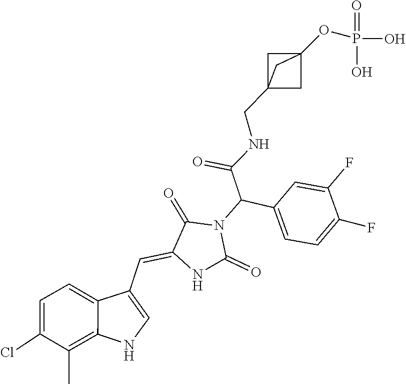 [SMILES: Cc1c(Cl)ccc2c(/C=C3\NC(=O)N(C(C(=O)NCC45CC(OP(=O)(O)O)(C4)C5)c4ccc(F)c(F)c4)C3=O)c[nH]c12] |
| 92 | 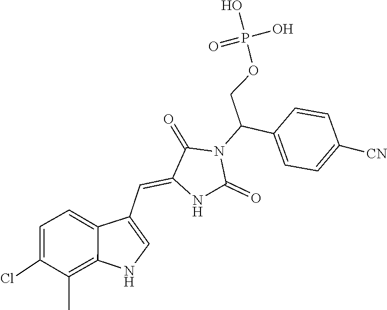 [SMILES: Cc1c(Cl)ccc2c(/C=C3\NC(=O)N(C(COP(=O)(O)O)c4ccc(C#N)cc4)C3=O)c[nH]c12] |
| 93 | 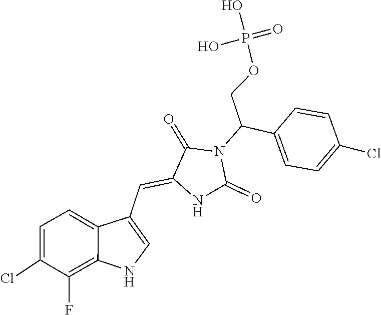 [SMILES: O=C1N/C(=C\c2c[nH]c3c(F)c(Cl)ccc23)C(=O)N1C(COP(=O)(O)O)c1ccc(Cl)cc1] |
| 94 | 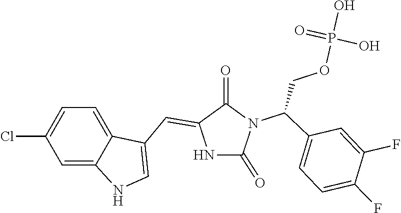 [SMILES: O=C1N/C(=C\c2c[nH]c3cc(Cl)ccc23)C(=O)N1[C@H](COP(=O)(O)O)c1ccc(F)c(F)c1] |
| 95 | 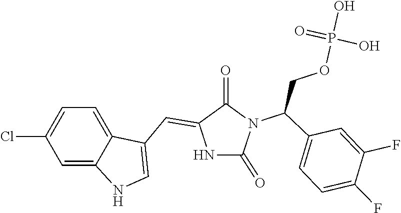 [SMILES: O=C1N/C(=C\c2c[nH]c3cc(Cl)ccc23)C(=O)N1[C@@H](COP(=O)(O)O)c1ccc(F)c(F)c1] |
| 96 | 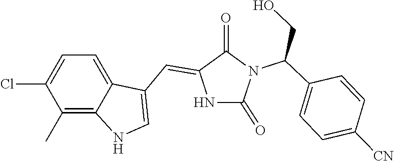 [SMILES: Cc1c(Cl)ccc2c(/C=C3\NC(=O)N([C@@H](CO)c4ccc(C#N)cc4)C3=O)c[nH]c12] |
| 97 | 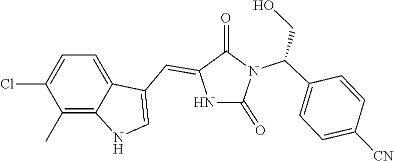 [SMILES: Cc1c(Cl)ccc2c(/C=C3\NC(=O)N([C@H](CO)c4ccc(C#N)cc4)C3=O)c[nH]c12] |
| 98 | 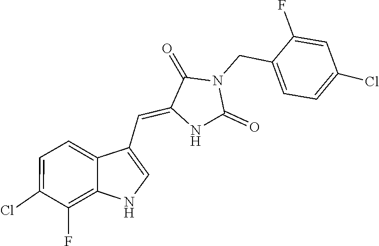 [SMILES: O=C1N/C(=C\c2c[nH]c3c(F)c(Cl)ccc23)C(=O)N1Cc1ccc(Cl)cc1F] |
| 99 | 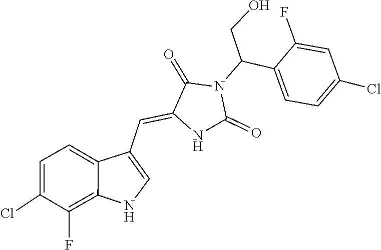 [SMILES: O=C1N/C(=C\c2c[nH]c3c(F)c(Cl)ccc23)C(=O)N1C(CO)c1ccc(Cl)cc1F] |
| 100 | 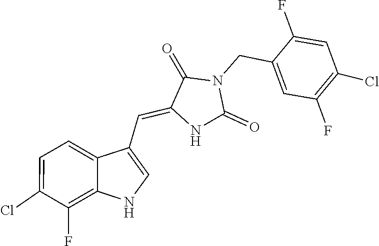 [SMILES: O=C1N/C(=C\c2c[nH]c3c(F)c(Cl)ccc23)C(=O)N1Cc1cc(F)c(Cl)cc1F] |
| 101 | 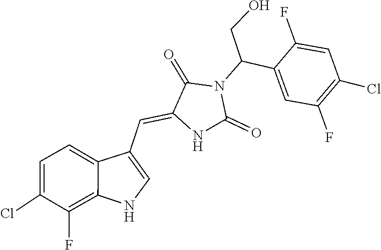 [SMILES: O=C1N/C(=C\c2c[nH]c3c(F)c(Cl)ccc23)C(=O)N1C(CO)c1cc(F)c(Cl)cc1F] |
| 102 | 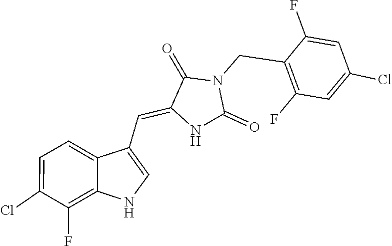 [SMILES: O=C1N/C(=C\c2c[nH]c3c(F)c(Cl)ccc23)C(=O)N1Cc1c(F)cc(Cl)cc1F] |
| 103 | 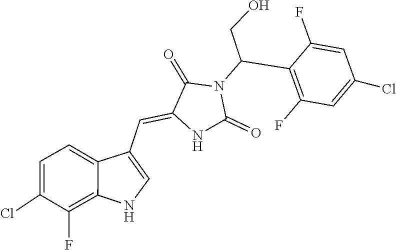 [SMILES: O=C1N/C(=C\c2c[nH]c3c(F)c(Cl)ccc23)C(=O)N1C(CO)c1c(F)cc(Cl)cc1F] |
| 104 | 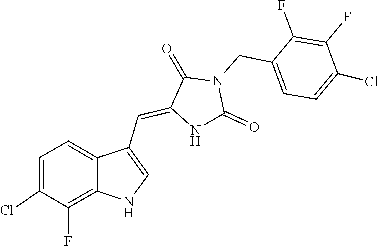 [SMILES: O=C1N/C(=C\c2c[nH]c3c(F)c(Cl)ccc23)C(=O)N1Cc1ccc(Cl)c(F)c1F] |
| 105 | 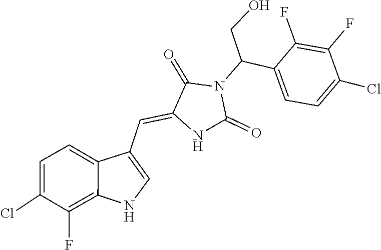 [SMILES: O=C1N/C(=C\c2c[nH]c3c(F)c(Cl)ccc23)C(=O)N1C(CO)c1ccc(Cl)c(F)c1F] |
| 106 | 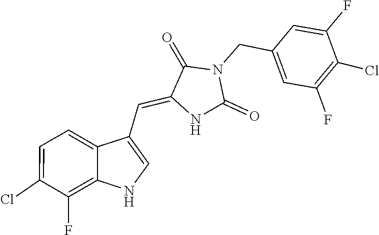 [SMILES: O=C1N/C(=C\c2c[nH]c3c(F)c(Cl)ccc23)C(=O)N1Cc1cc(F)c(Cl)c(F)c1] |
| 107 | 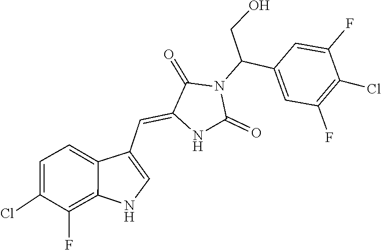 [SMILES: O=C1N/C(=C\c2c[nH]c3c(F)c(Cl)ccc23)C(=O)N1C(CO)c1cc(F)c(Cl)c(F)c1] |
| 108 | 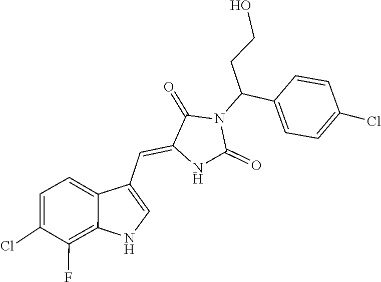 [SMILES: O=C1N/C(=C\c2c[nH]c3c(F)c(Cl)ccc23)C(=O)N1C(CCO)c1ccc(Cl)cc1] |
| 109 | 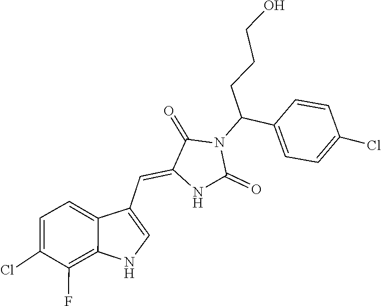 [SMILES: O=C1N/C(=C\c2c[nH]c3c(F)c(Cl)ccc23)C(=O)N1C(CCCO)c1ccc(Cl)cc1] |
| 110 | 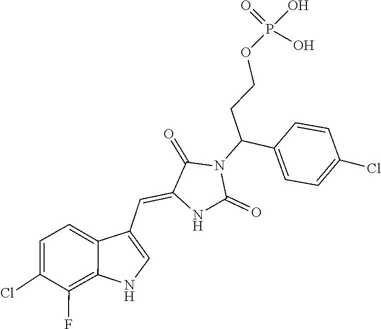 [SMILES: O=C1N/C(=C\c2c[nH]c3c(F)c(Cl)ccc23)C(=O)N1C(CCOP(=O)(O)O)c1ccc(Cl)cc1] |

The disclosure further provides a method for preparing the compound shown in formula (I), including a method I and/or a method II:
method 1: performing a reaction on a compound a and a compound b to obtain the compound shown in formula (I);

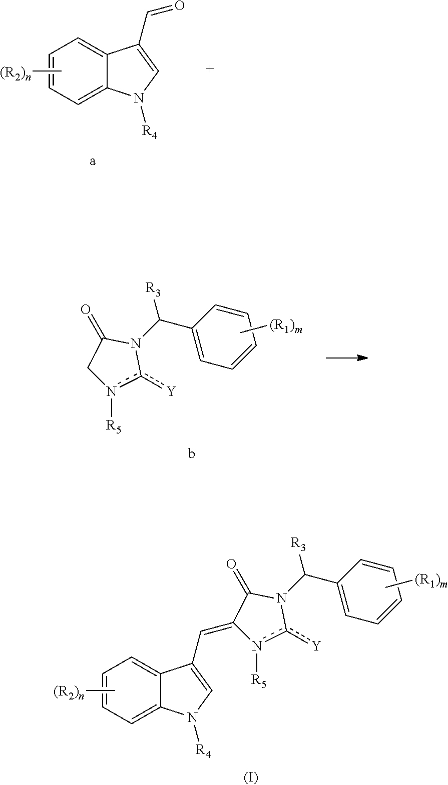

where R₁, R₂, R₃, R₄, R₅, Y, m, and n independently have definitions described above;
method II: performing a reaction on a compound c and R_{3b}—NH₂ to obtain the compound shown in formula (I);

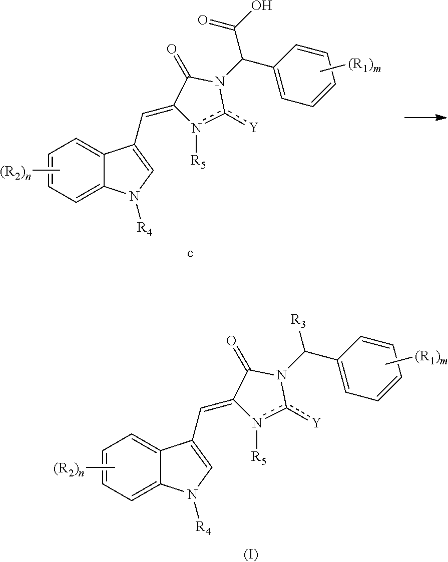

where R₁, R₂, R₃, R₄, R₅, R_{3b}, Y, m, and n independently have definitions described above.

The disclosure further provides a pharmaceutical composition, including a therapeutically effective amount of at least one of the compound shown in formula (I), and the raceme, the stereoisomer, the tautomer, the isotopic label, the solvate, the polymorph, the pharmaceutically acceptable salt or the prodrug compound thereof.

According to an embodiment of the disclosure, the pharmaceutical composition further includes one or more pharmaceutically acceptable accessories.

According to an embodiment of the disclosure, the pharmaceutical composition may further include one or more extra therapeutic agents.

The disclosure further provides a method for treating a neoplastic disease and/or idiopathic pulmonary fibrosis, including administrating to a patient a prophylactically or therapeutically effective amount of at least one of the compound shown in formula (I), and the raceme, the stereoisomer, the tautomer, the isotopic label, the solvate, the polymorph, the pharmaceutically acceptable salt or the prodrug compound thereof.

The disclosure further provides a method for treating a neoplastic disease and/or idiopathic pulmonary fibrosis, including administrating to a patient a prophylactically or therapeutically effective amount of the pharmaceutical composition.

According to an embodiment of the disclosure, the neoplastic disease may be a neoplastic disease induced by overexpression of MDM2 and MDMX.

According to an embodiment of the disclosure, the neoplastic disease includes at least one of leukemia, chronic myeloid leukemia, acute myelocytic leukemia, lymphocytic leukemia, hairy cell leukemia, T lymphoblastic leukemia, lymphoma, B-cell lymphoma, burkitt lymphoma, hodgkin lymphoma, chondrosarcoma, fibrosarcoma, ewing sarcoma, rhabdomyosarcoma, small cell lung cancer, non-small cell lung cancer, myeloma, glioma, prostatic cancer, breast cancer, bone cancer, neuroblastoma, gastric cancer, ovarian cancer, intestinal cancer, renal cancer, medulloblastoma, pancreatic cancer, thyroid cancer, mesothelioma, cervical cancer, bladder cancer, liver cancer, skin cancer, and tumors in the central system or the peripheral nervous system.

In some embodiments, the patient includes a mammal, preferably, a person.

The disclosure further provides at least one of a compound shown in formula (I), and a raceme, a stereoisomer, a tautomer, an isotopic label, a solvate, a polymorph, a pharmaceutically acceptable salt or a prodrug compound thereof, or a pharmaceutical composition thereof for treating a neoplastic disease and/or idiopathic pulmonary fibrosis.

The disclosure further provides use of at least one of a compound shown in formula (I), and a raceme, a stereoisomer, a tautomer, an isotopic label, a solvate, a polymorph, a pharmaceutically acceptable salt or a prodrug compound thereof in preparation of a drug.

According to an embodiment of the disclosure, the use may be use in preparation of a drug for treating an MDM2 and/or MDMX-mediated symptom and/or disease, for example, use in preparation of an MDM2 and/or MDMX inhibitor drug.

According to an embodiment of the disclosure, the use may be use in preparation of a drug for treating idiopathic pulmonary fibrosis.

According to an embodiment of the disclosure, the disease is, for example, a neoplastic disease. The neoplastic disease includes at least one of leukemia, chronic myeloid leukemia, acute myelocytic leukemia, lymphocytic leukemia, hairy cell leukemia, T lymphoblastic leukemia, lymphoma, B-cell lymphoma, burkitt lymphoma, hodgkin lymphoma, chondrosarcoma, fibrosarcoma, ewing sarcoma, rhabdomyosarcoma, small cell lung cancer, non-small cell lung cancer, myeloma, glioma, prostatic cancer, breast cancer, bone cancer, neuroblastoma, gastric cancer, ovarian cancer, intestinal cancer, renal cancer, medulloblastoma, pancreatic cancer, thyroid cancer, mesothelioma, cervical cancer, bladder cancer, liver cancer, skin cancer, and tumors in the central system or the peripheral nervous system.

### Beneficial Effects

The compound provided by the disclosure has a good MDM2 and/or MDMX inhibiting effect and may be used for treating MDM2 and/or MDMX-mediated symptoms and/or diseases such as neoplastic diseases and/or idiopathic pulmonary fibrosis and preparing drugs for such symptoms or diseases. In particular, a compound containing a phosphoric acid (phosphate) structure has higher solubility. As the prodrug, the compound can release an MDM2 and/or MDMX double-target inhibitor compound with high activity in an animal body. Moreover, the preferred compound provided by the disclosure also has preferred pk activity. It solves the problem that such double-target inhibitors are hardly prepared into drugs.

### Definitions and Descriptions of Terms

Unless otherwise specified, radical and term definitions recorded in the description and claims of this application include definitions thereof as examples, exemplary definitions, preferred definitions, definitions recorded in a table, definitions of specific compounds in the examples, and the like, which may be arbitrarily combined and incorporated with each other. Such combined and incorporated radial definitions and compound structures should be construed as falling with the scope recorded by the description and/or claims of this application.

Unless otherwise specified, the numerical ranges recorded in the description and claims at least equivalently record each specific integer value. For example, the numerical value “1-8” equivalently records each integer value in the numerical value “1-8”, i.e., 1, 2, 3, 4, 5, 6, 7 or 8.

The term “optional” (or “optionally”, “option”) in the definitions of the general formula in this application means that the radical is substituted with zero, one or more substituents. For example, “optionally substituted with one, two or more R” means that the radial may not be substituted with R (unsubstituted) or may be selectively substituted with one, two or more R.

“More” represents three or more than three.

The term “C₁₋₆ alkyl” should be construed as representing straight-chain alkyl and branched alkyl having 1, 2, 3, 4, 5 or 6 carbon atoms. The alkyl is, for example, methyl, ethyl, propyl, butyl, amyl, hexyl, isopropyl, isobutyl, sec-butyl, tertiary butyl, isoamyl, 2-methyl butyl, 1-methyl butyl, 1-ethyl propyl, 1,2-dimethyl propyl, neopentyl, 1,1-dimethyl propyl, 4-methyl amyl, 3-methyl amyl, 2-methyl amyl, 1-methyl amyl, 2-ethyl butyl, 1-ethyl butyl, 3,3-dimethyl butyl, 2,2-dimethyl butyl, 1,1-dimethyl butyl, 2,3-dimethyl ethyl, 1,3-dimethyl butyl or 1,2-dimethyl butyl, and the like or their isomers.

The term “C₂₋₆ alkenyl” should be construed as representing monovalent hydrocarbyl of a straight chain or a branched chain, including one or more double bonds and having 2-6 carbon atoms, for example, 2 or 3 carbon atoms (i.e., C₂₋₃ alkenyl). It should be understood that the alkenyl includes more than one double bond, and the double bonds may be separated from each other or may be conjugated. The alkenyl is, for example, vinyl, allyl, (E)-2-methyl vinyl, (Z)-2-methyl vinyl, (E)-2-butenyl, (Z)-2-butenyl, (E)-1-butenyl, (Z)-1-butenyl, 4-pentenyl, (E)-3-pentenyl, (Z)-3-pentenyl, (E)-2-pentenyl, (Z)-2-pentenyl, (E)-1-pentenyl, (Z)-1-pentenyl, 5-hexenyl, (E)-4-hexenyl, (Z)-4-hexenyl, (E)-3-hexenyl, (Z)-3-hexenyl, (E)-2-hexenyl, (Z)-2-hexenyl, (E)-1-hexenyl, (Z)-1-hexenyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, and 1-isopropylvinyl.

The term “C₂₋₆ alkynyl” should be construed as representing monovalent hydrocarbyl of a straight chain or a branched chain, including one or more triple bonds and having 2-6 carbon atoms, for example, 2 or 3 carbon atoms (i.e., C₂₋₃ alkynyl). The alkynyl is, for example, acetenyl, 1-propinyl, 2-propinyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl.

The term “C₃₋₈ cycloalkyl” should be construed as representing a saturated monovalent monocyclic, bicyclic hydrocarbon ring (for example, a condensed ring, a bridge ring, and a spiral ring) or tricyclic alkane, having 3-8 carbon atoms. The C₃₋₈ cycloalkyl may be monocyclic hydrocarbyl such as cyclopropyl, cyclobutyl, cyclopentyl, cyclohexyl, cycloheptyl or cyclooctyl, or may be bridged hydrocarbyl such as bicyclo[2.1.1]hexyl, bicyclo[2.2.1]heptyl, bicyclo[2.2.1]heptenyl, 6,6-dimethyl-bicyclo[3.1.1]heptyl, 2,6,6-trimethyl bicycle[3.1.1]heptyl, bicycle[2.2.2]octyl, 2,7-diazaspiro[3,5]nonyl, and 2,6-diazaspiro[3,4]octyl.

Persons skilled in the art may understand that the compound shown in formula (I) may exist in the form of various pharmaceutically acceptable salts. If these compounds have alkaline centers, they may form acid addition salts; if these compounds have acidic centers, they may form alkali addition salts; and if these compounds not only include the acidic centers (for example, carboxyl) but also include the alkaline centers (for example, amino), they also may form inner salts.

The compound provided by the disclosure may exist in the form of the solvate (for example, a hydrate). The compound provided by the disclosure includes a polar solvent as a structural element of the compound lattices, particularly, for example, water, methanol or ethanol. The amount of the polar solvent, particularly, water may exist in the form of a stoichiometric ratio or a non-stoichiometric ratio.

According to its molecular structure, the compound provided in the disclosure may be chiral. Therefore, various enantiomer forms may exist. Therefore, these compounds may exist in the form of racemes or in an optical activity form. The compound provided by the disclosure covers various isomers with chiral carbon in an R or S configuration, a mixture thereof, and racemes. The compound provided by the disclosure or an intermediate thereof may be separated as enantiomer compounds by chemical or physical methods known to persons skilled in the art or may be used for synthesis in this form. In a case of amine of the raceme, a diastereoisomers is prepared from the mixture through a reaction with an optically active splitting reagent. An example of a proper splitting reagent is an optically active acid, for example, tartaric acid, diacetyl tartaric acid, dibenzoyltartaric acid, mandelic acid, malic acid, and lactic acid in R and S forms, proper N-protected amino acids (for example, N-benzoyl proline or N-phenyl sulfonyl proline) or various optically active camphorsulfonic acids. Chromatographic enantiomers may also be favorably split by virtue of the optically active splitting reagents (for example, dinitrobenzoylphenyl glycine, cellulose triacetate or derivatives of other carbohydrates or chirally derivatized methacrylic ester polymers fixed on a silica gel). A proper eluent for this purpose is a water or alcohol-containing solvent mixture, for example, hexane/isopropanol/acetonitrile.

Corresponding stable isomers may be separated according to known methods such as extraction, filtering or column chromatography.

The term “patient” refers to any animal including mammals, preferably, a mouse, a rat, another rodent, a rabbit, a dog, a cat, a pig, a cow, a goat, a horse or a primate, and most preferably a person.

The term “therapeutically effective amount” refers to a dose of an active compound or a drug inducing a biological or medical response sought by researchers, veterinarians, physicians or other clinical physicians in tissues, systems, animals, individuals or persons, including the following one or more: (1) preventing diseases: for example, preventing diseases, disorders or symptoms in individuals susceptible to diseases, disorders or symptoms without experiencing or having disease pathology or symptoms; (2) suppressing diseases: for example, suppressing diseases, disorders or symptoms (i.e., preventing further development of pathology and/or symptoms) in individuals experiencing or having pathology and/or symptoms of diseases, disorders or symptoms; and (3) relieving diseases: for example, relieving diseases, disorders or symptoms (i.e., reversing pathology and/or symptoms) in individuals experiencing or having pathology and/or symptoms of diseases, disorders or symptoms.

### DETAILED DESCRIPTION

The technical solutions of the disclosure will be further described in detail below in conjunction with specific examples. It should be understood that the examples below merely describe and explain the disclosure exemplarily and should not be explained as a limitation to the protection scope of the disclosure. Technologies realized based on the above content of the disclosure should fall within the scope to be protected by the disclosure.

Unless otherwise specified, raw materials and reagents used in the examples below all are marketed commodities or may be prepared by known methods.

### INTERMEDIATES

### Intermediate I-1: Synthesis of 3-(3,4-difluorobenzyl)imidazolidine-2,4-dione

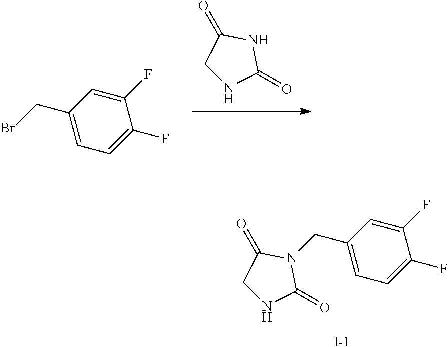 [SMILES: Fc1ccc(CBr)cc1F ; O=C1CNC(=O)N1 ; O=C1CNC(=O)N1Cc1ccc(F)c(F)c1 ; [I-]]

At room temperature, 4-(bromomethyl)-1,2-difluorobenzene (2 g, 9.178 mmol) and potassium carbonate (3.17 g, 22.945 mmol) are dissolved in N,N-dimethylformamide (20 mL). Hydantoin (1.35 g, 12.849 mmol) is added in batches into a mixed solution while the mixed solution is stirred. After being stirred at room temperature for 16 h, the mixture is poured into water (30 mL) and extracted with ethyl acetate (30 mL) three times, and organic phases are combined and washed with saturated saline. After being dried with anhydrous sodium sulfate, the organic phases are filtered, a filtrate is concentrated in vacuum to obtain a white solid crude product intermediate I-1 (1 g, 41.72%), and the crude product is not further purified and is directly used for a next reaction.

LC-MS m/z (ESI): 227.15 [M+H]⁺.

### Intermediate I-2: Synthesis of 3-(4-chlorobenzyl)imidazolidine-2,4-dione

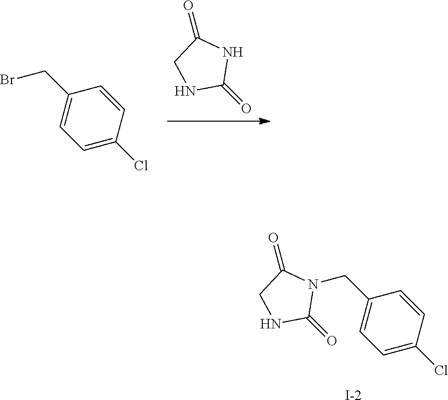

By using the method for the intermediate I-1, the white solid crude product intermediate I-2 (500 mg, 92.78%) is prepared by using 1-(bromomethyl)-4-chlorobenzene and hydantoin, and the crude product is not further purified and is directly used for a next reaction.

### Intermediate I-3: Synthesis of 4-((2,5-dioxoimidazolidin-1-yl)methyl)benzonitrile

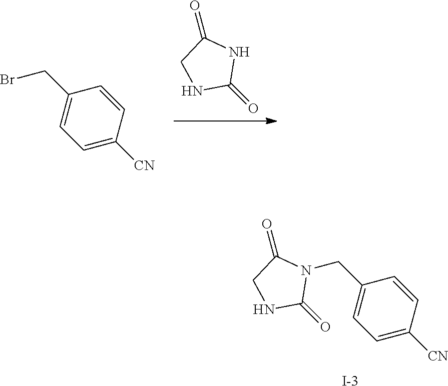

By using the method for the intermediate I-1, the white solid crude product intermediate I-3 (2.26 g, 84%) is prepared by using 4-(bromomethyl)benzonitrile and hydantoin, and the crude product is not further purified and is directly used for a next reaction.

### Intermediate I-4: Synthesis of 4-((2,5-dioxoimidazolidin-1-yl)methyl)-2-fluorobenzonitrile

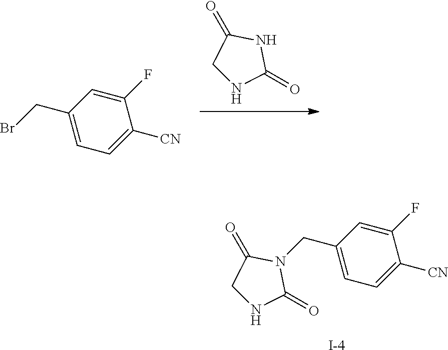

By using the method for the intermediate I-1, the white solid crude product intermediate I-4 (500 mg, 81%) is prepared by using 1-(bromomethyl)-4-chlorobenzene and hydantoin, and the crude product is not further purified and is directly used for a next reaction.

LC-MS m/z (ESI): 232.00 [M−H]⁻.

### Intermediate I-5: Synthesis of 6-chloro-7-methoxyl-1H-indole-3-formaldehyde

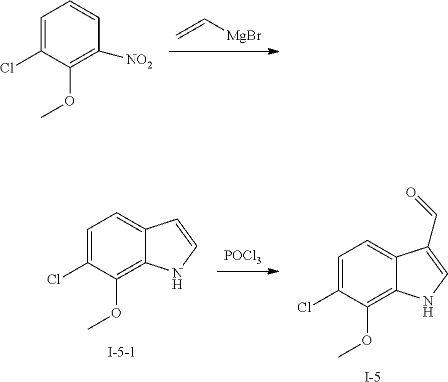

### Intermediate I-5-1: 6-chloro-7-methoxyl-1H-indole

At −20° C., a bromovinyl magnesium bromide-tetrahydrofuran (1N, 160 mL) solution is slowly dropwise added into a solution of 1-chloro-2-methoxyl-3-nitrobenzene (10 g, 53.31 mmol) in tetrahydrofuran (200 mL). After being stirred at room temperature for 3 h, the mixture is poured into ice water (200 ml) and extracted with ethyl acetate (500 ml) twice. Organic phases are combined, washed with saturated saline, dried with anhydrous sodium sulfate, and concentrated in vacuum. The mixture is purified by flash chromatography to obtain the white solid I-5-1 (2.1 g, 19.52%).

LC-MS m/z (ESI): 182.1 [M+H]⁺.

¹H NMR (400 MHz, CDCl₃) δ 8.39 (s, 1H), 7.30 (dd, J=8.4, 0.5 Hz, 1H), 7.19 (dd, J=3.1, 2.5 Hz, 1H), 7.07 (d, J=8.4 Hz, 1H), 6.53 (dd, J=3.1, 2.2 Hz, 1H), 4.02 (s, 3H).

### I-5: 6-chloro-7-methoxyl-1H-indole-3-formaldehyde

At 0° C., phosphorus oxychloride (2.12 g, 13.87 mmol) is added into dimethylformamide (20 ml). The mixture is stirred at 0° C. for 10 min. Then, a solution of 6-chloro-7-methoxyl-1H-indole (2.1 g, 11.56 mmol) in dichloromethane (5 mL) is added. A mixed solution is stirred at room temperature for 1 h, sodium hydroxide (1N, 30 ml) is added into the mixed solution, and the mixed solution is stirred for 30 min. The mixed solution is extracted with ethyl acetate (30 ml) three times. Organic phases are combined, washed with anhydrous sodium sulfate, and concentrated in vacuum. The mixture is purified by flash chromatography to obtain the white solid I-5 (0.9 g, 37.1%).

LC-MS m/z (ESI): 210.1 [M+H]⁺. ¹H NMR (400 MHz, DMSO-d6) δ 12.57 (s, 1H), 9.94 (s, 1H), 8.35 (s, 1H), 7.82 (d, J=8.4 Hz, 1H), 7.26 (d, J=8.4 Hz, 1H), 3.94 (s, 3H).

### Intermediate I-6: Synthesis of 3-[(4-acetenyl phenyl)methyl]imidazolidine-2,4-dione

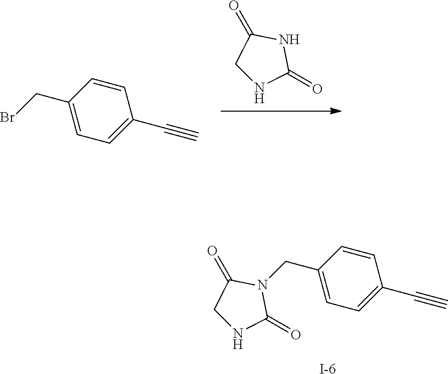

By using the method for the intermediate I-1, the white solid intermediate I-6 (122 mg, 26%) is prepared by using 1-(bromomethyl)-4-acetylenylbenzene and hydantoin.

LC-MS m/z (ESI): 215.1 [M+H]⁺.

### Intermediate I-7: Synthesis of 3-[4-(8-amino octyl)-1-oxo-3H-isoindol-2-yl]piperidine-2,6-dione

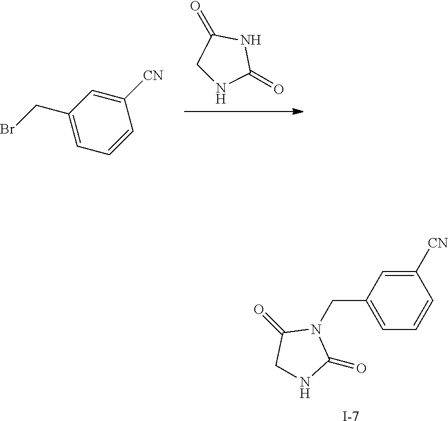

By using the method for the intermediate I-1, the brown solid intermediate I-7 (129 mg, 29%) is prepared by using 3-(bromomethyl)benzonitrile and hydantoin.

LC-MS m/z (ESI): 214.0 [M−H]⁻.

### Intermediate I-8: Synthesis of 6-chloro-7-methyl-1H-indole-3-formaldehyde

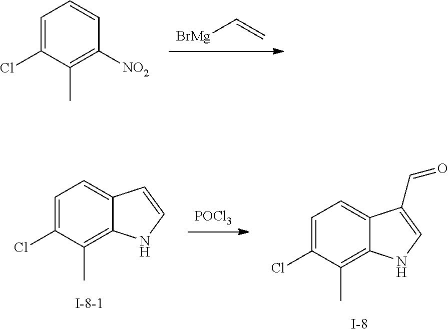

### I-8-1: 6-chloro-7-methyl-1H-indole

Under nitrogen protection, 1-chloro-2-methyl-3-nitrobenzene (2.0 g, 11.656 mmol) is dissolved in a tetrahydrofuran (20 ml) solution, the temperature of the solution is decreased to −78° C., vinyl magnesium bromide (1N, 46.6 ml, 46.626 mmol) is slowly dropwise added, and the mixture is continuously stirred for 2 h. A saturated ammonium chloride (100 ml) solution is slowly added into the mixture and the temperature of the system is maintained not higher than −60° C. A reaction is performed to raise the temperature to room temperature, the mixture is extracted with ethyl acetate (100 ml) three times, and combined organic phases are washed with saturated saline (100 ml) and dried with anhydrous sodium sulfate. After the organic phases are filtered, a filtrate is concentrated in vacuum. A residue is purified by silica gel column chromatography and eluted with petroleum ether/ethyl acetate (5:1) to obtain the brown oily I-8-1 (1.08 g, 56%).

LC-MS m/z (ESI): 166.2 [M+H]⁺.

### I-8: 6-chloro-7-methyl-1H-indole-3-formaldehyde

At 0° C., phosphorus oxychloride (2.4 g, 16.094 mol) is dropwise added into a dimethylformamide (2.5 ml) solution. The obtained mixed solution is continuously stirred under nitrogen protection for 30 min and maintained at 0° C. A solution of 6-chloro-7-methyl-1H-indole (540 mg, 3.260 mmol) in dimethylformamide (2.5 ml) is added into the solution. After being stirred at 40° C. for 1 h, the mixture is cooled to room temperature and water (20 ml) is added to quench the reaction. The pH value of the system is adjusted to 8 with a sodium hydroxide (30%) solution. The obtained mixture is extracted with ethyl acetate (50 ml) three times, and combined organic phases are washed with saturated saline (50 ml) and dried with anhydrous sodium sulfate. After the organic phases are filtered, a filtrate is concentrated in vacuum. A residue is purified by silica gel column chromatography and eluted with petroleum ether/ethyl acetate (1:1) to obtain the brown oily I-8 (623 mg, 96%).

LC-MS m/z (ESI): 194.1 [M+H]⁺.

### Intermediate I-9: Synthesis of 6-chloro-7-(methyl sulfonyl)-1H-indole-3-formaldehyde

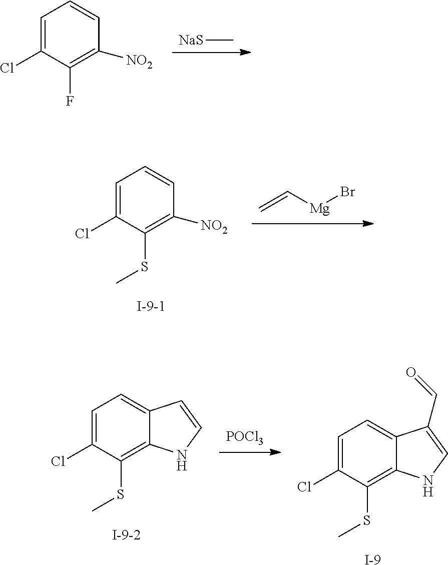

### I-9-1: 1-chloro-2-methylthio-3-nitrobenzene

Under nitrogen protection at room temperature, 1-chloro-2-fluoro-3-nitrobenzene (2 g, 10.824 mmol) is dissolved in a DMF (40 mL) solution, sodium thiomethoxide (0.88 g, 11.906 mmol) is added into the solution, and the mixed solution is maintained stirred. The mixture is heated to 120° C. and stirred for 12 h, and after the reaction is completed, the mixture is cooled to room temperature. The obtained mixed solution is concentrated in vacuum. A residue is purified by silica gel column chromatography and eluted with petroleum ether/ethyl acetate (5/95) to obtain the reddish brown oily I-9-1 (770 mg, 29%).

¹H NMR (300 MHz, CDCl₃) δ 7.68-7.60 (m, 1H), 7.55-7.47 (m, 1H), 7.41-7.38 (m, 1H), 2.46 (s, 3H).

### I-9-2: 6-chloro-7-(methyl sulfonyl)-1H-indole

Under nitrogen protection, 1-chloro-2-methylthio-3-nitrobenzene (230 mg, 0.932 mol) is dissolved in a tetrahydrofuran (5 ml) solution, the temperature of the solution is decreased to −78° C., vinyl magnesium bromide (1N, 3.7 ml, 3.7 mmol) is slowly dropwise added, and the mixture is continuously stirred for 3 h. A saturated ammonium chloride (50 ml) solution is slowly added into the mixture and the temperature of the system is maintained not higher than −60° C. A reaction is performed to raise the temperature to room temperature, the mixture is extracted with ethyl acetate (50 ml) twice, and combined organic phases are washed with saturated saline (50 ml) and dried with anhydrous sodium sulfate. After the organic phases are filtered, a filtrate is concentrated in vacuum. A residue is purified by preparative high performance liquid chromatography to obtain the brown oily I-9-2 (180 mg, 81%).

LC-MS m/z (ESI): 198.1 [M+H]⁺.

### I-9: 6-chloro-7-(methylsulfonyl)-1H-indole-3-formaldehyde

At 0° C., phosphorus oxychloride (0.178 g, 1.115 mol) is dropwise added into a dimethylformamide (1.5 ml) solution. The obtained mixed solution is continuously stirred under nitrogen protection for 30 min and maintained at 0° C. A solution of 6-chloro-7-(methylsulfonyl)-1H-indole (180 mg, 0.76 mmol) in dimethylformamide (1.5 ml) is added into the solution. After being stirred at 40° C. for 1 h, the mixture is cooled to room temperature and water (5 ml) is added to quench the reaction. The pH value of the system is adjusted to 8 with a sodium hydroxide (30%) solution. The obtained mixture is extracted with ethyl acetate (20 ml) three times, and combined organic phases are washed with saturated saline (30 ml) and dried with anhydrous sodium sulfate. After the organic phases are filtered, a filtrate is concentrated in vacuum to obtain the light yellow solid I-9 (160 mg, 89%). A crude product is directly left for a next step without further purification.

LC-MS m/z (ESI): 226.0 [M+H]⁺.

### Intermediate I-10: Synthesis of 6-chloro-7-fluoro-1H-indole-3-formaldehyde

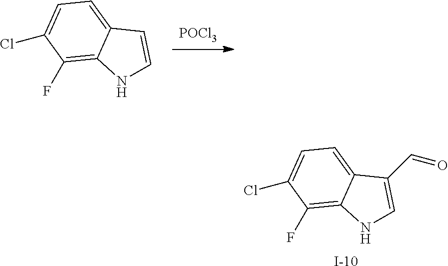

At 0° C., phosphorus oxychloride (0.18 ml, 1.117 mol) is dropwise added into a dimethylformamide (1.5 ml) solution. The obtained mixed solution is continuously stirred under nitrogen protection for 30 min and maintained at 0° C. A solution of 6-chloro-7-fluoro-1H-indole (75 mg, 0.44 mmol) in dimethylformamide (1.5 ml) is added into the solution. After being stirred at 40° C. for 1 h, the mixture is cooled to room temperature and water (5 ml) is added to quench the reaction. The pH value of the system is adjusted to 8 with a sodium hydroxide (30%) solution. The obtained mixture is extracted with ethyl acetate (50 ml) three times, and combined organic phases are washed with saturated saline (30 ml) and dried with anhydrous sodium sulfate. After the organic phases are filtered, a filtrate is concentrated in vacuum to obtain the white-like solid I-10 (75 mg, 44.07%). A crude product is directly left for a next step without further purification.

### Intermediate I-11: Synthesis of 6-chloro-7-(fluoromethoxyl)-1H-indole-3-formaldehyde

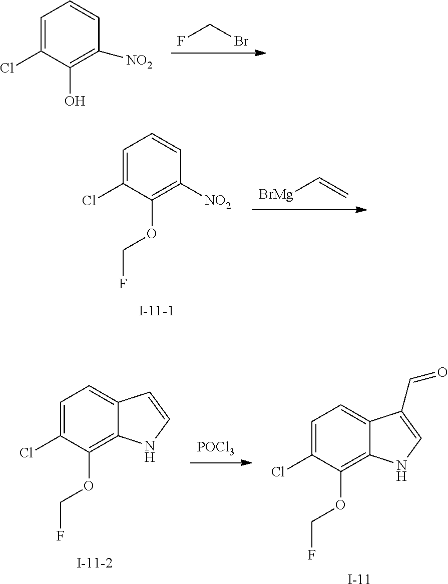

### I-11-1: 1-chloro-2-(fluoromethoxyl)-3-nitrobenzene

At room temperature, 6-chloro-2-nitrophenol (1 g, 5.474 mmol) and potassium carbonate (1.59 g, 10.929 mmol) are added into a dimethylformamide (15 ml) solution and are maintained stirred, and bromofluoromethane (6.51 g, 54.764 mmol) is added into the mixture. The mixture is stirred overnight under nitrogen protection at room temperature, and after the reaction is completed, the mixed solution is poured into water (50 ml). The obtained mixture is extracted with ethyl acetate (30 ml) three times and washed with saturated saline (20 ml), and organic phases are combined and dried with anhydrous sodium sulfate. After the organic phases are filtered, a filtrate is concentrated in vacuum. A residue is purified by silica gel column chromatography and eluted with petroleum ether/ethyl acetate (5:1) to obtain the yellow oily I-11-1 (430 mg, 38.14%).

¹H NMR (300 MHz, DMSO-d₆) δ 8.02-7.95 (m, 2H), 7.52 (d, J=8.1 Hz, 1H), 5.87 (s, 1H), 5.70 (s, 1H).

### I-11-2: 6-chloro-7-(fluoromethoxyl)-1H-indole

Under nitrogen protection, 1-chloro-2-(fluoromethoxyl)-3-nitrobenzene (590 mg, 2.864 mol) is dissolved in a tetrahydrofuran (8 ml) solution, the temperature of the solution is decreased to −78° C., vinyl magnesium bromide (1N, 8 ml, 8 mmol) is slowly dropwise added, and the mixture is continuously stirred for 3 h. A saturated ammonium chloride (50 ml) solution is slowly added into the mixture and the temperature of the system is maintained not higher than −60° C. A reaction is performed to raise the temperature to room temperature, the mixture is extracted with ethyl acetate (50 ml) twice, and combined organic phases are washed with saturated saline (50 ml) and dried with anhydrous sodium sulfate. After the organic phases are filtered, a filtrate is concentrated in vacuum. A residue is purified by preparative high performance liquid chromatography to obtain the gray oily I-11-2 (690 mg, 81%).

LC-MS m/z (ESI): 198.0 [M−H]⁻.

### I-11: 6-chloro-7-(fluoromethoxyl)-1H-indole-3-formaldehyde

At 0° C., phosphorus oxychloride (1.25 g, 7.848 mol) is dropwise added into a dimethylformamide (13 ml) solution. The obtained mixed solution is continuously stirred under nitrogen protection for 30 min and maintained at 0° C. A solution of 6-chloro-7-(fluoromethoxyl)-1H-indole (640 mg, 3.2 mmol) in dimethylformamide (13 ml) is added into the solution. After being stirred at 40° C. for 1 h, the mixture is cooled to room temperature and water (4 ml) is added to quench the reaction. The pH value of the system is adjusted to 8 with a sodium hydroxide (30%) solution. The obtained mixture is extracted with ethyl acetate (30 ml) three times, and combined organic phases are washed with saturated saline (30 ml) and dried with anhydrous sodium sulfate. After the organic phases are filtered, a filtrate is concentrated in vacuum to obtain the brown oily I-11 (380 mg, 51.56%). A crude product is directly left for a next step without further purification.

LC-MS m/z (ESI): 228.2 [M+H]⁺.

### Intermediate I-12: Synthesis of 2-(4-chlorophenyl)-2-(2,5-dioxoimidazolidine-1-yl)acetic acid

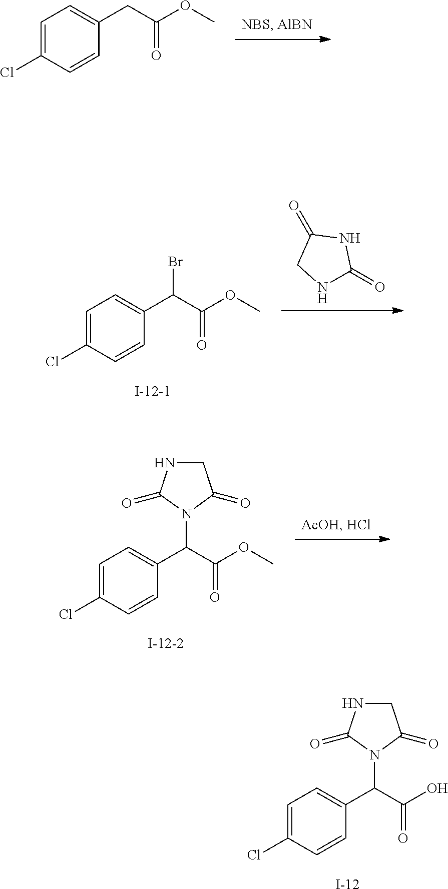

### I-12-1: Methyl 2-bromo-2(4-chlorophenyl)acetate

N-bromosuccinimide (1.93 g, 10.83 mmol) and azodiisobutyronitrile (1.78 g, 10.83 mmol) are added into a solution of methyl 2-(4-chlorophenyl)acetate (2 g, 10.83 mmol) in carbon tetrachloride (20 mL). A mixed solution is stirred at 80° C. for 5 h. The mixture is dried in vacuum to obtain the yellow oily I-12-1 (1.7 g, 56%).

¹H NMR (300 MHz, CDCl₃) δ 7.50-7.47 (m, 2H), 7.36-7.33 (m, 2H), 5.32 (s, 1H), 3.79 (s, 3H).

### I-12-2: Methyl 2-(4-chlorophenyl)-2-(2,5-dioxoimidazolidin-1-yl)acetate

At room temperature, hydantoin (0.89 g, 8.55 mmol) and potassium carbonate (1.63 g, 11.8 mmol) are respectively added into a solution of methyl 2-bromo-2(4-chlorophenyl)acetate (1.56 g, 5.9 mmol) in dimethylformamide (20 mL). A mixture is stirred at 25° C. for 16 h. After the reaction is finished, the mixture is diluted with water and extracted with ethyl acetate (50 mL) three times. Organic phases are combined, washed with saturated saline, dried with anhydrous sodium sulfate, filtered and concentrated in vacuum. The organic phases are purified by liquid chromatography to obtain the yellow solid I-12-2 (1.85 g, 88%).

¹H NMR (300 MHz, CDCl₃) δ 7.45 (d, J=8.5 Hz, 2H), 7.34-7.26 (m, 2H), 5.75 (s, 1H), 5.71 (s, 1H), 4.01 (s, 2H), 3.80 (s, 3H).

### I-12: (4-chlorophenyl)(2,5-dioxoimidazolidin-1-yl)acetic acid

At room temperature, methyl 2-(4-chlorophenyl)-2-(2,5-dioxoimidazolidin-1-yl)acetate (1.17 g, 4.139 mmol) is added into 2.5 mL of concentrated hydrochloric acid and maintained stirred, and acetic acid (7.5 mL) is dropwise added into a mixed solution. The obtained mixture is heated to 120° C. and then stirred for 3 h. After the reaction is finished, the reaction mixture is cooled to room temperature. The obtained mixture is concentrated at a reduced pressure to obtain a crude product I-12 (1.1 g, 86.7%) in a white solid form. The purified crude product is used for a next step.

LC-MS m/z (ESI): 269.0 [M+H]⁺.

### Intermediate I-13: Synthesis of (Z)-2-(4-((6-chloro-1H-indol-3-yl)methylene)-2,5-dioxoimidazolidin-1-yl)-2-(4-chlorophenyl)acetic acid

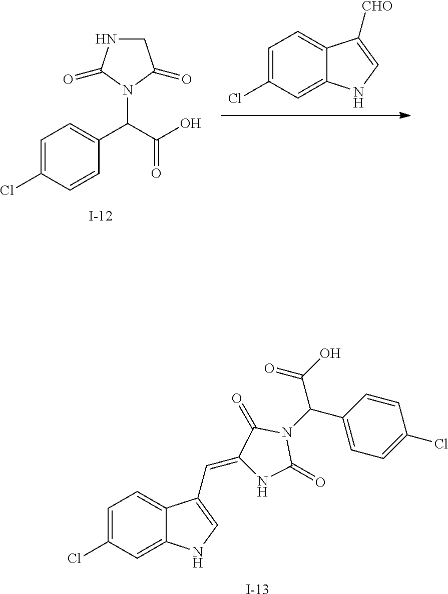

methyl 2-(4-chlorophenyl)-2-(2,5-dioxoimidazolidin-1-yl)acetate (1.56 g, 5.5 mmol) is added into an ethyl acetate (10 mL) solution, and then concentrated hydrochloric acid (3 mL) is then added. The obtained mixture is reflowed till it is completely dissolved. The mixture is concentrated to obtain an intermediate. The intermediate is dissolved in ethyl acetate (15 mL) and piperidine (2 mL) for being further reflowed for 2 h, and then 6-chloro-1H-indole-3-formaldehyde (0.99 g, 5.5 mmol) is added. The mixture is stirred and reflowed for 16 h. After the reaction is finished, the reaction mixture is cooled to room temperature. The mixture is filtered to collect a yellow precipitant, so as to obtain a crude product in a piperidine salt form. The crude product is dissociated with hydrochloric acid to obtain the I-13 (0.56 g, 21.82%). The purified crude product is used for a next step.

LC-MS m/z (ESI): 429.9 [M+H]⁺.

### Intermediate I-14: Synthesis of [(4Z)-4-[(6-chloro-7-fluoro-1H-indol-3-yl)methylene]-2,5-dioxoimidazolidin-1-yl](4-chlorophenyl)acetic acid

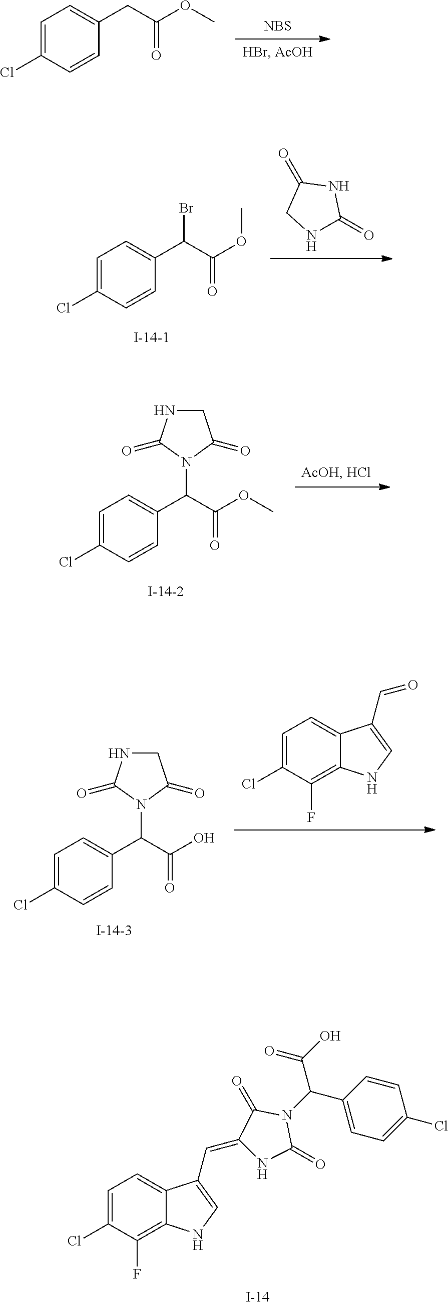

### I-14-1: Methyl 2-bromo-2-(4-chlorophenyl)acetate

At room temperature, hydrobromic acid in acetic acid (7.04 g, 35.208 mmol) is dropwise added into a stirred solution of methyl 2-(4-chlorophenyl)acetate (5 g, 27.083 mmol) and NBS (6.27 g, 35.208 mmol) in DCE (50 mL). The obtained mixture is then stirred at 85° C. for 12 h. After being cooled to room temperature, the obtained mixture is concentrated in vacuum to obtain a white solid crude product I-14-1 (8 g), and the crude product is directly used for a next step without further purification.

### I-14-2: Methyl 2-(4-chlorophenyl)-2-(2,5-dioxoimidazolidin-1-yl)acetate

At room temperature, methyl 2-bromo-2-(4-chlorophenyl)acetate (2 g) and potassium carbonate (13.1 g, 9.488 mmol) are respectively added into a DMF (15 mL) solution, and then hydantoin (570 mg, 5.692 mmol) is added. The obtained mixture is then stirred at room temperature for 12 h. The obtained mixture is concentrated in vacuum. A residue is purified by silica gel column chromatography and eluted with petroleum ether/ethyl acetate (5:1) to obtain the I-14-2 (1.17 g, 93.45%) in a white solid form.

LC-MS m/z (ESI): 283.1 [M+H]⁺.

### I-14-3: (4-chlorophenyl)(2,5-dioxoimidazolidin-1-yl)acetic acid

At room temperature, methyl 2-(4-chlorophenyl)-2-(2,5-dioxoimidazolidin-1-yl)acetate (1.17 g, 4.139 mmol) is added into 2.5 mL of concentrated hydrochloric acid and maintained stirred, and acetic acid (7.5 mL) is continuously dropwise added into a mixed solution. The obtained mixture is heated to 120° C. and then stirred for 3 h. A product is cooled to room temperature. The obtained mixture is concentrated in vacuum to obtain a white solid crude product I-14-3 (1.1 g), and the crude product is directly used for a next step without further purification.

LC-MS m/z (ESI): 269 [M+H]⁺.

### I-14: [(4Z)-4-[(6-chloro-7-fluoro-1H-indol-3-yl)methylene]-2,5-dioxoimidazolidin-1-yl](4-chlorophenyl)acetic acid

At room temperature, (4-chlorophenyl)(2,5-dioxoimidazolidin-1-yl)acetic acid (400 mg, 1.487 mmol) and 6-chloro-7-fluoro-1H-indole-3-formaldehyde (294 mg, 1.489 mmol) are respectively added into ethanol (6 mL), and then piperidine (0.54 mL) is added into a mixed solution. The obtained mixture is heated to 100° C. and stirred for 12 h. After the product is cooled to room temperature, a residue is purified by silica gel column chromatography and eluted with petroleum ether/ethyl acetate (2:1) to obtain the yellow solid I-14 (196 mg, 31%).

LC-MS m/z (ESI): 446.0 [M−H]⁻.

### Intermediate I-15: Synthesis of (4-cyanophenyl)(2,5-dioxypyrimidin-1-yl)acetic acid

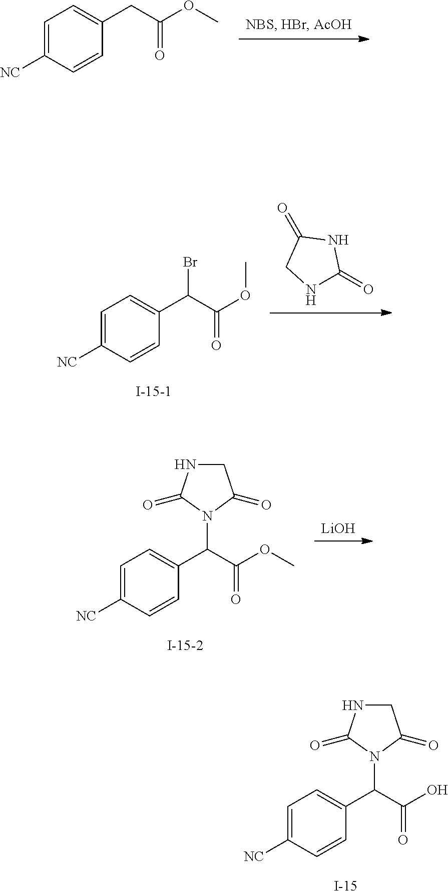

### I-15-1: Methyl 2-bromo-2(4-cyanophenyl)acetate

At room temperature, methyl 2-(4-cyanophenyl)acetate (1200 mg, 6.507 mmol) and NBS (1552 mg, 8.719 mmol) are respectively added into a DCE (12 mL) solution, and a hydrobromic acid solution (0.27 mL, 8.784 mmol) is continuously dropwise added into a mixed system and maintained stirred. After the obtained mixture is heated to 85° C. and stirred for 12 h, the mixture is cooled to room temperature. The obtained mixture is concentrated in vacuum. A residue is dissolved in ethyl acetate (50 mL). The residue is washed with saturated saline (30 mL) three times and dried with anhydrous sodium sulfate. After the residue is filtered, a filtrate is concentrated at a reduced pressure to obtain a light yellow solid crude product I-15-1 (1.46 g), and the crude product may be used for a next step without purification.

### I-15-2: Methyl 2-(4-cyanophenyl)-2-(2,5-dioxoimidazolidin-1-yl)acetate

At room temperature, methyl 2-bromo-2-(4-cyanophenyl)acetate (1.46 g) and hydantoin (1.52 g, 14.429 mmol) are respectively added into a dimethylformamide (20 mL) solution, and potassium carbonate (2.10 g, 14.471 mmol) is continuously added into the solution. A mixture is stirred under nitrogen protection at room temperature for 12 h, reacted, and poured into a saturated ammonium chloride solution (150 mL). The mixture is extracted with ethyl acetate (100) three times. Organic phases are combined, washed with saturated saline (100 mL) twice, and dried with anhydrous sodium sulfate. After the organic phases are filtered, a filtrate is concentrated at a reduced pressure. A residue is purified by reversed-phase chromatography to obtain the I-15-2 (400 mg, 24.3%) in a light yellow solid form.

LC-MS m/z (ESI): 274.2 [M+H]⁺.

### I-15: (4-cyanophenyl)(2,5-dioxoimidazolidin-1-yl)acetic acid

methyl 2-(4-cyanophenyl)-2-(2,5-dioxoimidazolidin-1-yl)acetate (400 mg, 1421 mmol) is dissolved in a mixed solution of tetrahydrofuran (15 mL) and water (15 mL), and a lithium hydroxide aqueous solution (2.1 mL, 2.100 mmol) is dropwise added into the solution. The obtained mixture is then stirred at room temperature under nitrogen protection for 0.5 h. A reside is acidized with hydrochloric acid till the pH value is equal to 5. The obtained mixture is concentrated in vacuum. A residue is purified by reversed-phase chromatography to obtain the I-15 (380 mg, 42%) in a white solid form.

LC-MS m/z (ESI): 260.1 [M+H]⁺.

### Intermediate I-16: Synthesis of [(4Z)-4-[(6-chloro-7-fluoro-1H-indol-3-yl)methylene]-2,5-dioxoimidazolidin-1-yl](4-cyanophenyl)acetic acid

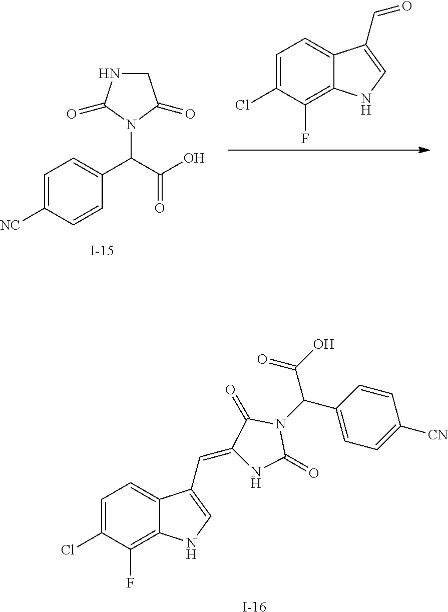

At room temperature, (4-cyanophenyl)(2,5-dioxoimidazolidin-1-yl)acetic acid (200 mg, 0.734 mmol) is added into ethanol (4 mL), and piperidine (263 mg, 2.936 mmol) is continuously added into a solution. The obtained mixture is heated to 80° C. under nitrogen protection and stirred for 0.5 h. The reaction is reduced to room temperature, 6-chloro-7-fluoro-1H-indole-3-formaldehyde (137 mg, 0.661 mmol) is added in batches into the mixture, and the obtained mixture is heated to 80° C. and then stirred for 12 h. The mixture is concentrated at a reduced pressure. A residue is purified by reversed-phase chromatography to obtain the yellow solid I-16 (60 mg, 24%).

LC-MS m/z (ESI): 439.1 [M+H]⁺.

### Intermediate I-17: Synthesis of [(4Z)-4-[(6-chloro-7-methyl-1H-indol-3-yl)methylene]-2,5-dioxoimidazolidin-1-yl](3,4-difluorophenyl)acetic acid

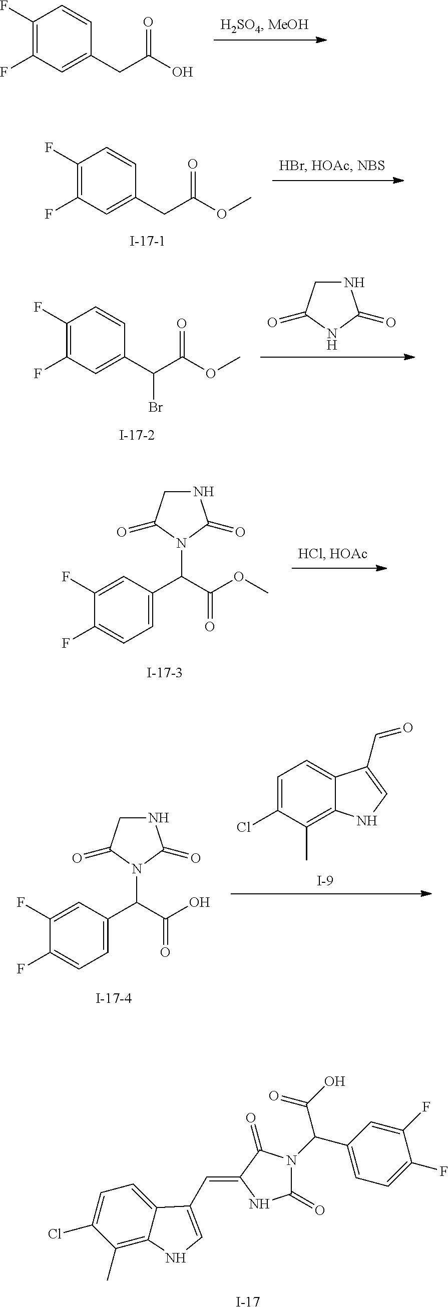

### I-17-1: Methyl 2-(3,4-difluorophenyl)acetate

(3,4-difluorophenyl)acetic acid (10 g, 58 mmol) is added into a methanol (100 mL) solution, and sulfuric acid (10 mL) is slowly dropwise added into the mixed solution. The mixed solution is reacted and reflowed overnight, cooled to room temperature, and poured into ice water (100 mL). The mixture is extracted with ethyl acetate (100 mL) three times. Organic phases are combined and dried with anhydrous Na₂SO₄, filtered, and concentrated in vacuum to obtain a crude product. The crude product is purified by column chromatography (petroleum ether/ethyl acetate) to obtain the yellow oily I-17-1 (10 g, 88%).

LC-MS m/z (ESI): 187.0 [M+H]⁺.

### I-17-2: Methyl 2-bromo-2-(3,4-difluorophenyl)acetate

methyl 2-(3,4-difluorophenyl)acetate (3.0 g, 16 mmol) is added into a carbon tetrachloride (30 mL) solution, and NBS (2.6 g, 14 mmol) and 12 drops of a solution of hydrobromic acid in acetic acid are added into the mixed solution. A reaction mixture is heated to 70° C. and stirred for 10 min. The temperature of the reaction is raised to 85° C. and the mixture is continuously stirred for 2.5 h till thin-layer chromatographic tracking shows that raw materials are consumed completely. After being cooled to room temperature, the mixture is concentrated in vacuum to obtain a crude product. The crude product is purified by column chromatography (petroleum ether/ethyl acetate) to obtain the yellow oily I-17-2 (1.1 g, 72%).

¹H NMR (400 MHz, CDCl₃) δ 7.49-7.44 (m, 1H), 7.28-7.24 (m, 1H), 7.18-7.11 (m, 1H), 5.31 (s, 1H), 3.80 (s, 3H).

### I-17-3: Methyl 2-(3,4-difluorophenyl)-2-(2,5-dioxoimidazolidin-1-yl)acetate

Imidazolidine-2,4-dione (1.71 g, 17 mmol) and potassium carbonate (3.93 g, 28.4 mmol) are added into a solution of methyl 2-bromo-2-(3,4-difluorophenyl)acetate (3.8 g, 14 mmol) in dimethylformamide (37 mL). A reaction mixture is then stirred at room temperature for 14 h. After the reaction is completed, the reaction mixture is diluted with water (100 mL) and extracted with ethyl acetate (50 mL) three times. Organic phases are combined, washed with anhydrous sodium sulfate, filtered and concentrated in vacuum. A crude product is purified by column chromatography (petroleum ether: ethyl acetate=2:1) to obtain the brown solid I-17-3 (1.6 g, 37%).

LC-MS m/z (ESI): 285.0 [M+H]⁺.

### I-17-4: 2-(3,4-difluorophenyl)-2-(2,5-dioxoimidazolidin-1-yl)acetic acid

methyl 2-(3,4-difluorophenyl)-2-(2,5-dioxoimidazolidin-1-yl)acetate (0.85 g, 3.0 mmol) is added into a mixed solution of concentrated hydrochloric acid (26 mL) and ethyl acetate (85 mL), and the mixed solution is heated to 120° C. and stirred for 1 h. A mixture is concentrated in vacuum to obtain the yellow oily crude product I-17-4 (0.80 g, 94%), and the crude product is directly used for a next step without further purification.

LC-MS m/z (ESI): 271.0 [M+H]⁺.

### I-17: (Z)-2-(4-((6-chloro-7-methyl-1H-indol-3-yl)methylene)-2,5-dioxoimidazolidin-1-yl)-2-(3,4-difluorophenyl)acetic acid

2-(3,4-difluorophenyl)-2-(2,5-dioxoimidazolidin-1-yl)acetic acid (0.60 g, 2.2 mmol) is added into ethanol (20 mL), and piperidine (1.9 g, 22 mmol) is added into a mixed system. A mixture is heated and reflowed for 2 h, and then 6-chloro-7-methyl-1H-indole-3-formaldehyde (0.43 g, 2.2 mmol) is added. The mixture is continuously reflowed and stirred for 24 h. The mixture is filtered and extracted with ethyl acetate (2 mL). A solid is dried in vacuum to obtain the yellow solid I-17 (0.7 g, 71%), and the solid is directly used for a next step without further purification.

LC-MS m/z (ESI): 446.0 [M+H]⁺. ¹H NMR (400 MHz, DMSO-d₆) δ 11.95 (brs, 1H), 8.75 (brs, 1H), 8.16 (brs, 1H), 7.62-7.58 (m, 2H), 7.33-7.19 (m, 2H), 7.14 (d, J=8.4 Hz, 1H), 6.74 (s, 1H), 6.67 (s, 1H), 5.31 (s, 1H), 2.49 (s, 3H).

### Intermediate I-18: Synthesis of [(4Z)-4-[(6-chloro-1H-indol-3-yl)methylene]-2,5-dioxoimidazolidin-1-yl](4-cyanophenyl)acetic acid

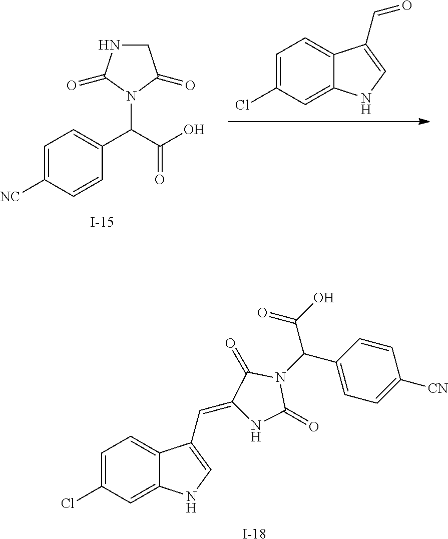

At room temperature, (4-cyanophenyl)(2,5-dioxoimidazolidin-1-yl)acetic acid (200 mg, 0.772 mmol) and piperazine (279.82 mg, 3.088 mmol) are added into an ethanol (3 mL) solution. The obtained mixture is heated to 65° C. and stirred for 20 min. 6-chloro-1H-indole-3-formaldehyde (102 mg, 0.540 mmol) is added in batches into the mixture. The mixture is then stirred at 65° C. for 12 h. Through vacuum concentration, a residue is purified by reversed-phase chromatography to obtain the yellow solid I-18 (102 mg, 31%).

LC-MS m/z (ESI): 419.0 [M−H]⁻.

### Intermediate I-19: Synthesis of [(4Z)-4-[(6-chloro-7-methyl-1H-indol-3-yl)methylene]-2,5-dioxoimidazolidin-1-yl](4-cyanophenyl)acetic acid

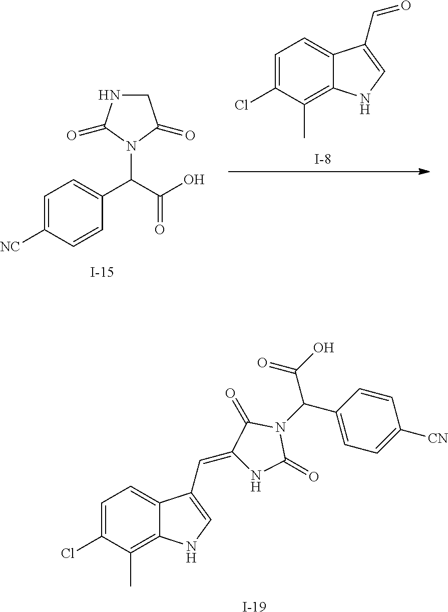

At room temperature, (4-cyanophenyl)(2,5-dioxoimidazolidin-1-yl)acetic acid (300 mg, 1.157 mmol) and piperazine (420 mg, 4.628 mmol) are added into an ethanol (3 mL) solution, and the mixture is heated to 65° C. and stirred for 20 min. After the mixture is cooled to room temperature, 6-chloro-7-methyl-1H-indole-3-formaldehyde (157 mg, 0.810 mmol) is added in batches, and the obtained mixture is heated to 65° C. and then stirred for 12 h. Through vacuum concentration, a residue is purified by reversed-phase chromatography to obtain the yellow solid I-19 (270 mg, 59%).

LC-MS m/z (ESI): 433.0 [M−H]⁻.

### I-20: Synthesis of (Z)-2-(4-((6-chloro-1H-indol-3-yl)methylene)-2,5-dioxoimidazolidin-1-yl)-2-(3,4-difluorophenyl)acetic acid

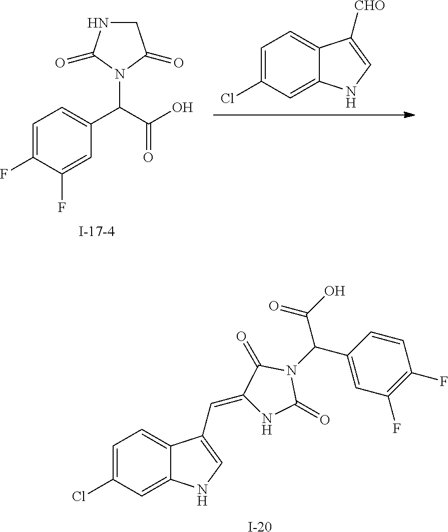

By using the method for the intermediate I-19, the red solid I-20 (1.5 g, 84.77%) is prepared by using the I-17-4 and corresponding aldehyde.

LC-MS m/z (ESI): 432.1 [M+H]⁺.

### I-21: Synthesis of 2-(4-cyano-3-fluorophenyl)-2-(2,5-dioxoimidazolidin-1-yl)acetic acid

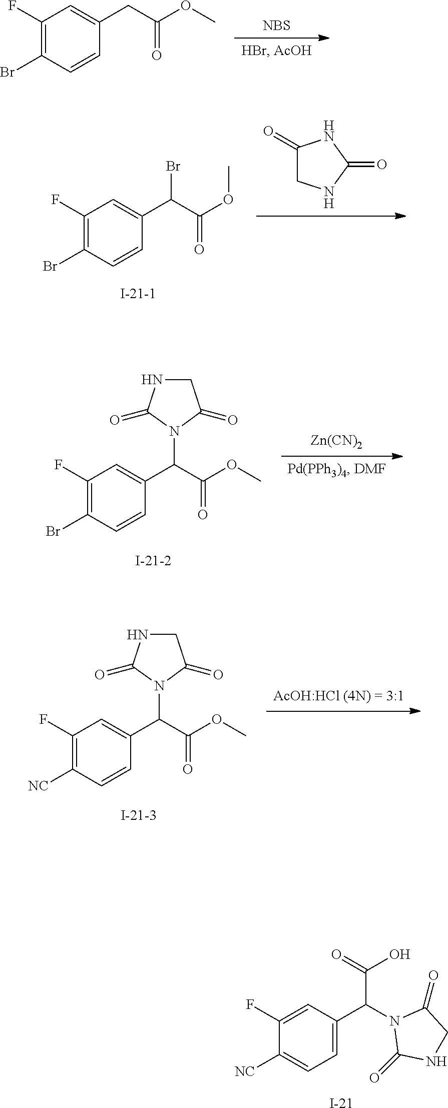

### I-21-1: Methyl 2-bromo-2-(4-bromo-3-fluorophenyl)acetate

At room temperature, a solution of HBr in AcOH (2.3 mL, 16.261 mmol) is added into a solution of methyl 2-(4-bromo-3-fluorophenyl)acetate (3 g, 11.536 mmol) and NBS (2.81 g, 14.997 mmol) in DCE (30 mL), and the obtained mixture is stirred at 85° C. for 12 h. The mixture is concentrated in vacuum, and a residue is diluted with water (100 mL). The obtained mixture is extracted with EtOAc (100 mL×3), and combined organic layers are washed with saline and dried with anhydrous Na₂SO₄. After the organic layers are filtered, a filtrate is concentrated in vacuum. A residue is purified by silica gel column chromatography and eluted with petroleum PE/EA (8:1) to obtain the yellow oily I-21-1 (3.2 g, 77%).

### I-21-2: Methyl 2-(4-bromo-fluorophenyl)-2-(2,5-dioxoimidazolidin-1-yl)acetate

At room temperature, hydantoin (4.36 g, 41.388 mmol) is added in batches into a stirred solution of methyl 2-bromo-2-(4-bromo-3-fluorophenyl)acetate (10 g, 27.611 mmol) and potassium carbonate (8.03 g, 55.222 mmol) in DMF (100 mL), and the obtained mixture is stirred under nitrogen protection at room temperature for 12 h. The obtained mixture is diluted with water (100 mL) and extracted with EtOAc (100 mL×3), and combined organic layers are washed with saline and dried with anhydrous Na₂SO₄. After the organic layers are filtered, a filtrate is concentrated in vacuum. A residue is purified by fast reversed-phase chromatography to obtain the white solid I-21-2 (3.9 g, 36.38%).

LC-MS m/z (ESI): 344.9 [M+H]⁺.

### I-21-3: Methyl 2-(4-cyano-3-fluorophenyl)-2-(2,5-dioxoimidazolidin-1-yl)acetate

At room temperature, zinc cyanide (318 mg, 2.576 mmol) is added in batches into a stirred solution of methyl 2-(4-bromo-3-fluorophenyl)-2-(2,5-dioxopyrrolidin-1-yl)acetate (1000 mg, 2.576 mmol) and tetrakis(triphenylphosphine)palladium (313 mg, 0.258 mmol) in DMF (10 mL), and the obtained mixture is stirred under nitrogen protection at 120° C. for 16 h. The obtained mixture is concentrated in vacuum, and a residue is purified by fast reversed-phase chromatography to obtain the yellow solid I-21-3 (600 mg, 79%).

LC-MS m/z (ESI): 290.0 [M−H]⁻.

### I-21: (4-cyano-3-fluorophenyl)(2,5-dioxoimidazolidin-1-yl)acetic acid

At room temperature, HCl (2 mL, 4N) is added in batches into a solution of methyl 2-(4-cyano-3-fluorophenyl)-2-(2,5-dioxopyrrolidin-1-yl)acetate (800 mg, 2.747 mmol) in AcOH (6 mL), and the obtained mixture is stirred under nitrogen protection at 100° C. for 1 h. A reaction solution is concentrated to obtain white solid I-21 (700 mg, 92%). A crude product is directly used for a next step without further purification.

LC-MS m/z (ESI): 278.1 [M+H]⁺.

### EXAMPLES

### Example 1

Compound 1: (Z)-5-((6-chloro-1H-indol-3-yl)methylene)-3-(3,4-difluorobenzyl)imidazolidine-2,4-dione

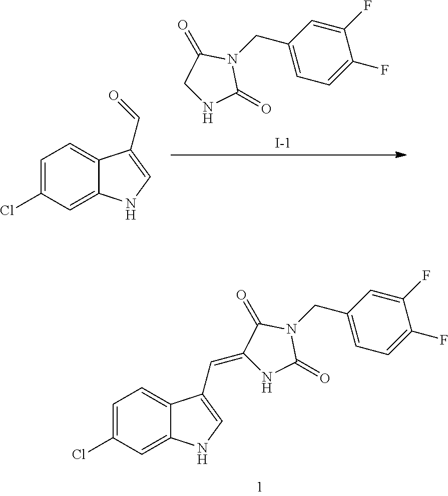 [SMILES: O=Cc1c[nH]c2cc(Cl)ccc12 ; O=C1CNC(=O)N1Cc1ccc(F)c(F)c1 ; [I-] ; O=C1N/C(=C\c2c[nH]c3cc(Cl)ccc23)C(=O)N1Cc1ccc(F)c(F)c1]

At room temperature, an intermediate I-1 (119 mg, 0.505 mmol) and 6-chloro-1H-indole-3-formaldehyde (90 mg, 0.476 mmol) were dissolved in ethanol (5 ml). Under stirring, acetic acid (150 mg, 2.370 mmol) and piperidine (0.5 ml) were slowly added into the mixture, and a reaction solution was heated under nitrogen protection to 100° C. and stirred for 2 h. After being cooled to room temperature, the reaction solution was concentrated in vacuum. A crude product was purified by a preparative liquid chromatography to obtain a yellow solid compound 1 (33.5 mg, 17.61%).

LC-MS m/z (ESI): 385.75 [M−H]−. ¹H NMR (400 MHz, DMSO-d₆) δ 11.99 (brs, 1H), 10.51 (brs, 1H), 8.19 (s, 1H), 7.83 (d, J=8.4 Hz, 1H), 7.48 (d, J=2.0 Hz, 1H), 7.45-7.35 (m, 2H), 7.19-7.11 (m, 2H), 6.88 (s, 1H), 4.68 (s, 2H).

### Example 2

### Compound 2: (Z)-5-((6-chloro-1H-indol-3-yl)methylene)-3-(4-chlorobenzyl)imidazolidine-2,4-dione

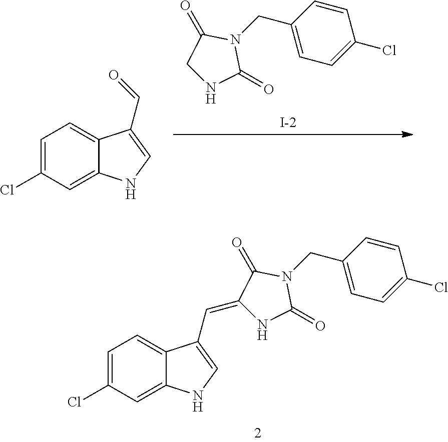

By using the method in Example 1, the yellow solid compound 2 (104.6 mg, 32.42%) was prepared by using the 1-2 and corresponding aldehyde.

LC-MS m/z (ESI): 386.60 [M+H]⁺. ¹H NMR (400 MHz, DMSO-d6) δ 11.96 (s, 1H), 10.54-10.30 (m, 1H), 8.16 (d, J=1.9 Hz, 1H), 7.80 (d, J=8.5 Hz, 1H), 7.45 (d, J=1.8 Hz, 1H), 7.40-7.36 (m, 2H), 7.32-7.27 (m, 2H), 7.10 (dd, J=8.5, 1.9 Hz, 1H), 6.85-6.83 (m, 1H), 4.64 (s, 2H).

### Example 3

### Compound 3: (Z)-5-((6-chloro-1H-indol-3-yl)methylene)-3-(4-fluorobenzyl)imidazolidine-2,4-dione

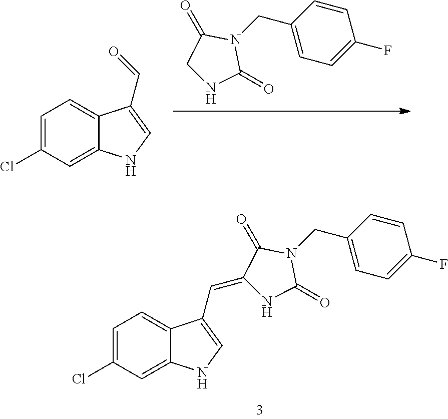 [SMILES: O=Cc1c[nH]c2cc(Cl)ccc12 ; O=C1CNC(=O)N1Cc1ccc(F)cc1 ; O=C1N/C(=C\c2c[nH]c3cc(Cl)ccc23)C(=O)N1Cc1ccc(F)cc1]

By using the method in Example 1, the yellow solid compound 3 (75.8 mg, 14.24%) was prepared by using 3-(4-fluorobenzyl)imidazolidine-2,4-dione and corresponding aldehyde.

LC-MS m/z (ESI): 369.70 [M+H]⁺. ¹H NMR (400 MHz, DMSO-d6) δ 11.99 (s, 1H), 10.50 (s, 1H), 8.19 (s, 1H), 7.83 (d, J=8.5 Hz, 1H), 7.48 (d, J=1.7 Hz, 1H), 7.36 (dd, J=8.6, 5.6 Hz, 2H), 7.24-7.10 (m, 3H), 6.87 (s, 1H), 4.67 (s, 2H).

### Example 4

Compound 4: (Z)-4-((4-((6-chloro-1H-indol-3-yl)methylene)-2,5-dioxoimidazolidin-1-yl)methyl)benzonitrile

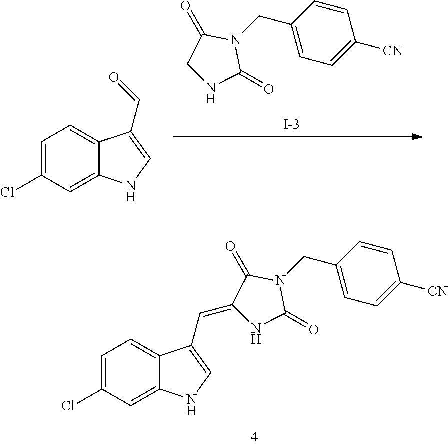

By using the method in Example 1, the white solid compound 4 (12.3 mg, 2.34%) was prepared by using the 1-3 and corresponding aldehyde.

LC-MS m/z (ESI): 376.70 [M+H]⁺. ¹H NMR (400 MHz, DMSO-d₆) δ 12.01 (s, 1H), 10.56 (s, 1H), 8.21 (d, J=2.5 Hz, 1H), 7.84 (dd, J=8.3, 1.6 Hz, 3H), 7.50 (d, J=8.2 Hz, 3H), 7.14 (dd, J=8.5, 1.8 Hz, 1H), 6.90 (s, 1H), 4.78 (s, 2H).

### Example 5

### Compound 5: (Z)-5-((6-bromo-1H-indol-3-yl)methylene)-3-(4-chlorobenzyl)imidazolidine-2,4-dione

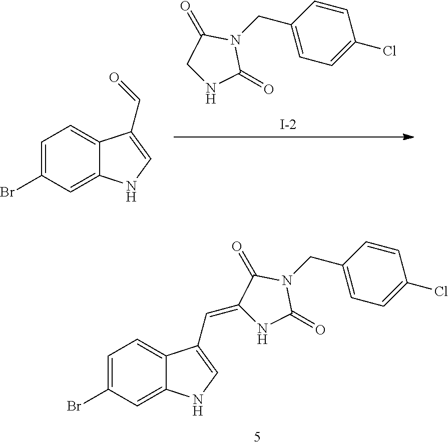

By using the method in Example 1, the yellow solid compound 5 (15.6 mg, 6.73%) was prepared by using the I-2 and corresponding aldehyde.

LC-MS m/z (ESI): 430.50 [M+H]⁺. ¹H NMR (400 MHz, DMSO-d6) δ 12.00 (s, 1H), 10.45 (s, 1H), 8.18 (s, 1H), 7.78 (d, J=8.5 Hz, 1H), 7.62 (d, J=1.3 Hz, 1H), 7.42 (d, J=8.4 Hz, 2H), 7.33 (d, J=8.4 Hz, 2H), 7.28-7.23 (m, 1H), 6.87 (s, 1H), 4.68 (s, 2H).

### Example 6

### Compound 6: (Z)-5-((6-chloro-7-(fluoromethoxyl)-1H-indol-3-yl)methylene)-3-(3,4-difluorobenzyl)imidazolidine-2,4-dione

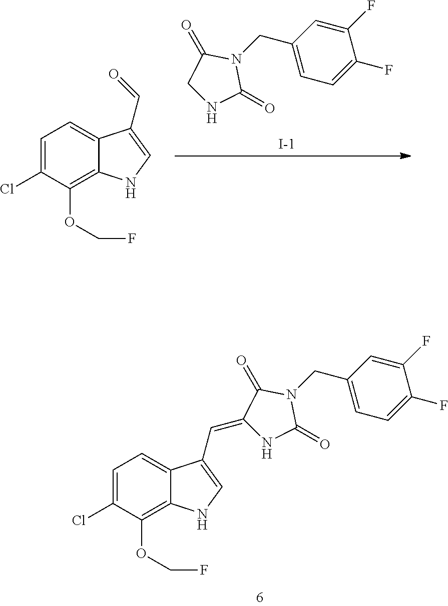 [SMILES: O=Cc1c[nH]c2c(OCF)c(Cl)ccc12 ; O=C1CNC(=O)N1Cc1ccc(F)c(F)c1 ; [I-] ; O=C1N/C(=C\c2c[nH]c3c(OCF)c(Cl)ccc23)C(=O)N1Cc1ccc(F)c(F)c1]

By using the method in Example 1, the yellow solid compound 6 (32.5 mg, 34.43%) was prepared by using the I-1 and corresponding aldehyde.

¹H NMR (400 MHz, DMSO-d6) δ 12.20 (s, 1H), 10.57 (s, 1H), 8.22 (s, 1H), 7.70 (m, 1H), 7.45-7.37 (m, 2H), 7.21 (d, J=8.5 Hz, 1H), 7.16 (s, 1H), 6.86 (s, 1H), 5.90 (s, 1H), 5.77 (s, 1H), 4.68 (s, 2H).

### Example 7

### Compound 7: (Z)-5-((6,7-dichloro-1H-indol-3-yl)methylene)-3-(3,4-difluorobenzyl)imidazolidine-2,4-dione

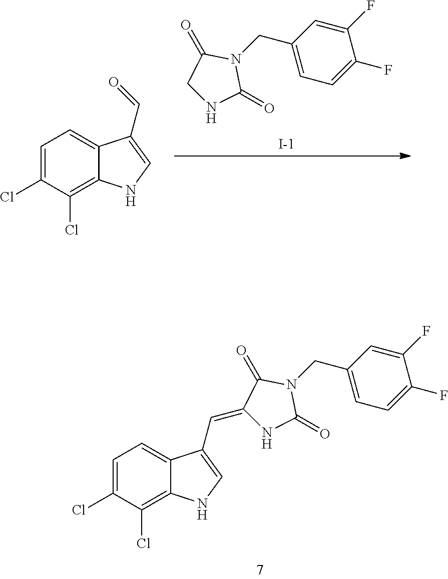 [SMILES: O=Cc1c[nH]c2c(Cl)c(Cl)ccc12 ; O=C1CNC(=O)N1Cc1ccc(F)c(F)c1 ; [I-] ; O=C1N/C(=C\c2c[nH]c3c(Cl)c(Cl)ccc23)C(=O)N1Cc1ccc(F)c(F)c1]

By using the method in Example 1, the yellow solid compound 7 (21.6 mg, 4.95%) was prepared by using the compound 1-1 and corresponding aldehyde.

LC-MS m/z (ESI): 420.05 [M−H]⁻. ¹H NMR (400 MHz, DMSO-d6) δ 12.36 (s, 1H), 10.62 (s, 1H), 8.27 (s, 1H), 7.85 (d, J=8.5 Hz, 1H), 7.41 (ddd, J=13.5, 10.0, 6.9 Hz, 2H), 7.31 (d, J=8.5 Hz, 1H), 7.16 (s, 1H), 6.86 (s, 1H), 4.68 (s, 2H).

### Example 8

### Compound 8: (Z)-5-((6-chloro-7-methoxyl-1H-indol-3-yl)methylene)-3-(3,4-difluorobenzyl)imidazolidine-2,4-dione

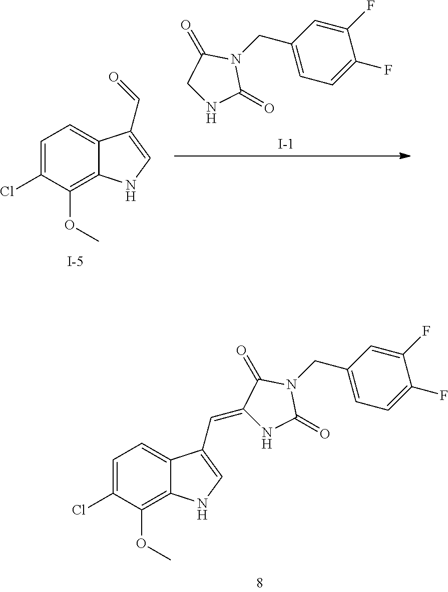

By using the method in Example 1, the yellow solid compound 8 (40 mg, 16.56%) was prepared by using the intermediates I-5 and I-1.

LC-MS m/z (ESI): 417.9 [M+H]⁺. ¹H NMR (400 MHz, DMSO-d₆) δ 12.19 (d, J=2.4 Hz, 1H), 10.53 (s, 1H), 8.17 (d, J=2.6 Hz, 1H), 7.54 (d, J=8.5 Hz, 1H), 7.37 (ddt, J=10.0, 8.1, 5.3 Hz, 2H), 7.14-7.08 (m, 2H), 6.81 (s, 1H), 4.64 (s, 2H), 3.89 (s, 3H).

### Example 9

### Compound 9: (Z)-5-((6-chloro-7-(methylsulfonyl)-1H-indol-3-yl)methylene)-3-(3,4-difluorobenzyl)imidazolidine-2,4-dione

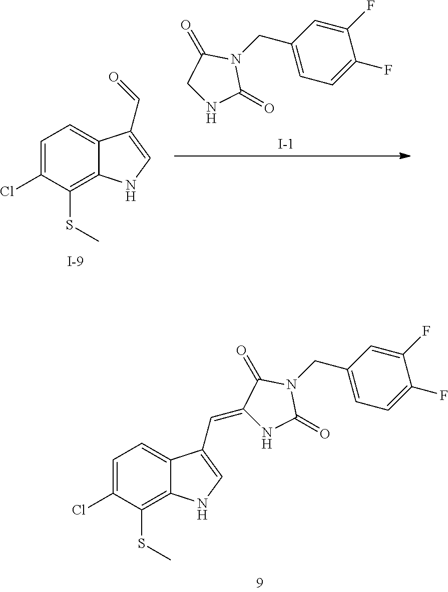

By using the method in Example 1, the yellow solid compound 9 (12.7 mg, 33.28%) was prepared by using the intermediates I-9 and I-1.

LC-MS m/z (ESI): 433.9 [M+H]⁺. ¹H NMR (300 MHz, DMSO-d₆) 12.06 (s, 1H), 10.63 (s, 1H), 8.25 (s, 1H), 7.86-7.83 (m, 1H), 7.44-7.29 (m, 3H), 7.26-7.16 (m, 1H), 6.86 (s, 1H), 4.68 (s, 2H), 2.43 (s, 3H).

### Example 10

### Compound 10: (Z)-5-((6-chloro-7-fluoro-1H-indol-3-yl)methylene)-3-(3,4-difluorobenzyl)imidazolidine-2,4-dione

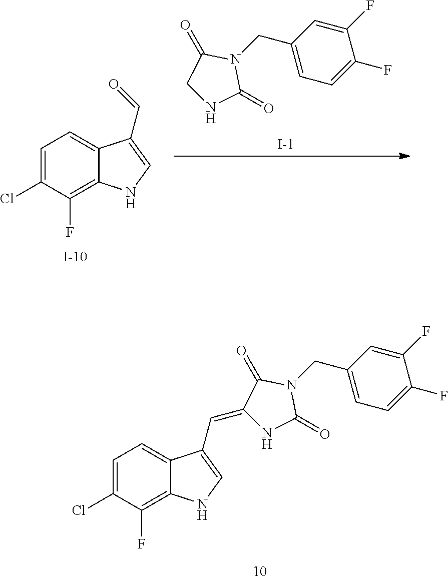

By using the method in Example 1, the yellow solid compound 10 (19.5 mg, 13.35%) was prepared by using the intermediates I-10 and I-1.

LC-MS m/z (ESI): 403.8 [M−H]⁻. ¹H NMR (300 MHz, DMSO-d₆) δ 12.54 (s, 1H), 10.57 (s, 1H), 8.25 (s, 1H), 7.68 (d, J=8.5 Hz, 1H), 7.46-7.36 (m, 2H), 7.23-7.18 (m, 2H), 6.86 (s, 1H), 4.68 (s, 2H).

### Example 11

### Compound 11: (Z)-4-((4-((6-chloro-7-(fluoromethoxyl)-1H-indol-3-yl)methylene)-2,5-dioxoimidazolidin-1-yl)methyl)-2-fluorobenzonitrile

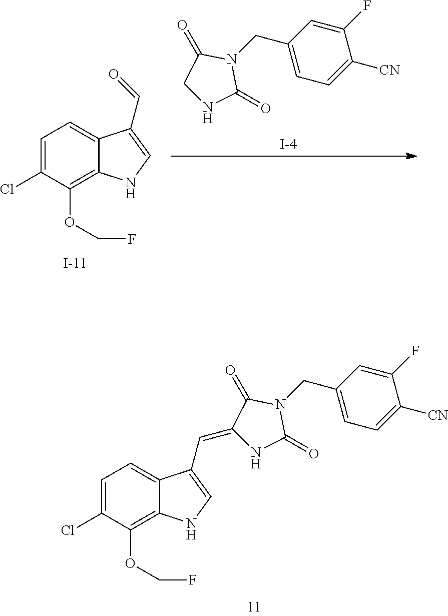

By using the method in Example 1, the yellow solid compound 11 (9.8 mg, 2.2%) was prepared by using the intermediates I-11 and I-4.

LC-MS m/z (ESI): 443.1 [M+H]⁺. ¹H NMR (300 MHz, DMSO-d₆) δ 12.21 (s, 1H), 10.63 (s, 1H), 8.24 (s, 1H), 7.94-7.89 (m, 1H), 7.73-7.70 (m, 1H), 7.52-7.48 (m, 1H), 7.35-7.33 (m, 1H), 7.23-7.20 (m, 1H), 6.88 (s, 1H), 5.93 (s, 1H), 5.75 (s, 1H), 4.80 (s, 2H).

### Example 12

### Compound 12: (Z)-5-((6-chloro-7-(fluoromethoxyl)-1H-indol-3-yl)methylene)-3-(4-chlorobenzyl)imidazolidine-2,4-dione

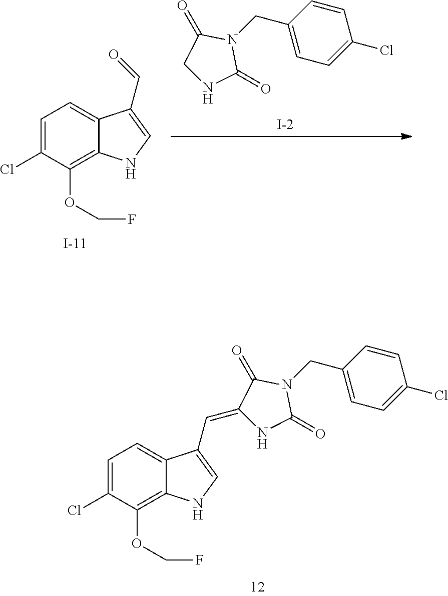

By using the method in Example 1, the yellow solid compound 12 (8.9 mg, 5.18%) was prepared by using the intermediates I-11 and I-2.

LC-MS m/z (ESI): 431.8 [M−H]⁻. ¹H NMR (300 MHz, DMSO-d₆) δ 12.20 (s, 1H), 10.58 (s, 1H), 8.22 (s, 1H), 7.72 (d, J=8.6 Hz, 1H), 7.44-7.40 (m, 2H), 7.35-7.22 (m, 2H), 7.17-7.27 (m, 1H), 6.86 (s, 1H), 5.93 (s, 1H), 5.75 (s, 1H), 4.68 (s, 2H).

### Example 13

### Compound 13: (Z)-4-((4-((6-chloro-7-(fluoromethoxyl)-1H-indol-3-yl)methylene)-2,5-dioxoimidazolidin-1-yl)methyl)benzonitrile

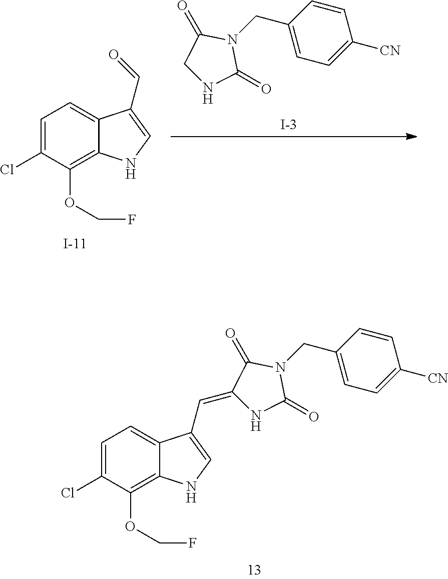

By using the method in Example 1, the yellow solid compound 13 (8.1 mg, 4.68%) was prepared by using the intermediates I-11 and I-3.

LC-MS m/z (ESI): 422.8 [M−H]−. ¹H NMR (300 MHz, DMSO-d₆) δ 12.21 (s, 1H), 10.62 (s, 1H), 8.23 (m, 1H), 7.85-7.82 (m, 2H), 7.72-7.69 (m, 1H), 7.51-7.48 (m, 2H), 7.22-7.20 (m, 1H), 6.87 (s, 1H), 5.93 (s, 1H), 5.75 (s, 1H), 4.78 (s, 2H).

### Example 14

### Compound 14: (Z)-4-((4-((6-chloro-7-fluoro-1H-indol-3-yl)methylene)-2,5-dioxoimidazolidin-1-yl)methyl)-2-fluorobenzonitrile

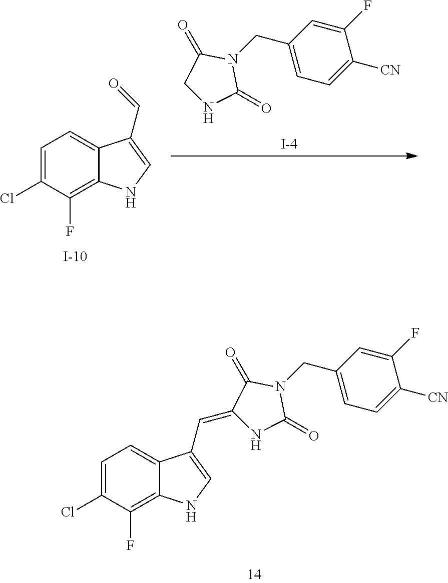

By using the method in Example 1, the yellow solid compound 14 (6.6 mg, 4.89%) was prepared by using the intermediates I-10 and I-4.

LC-MS m/z (ESI): 410.9 [M−H]⁻. ¹H NMR (400 MHz, DMSO-d₆) δ 12.57 (s, 1H), 10.63 (s, 1H), 8.28 (s, 1H), 7.95-7.90 (m, 1H), 7.71-7.68 (m, 1H), 7.52-7.48 (m, 1H), 7.35-7.32 (m, 1H), 7.24-7.19 (m, 1H), 6.87 (s, 1H), 4.80 (s, 2H).

### Example 15

### Compound 15: (Z)-5-((6-chloro-7-fluoro-1H-indol-3-yl)methylene)-3-(4-chlorobenzyl)imidazolidine-2,4-dione

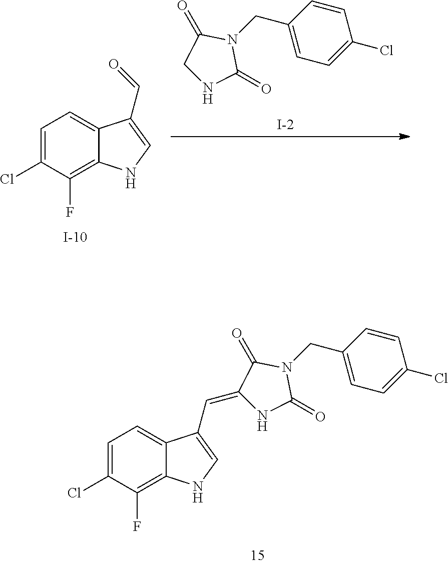

By using the method in Example 1, the yellow solid compound 15 (9.4 mg, 7.51%) was prepared by using the intermediates I-10 and I-2.

LC-MS m/z (ESI): 401.70 [M−H]⁻. ¹H NMR (400 MHz, DMSO-d₆) 12.53 (s, 1H), 10.56 (s, 1H), 8.25 (s, 1H), 7.67 (d, J=8.4 Hz, 1H), 7.43-7.33 (m, 4H), 7.22-7.19 (m, 1H), 6.85 (s, 1H), 4.68 (s, 2H).

### Example 16

### Compound 16: (Z)-4-((4-((6-chloro-7-fluoro-1H-indol-3-yl)methylene)-2,5-dioxoimidazolidin-1-yl)methyl)benzonitrile

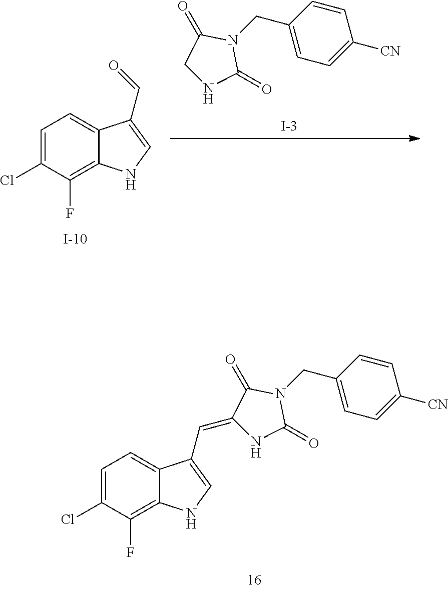

By using the method in Example 1, the yellow solid compound 16 (25.9 mg, 12.16%) was prepared by using the intermediates I-10 and I-3.

LC-MS m/z (ESI): 392.8 [M−H]⁻. ¹H NMR (400 MHz, DMSO-d₆) δ 12.54 (s, 1H), 10.60 (s, 1H), 8.26 (s, 1H), 7.83 (d, J=8.0 Hz 2H), 7.68 (d, J=8.4 Hz, 1H), 7.50 (d, J=8.4 Hz, 2H), 7.21 (t, J=6.8 Hz, 1H), 6.87 (s, 1H), 4.78 (s, 2H).

### Example 17

### Compound 17: (Z)-5-((6-chloro-7-fluoro-1H-indol-3-yl)methylene)-3-(4-acetenylbenzyl)imidazolidine-2,4-dione

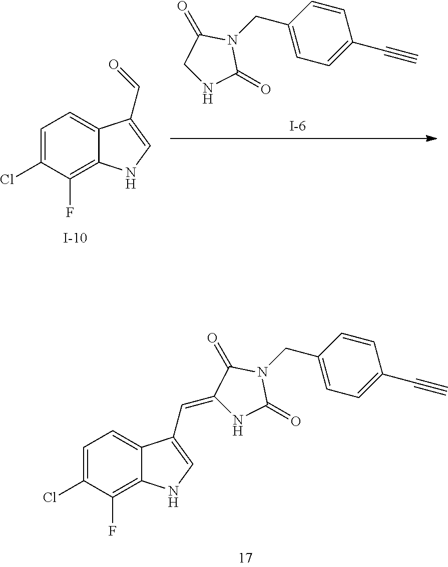

By using the method in Example 1, the yellow solid compound 17 (23.1 mg, 22.88%) was prepared by using the intermediates I-10 and I-6.

LC-MS m/z (ESI): 391.9 [M−H]⁻. ¹H NMR (300 MHz, DMSO-d₆) δ 12.56 (s, 1H), 10.59 (s, 1H), 8.26 (d, J=2.8 Hz, 1H), 7.67 (d, J=8.6 Hz, 1H), 7.50-7.43 (m, 2H), 7.31 (d, J=8.3 Hz, 2H), 7.20 (dd, J=8.5, 6.5 Hz, 1H), 6.85 (s, 1H), 4.70 (s, 2H), 4.18 (s, 1H).

### Example 18

### Compound 18: (Z)-3-((4-((6-chloro-7-fluoro-1H-indol-3-yl)methylene)-2,5-dioxoimidazolidin-1-yl)methyl)benzonitrile

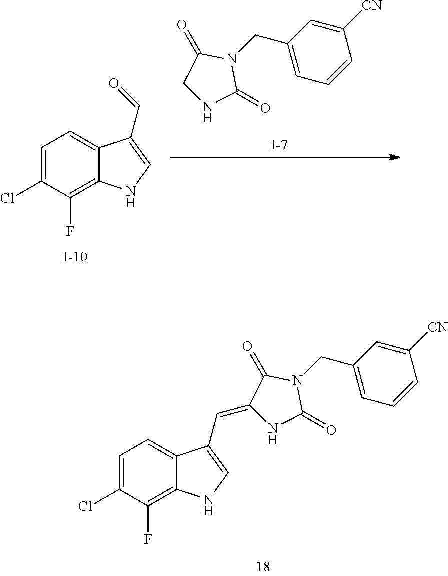

By using the method in Example 1, the yellow solid compound 18 (12.8 mg, 14.52%) was prepared by using the intermediates I-10 and I-7.

LC-MS m/z (ESI): 392.8 [M−H]⁻. ¹H NMR (300 MHz, DMSO-d₆) δ 12.60-12.39 (m, 1H), 10.58 (s, 1H), 8.82-8.21 (m, 1H), 7.82-7.73 (m, 2H), 7.72-7.57 (m, 3H), 7.33-7.10 (m, 1H), 6.80 (s, 1H), 4.75 (s, 2H).

### Example 19

### Compound 19: (Z)-4-((4-((6-chloro-7-methyl-1H-indol-3-yl)methylene)-2,5-dioxoimidazolidin-1-yl)methyl)benzonitrile

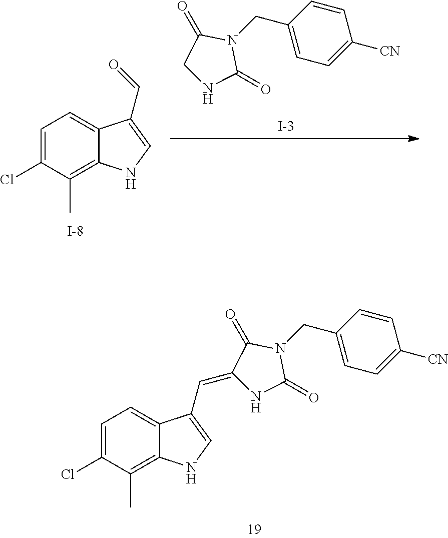

By using the method in Example 1, the yellow solid compound 19 (47 mg, 23%) was prepared by using the intermediates I-3 and I-8.

LC-MS m/z (ESI): 388.8 [M−H]⁻. ¹H NMR (300 MHz, DMSO-d₆) δ 12.00 (s, 1H), 10.55 (s, 1H), 8.21 (d, J=2.5 Hz, 1H), 7.84-7.78 (m, 2H), 7.64 (d, J=8.5 Hz, 1H), 7.54-7.45 (m, 2H), 7.14 (d, J=8.5 Hz, 1H), 6.87 (d, J=0.6 Hz, 1H), 4.78 (s, 2H), 2.52 (s, 3H).

### Example 20

### Compound 20: (Z)-4-((4-((6-chloro-1H-indol-3-yl)methylene)-2,5-dioxoimidazolidin-1-yl)methyl)-2-fluorobenzonitrile

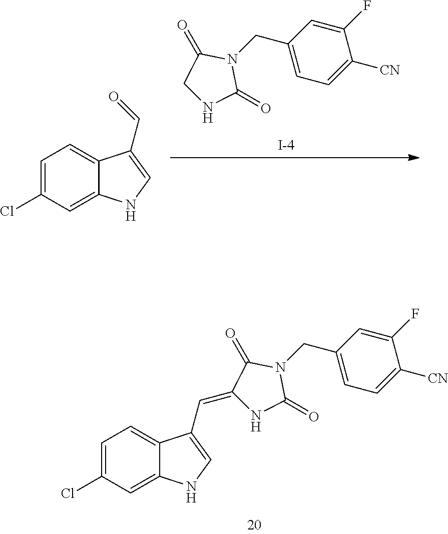

By using the method in Example 1, the yellow solid compound 20 (27 mg, 12%) was prepared by using the intermediate I-4 and corresponding aldehyde.

LC-MS m/z (ESI): 393.0 [M+H]⁻. ¹H NMR (400 MHz, DMSO-d6) δ 12.01-11.75 (m, 1H), 10.53 (m, 1H), 8.48 (m, 1H), 7.91 (t, J=7.5 Hz, 1H), 7.83 (d, J=8.5 Hz, 1H), 7.67-7.45 (m, 2H), 7.33 (d, J=8.1 Hz, 1H), 7.15 (m, 1H), 6.84 (m, 1H), 4.79 (s, 2H).

### Example 21

### Compound 21: (Z)-4-((4-((6-chloro-7-methyl-1H-indol-3-yl)methylene)-2,5-dioxoimidazolidin-1-yl)methyl)-2-fluorobenzonitrile

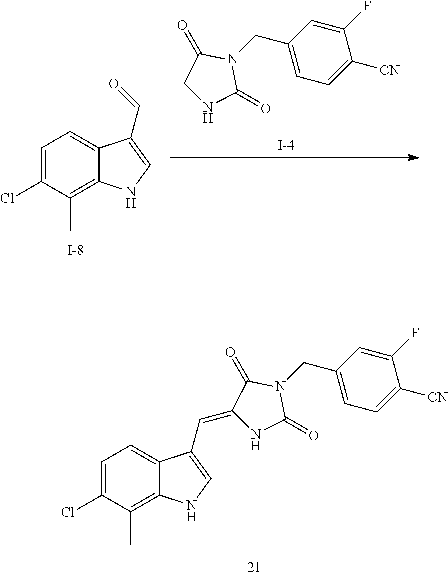

By using the method in Example 1, the yellow solid compound 21 (42 mg, 18%) was prepared by using the intermediates I-4 and I-8.

LC-MS m/z (ESI): 407.1 [M−H]⁻. ¹H NMR (300 MHz, DMSO-d₆) δ 12.02 (s, 1H), 10.57 (s, 1H), 8.22 (d, J=2.9 Hz, 1H), 7.91 (t, J=7.4 Hz, 1H), 7.65 (d, J=8.5 Hz, 1H), 7.54-7.44 (m, 1H), 7.33 (dd, J=8.1, 1.6 Hz, 1H), 7.15 (d, J=8.5 Hz, 1H), 6.88 (s, 1H), 4.80 (s, 2H), 2.52 (s, 3H).

### Example 22

### Compound 22: (Z)-2-(4-((6-chloro-1H-indol-3-yl)methylene)-2,5-dioxoimidazolidin-1-yl)-2-(4-chlorophenyl)-N-(1,3-dihydroxypropan-2-yl)acetamide

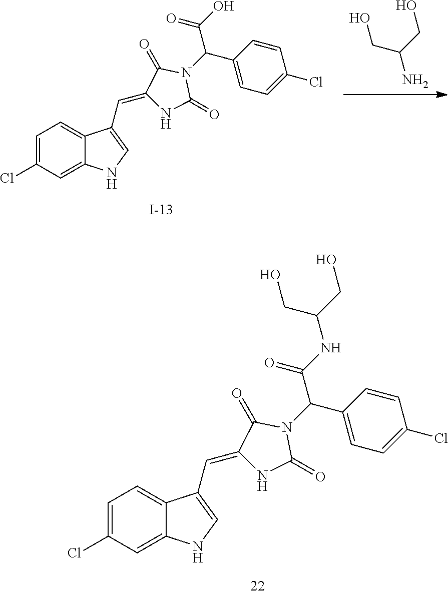

The intermediate I-13 (150 mg, 0.35 mmol), HATU (199 mg, 0.52 mmol), and N,N-diisopropylethylamine (90 mg, 0.7 mmol) were added into a dimethylformamide (5 mL) solution. The mixture was stirred at 25° C. for 10 min, and then 2-aminopropane-1,3-diol (47 mg, 0.52 mmol) was added into the mixture. The mixture was continuously stirred at 25° C. for 2 h, concentrated in vacuum, and purified by preparative thin-layer chromatography to obtain a yellow solid compound 22 (160 mg, 90%).

LC-MS m/z (ESI): 502.9 [M+H]⁺. ¹H NMR (400 MHz, DMSO-d₆) δ 11.98 (d, J=2.0 Hz, 1H), 10.53 (s, 1H), 8.19 (d, J=2.5 Hz, 1H), 7.82 (d, J=8.5 Hz, 1H), 7.75 (d, J=8.2 Hz, 1H), 7.48 (d, J=1.8 Hz, 1H), 7.46-7.39 (m, 4H), 7.13 (dd, J=8.5, 1.9 Hz, 1H), 6.83 (s, 1H), 5.77 (s, 1H), 4.63 (t, J=5.5 Hz, 1H), 4.49 (t, J=5.7 Hz, 1H), 3.85-3.77 (m, 1H), 3.47-3.35 (m, 4H).

### Example 23

### Compound 23: (Z)-2-(4-((6-chloro-1H-indol-3-yl)methylene)-2,5-dioxoimidazolidin-1-yl)-2-(4-chlorophenyl)-N-(2-(diethylamino)ethyl)acetamide

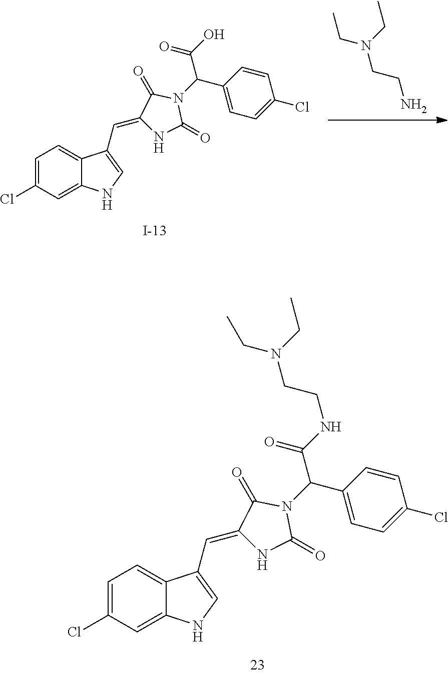

By using the method in Example 22, the yellow solid compound 23 (180 mg, 58.61%) was prepared by using the intermediate I-13 and N,N-diethenylethane-1,2-diamine.

LC-MS m/z (ESI): 527.9 [M+H]⁺. ¹HNMR (400 MHz, DMSO-d₆) δ 12.08 (s, 1H), 10.68 (s, 1H), 8.26 (d, J=2.7 Hz, 2H), 7.87 (d, J=8.6 Hz, 1H), 7.54 (d, J=1.8 Hz, 1H), 7.51 (s, 4H), 7.20 (dd, J=8.5, 1.9 Hz, 1H), 6.93 (s, 1H), 5.86 (s, 1H), 3.51 (m, 2H), 3.27-3.15 (m, 6H), 1.24 (t, J=7.1 Hz, 6H).

### Example 24

### Compound 24: (Z)-2-(4-((6-chloro-1H-indol-3-yl)methylene)-2,5-dioxoimidazolidin-1-yl)-2-(3,4-difluorophenyl)-N-(1,3-dihydroxypropan-2-yl)acetamide

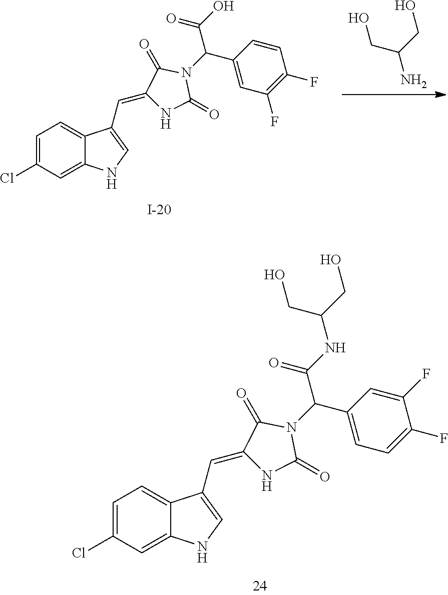

By using the method in Example 22, the yellow solid compound 24 (30 mg, 24.98%) was prepared by using the intermediate I-20 and 2-aminopropane-1,3-diol.

LC-MS m/z (ESI): 502.9 [M−H]⁻. ¹HNMR (400 MHz, DMSO-d₆) δ 11.98 (s, 1H), 10.53 (s, 1H), 8.19 (s, 1H), 7.87-7.77 (m, 2H), 7.55-7.36 (m, 3H), 7.30-7.28 (m, 1H), 7.18-7.10 (m, 1H), 6.84 (s, 1H), 5.79 (s, 1H), 4.64-4.61 (m, 1H), 4.50-4.47 (m, 1H), 3.85-3.80 (m, 1H), 3.52-3.43 (m, 4H).

### Example 25

### Compound 25: (Z)-5-((6-chloro-7-methoxyl-1H-indol-3-yl)methylene)-3-(1-(3,4-difluorophenyl)ethyl)imidazolidine-2,4-dione

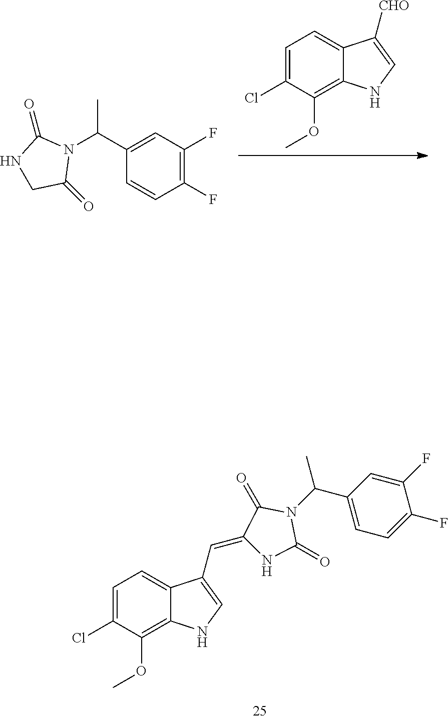 [SMILES: CC(c1ccc(F)c(F)c1)N1C(=O)CNC1=O ; COc1c(Cl)ccc2c(C=O)c[nH]c12 ; COc1c(Cl)ccc2c(/C=C3\NC(=O)N(C(C)c4ccc(F)c(F)c4)C3=O)c[nH]c12]

By using the method in Example 1, the yellow solid compound 25 (7.3 mg, 0.76%) was prepared.

LC-MS m/z (ESI): 432.0 [M+H]⁺. ¹HNMR (400 MHz, DMSO-d₆) δ 12.19 (s, 1H), 10.51 (s, 1H), 8.18 (d, J=2.5 Hz, 1H), 7.55 (d, J=8.6 Hz, 1H), 7.52-7.36 (m, 2H), 7.23 (s, 1H), 7.14 (d, J=8.6 Hz, 1H), 6.79 (s, 1H), 5.41-5.29 (m, 1H), 3.93 (s, 3H), 1.78 (d, J=7.2 Hz, 3H).

### Example 26

### Compound 26: 6-chloro-3-{[(4Z)-1-[1-(3,4-difluorophenyl)-2-hydroxyethyl]-2,5-dioxoimidazolidin-4-methylene]methyl}indol-1-carboxylate

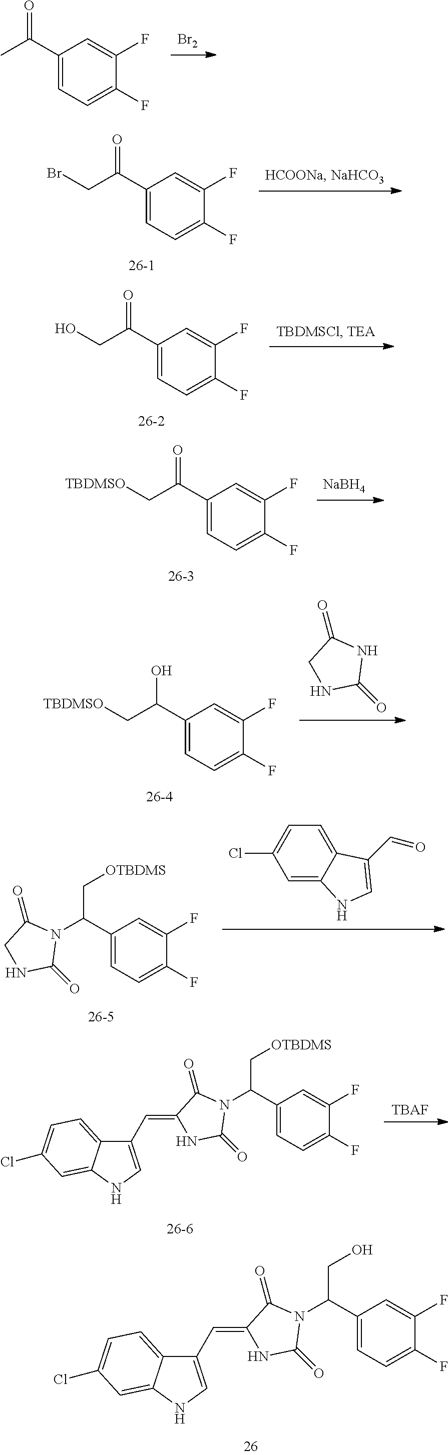

### 26-1: 2-bromo-1-(3,4-difluorophenyl)ethanone

At 0° C., bromine (2 g, 11.889 mmol) was slowly added into a solution of 1-(3,4-difluorophenyl)ethanone (2 g, 12.169 mmol) in dichloromethane (20 mL). After the reaction was finished, the mixture was poured into ice water. The obtained mixture was extracted with dichloromethane (40 mL) three times. Organic phases were combined, washed with saturated saline (20 mL), and dried with anhydrous sodium sulfate. After the organic phases were filtered, a filtrate was concentrated at a reduced pressure. A residue was purified by silica gel column chromatography and eluted with DCM/PE (1:20) to obtain the white solid 26-1 (1.68 g, 58%).

¹H NMR (300 MHz, DMSO-d₆) δ 8.15-8.05 (m, 1H), 7.98-7.86 (m, 1H), 7.70-7.60 (m, 1H), 4.95 (s, 2H).

### 26-2: 1-(3,4-difluorophenyl)-2-hydroxyethanone

At room temperature, 2-bromo-1-(3,4-difluorophenyl)ethanone (2 g, 8.399 mmol), sodium formate (6.97 g, 97.365 mmol), and sodium hydrogen carbonate (800 mg, 9.047 mmol) were added into a mixed solution of acetonitrile (8 mL) and water (4 mL). The obtained mixture was heated to 65° C. under nitrogen protection and stirred for 18 h. After the reaction was completed, the mixture was cooled to room temperature. The obtained mixture was poured into water (5 mL). The mixture was extracted with ethyl acetate (20 mL) three times. Organic phases were combined, washed with saturated saline (10 mL) three times, and dried with anhydrous sodium sulfate. After the organic phases were filtered, a filtrate was concentrated in vacuum. A residue was purified by silica gel column chromatography and eluted with petroleum ether/ethyl acetate (2:1) to obtain the yellow solid 26-2 (610 mg, 39%).

¹HNMR (300 MHz, DMSO-d6) δ 8.05-7.95 (m, 1H), 7.90-7.80 (m, 1H), 7.75-7.62 (m, 1H), 5.26-5.18 (m, 1H), 4.82-4.74 (m, 2H).

### 26-3: 2-[(tert-butyldimethylsilyl)oxy]-1-(3,4-difluorophenyl)ethanone

At room temperature, 1-(3,4-difluorophenyl)-2-hydroxyethanone (2.1 g, 11.346 mmol) and triethylamine (1.48 g, 13.894 mmol) were added into a dichloromethane (20 mL) solution, and tert-butyl dimethyl chlorosilane (2.76 g, 17.396 mmol) was added into the solution when the solution was stirred. The obtained mixture was stirred at room temperature under nitrogen protection for 16 h. The mixture was poured into ice water, extracted with ethyl acetate (80 mL) three times, and combined organic phases were washed with saturated saline (50 mL) three times and dried with anhydrous sodium sulfate. After the organic layers were filtered, a filtrate was concentrated in vacuum. A residue was purified by silica gel column chromatography and eluted with DCM/PE (1:2) to obtain the yellow oily 26-3 (2.48 g, 69%).

LC-MS m/z (ESI): 285.0 [M−H]⁻.

### 26-4: 2-[(tert-butyldimethylsilyl)oxy]-1-(3,4-difluorophenyl)ethanol

Under nitrogen protection, 2-[(tert-butyldimethylsilyl)oxy]-1-(3,4-difluorophenyl)ethanone (2.0 g, 6.299 mmol) was added into an ethanol (25 mL) solution, and at 0° C., sodium borohydride (800 mg, 20.090 mmol) was added in batches. After being stirred at room temperature for 0.5 h, the mixture was poured into ice water and extracted with ethyl acetate (10 mL) three times. Organic phases were combined, washed with saturated saline (5 mL) three times, and dried with anhydrous sodium sulfate. After the organic phases were filtered, a filtrate was concentrated in vacuum. A residue was purified by silica gel column chromatography and eluted with petroleum ether/ethyl acetate (1:20) to obtain the brown oily compound 26-4 (1.56 g, 86%).

¹HNMR (400 MHz, DMSO-d6) δ 7.45-7.35 (m, 2H), 7.30-7.20 (m, 1H), 5.56-5.50 (m, 1H), 4.70-4.60 (m, 1H), 3.80-3.70 (m, 1H), 3.65-3.55 (m, 1H) 0.86 (s, 9H), 0.07 (s, 6H).

### 26-5: 3-{2-[(tert-butyldimethylsilyl)oxy]-1-(3,4-difluorophenyl)ethyl}imidazolidine-2,4-dione

At 0° C., 2-[(tert-butyldimethylsilyl)oxy]-1-(3,4-difluorophenyl)ethanol (1.56 g, 5.404 mmol) and triphenylphosphine (2.84 g, 10.286 mmol) were added into a THF (20 mL) solution, and under nitrogen protection, diethyl azodicarbonate (1.88 g, 10.255 mmol) was added into the mixture. The obtained mixture was heated to 40° C. and stirred for 16 h. After the system was cooled to room temperature, the obtained mixture was concentrated in vacuum. A residue was purified by reversed-phase chromatography to obtain the brown oily compound 26-5 (640 mg, 25%).

LC-MS m/z (ESI): 371.2 [M+H]⁺.

### 26-6: (5Z) 3-{2-[(tert-butyldimethylsilyl)oxy]-1-(3,4-difluorophenyl)ethyl}-5-[(6-chloro-1H-indol-3-yl)methylene]imidazolidine-2,4-dione

At room temperature, 3-{2-[(tert-butyldimethylsilyl)oxy]-1-(3,4-difluorophenyl)ethyl}imidazolidine-2,4-dione (440 mg, 0.927 mmol) and 6-chloro-1H-indole-3-formaldehyde (236 mg, 1.25 mmol) were added into an ethanol (5 mL) solution, and piperidine (0.5 mL) was dropwise added into the system at room temperature. The obtained mixture was heated to 100° C. and stirred for 4 h. After being cooled to room temperature, the mixture was concentrated in vacuum. A residue was purified by reversed-phase chromatography to obtain the yellow solid compound solid 26-6 (325 mg, 37%).

LC-MS m/z (ESI): 532.3 [M+H]⁺.

### Compound 26: 6-chloro-3-{[(4Z)-1-[1-(3,4-difluorophenyl)-2-hydroxyethyl]-2,5-dioxoimidazolidin-4-methylene]methyl}indol-1-carboxylate

At room temperature, (Z)-3-(2-(tert-butyldimethylsilyloxy)-1-(3,4-difluorophenyl)ethyl)-5-((6-chloro-1H-indol-3-yl)methylene)imidazolidine-2,4-dione (263 mg, 0.457 mmol) was added into a tetrahydrofuran (6 mL) solution, and tetramethylammonium fluoride (3.66 mL, 3.66 mmol, 1N) was dropwise added into the solution. The obtained mixture was stirred at room temperature for 1 h. The obtained mixture was concentrated in vacuum. A residue was purified by preparative high performance liquid chromatography to obtain the yellow solid compound solid 26 (2.4 mg, 29.66%).

LC-MS m/z (ESI): 416.1 [M−H]⁻. ¹HNMR (300 MHz, DMSO-d6) δ 12.03-11.73 (m, 1H), 10.50 (s, 1H), 8.79-8.14 (m, 1H), 7.86-7.61 (m, 1H), 7.56-7.36 (m, 3H), 7.27 (dd, J=5.2, 3.0 Hz, 1H), 7.18-7.10 (m, 1H), 6.84 (s, 1H), 5.20 (dt, J=10.7, 5.7 Hz, 2H), 4.31 (td, J=10.3, 5.4 Hz, 1H), 3.94 (dt, J=11.2, 5.8 Hz, 1H).

### Example 27

### Compound 27: (Z)-2-(4-((6-chloro-7-fluoro-1H-indol-3-yl)methylene)-2,5-dioxoimidazolidin-1-yl)-2-(4-cyano-3-fluorophenyl)-N-(1,3-dihydroxypropan-2-yl)acetamide

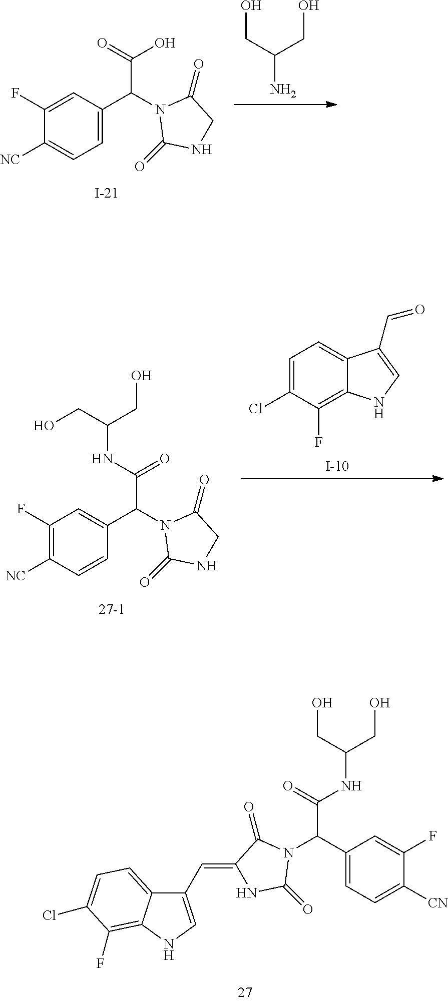

### 27-1: 2-(4-cyano-3-fluorophenyl)-N-(1,3-dihydroxypropan-2-yl)-2-(2,5-dioxoimidazolidin-1-yl)acetamide

At room temperature, 2-aminopropane-1,3-diol (62 mg, 0.686 mmol) and HATU (261 mg, 0.686 mmol) were added into dimethylformamide (1 mL), and (4-cyano-3-fluorophenyl)(2,5-dioxoimidazolidin-1-yl)acetic acid (100 mg, 0.343 mmol) and diisopropylethylamine (74 mg, 0.686 mmol) were added in batches into the solution. The obtained mixture was heated to 40° C. under nitrogen protection and stirred for 12 h. The mixture was concentrated in vacuum, and a residue was purified by reversed-phase chromatography to obtain the yellow solid compound solid 27-1 (60 mg, 50%).

LC-MS m/z (ESI): 349.0 [M−H]⁻.

### Compound 27: (Z)-2-(4-((6-chloro-7-fluoro-1H-indol-3-yl)methylene)-2,5-dioxoimidazolidin-1-yl)-2-(4-cyano-3-fluorophenyl)-N-(1,3-dihydroxypropan-2-yl)acetamide

At room temperature, 2-(4-cyano-3-fluorophenyl)-N-(1,3-dihydroxypropan-2-yl)-2-(2,5-dioxoimidazolidin-1-yl) acetamide (20 mg, 0.051 mmol) was added into ethyl acetate (1 mL), piperidine (18.42 mg, 0.204 mmol) was added in batches into the mixed solution, and the obtained mixture was reflowed for 20 min. After the mixture was cooled to room temperature, 6-chloro-7-fluoro-1H-indole-3-formaldehyde (8 mg, 0.036 mmol) was added in batches. The obtained mixture was then reflowed for 12 h, and after being cooled to room temperature, the mixture was concentrated in vacuum. A residue was purified by preparative high performance liquid chromatography to obtain the yellow solid compound solid 27 (4.3 mg, 15.35%).

LC-MS m/z (ESI): 528.1 [M−H]⁻. ¹H NMR (400 MHz, DMSO-d₆) δ 12.55 (d, J=3.0 Hz, 1H), 10.63 (s, 1H), 8.71-8.20 (m, 1H), 8.04 (d, J=8.2 Hz, 1H), 7.96-7.80 (m, 1H), 7.76-7.55 (m, 2H), 7.43 (d, J=8.1 Hz, 1H), 7.25-7.16 (m, 1H), 6.83 (s, 1H), 5.91 (s, 1H), 4.58 (dt, J=57.7, 5.7 Hz, 2H), 4.10-3.77 (m, 1H), 3.56-3.35 (m, 4H).

### Example 28

### Compound 28: (Z)-2-(4-((6-chloro-7-fluoro-1H-indol-3-yl)methylene)-2,5-dioxoimidazolidin-1-yl)-2-(4-chlorophenyl)-N-(1,3-dihydroxypropan-2-yl)acetamide

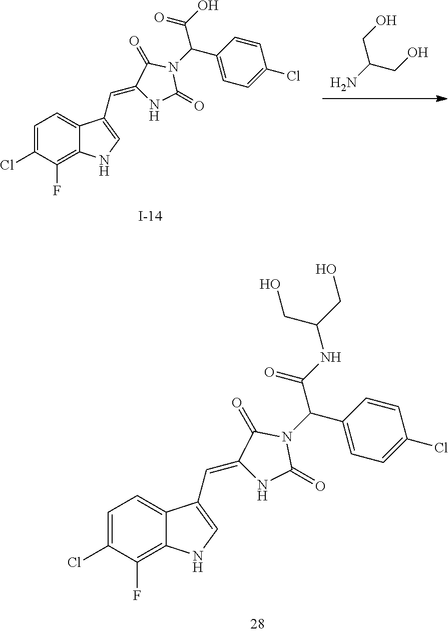

By using the method in Example 22, the yellow solid compound 28 (54.1 mg, 29%) was prepared by using the intermediate I-14 and 2-aminopropane-1,3-diol.

LC-MS m/z (ESI): 521.1 [M+H]⁺. ¹HNMR (300 MHz, DMSO-d₆) δ 12.52 (s, 1H), 10.57 (s, 1H), 8.24 (d, J=2.8 Hz, 1H), 7.69 (dd, J=17.6, 8.4 Hz, 2H), 7.42 (d, J=1.2 Hz, 5H), 7.20 (dd, J=8.6, 6.5 Hz, 1H), 6.81 (s, 1H), 5.77 (s, 1H), 4.60-4.30 (m, 1H), 3.86-3.75 (m, 1H), 3.48-3.33 (m, 5H).

### Example 29

### Compound 29: (Z)-2-(4-((6-chloro-7-fluoro-1H-indol-3-yl)methylene)-2,5-dioxoimidazolidin-1-yl)-2-(4-cyanophenyl)-N-(1,3-dihydroxypropan-2-yl)acetamide

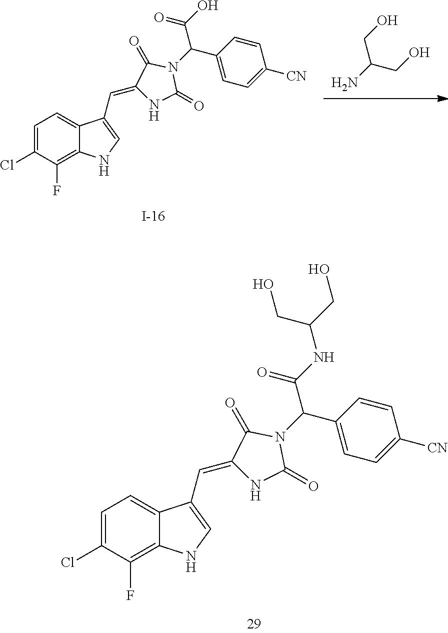

By using the method in Example 22, the yellow solid compound 29 (34 mg, 21%) was prepared by using the intermediate I-16 and 2-aminopropane-1,3-diol.

LC-MS m/z (ESI): 510.1 [M−H]⁻. ¹HNMR (400 MHz, DMSO-d₆) δ 12.59-12.37 (m, 1H), 10.62 (s, 1H), 8.25 (d, J=2.8 Hz, 1H), 7.94 (d, J=8.2 Hz, 1H), 7.82 (d, J=8.1 Hz, 2H), 7.67 (d, J=8.6 Hz, 1H), 7.59 (d, J=8.1 Hz, 2H), 7.20 (dd, J=8.5, 6.4 Hz, 1H), 6.82 (s, 1H), 5.87 (s, 1H), 3.82 (q, J=6.5 Hz, 2H), 3.53-3.31 (m, 5H).

### Example 30

### Compound 30: (Z)-2-(4-((6-chloro-7-methyl-1H-indol-3-yl)methylene)-2,5-dioxoimidazolidin-1-yl)-2-(3,4-difluorophenyl)-N-(1,3-dyhydroxl-2-(hydroxymethyl)-2-propyl)acetamide

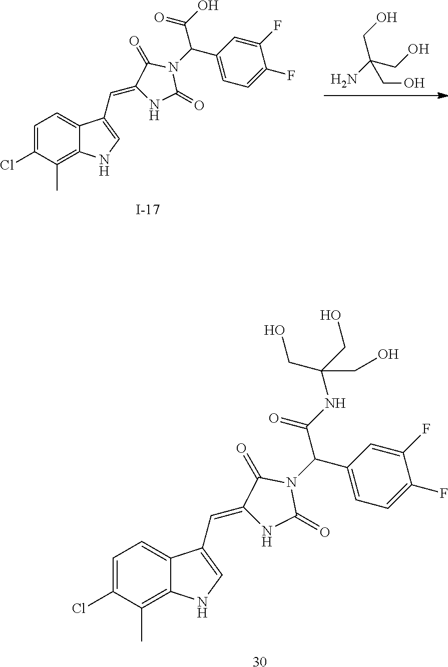

By using the method in Example 22, the yellow solid compound 30 (4.4 mg, 4.57%) was prepared by using the intermediate I-17 and 2-amino-2-(hydroxymethyl)propane-1,3-diol.

LC-MS m/z (ESI): 549.1 [M+H]⁺. ¹HNMR (300 MHz, DMSO-d₆) δ 12.07 (s, 1H), 10.64 (s, 1H), 8.24 (s, 1H), 7.69-7.31 (m, 4H), 7.19-7.14 (m, 2H), 6.86 (s, 1H), 5.86 (s, 1H), 4.54 (br, 2H), 3.70-3.40 (m, 7H).

### Example 31

### Compound 31: (Z)-2-(4-((6-chloro-1H-indol-3-yl)methylene)-2,5-dioxoimidazolidin-1-yl)-2-(4-cyanophenyl)-N-(1,3-dihydroxypropan-2-yl)acetamide

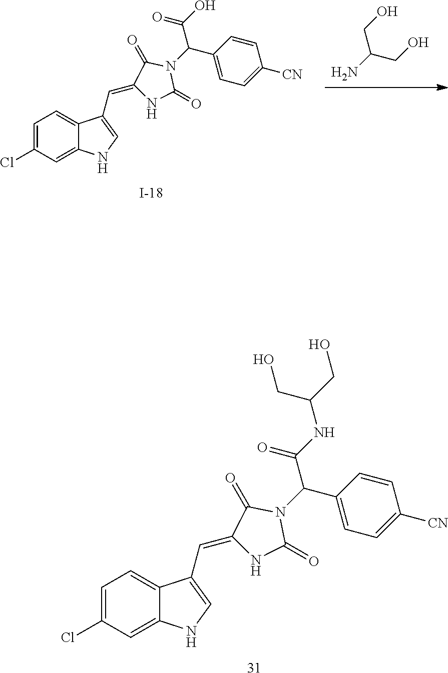

By using the method in Example 22, the yellow solid compound 31 (6.4 mg, 4.28%) was prepared by using the intermediate I-18 and 2-aminopropane-1,3-diol.

LC-MS m/z (ESI): 494.1 [M+H]⁺. ¹HNMR (400 MHz, DMSO-d₆) δ 12.07 (d, J=2.8 Hz, 1H), 10.76 (s, 1H), 8.24 (d, J=2.5 Hz, 1H), 8.00 (s, 3H), 7.86 (dd, J=21.8, 8.3 Hz, 3H), 7.64 (d, J=8.0 Hz, 2H), 7.50 (d, J=1.9 Hz, 1H), 7.15 (dd, J=8.5, 1.9 Hz, 1H), 6.95 (s, 1H), 6.24 (s, 1H), 5.40 (s, 1H), 4.31 (q, J=6.9, 6.0 Hz, 2H), 3.62-3.53 (m, 1H), 3.51-3.45 (m, 1H).

### Example 32

### Compound 32: (Z)-2-(4-((6-chloro-7-methyl-1H-indol-3-yl)methylene)-2,5-dioxoimidazolidin-1-yl)-2-(4-cyanophenyl)-N-(1,3-dihydroxypropan-2-yl)acetamide

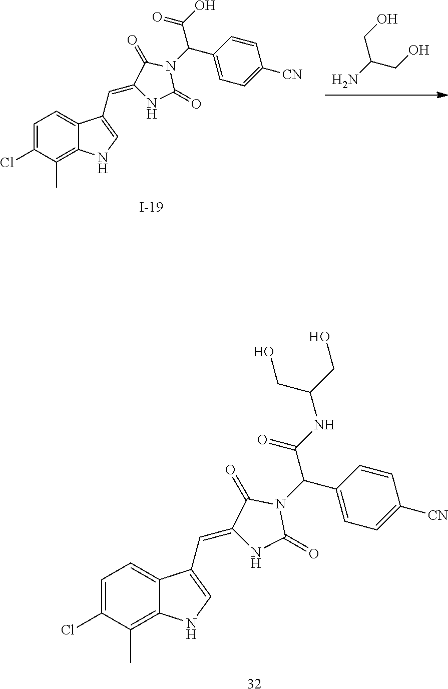

By using the method in Example 22, the yellow solid compound 32 (58 mg, 21%) was prepared by using the intermediate I-19 and 2-aminopropane-1,3-diol.

LC-MS m/z (ESI): 508.0 [M+H]⁺. ¹HNMR (400 MHz, DMSO-d₆) δ 12.01 (d, J=3.0 Hz, 1H), 10.57 (s, 1H), 8.21 (d, J=2.9 Hz, 1H), 7.94 (d, J=8.2 Hz, 1H), 7.82 (d, J=8.1 Hz, 2H), 7.64 (d, J=8.5 Hz, 1H), 7.59 (d, J=8.1 Hz, 2H), 7.14 (d, J=8.5 Hz, 1H), 6.83 (s, 1H), 5.87 (s, 1H), 4.18-3.65 (m, 3H), 3.53-3.31 (m, 4H), 2.51 (s, 3H).

### Example 33

### Compound 33: (Z)-4-(1-(4-((6-chloro-7-fluoro-1H-indol-3-yl)methylene)-2,5-dioxoimidazolidin-1-yl)-2-oxyethyl)-2-fluorobenzonitrile

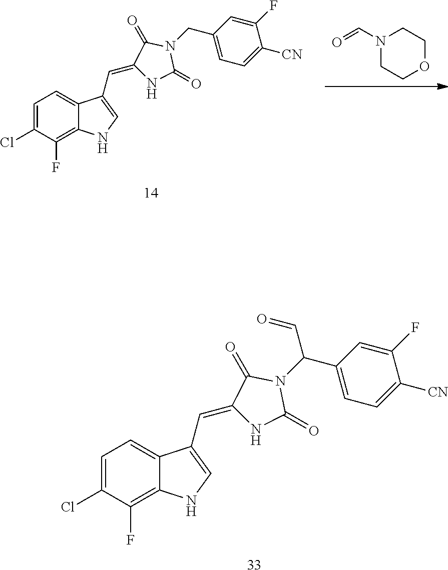 [SMILES: N#Cc1ccc(CN2C(=O)N/C(=C\c3c[nH]c4c(F)c(Cl)ccc34)C2=O)cc1F ; O=CN1CCOCC1 ; "14" ; N#Cc1ccc(C(C=O)N2C(=O)N/C(=C\c3c[nH]c4c(F)c(Cl)ccc34)C2=O)cc1F ; "33"]

Under nitrogen protection at −78° C., Li MDS (4.8 mL, 4.8 mmol, 5 equiv, 1 N) was added into a solution of the compound 14 (400 mg, 0.958 mmol, 1 equiv, 98.9%) in tetrahydrofuran (24.5 mL), and the mixture was stirred at −78° C. for 1 h. Then formyl morpholine (1.4 g, 11.552 mmol, 12.05 equiv, 95%) was added, and the mixture was stirred at −80° C. for 4 h. A solution of HOAc (1 mL) in THF (3 mL) was added at −80° C. to terminate the reaction, the mixture was heated to −20° C., and water (100 mL) was added in the reaction. The obtained mixture was extracted with EtOAc (3×50 mL), and combined organic layers were washed with saline (100 mL) and dried with anhydrous sodium sulfate. After the organic layers were filtered, a filtrate was concentrated at a reduced pressure. A residue was purified by reverse HPLC to obtain the yellow solid compound 33 (14.2 mg, 3.3%).

LC-MS m/z (ESI): 439.0 [M−H]⁻. ¹H NMR (300 MHz, DMSO-d₆) δ 12.55 (s, 1H), 11.08 (s, 1H), 10.62 (s, 1H), 8.30 (s, 1H), 7.93 (s, 1H), 7.87-7.62 (m, 2H), 7.58-7.48 (m, 1H), 7.44-7.16 (m, 2H), 6.87 (s, 1H).

### Example 34

### Compound 34: (Z)-4-((4-((6-chloro-7-fluoro-1H-indol-3-yl)methylene)-2,5-dioxoimidazolidin-1-yl)-2,3-difluorobenzonitrile

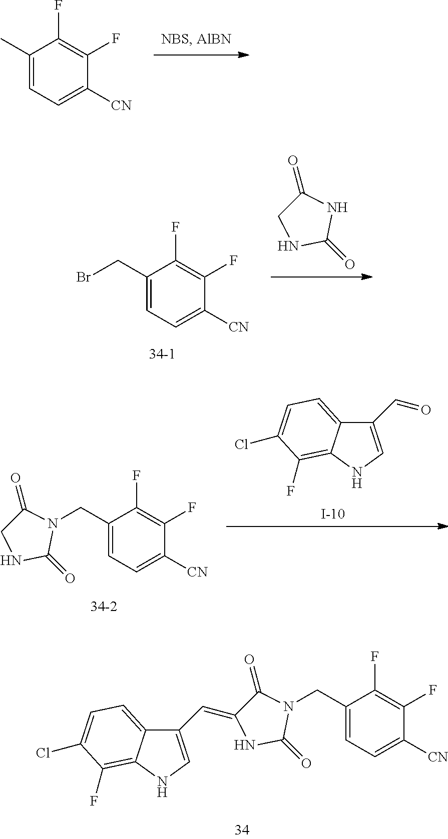

### 34-1: 4-(bromomethyl)-2,3-difluorobenzonitrile

At room temperature, AIBN (180 mg, 1.061 mmol, 1.0 equiv, 95%) was added in batches into a solution of 2,3-difluoro-4-methyl benzonitrile (2 g, 11.787 mmol, 1.0 equiv, 95%) and NBS (3 g, 16.738 mmol, 1.4 equiv, 95%) in CHCl₃ (95 mL), and the obtained mixture was stirred under nitrogen protection at 80° C. for 8 h. The mixture was cooled to room temperature, and water was added to terminate the reaction. The obtained mixture was extracted with EtOAc (3×200 mL), and combined organic layers were washed with saline (3×200 mL) and dried with anhydrous sodium sulfate. After the organic layers were filtered, a filtrate was concentrated at a reduced pressure. A residue was purified by silica gel column chromatography and eluted with PE/EA (1:1) to obtain the yellow oily 34-1 (1.7 g, 67.5%).

LC-MS m/z (ESI): 299.8 [M−H]⁻.

### 34-2: 4-[(2,5-dioxoimidazolidin-1-yl)methyl]-2,3-difluorobenzonitrile

At room temperature, hydantoin (1.0 g, 9.398 mmol, 1.3 equiv, 95%) was added into a solution of 34-1 (1.7 g, 7.241 mmol, 1.0 equiv, 99%) and potassium carbonate (2.0 g, 13.748 mmol, 1.9 equiv, 95%) in DMF (15 mL), and the obtained mixture was stirred under nitrogen protection at room temperature for 12 h. Water was added to terminate the reaction, the obtained mixture was extracted with EtOAc (3×100 mL), and combined organic layers were washed with saline (3×60 mL) and dried with anhydrous sodium sulfate. After the organic layers were filtered, a filtrate was concentrated at a reduced pressure to obtain the white solid 34-2 (1.2 g, 67%).

LC-MS (M−H)⁻=250.0

### Compound 34: 4-{[(4Z)-4-[(6-chloro-7-fluoro-1H-indol-3-yl)methylene]-2,5-dioxoimidazolidin-1-yl]methyl}-2,3-difluorobenzonitrile

By using the method in Example 1, the yellow solid compound 34 (31.7 mg, 14.6%) was prepared by using 34-2 and I-10.

LC-MS (M−H)⁻=428.8. ¹H NMR (400 MHz, DMSO-d₆) δ 12.56 (s, 1H), 10.64 (s, 1H), 8.26 (s, 1H), 7.79-7.62 (m, 2H), 7.41-7.31 (m, 1H), 7.27-7.16 (m, 1H), 6.86 (s, 1H), 4.84 (s, 2H).

### Example 35

### Compound 35: (Z)-4-((4-((6-chloro-7-fluoro-1H-indol-3-yl)methylene)-2,5-dioxoimidazolidin-1-yl)-2,6-difluorobenzonitrile

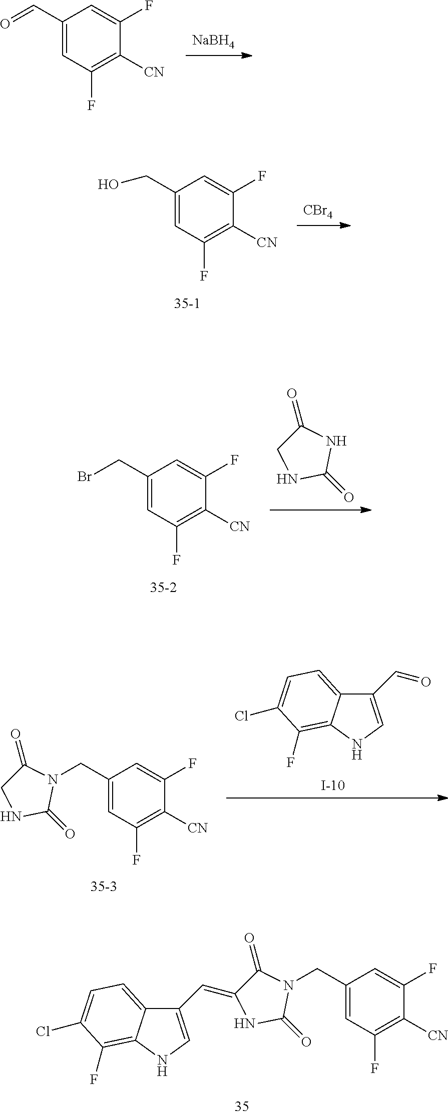

### 35-1: 2,6-difluoro-4-(hydroxymethyl)benzonitrile

At 0° C. and under nitrogen protection, sodium borohydride (257 mg, 6.458 mmol, 1.00 equiv) was added in batches into a solution of 2,6-difluoro-4-formyl benzonitrile (11.40 g, 6.458 mmol, 1.00 equiv) in methanol (25 mL), and the obtained mixture was stirred at 0° C. for 1 h. Water was added to terminate the reaction, the obtained mixture was extracted with EtOAc (3×100 mL), and combined organic layers were washed with saline (3×50 mL) and dried with anhydrous sodium sulfate. After the organic layers were filtered, a filtrate was concentrated at a reduced pressure. A residue was purified by silica gel column chromatography and eluted with CH₂Cl₂/EA (5:1) to obtain the yellow oily 35-1 (936 mg, 3.682 mmol, 84.7%).

LC-MS m/z (ESI): 168.0 [M−H]⁻

### 35-2: 4-(bromomethyl)-2,6-difluorobenzonitrile

At 0° C. and under nitrogen protection, carbon tetrabromide (2.30 g, 6.917 mmol, 1.30 equiv) was added in batches into a solution of 35-1 (936 mg, 5.534 mmol, 1.00 equiv) and PPI (1.33 g, 6.032 mmol, 1.10 equiv) in THF (33.0 mL), and the obtained mixture was stirred at room temperature for 20 h. The mixture was filtered, a reaction solution was extracted with EtOAc (3×50 mL), and combined organic layers were washed with saline (3×30 mL) and dried with anhydrous sodium sulfate. After the organic layers were filtered, a filtrate was concentrated at a reduced pressure. A residue was purified by silica gel column chromatography and eluted with PE/EA (5:1) to obtain the yellow oily 35-2 (218 mg, 0.930 mmol, 16.8%).

LC-MS m/z (ESI): 247.9 [M−H+18]⁻

### 35-3: Synthesis of 4-[(2,5-dioxoimidazolidin-1-yl)methyl]-2,6-difluorobenzonitrile

By using the method for 34-2, the white solid compound 35-3 (164 mg, 0.648 mmol, 75.9%) was prepared by using 35-2.

LC-MS m/z (ESI): 250.0 [M−H]⁻

### Compound 35: 4-{[(4Z)-4-[(6-chloro-7-fluoro-1H-indol-3-yl)methylene]-2,5-dioxoimidazolidin-1-yl]methyl}-2,6-difluorobenzonitrile

By using the method in Example 1, the yellow solid compound 35 (5.1 mg, 0.011 mmol, 2.95%) was prepared by using 35-3 and I-10.

LC-MS m/z (ESI): 428.8[M−H]⁻. ¹H NMR: (400 MHz, DMSO-d₆) δ 12.55 (s, 1H), 10.62 (s, 1H), 8.27 (s, 1H), 7.73-7.63 (m, 1H), 7.55-7.35 (m, 2H), 7.27-7.17 (m, 1H), 6.87 (s, 1H), 4.80 (s, 2H).

### Example 36

### Compound 36: (Z)-4-((4-((6-chloro-7-fluoro-1H-indol-3-yl)methylene)-2,5-dioxoimidazolidin-1-yl)-2,5-difluorobenzonitrile

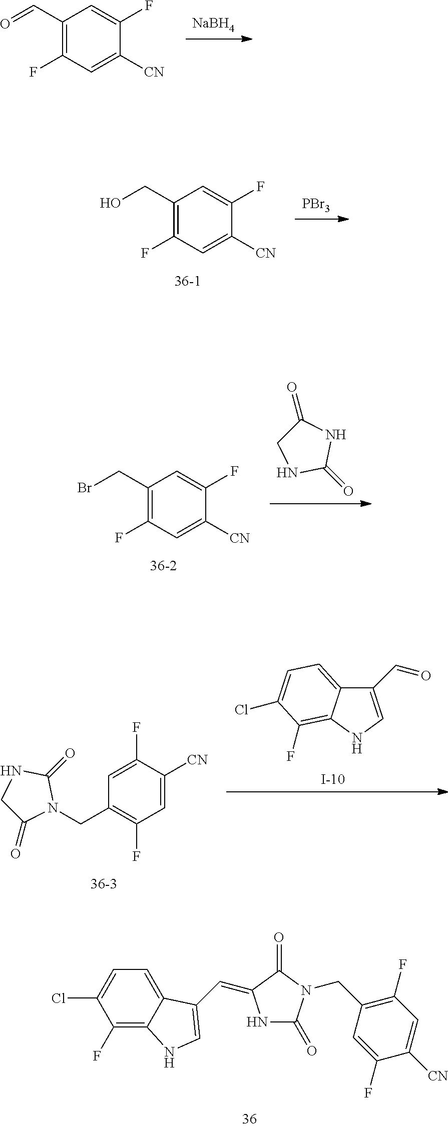

### 36-1: Synthesis of 2,5-difluoro-4-(hydroxymethyl)benzonitrile

By using the method for 35-1, the white solid compound 36-1 (950 mg, 5.611 mmol, 96.6%) was prepared by using 2,5-difluoro-4-formyl benzonitrile.

LC-MS m/z (ESI): 168.0 [M−H]⁻

### 36-2: 4-(bromomethyl)-2,5-difluorobenzonitrile

Under nitrogen protection, phosphorus tribromide (1.55 g, 5.804 mmol, 1.10 equiv) was dropwise added into a solution of 2,5-difluoro-4-(hydroxymethyl)benzonitrile (920 mg, 5.276 mmol, 1.00 equiv) in DCM (5 mL) at 0° C., and the obtained mixture was stirred at room temperature for 20 h. The reaction solution was diluted with DCM (50 mL) and extracted with sodium hydrogen carbonate (3×70 mL), and organic phases were dried with anhydrous sodium sulfate. After the organic phases were filtered, a filtrate was concentrated at a reduced pressure to obtain the white solid 36-2 (545 mg, 1.966 mmol, 40.3%).

LC-MS m/z (ESI): 232.00 [M+H]⁺

### 36-3: 4-[(2,5-dioxoimidazol-1-yl)methyl]-2,5-difluorobenzonitrile

By using the method for 34-2, the yellow solid compound 36-3 (183 mg, 0.626 mmol, 29.1%) was prepared by using 36-2.

LC-MS m/z (ESI): 250.0 [M−H]⁻

### Compound 36: 4-{[(4Z)-4-[(6-chloro-7-fluoro-1H-indol-3-yl)methylene]-2,5-dioxoimidazolidin-1-yl]methyl}-2,5-difluorobenzonitrile

By using the method in Example 1, the yellow solid compound 36 (18.6 mg, 0.041 mmol, 13.6%) was prepared by using the 36-3 and corresponding aldehyde.

LC-MS m/z (ESI): 428.8 [M−H]−. ¹H NMR: (400 MHz, CDCl₃) δ12.55 (s, 1H), 10.61 (s, 1H), 8.27 (s, 1H), 8.07-7.97 (m, 1H), 7.78-7.68 (m, 1H), 7.65-7.45 (m, 1H), 7.28-7.18 (m, 1H), 6.86 (s, 1H), 4.79 (s, 2H).

### Example 37

### Compound 37: (Z)-4-(1-(4-((6-chloro-7-fluoro-1H-indol-3-yl)methylene)-2,5-dioxoimidazolidin-1-yl)-2-hydroxyethyl)-2-fluorobenzonitrile

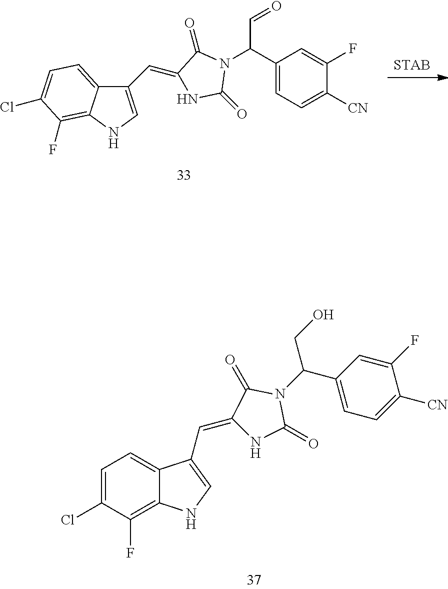

STAB (38 mg, 0.174 mmol, 2.63 equiv) was added in batches into a stirred solution of 4-{1-[(4Z)-4-[(6-chloro-7-fluoro-1H-indol-3-yl)methylene]-2,5-dioxoimidazolidin-1-yl]-2-oxyethyl}-2-fluorobenzonitrile (30 mg, 0.066 mmol, 1 equiv) and AcOH (10 mg, 0.153 mmol, 2.32 equiv) in DCE (1 mL) at 0° C., and the obtained mixture was stirred at 40° C. for 1 h. The mixture was cooled to room temperature and concentrated in vacuum. A crude product was purified by preparative HPLC to obtain the yellow solid compound 37 (6.9 mg, 0.012 mmol, 18.8%).

LC-MS m/z (ESI): 440.9 [M−H]⁻. ¹H NMR (400 MHz, DMSO-d₆) δ 12.54 (s, 1H), 10.61 (s, 1H), 8.31-8.21 (m, 1H), 7.98-7.88 (m, 1H), 7.73-7.63 (m, 1H), 7.65-7.55 (m, 1H), 7.50-7.40 (m, 1H), 7.27-7.17 (m, 1H), 6.84 (s, 1H), 5.35-5.25 (m, 2H), 4.33-4.23 (m, 1H), 4.09-3.99 (m, 1H).

### Example 38

### Compound 38: (Z)-4-(1-(4-((6-chloro-1H-indol-3-yl)methylene)-2,5-dioxoimidazolidin-1-yl)-2-hydroxyethyl)benzonitrile

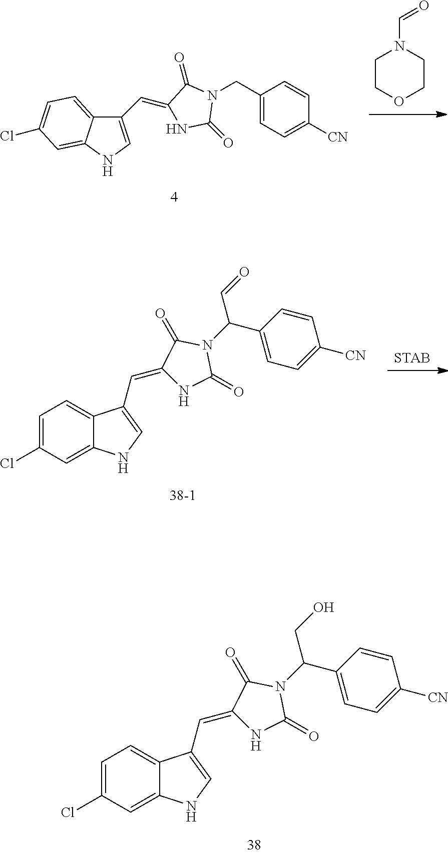

### 38-1: 4-{1-[(4Z)-4-[(6-chloro-1H-indol-3-yl)methylene]-2,5-dioxoimidazolidin-1-yl]-2-hydroxyethyl}benzonitrile

By using the method in Example 33, the yellow solid 38-1 (220 mg, 0.327 mmol, 9.7%) was prepared by using the compound 4.

LC-MS m/z (ESI): 403.1 [M−H]⁻

### Compound 38: (Z)-4-(1-(4-((6-chloro-1H-indol-3-yl)methylene)-2,5-dioxoimidazolidin-1-yl]-2-hydroxyethyl}benzonitrile

By using the method in Example 37, the yellow solid 38 (13.6 mg, 0.032 mmol, 40%) was prepared by using 38-1.

LC-MS m/z (ESI): 407.1 [M+H]⁺. ¹H NMR (300 MHz, DMSO-d₆) δ 11.97 (s, 1H), 10.51 (brs, 1H), 8.19 (s, 1H), 7.88-7.68 (m, 3H), 7.66-7.56 (m, 2H), 7.55-7.45 (m, 1H), 7.21-7.09 (m, 1H), 6.85 (s, 1H), 5.33-5.17 (m, 2H), 4.37-4.27 (m, 1H), 4.07-3.97 (m, 1H).

### Example 39

### Compound 39: (Z)-5-((6-chloro-7-methyl-1H-indol-3-yl)methylene)-3-(1-(3,4-difluorophenyl)-2-hydroxyethyl)imidazolidine-2,4-dione

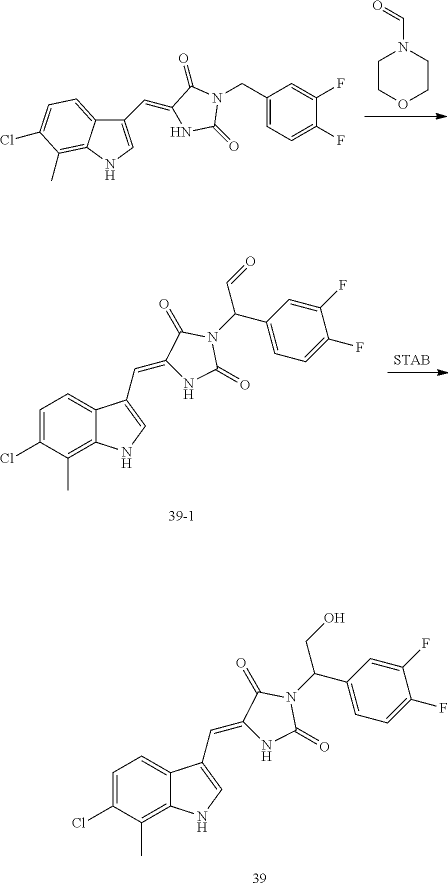

### 39-1: 2-[(4Z)-4-[(6-chloro-7-methyl-1H-indol-3-yl)methylene]-2,5-dioxoimidazolidin-1-yl]-2-(3,4-difluorophenyl)acetaldehyde

By using the method in Example 33, the yellow solid 39-1 (80 mg, 0.219 mmol, 12.7%) was prepared by using (Z)-5-((6-chloro-7-methyl-1H-indol-3-yl)methylene)-3-(3,4-difluorobenzyl)imidazolidine-2,4-dione.

LC-MS m/z (ESI): 430.0 [M+H]⁺

### Compound 39: (Z)-5-((6-chloro-7-methyl-1H-indol-3-yl)methylene)-3-(1-(3,4-difluorophenyl)-2-hydroxyethyl)imidazolidine-2,4-dione

By using the method in Example 37, the yellow solid 39 (8.5 mg, 0.019 mmol, 9.5%) was prepared by using 39-1.

LC-MS m/z (ESI): 430.0 [M−H]⁻. ¹H NMR (300 MHz, DMSO-d₆) δ 11.99 (s, 1H), 10.51 (s, 1H), 8.21 (s, 1H), 7.69-7.59 (m, 1H), 7.58-7.35 (m, 2H), 7.33-7.10 (m, 2H), 6.83 (s, 1H), 5.26-5.15 (m, 2H), 4.37-4.27 (m, 1H), 4.00-3.90 (m, 1H). 2.45 (s, 3H).

### Example 40

### Compound 40: (Z)-2-(4-((6-chloro-1H-indol-3-yl)methylene)-2,5-dioxoimidazolidin-1-yl)-2-(4-cyanophenyl)ethyl dihydrophosphate

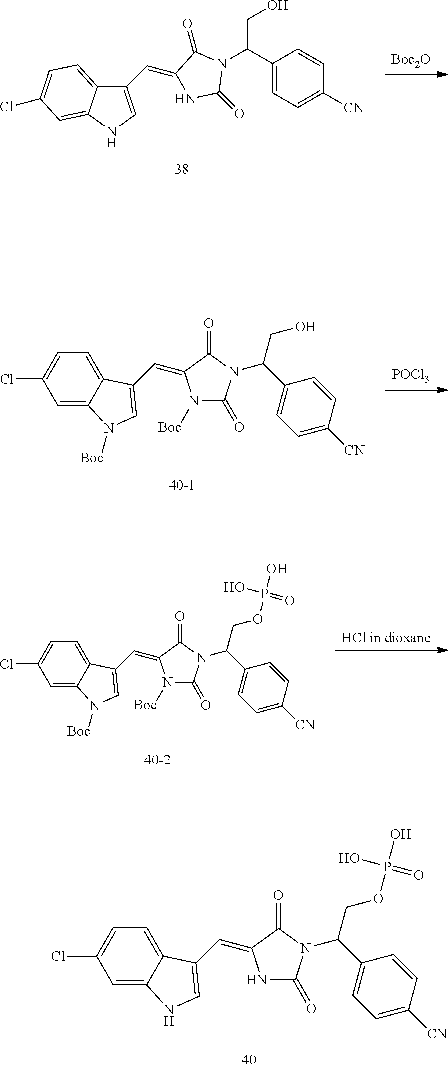

### 40-1: tert-butyl 3-{[(4Z)-3-(tert-butoxycarbonyl)-1-[1-(4-cyanophenyl)-2-hydroxyethyl]-2,5-dioxoimidazolidin-4-ylene]methyl}-6-chloroindole-1-carboxylate

DIEA (104 mg, 0.763 mmol, 2.12 equiv) was added into a stirred solution of 4-{1-[(4Z)-4-[(6-chloro-1H-indol-3-yl)methylene]-2,5-dioxoimidazolidin-1-yl]-2-hydroxyethyl}benzonitrile (155 mg, 0.360 mmol, 1 equiv), DMAP (9 mg, 0.068 mmol, 0.19 equiv), and Boc₂O (243 mg, 1.058 mmol, 2.94 equiv) in DMF (3 mL), and the obtained mixture was stirred in a nitrogen atmosphere at 40° C. for 2 h. The mixture was cooled to room temperature and concentrated in vacuum. A residue was purified by fast reversed-phase chromatography to obtain the yellow solid 40-1 (20 mg, 0.033 mmol, 9.13%).

LC-MS m/z (ESI): 505.1 [M−H−100]⁻

### 40-2: 2-[(4Z)-3-(tert-butoxycarbonyl)-4-{[1-(tert-butoxycarbonyl)-6-chloroindol-3-yl]methylene}-2,5-dioxoimidazolidin-1-yl]-2-(4-cyanophenyl)ethoxyl phosphonic acid

Phosphorus oxychloride (125 mg, 0.772 mmol, 0.16 mL, 18.83 equiv) was added into a stirred solution of tert-butyl 3-{[(4Z)-3-(tert-butoxycarbonyl)-1-[1-(4-cyanophenyl)-2-hydroxyethyl]-2,5-dioxoimidazolidin-4-ylene]methyl}-6-chloroindol-1-carboxylate (25 mg, 0.041 mmol, 1 equiv) and TEA (150 mg, 1.403 mmol, 0.20 MI, 34.23 equiv) in THF (1 mL) at 0° C., and the obtained mixture was stirred at room temperature for 18 h. Then, water (0.5 mL) was added into a reaction solution at 0° C., and the obtained mixture was stirred at room temperature for 2 h. The obtained mixture was concentrated in vacuum to obtain the yellow solid 40-2 (40 mg, crude product). The crude product was directly used for a next step without further purification.

LC-MS m/z (ESI): 685.1 [M−H]⁻

### Compound 40: 2-[(4Z)-4-[(6-chloro-1H-indol-3-yl)methylene]-2,5-dioxoimidazolidin-1-yl]-2-(4-cyanophenyl)ethoxyl phosphonic acid

A 1,4-dioxane (1 mL, 4.000 mmol, 4 N) in HCl was added into 2-[(4Z)-3-(tert-butoxycarbonyl)-4-{[1-(tert-butoxycarbonyl)-6-chloroindol-3-yl]methylene}-2,5-dioxoimidazol-1-yl]-2-(4-cyanophenyl)ethoxyl phosphonic acid (40 mg, crude product), and the obtained mixture was stirred at room temperature for 12 h. The obtained mixture was concentrated in vacuum. The crude product was purified by reversed-phase HPLC to obtain the white solid compound 40 (2.2 mg, 0.004 mmol, 29.11%).

LC-MS m/z (ESI): 484.9 [M−H]⁻.

¹H NMR (300 MHz, DMSO-d₆) δ 8.20 (s, 1H), 7.85-7.75 (m, 3H), 7.71-7.57 (m, 2H), 7.54-7.44 (m, 1H), 7.13 (d, J=8.5 Hz, 1H), 6.82 (s, 1H), 5.47-5.37 (m, 1H), 4.46-4.36 (m, 2H).

### Example 41

### Compound 41: (Z)-2-(4-((6-chloro-7-fluoro-1H-indol-3-yl)methylene)-2,5-dioxoimidazolidin-1-yl)-2-(4-cyano-3-fluorophenyl)-N-(2-hydroxyethyl)acetamide

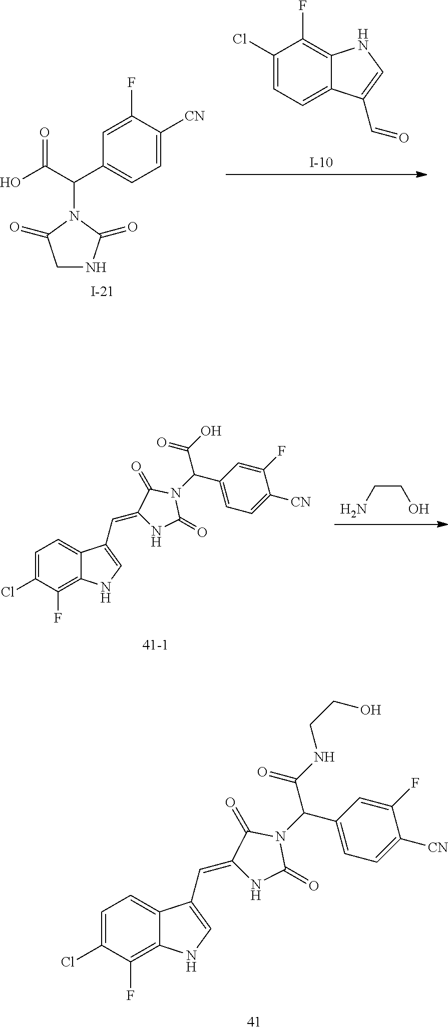

### 41-1: (Z)-2-(4-((6-chloro-7-fluoro-1H-indol-3-yl)methylene)-2,5-dioxoimidazolidin-1-yl)-2-(4-cyano-3-fluorophenyl)acetic acid

Pyridine (341 mg, 4.100 mmol, 4.0 equiv, 95%) was added in batches into a solution of methyl 2-(4-cyano-3-fluorophenyl)-2-(2,5-dioxoimidazolidin-1-yl)acetate (300 mg, 1.020 mmol, 1.0 equiv, 99%) and 6-chloro-7-fluoro-1H-indole-3-formaldehyde (409 mg, 2.050 mmol, 2.0 equiv, 99%) in ethanol (5 mL) at room temperature, and the obtained mixture was stirred under nitrogen protection at 60° C. for 4 h. A residue was purified by fast reversed-phase chromatography to obtain the yellow solid compound 41-1 (120 mg, 23%).

LC-MS (M−H)⁻=455.0.

### Compound 41: (Z)-2-(4-((6-chloro-7-fluoro-1H-indol-3-yl)methylene)-2,5-dioxoimidazol-1-yl)-2-(4-cyano-3-fluorophenyl)-N-(2-hydroxyethyl)acetamide

Cholamine (27 mg, 0.420 mmol, 9.80 equiv) was dropwise added into a stirred solution of [(4Z)-4-[(6-chloro-7-fluoro-1H-indol-3-yl)methylene]-2,5-dioxoimidazolidin-1-yl](4-cyano-3-fluorophenyl)acetic acid (20 mg, 0.043 mmol, 1 equiv), HATU (30 mg, 0.075 mmol, 1.75 equiv), and DIEA (27 mg, 0.198 mmol, 4.63 equiv) in DMF (0.5 mL), and the obtained mixture was stirred under nitrogen protection at 40° C. for 12 h. The mixture was cooled to room temperature and purified by Prep-HPLC to obtain the yellow solid compound 41 (1.6 mg, 0.003 mmol, 7.55%).

LC-MS m/z (ESI): 500.1 [M+H]⁺.

¹H NMR (400 MHz, DMSO-d₆) δ 12.56 (s, 1H), 10.64 (s, 1H), 8.29-8.18 (m, 2H), 7.97-7.89 (m, 1H), 7.69 (d, J=8.6 Hz, 1H), 7.59 (d, J=10.6 Hz, 1H), 7.47 (d, J=8.6 Hz, 1H), 7.28-7.17 (m, 1H), 6.84 (s, 1H), 5.89 (s, 1H), 4.67-4.60 (m, 1H), 3.46-3.39 (m, 2H), 3.26-3.19 (m, 1H), 3.18-3.09 (m, 1H).

### Example 42

### Compound 42: (Z)-2-(4-((6-chloro-1H-indol-3-yl)methylene)-2,5-dioxoimidazolidin-1-yl)-2-(3,4-difluorophenyl)ethyl dihydrophosphate

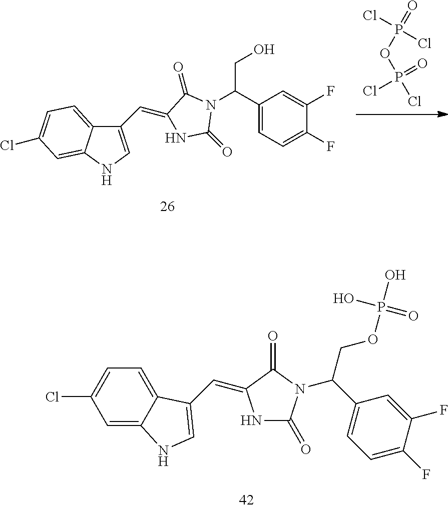 [SMILES: O=C1N/C(=C\c2c[nH]c3cc(Cl)ccc23)C(=O)N1C(CO)c1ccc(F)c(F)c1 ; O=P(Cl)(Cl)OP(=O)(Cl)Cl ; "26" ; O=C1N/C(=C\c2c[nH]c3cc(Cl)ccc23)C(=O)N1C(COP(=O)(O)O)c1ccc(F)c(F)c1]

Dichlorophosphonic anhydride (35 mg, 0.14 mmol, 3.91 eq) was dropwise added into a stirred solution of (5Z)-5-[(6-chloro-1H-indol-3-yl)methylene]-3-[1-(3,4-difluorophenyl)-2-hydroxyethyl]imidazolidine-2,4-dione (15 mg, 0.036 mmol, 1 eq) in ACN (1 mL) at 0° C., and the obtained mixture was then stirred at 0° C. for 3 h. The mixture was alkalized with sodium hydrogen carbonate (1 M) at 0° C. till the pH=8-9, and the mixture was stirred at 0° C. for 30 min. Then, the mixture was acidized with HCl (12 M) till pH=1, and the mixture was stirred at 0° C. for 1.5 h. The obtained mixture was extracted with EtOAc (3×25 mL), and combined organic layers were washed with saline (1×25 mL) and dried with anhydrous sodium sulfate. After the organic layers were filtered, a filtrate was concentrated at a reduced pressure. The product was purified by Prep-HPLC to obtain the yellow solid compound 42 (4.4 mg, 23.0%).

LC-MS m/z (ESI): 496.0 [M−H]⁻. ¹H NMR (300 MHz, DMSO-d₆) δ 12.09 (s, 1H), 8.20 (s, 1H), 7.79 (d, J=8.5 Hz, 1H), 7.60-7.20 (m, 4H), 7.19-7.05 (m, 1H), 6.84 (s, 1H), 5.42-5.28 (m, 1H), 4.60-4.30 (m, 2H).

### Example 43

### Compound 43: (Z)-2-(4-((6-chloro-7-methyl-1H-indol-3-yl)methylene)-2,5-dioxoimidazolidin-1-yl)-2-(3,4-difluorophenyl)ethyl dihydrophosphate

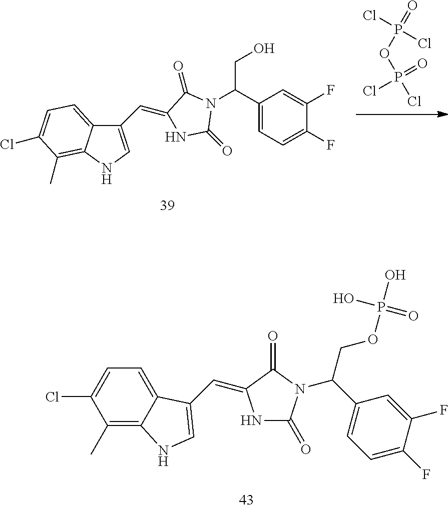 [SMILES: Cc1c(Cl)ccc2c(/C=C3\NC(=O)N(C(CO)c4ccc(F)c(F)c4)C3=O)c[nH]c12 ; O=P(Cl)(Cl)OP(=O)(Cl)Cl ; "39" ; Cc1c(Cl)ccc2c(/C=C3\NC(=O)N(C(COP(=O)(O)O)c4ccc(F)c(F)c4)C3=O)c[nH]c12]

By using the method in Example 42, the yellow solid compound 43 (10.9 mg, 17.4%) was prepared by using the compound 39.

LC-MS m/z (ESI): 510.1 [M−H]⁻. ¹H NMR (300 MHz, DMSO-d₆) δ 12.09 (s, 1H), 8.21 (s, 1H), 7.62 (d, J=8.5 Hz, 1H), 7.57-7.32 (m, 2H), 7.30-7.21 (m, 1H), 7.14 (d, J=8.5 Hz, 1H), 6.82 (s, 1H), 5.40-5.29 (m, 1H), 4.56-4.25 (m, 2H), 2.54 (s, 3H).

### Example 44

### Compound 44: (Z)-2-(2-(4-((6-chloro-7-methyl-1H-indol-3-yl)methylene)-2,5-dioxoimidazolidin-1-yl)-2-(3,4-difluorophenyl)acetamido)dihydroethyl phosphate

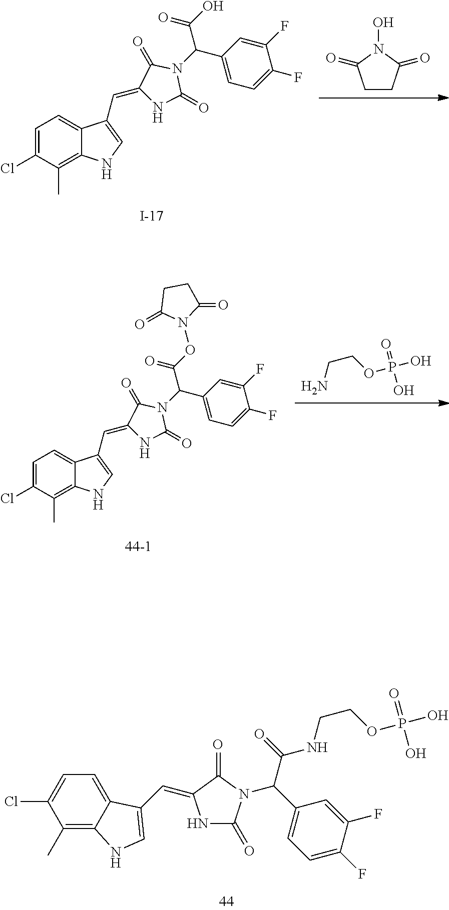

### 44-1:2,5-dioxopyrrolidin-1-yl(z)-2-(4-((6-chloro-7-methyl-1H-indol-3-yl)methylene)-2,5-dioxoimidazolidin-1-yl)-2-(3,4-difluorophenyl)acetate

Under nitrogen protection at 0° C., DCC (30 mg, 0.138 mmol) was added into a stirred solution of [(4Z)-4-[(6-chloro-7-methyl-1H-indol-3-yl)methylene]-2,5-dioxoimidazolidin 1-yl](3,4-difluorophenyl)acetic acid (60 mg, 0.128 mmol, 1 equiv) and N-hydroxysuccinimide (16 mg, 0.132 mmol, 1.03 equiv, 95%) in DCM (2 mL), and the obtained mixture was stirred at room temperature for 12 h. The reaction was terminated with ice water. The obtained mixture was extracted with DCM (3×10 mL). Combined organic layers were washed with saline (3×5 mL) and dried with anhydrous sodium sulfate. After the organic layers were filtered, a filtrate was concentrated at a reduced pressure to obtain the yellow solid 44-1 (100 mg, 99.12%). The crude product was directly used for a next step without further purification.

LC-MS m/z (ESI): 541.0 [M−H]⁻.

### Compound 44: (Z)-2-(2-(4-((6-chloro-7-methyl-1H-indol-3-yl)methylene)-2,5-dioxoimidazolidin-1-yl)-2-(3,4-difluorophenyl)acetamido)dihydroethyl phosphate

Under nitrogen protection at 0° C., TEA (33 mg, 0.308 mmol, 2.37 equiv) was added into a stirred solution of 2,5-pyrrolidin-1-yl-2-[(4Z)-4-[(6-chloro-1H-indol-3-yl)methylene]-2,5-dioxoimidazolidin-1-yl]-2-(3,4-difluorophenyl)acetate (100 mg, 0.130 mmol, 1 equiv) and O-phosphorylethanolamine (33 mg, 0.221 mmol, 1.70 equiv) in DMF (3 mL) and water (2 mL), and the obtained mixture was stirred at room temperature for 16 h. The obtained mixture was concentrated in vacuum. The crude product was purified by Prep-HPLC to obtain the yellow solid compound 44 (17.3 mg, 19.85%).

LC-MS m/z (ESI): 569.1 [M+H]⁺. ¹H NMR (300 MHz, DMSO-d₆) δ 12.02 (s, 1H), 8.52-8.46 (m, 1H), 8.21 (s, 1H), 7.63 (d, J=8.5 Hz, 1H), 7.55-7.21 (m, 3H), 7.14 (d, J=8.5 Hz, 1H), 6.82 (s, 1H), 5.75 (s, 1H), 3.75-3.69 (m, 2H), 3.27-3.08 (m, 2H), 2.51 (s, 3H).

### Example 45

### Compound 45: (Z)-5-((6-chloro-1H-indol-3-yl)methylene)-3-(1-(4-chlorophenyl)-2-hydroxyethyl)imidazolidine-2,4-dione

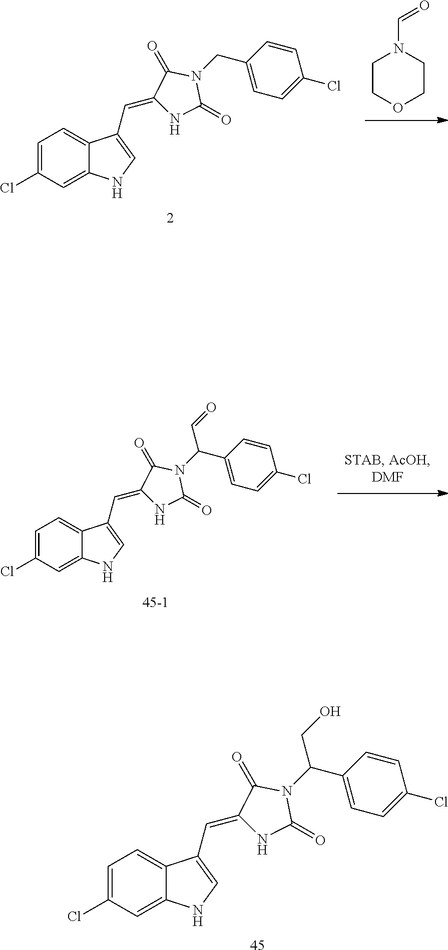

### 45-1: (Z)-2-(4-((6-chloro-1H-indol-3-yl)methylene)-2,5-dioxoimidazolidin-1-yl)-2-(4-chlorophenyl)acetaldehyde

By using the method for the compound 33, the yellow solid compound 45-1 (205 mg, 38.2%) was prepared by using the compound 2.

LC-MS m/z (ESI): 411.9 [M−H]⁻.

### Compound 45: (Z)-5-((6-chloro-1H-indol-3-yl)methylene)-3-(1-(4-chlorophenyl)-2-hydroxyethyl)imidazolidine-2,4-dione

By using the method for the compound 37, the yellow solid compound 45 (8.9 mg, 40.3%) was prepared by using the compound 45-1.

LC-MS m/z (ESI): 413.9 [M−H]⁻. ¹H NMR (300 MHz, DMSO-d₆) δ 11.96 (s, 1H), 10.47 (s, 1H), 8.18 (s, 1H), 7.81 (d, J=8.5 Hz, 1H), 7.54-7.40 (m, 5H), 7.13 (d, J=8.5 Hz, 1H), 6.83 (s, 1H), 5.24-5.13 (m, 2H), 4.42-4.28 (m, 1H), 4.01-3.87 (m, 1H).

### Example 46

### Compound 46: (Z)-2-(4-((6-chloro-1H-indol-3-yl)methylene)-2,5-dioxoimidazolidin-1-yl)-2-(4-chlorophenyl)ethyl dihydrophosphate

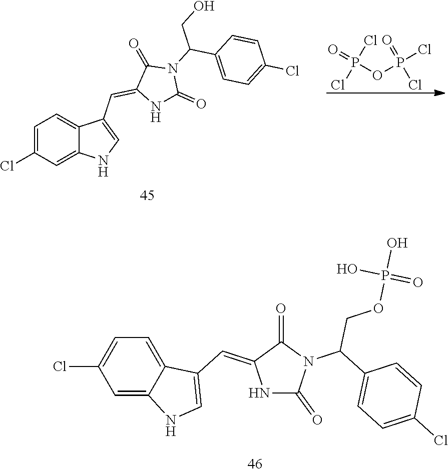 [SMILES: O=C1N/C(=C\c2c[nH]c3cc(Cl)ccc23)C(=O)N1C(CO)c1ccc(Cl)cc1 ; O=P(Cl)(Cl)OP(=O)(Cl)Cl ; O=C1N/C(=C\c2c[nH]c3cc(Cl)ccc23)C(=O)N1C(COP(=O)(O)O)c1ccc(Cl)cc1]

By using the method for the compound 42, the yellow solid compound 46 (13.3 mg, 31.6%) was prepared by using the compound 45.

LC-MS m/z (ESI): 496.0 [M+H]⁺. ¹H NMR (400 MHz, DMSO-d₆) δ 12.13 (s, 1H), 8.21 (s, 1H), 7.80 (d, J=8.5 Hz, 1H), 7.51-7.37 (m, 5H), 7.13 (d, J=8.5 Hz, 1H), 6.83 (s, 1H), 5.40-5.30 (m, 1H), 4.56-4.47 (m, 1H), 4.39-4.27 (m, 1H).

### Example 47

### Compound 47: (Z)-2-(4-((6-chloro-7-fluoro-1H-indol-3-yl)methylene)-2,5-dioxoimidazolidin-1-yl)-2-(4-cyano-3-fluorophenyl)ethyl dihydrophosphate

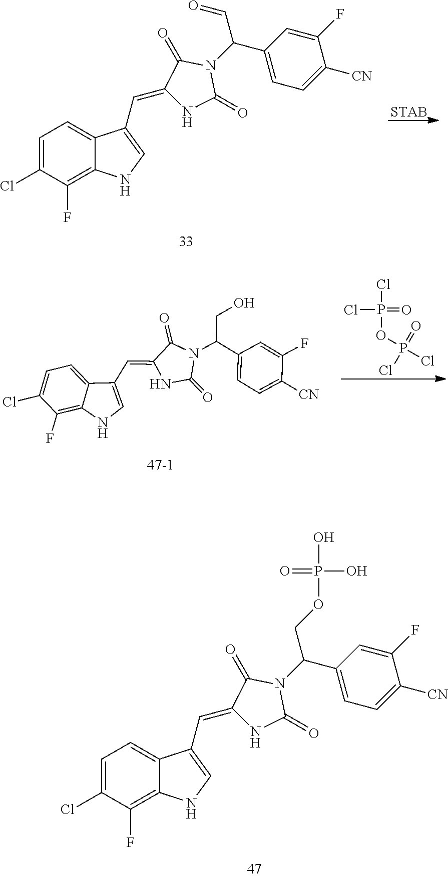

### 47-1: (Z)-4-(1-(4-((6-chloro-7-fluoro-1H-indol-3-yl)methylene)-2,5-dioxoimidazolidin-1-yl)-2-hydroxyethyl)-2-fluorobenzonitrile

By using the method for the compound 37, the yellow solid compound 47-1 (19 mg, 39.4%) was prepared by using the compound 33.

LC-MS m/z (ESI): 441.0[M−H]⁻

### Compound 47: (Z)-2-(4-((6-chloro-7-fluoro-1H-indol-3-yl)methylene)-2,5-dioxoimidazolidin-1-yl)-2-(4-cyano-3-fluorophenyl)ethyl dihydrophosphate

By using the method for the compound 42, the yellow solid compound 44 (2.8 mg, 17.8%) was prepared by using the compound 47-1.

LC-MS m/z (ESI): 521.0[M−H]⁻. ¹HNMR (400 MHz, DMSO-d₆) δ 12.57 (s, 1H), 10.67 (s, 1H), 8.26 (s, 1H), 7.98-7.90 (m, 1H), 7.69-7.55 (m, 2H), 7.53-7.40 (m, 1H), 7.25-7.17 (m, 1H), 6.84 (s, 1H), 5.57-5.45 (m, 1H), 4.65-4.46 (m, 2H), 3.40-3.30 (s, 2H).

### Example 48

### Compound 48: (Z)-2-(4-((6-chloro-7-methyl-1H-indol-3-yl)methylene)-2,5-dioxoimidazolidin-1-yl)-2-(4-chlorophenyl)-N-(2-hydroxyethyl)acetamide

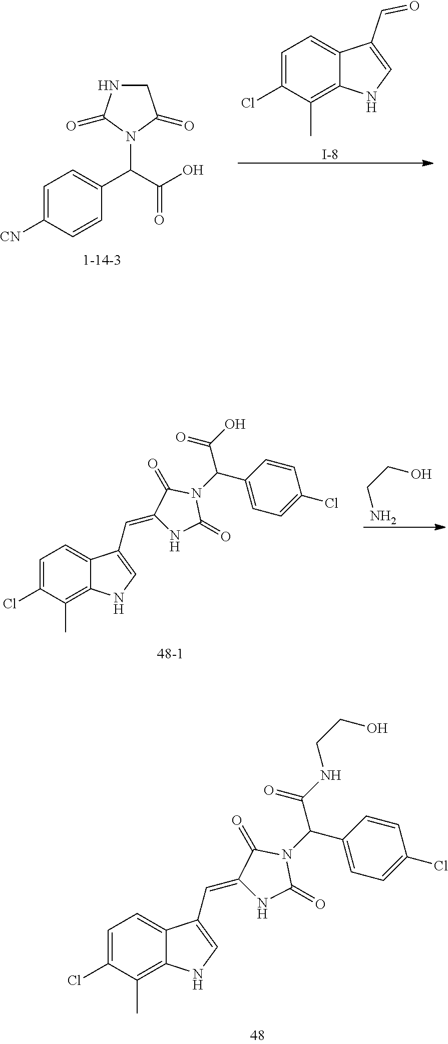

### 48-1: (Z)-2-(4-((6-chloro-7-methyl-1H-indol-3-yl)methylene)-2,5-dioxoimidazolidin-1-yl)-2-(4-chlorophenyl)acetic acid

By using the method for the 1-19, the yellow solid compound 48-1 (16 g, 52%) was prepared by using I-14-3 and I-8.

LC-MS m/z (ESI): 442.1.0[M−H]⁻.

### Compound 48: (Z)-2-(4-((6-chloro-7-methyl-1H-indol-3-yl)methylene)-2,5-dioxoimidazolidin-1-yl)-2-(4-chlorophenyl)-N-(2-hydroxyethyl)acetamide

By using the method for the compound 41, the yellow solid 48 (10 g, 57%) was prepared by using 48-1.

LC-MS m/z (ESI): 485.1 [M−H]⁻. ¹H NMR (400 MHz, DMSO-d6) δ 12.00 (s, 1H), 10.52 (s, 1H), 8.20 (s, 1H), 8.07-8.02 (m, 1H), 7.64-7.62 (m, 1H), 7.48-7.40 (m, 4H), 7.20-7.13 (m, 1H), 6.82 (s, 1H), 5.76-5.73 (m, 1H), 4.63-4.55 (m, 1H), 3.44-3.30 (m, 3H), 3.25-3.12 (m, 3H).

### Example 49

### Compound 49: (R,Z)-4-(1-(4-((6-chloro-1H-indol-3-yl)methylene)-2,5-dioxoimidazolidin-1-yl)-2-hydroxyethyl)benzonitrile

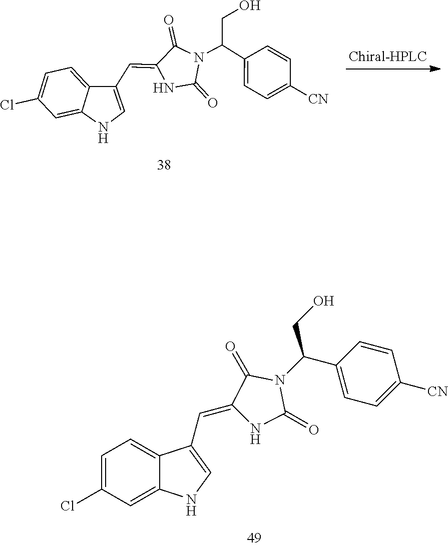 [SMILES: N#Cc1ccc(C(CO)N2C(=O)N/C(=C\c3c[nH]c4cc(Cl)ccc34)C2=O)cc1 ; N#Cc1ccc([C@H](CO)N2C(=O)N/C(=C\c3c[nH]c4cc(Cl)ccc34)C2=O)cc1]

The compound 38 was separated by chiral SFC according to the following condition to obtain the compound 49: column: CHIRALPAK ID-3 4.6×50 mm 3 um; mobile phase: Hex (0.1% DEA): EtOH=50:50, 20 min; detector, UV 254 nm.

LC-MS m/z (ESI): 404.8 [M−H]⁻. ¹H NMR (400 MHz, DMSO-d₆) δ 11.98 (s, 1H), 10.53 (s, 1H), 8.20 (s, 1H), 7.88-7.79 (m, 3H), 7.64-7.58 (m, 2H), 7.52-7.47 (m, 1H), 7.20-7.11 (m, 1H), 6.85 (s, 1H), 5.32-5.23 (m, 2H), 4.38-4.28 (m, 1H), 4.07-3.97 (m, 1H).

### Example 50

### Compound 50: (S,Z)-4-(1-(4-((6-chloro-1H-indol-3-yl)methylene)-2,5-dioxoimidazolidin-1-yl)-2-hydroxyethyl)benzonitrile

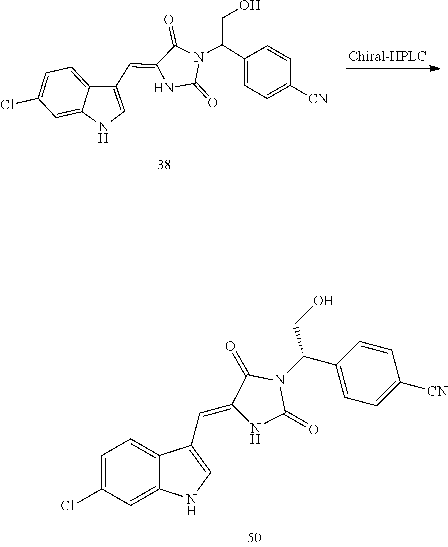 [SMILES: N#Cc1ccc(C(CO)N2C(=O)N/C(=C\c3c[nH]c4cc(Cl)ccc34)C2=O)cc1 ; N#Cc1ccc([C@@H](CO)N2C(=O)N/C(=C\c3c[nH]c4cc(Cl)ccc34)C2=O)cc1]

The compound 38 was separated by chiral SFC according to the following condition to obtain the compound 50: column: CHIRALPAK ID-3 4.6×50 mm 3 um; mobile phase: Hex (0.1% DEA): EtOH=50:50, 20 min; detector, UV 254 nm.

LC-MS m/z (ESI): 404.8 [M−H]⁻. ¹H NMR (400 MHz, DMSO-d₆) δ 11.99 (s, 1H), 10.53 (s, 1H), 8.20 (s, 1H), 7.89-7.79 (m, 3H), 7.63-7.59 (m, 2H), 7.50-7.46 (m, 1H), 7.22-7.11 (m, 1H), 6.86 (s, 1H), 5.32-5.24 (m, 2H), 4.39-4.28 (m, 1H), 4.07-3.98 (m, 1H).

### Example 51

### Compound 51: (R,Z)-2-(4-((6-chloro-1H-indol-3-yl)methylene)-2,5-dioxoimidazolidin-1-yl)-2-(4-cyanophenyl)ethyl dihydrophosphate

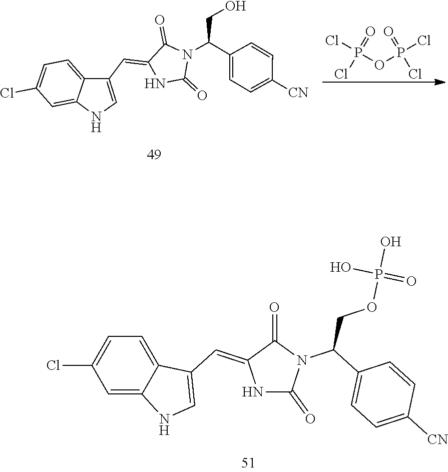 [SMILES: N#Cc1ccc([C@H](CO)N2C(=O)N/C(=C\c3c[nH]c4cc(Cl)ccc34)C2=O)cc1 ; O=P(Cl)(Cl)OP(=O)(Cl)Cl ; N#Cc1ccc([C@H](COP(=O)(O)O)N2C(=O)N/C(=C\c3c[nH]c4cc(Cl)ccc34)C2=O)cc1]

By using the method for the compound 42, the yellow solid compound 51 (17.3 mg, 53.0%) was prepared by using the compound 49.

LC-MS m/z (ESI): 484.9 [M−H]⁻. ¹H NMR (300 MHz, DMSO-d₆) δ 12.20 (s, 1H), 8.21 (s, 1H), 7.85-7.75 (m, 3H), 7.72-7.57 (m, 2H), 7.54-7.46 (m, 1H), 7.17-7.08 (m, 1H), 6.84 (s, 1H), 5.49-5.38 (m, 1H), 4.59-4.33 (m, 2H).

### Example 52

### Compound 52: (S,Z)-2-(4-((6-chloro-1H-indol-3-yl)methylene)-2,5-dioxoimidazolidin-1-yl)-2-(4-cyanophenyl)ethyl dihydrophosphate

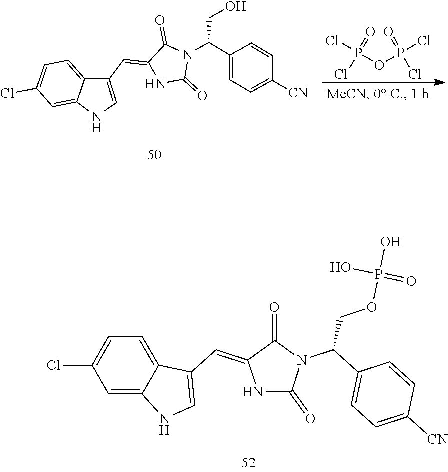 [SMILES: N#Cc1ccc([C@@H](CO)N2C(=O)N/C(=C\c3c[nH]c4cc(Cl)ccc34)C2=O)cc1 ; O=P(Cl)(Cl)OP(=O)(Cl)Cl ; N#Cc1ccc([C@@H](COP(=O)(O)O)N2C(=O)N/C(=C\c3c[nH]c4cc(Cl)ccc34)C2=O)cc1]

By using the method for the compound 42, the yellow solid compound 52 (17.9 mg, 59.1%) was prepared by using the compound 50.

LC-MS m/z (ESI): 487.0 [M+H]⁺. ¹H NMR (300 MHz, DMSO-d₆) δ 12.19 (s, 1H), 8.21 (s, 1H), 7.85-7.75 (m, 3H), 7.72-7.57 (m, 2H), 7.54-7.46 (m, 1H), 7.17-7.07 (m, 1H), 6.84 (s, 1H), 5.49-5.38 (m, 1H), 4.52-4.34 (m, 2H).

### Example 53

### Compound 53: (S,Z)-5-((6-chloro-1H-indol-3-yl)methylene)-3-(1-(3,4-difluorophenyl)-2-hydroxyethyl)imidazolidine-2,4-dione

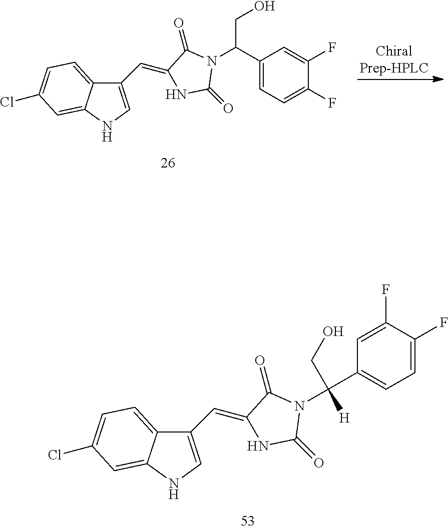 [SMILES: O=C1N/C(=C\c2c[nH]c3cc(Cl)ccc23)C(=O)N1C(CO)c1ccc(F)c(F)c1 ; "ChiralPrep-HPLC" ; O=C1N/C(=C\c2c[nH]c3cc(Cl)ccc23)C(=O)N1[C@H](CO)c1ccc(F)c(F)c1]

The compound 26 was separated by chiral HPLC according to the following condition to obtain the compound 53: CHIRALPAK IG, 2×25 cm; mobile phase, Hex (0.5% 2M NH3-MeOH) in MeOH, 50% isocratic in 15 min; detector, UV 254 nm.

LC-MS m/z (ESI): 415.8 [M−H]⁻. ¹H NMR (400 MHz, DMSO-d₆) δ 11.96 (s, 1H), 10.49 (s, 1H), 8.19 (s, 1H), 7.82 (d, J=8.5 Hz, 1H), 7.57-7.47 (m, 2H), 7.47-7.37 (m, 1H), 7.33-7.23 (m, 1H), 7.19-7.09 (m, 1H), 6.83 (s, 1H), 5.25-5.15 (m, 2H), 4.36-4.26 (m, 1H), 3.99-3.89 (m, 1H).

### Example 54

### Compound 54: (R,Z)-5-((6-chloro-1H-indol-3-yl)methylene)-3-(1-(3,4-difluorophenyl)-2-hydroxyethyl)imidazolidine-2,4-dione

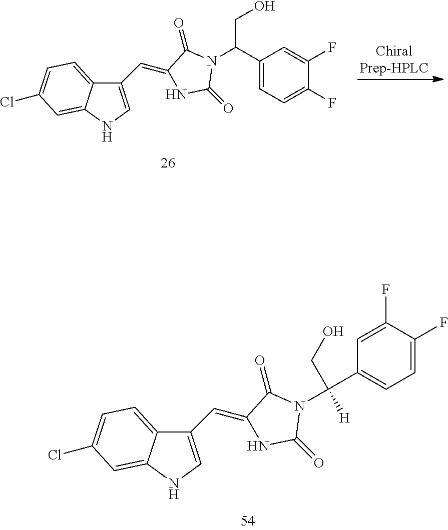 [SMILES: O=C1N/C(=C\c2c[nH]c3cc(Cl)ccc23)C(=O)N1C(CO)c1ccc(F)c(F)c1 ; "ChiralPrep-HPLC" ; O=C1N/C(=C\c2c[nH]c3cc(Cl)ccc23)C(=O)N1[C@@H](CO)c1ccc(F)c(F)c1]

The compound 26 was separated by chiral HPLC according to the following condition to obtain the compound 54: CHIRALPAK IG, 2×25 cm; mobile phase, Hex (0.5% 2M NH3-MeOH) in MeOH, 50% isocratic in 15 min; detector, UV 254 nm.

LC-MS m/z (ESI): 415.8 [M−H]⁻. ¹H NMR (400 MHz, DMSO-d6) δ 11.96 (s, 1H), 10.49 (s, 1H), 8.19 (s, 1H), 7.82 (d, J=8.5 Hz, 1H), 7.57-7.37 (m, 3H), 7.33-7.23 (m, 1H), 7.19-7.09 (m, 1H), 6.83 (s, 1H), 5.25-5.15 (m, 2H), 4.36-4.26 (m, 1H), 3.99-3.89 (m, 1H).

### Example 55

### Compound 55: (Z)-(6-chloro-3-((1-(3,4-difluorobenzyl)-2,5-dioxoimidazolidin-4-ylene)methyl)-1H-indol-1-yl)phosphonic acid

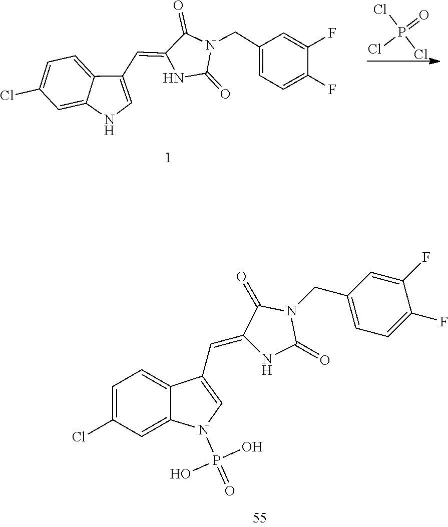 [SMILES: O=C1N/C(=C\c2c[nH]c3cc(Cl)ccc23)C(=O)N1Cc1ccc(F)c(F)c1 ; O=P(Cl)(Cl)Cl ; O=C1N/C(=C\c2cn(P(=O)(O)O)c3cc(Cl)ccc23)C(=O)N1Cc1ccc(F)c(F)c1]

NaH (68 mg, 2.84 mmol, 2.20 eq) was added into a solution of the compound 1 (500 mg, 1.29 mmol, 1.00 eq) in THF (10 mL) and DCM (20 mL), and the mixture was stirred under argon protection at room temperature for 15 min. Phosphorus oxychloride (988 mg, 6.44 mmol, 5.00 eq) was dropwise added into the mixture at 0° C., and the mixture was stirred under argon protection at 0° C. for 3 h. The obtained mixture was concentrated at a reduced pressure, and a residue was dissolved in acetonitrile (10 mL). The mixture was alkalized with cold saturated sodium hydrogen carbonate till pH=8, and the obtained mixture was stirred at 0° C. for 30 min. The mixture was acidized with concentrated hydrochloric acid till pH=1, and the obtained mixture was stirred at 0° C. for 30 min and extracted with EtOAc (3×30 mL). Combined organic layers were dried with anhydrous sodium sulfate. After the organic layers were filtered, a filtrate was concentrated at a reduced pressure. A crude product was purified by Prep-HPLC to obtain the yellow solid compound 55 (20.1 mg, 3.10%).

LC-MS m/z (ESI): 466.0[M−H]⁻. ¹H NMR (300 MHz, DMSO-d₆) δ 8.36 (s, 1H), 8.01 (s, 1H), 7.75-7.55 (m, 1H), 7.54-7.29 (m, 2H), 7.28-7.00 (m, 2H), 6.72 (s, 1H), 4.70 (s, 2H).

### Example 56

### Compound 56: (Z)-(6-chloro-3-((1-(1-(4-cyano-3-fluorophenyl)-2-((2-hydroxyethyl)amino)-2-oxyethyl)-2,5-dioxoimidazolidin-4-ylene)methyl)-7-fluoro-1H-indol-1-yl)phosphonic acid

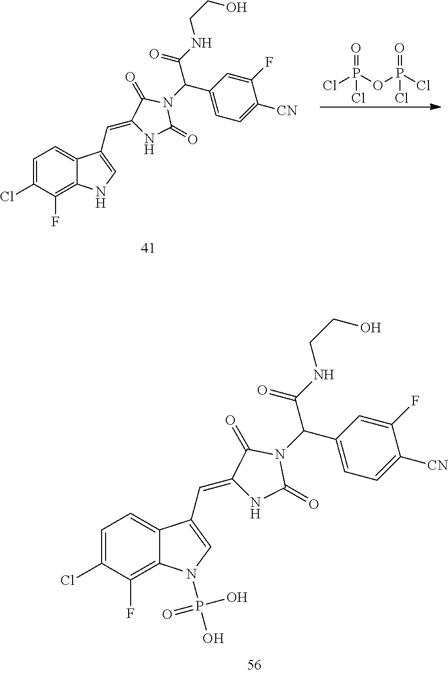 [SMILES: N#Cc1ccc(C(C(=O)NCCO)N2C(=O)N/C(=C\c3c[nH]c4c(F)c(Cl)ccc34)C2=O)cc1F ; O=P(Cl)(Cl)OP(=O)(Cl)Cl ; N#Cc1ccc(C(C(=O)NCCO)N2C(=O)N/C(=C\c3cn(P(=O)(O)O)c4c(F)c(Cl)ccc34)C2=O)cc1F]

By using the method for the compound 42, the yellow solid compound 56 (6.1 mg, 15.6%) was prepared by using the compound 41.

LC-MS m/z (ESI): 578.2 [M−H]⁻. ¹H NMR (300 MHz, DMSO-d₆) δ 8.19 (s, 1H), 7.85-7.75 (m, 1H), 7.59 (d, J=8.6 Hz, 1H), 7.49 (d, J=10.4 Hz, 1H), 7.39 (d, J=8.1 Hz, 1H), 7.20-7.09 (m, 1H), 6.76 (s, 1H), 4.50 (s, 1H), 3.70-3.61 (m, 2H), 3.36-3.17 (m, 2H).

### Example 57

### Compound 57: (Z)-2-(4-((6-chloro-7-methyl-1H-indol-3-yl)methylene)-2,5-dioxoimidazolidin-1-yl)-2-(3,4-difluorophenyl)-N-(3-hydroxyl bicyclo[1.1.1]pentan-1-yl)acetamide

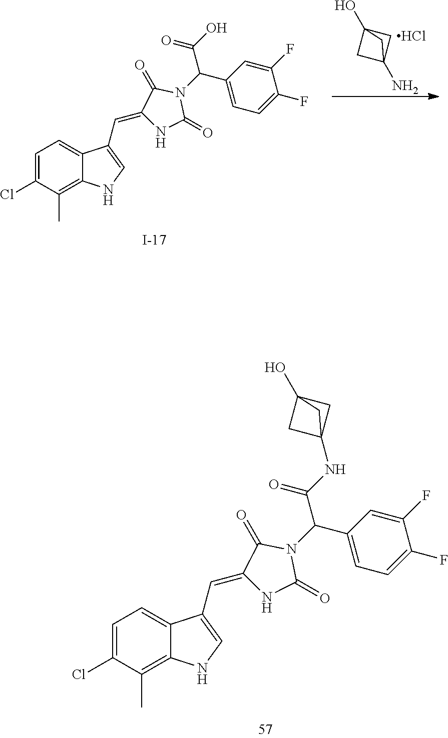

By using the method for the compound 41, the yellow solid compound 57 (12.4 mg, 34.5%) was prepared by using I-17.

LC-MS m/z (ESI): 527.2 [M+H]⁺. ¹H NMR (400 MHz, DMSO-d6) δ 11.99 (s, 1H), 10.51 (s, 1H), 8.68 (s, 1H), 8.20-8.19 (m, 1H), 7.67-7.64 (d, J=8.5 Hz, 1H), 7.50-7.35 (m, 2H), 7.22-7.13 (m, 2H), 6.82 (s, 1H), 6.18 (s, 1H), 5.68 (s, 1H), 2.54 (s, 3H), 2.02 (s, 6H).

### Example 58

### Compound 58: (Z)-2-(4-((6-chloro-7-fluoro-1H-indol-3-yl)methylene)-2,5-dioxoimidazolidin-1-yl)-2-(4-cyanophenyl)-N-(2-hydroxyethyl)acetamide

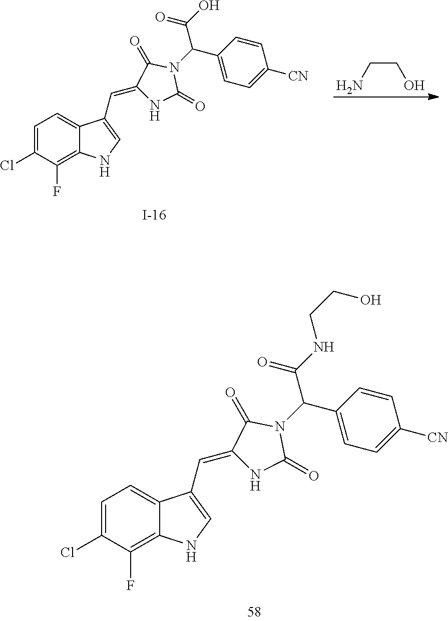

By using the method for the compound 41, the yellow solid compound 58 (11.2 mg, 29.9%) was prepared by using the 1-16.

LC-MS m/z (ESI): 480.0 [M−H]⁻. ¹H NMR (300 MHz, DMSO-d₆) δ 12.54 (s, 1H), 10.61 (s, 1H), 8.30-8.14 (m, 2H), 7.83 (d, J=7.9 Hz, 2H), 7.72-7.52 (m, 3H), 7.32-7.15 (m, 1H), 6.82 (s, 1H), 5.84 (s, 1H), 4.66-4.56 (m, 1H), 3.53-3.15 (m, 4H).

### Example 59

### Compound 59: (Z)-2-(4-((6-chloro-7-fluoro-1H-indol-3-yl)methylene)-2,5-dioxoimidazolidin-1-yl)-2-(4-chlorophenyl)-N-(2-hydroxyethyl)acetamide

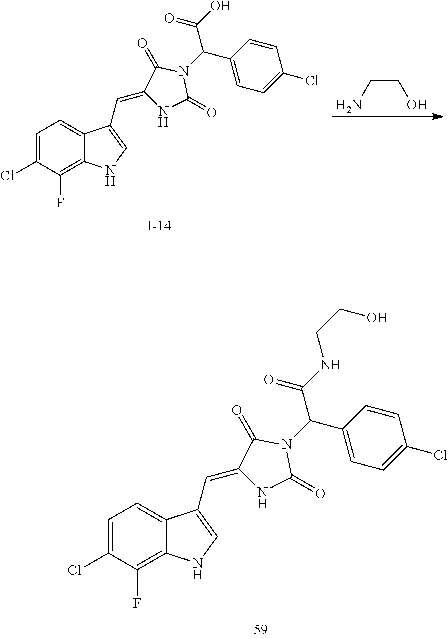

By using the method for the compound 41, the yellow solid compound 59 (6.2 mg, 17.0%) was prepared by using the I-14.

LC-MS m/z (ESI): 491.0 [M+H]⁺. ¹H NMR (400 MHz, DMSO-d₆) δ 12.54 (s, 1H), 10.59 (s, 1H), 8.25 (s, 1H), 8.09-8.00 (m, 1H), 7.67 (d, J=8.5 Hz, 1H), 7.52-7.38 (m, 4H), 7.25-7.17 (m, 1H), 6.82 (s, 1H), 5.74 (s, 1H), 4.65-4.58 (m, 1H), 3.46-3.39 (m, 2H), 3.28-3.10 (m, 2H).

### Example 60

### Compound 60: (Z)-2-(4-((6-chloro-7-methyl-1H-indol-3-yl)methylene)-2,5-dioxoimidazolidin-1-yl)-2-(4-cyanophenyl)-N-(2-hydroxyethyl)acetamide

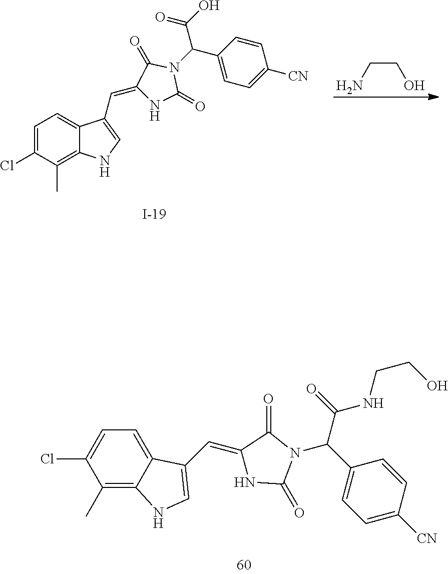

By using the method for the compound 41, the yellow solid compound 60 (12 mg, 42.1%) was prepared by using I-19.

LC-MS m/z (ESI): 476.1 [M−H]⁻. ¹H NMR (300 MHz, DMSO-d₆) δ 11.99 (s, 1H), 10.54 (s, 1H), 8.24-8.13 (m, 2H), 7.87-7.78 (m, 2H), 7.68-7.55 (m, 3H), 7.15 (d, J=8.5 Hz, 1H), 6.83 (s, 1H), 5.84 (s, 1H), 4.65-4.55 (m, 1H), 3.49-3.36 (m, 2H), 3.27-3.08 (m, 2H), 2.50 (s, 3H).

### Example 61

### Compound 61: (Z)-2-(4-((6-chloro-1H-indol-3-yl)methylene)-2,5-dioxoimidazolidin-1-yl)-2-(4-cyanophenyl)-N-(2-hydroxyethyl)acetamide

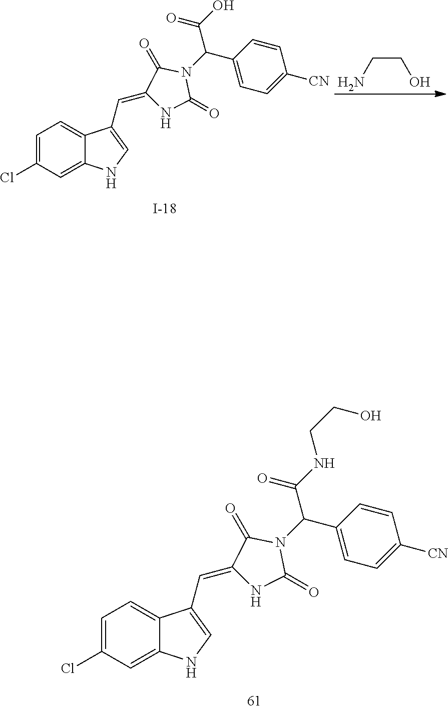

By using the method for the compound 41, the yellow solid compound 61 (14.1 mg, 29.9%) was prepared by using I-18.

LC-MS m/z (ESI): 464.1 [M+H]⁺. ¹H NMR (300 MHz, DMSO-d₆) δ 11.97 (s, 1H), 10.54 (s, 1H), 8.23-8.13 (m, 2H), 7.87-7.77 (m, 3H), 7.65-7.55 (m, 2H), 7.53-7.43 (m, 1H), 7.19-7.09 (m, 1H), 6.85 (s, 1H), 5.84 (s, 1H), 4.70-4.56 (m, 1H), 3.48-3.37 (m, 2H), 3.29-3.08 (m, 2H).

### Example 62

### Compound 62: (Z)-2-(4-((6-chloro-1H-indol-3-yl)methylene)-2,5-dioxoimidazolidin-1-yl)-2-(4-chlorophenyl)-N-(2-hydroxyethyl)acetamide

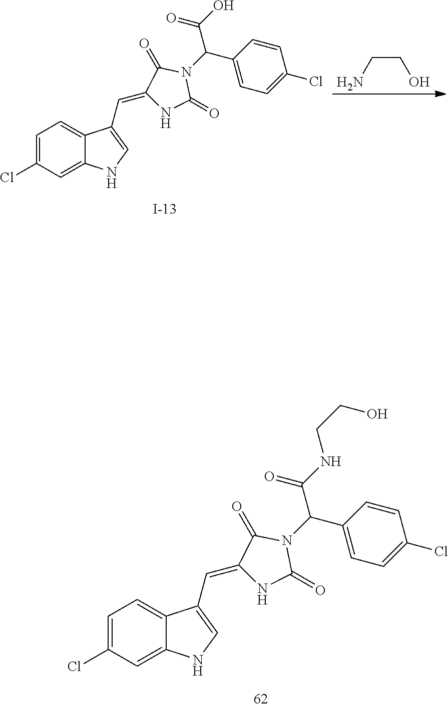

By using the method for the compound 41, the yellow solid compound 62 (12.1 mg, 38.5%) was prepared by using I-13.

LC-MS m/z (ESI): 473.0 [M+H]⁺. ¹H NMR (400 MHz, DMSO-d6) δ 11.95 (s, 1H), 10.50 (s, 1H), 8.18 (s, 1H), 8.05-8.03 (m, 1H), 7.82 (d, J=8.5 Hz, 1H), 7.48-7.39 (m, 5H), 7.13 (d, J=8.5 Hz, 1H), 6.83 (s, 1H), 5.73 (s, 1H), 4.61-4.57 (m, 1H), 3.44-3.39 (m, 2H), 3.25-3.13 (m, 2H).

### Example 63

### Compound 63: (Z)-2-(4-((6-chloro-1H-indol-3-yl)methylene)-2,5-dioxoimidazolidin-1-yl)-2-(3,4-difluorophenyl)-N-(2-hydroxyethyl)acetamide

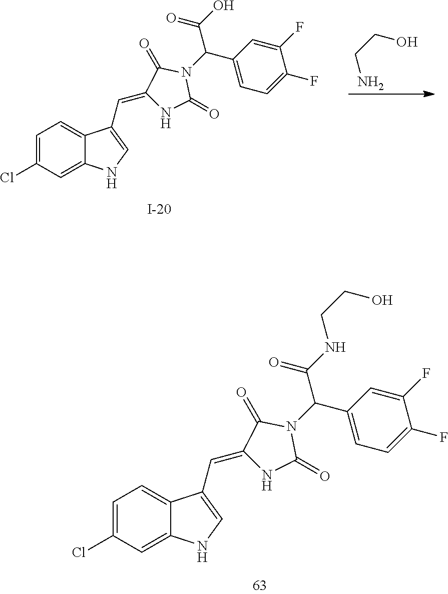

By using the method for the compound 41, the yellow solid compound 63 (11 mg, 14.5%) was prepared by using the 1-20.

LC-MS m/z (ESI): 475.0 [M+H]⁺. ¹H NMR (300 MHz, DMSO-d₆) δ 11.97 (s, 1H), 10.52 (s, 1H), 8.19 (s, 1H), 8.11-8.00 (m, 1H), 7.82 (d, J=8.6 Hz, 1H), 7.58-7.34 (m, 3H), 7.32-7.21 (m, 1H), 7.14 (d, J=8.6, 1H), 6.85 (s, 1H), 5.75 (s, 1H), 3.48-3.35 (m, 2H), 3.31-3.05 (m, 2H).

### Example 64

### Compound 64: (Z)-2-(4-((6-chloro-7-methyl-1H-indol-3-yl)methylene)-2,5-dioxoimidazolidin-1-yl)-2-(3,4-difluorophenyl)-N-((4-hydroxylbicyclo[2.2.2]octan-1-yl)methyl)acetamide

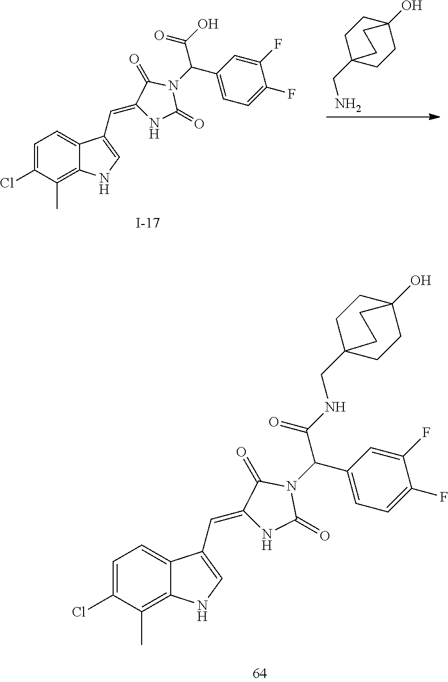

By using the method for the compound 41, the yellow solid compound 64 (19.1 mg, 47.0%) was prepared by using I-17.

LC-MS m/z (ESI): 581.0 [M−H]⁻. ¹H NMR (400 MHz, DMSO-d6) δ 12.00 (s, 1H), 10.53 (s, 1H), 8.28 (s, 1H), 7.97-7.94 (m, 1H), 7.63 (d, J=8.4 Hz, 1H), 7.51-7.26 (m, 2H), 7.30-7.22 (m, 1H), 7.15 (d, J=8.4 Hz, 1H), 6.82 (s, 1H), 5.74 (s, 1H), 4.22-4.12 (m, 1H), 2.89-2.78 (m, 2H), 2.51 (s, 3H), 1.43-1.42 (m, 12H).

### Example 65

### Compound 65: (Z)-2-(2-(4-((6-chloro-7-methyl-1H-indol-3-yl)methylene)-2,5-dioxoimidazolidin-1-yl)-2-(3,4-difluorophenyl)acetamido)-3-hydroxypropyl dihydrophosphate

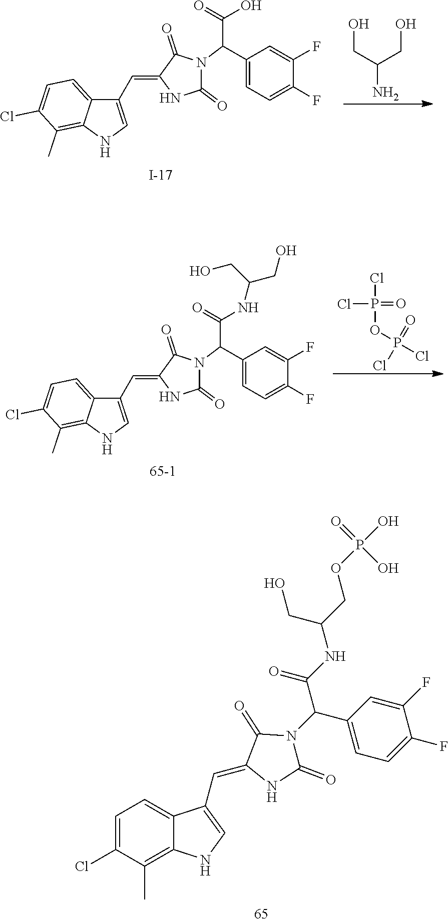

### 65-1: (Z)-2-(4-((6-chloro-7-methyl-1H-indol-3-yl)methylene)-2,5-dioxoimidazolidin-1-yl)-2-(3,4-difluorophenyl)-N-(1,3-dihydroxypropan-2-yl)acetamide

By using the method for the compound 41, the yellow solid compound 65-1 (23.4 mg, 41%) was prepared by using the I-17.

LC-MS m/z (ESI): 519.05 [M+H]⁺.

### Compound 65: (Z)-2-(2-(4-((6-chloro-7-methyl-1H-indol-3-yl)methylene)-2,5-dioxoimidazolidin-1-yl)-2-(3,4-difluorophenyl)acetamido)-3-hydroxypropyl dihydrophosphate

Under argon protection at 0° C., dichlorophosphate anhydride (243 mg, 0.96 mmol, 5.00 eq) was added into a stirred solution of the compound 65-1 (100 mg, 0.19 mmol, 1.00 eq) in ACN (5 mL) and THF (2.5 mL), and the obtained mixture was stirred at 0° C. for 3 h. Ice water (3 mL) was added to terminate the reaction, and the mixture was neutralized with saturated sodium hydrogen carbonate till pH=8. The obtained mixture was stirred under argon protection at 0° C. for 30 min. The mixture was acidized with concentrated hydrochloric acid till pH=1. The obtained mixture was stirred under argon protection at 0° C. for 1.5 h. The obtained mixture was extracted with EtOAc (3×30 mL), and combined organic layers were washed with saline (30 mL) and dried with anhydrous sodium sulfate. After the organic layers were filtered, a filtrate was concentrated at a reduced pressure. The crude product was purified by Prep-HPLC to obtain the yellow solid compound 65 (4.9 mg, 4.16%).

LC-MS m/z (ESI): 597.1[M−H]⁻. ¹HNMR (300 MHz, DMSO-d₆) δ 8.20 (s, 1H), 7.63 (d, J=8.5 Hz, 1H), 7.57-7.32 (m, 2H), 7.31-7.11 (m, 2H), 6.82 (s, 1H), 5.78 (s, 1H), 3.88-3.83 (m, 1H), 3.82-3.65 (m, 2H), 3.46-3.30 (m, 2H), 2.57 (s, 3H).

### Example 66

### Compound 66: (Z)-2-(4-((6-chloro-7-methyl-1H-indol-3-yl)methylene)-2,5-dioxoimidazolidin-1-yl)-2-(3,4-difluorophenyl)-N-(4-hydroxyl bicyclo[2.2.2]octan-1-yl)acetamide

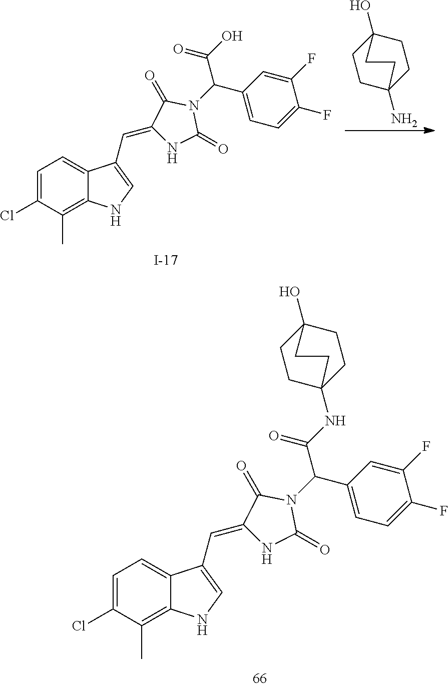

By using the method for the compound 41, the yellow solid compound 66 (2.2 mg, 49.5%) was prepared by using the I-17.

LC-MS m/z (ESI): 566.9 [M−H]⁻. ¹H NMR (400 MHz, DMSO-d6) δ 11.97 (s, 1H), 10.04 (s, 1H), 8.18 (s, 1H), 7.64-7.61 (m, 2H), 7.48-7.35 (m, 2H), 7.18-7.12 (m, 2H), 6.77 (s, 1H), 5.64 (s, 1H), 4.27 (s, 1H), 2.45 (s, 3H), 1.92-1.87 (m, 6H), 1.59-1.53 (m, 6H).

### Example 67

### Compound 67: (Z)-2-(4-((6-chloro-7-methyl-1H-indol-3-yl)methylene)-2,5-dioxoimidazolidin-1-yl)-2-(4-cyano-3-fluorophenyl)-N-(2-hydroxyethyl)acetamide

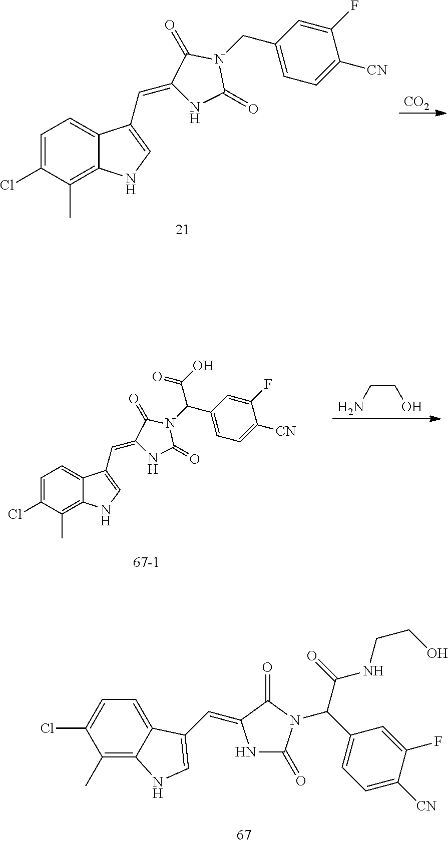

### 67-1: (Z)-2-(4-((6-chloro-7-methyl-1H-indol-3-yl)methylene)-2,5-dioxoimidazolidin-1-yl)-2-(4-cyano-3-fluorophenyl)acetic acid

Under nitrogen protection at −78° C., LiHMDS (1 mL, 1.000 mmol, 5.4 equiv) was added into a stirred solution of the compound 21 (80 mg, 0.186 mmol, 1.0 equiv) in THF (3 mL), and the obtained mixture was stirred at −78° C. for 1 h. Then, the mixture was stirred in a CO₂ atmosphere to react for 15 min. Then, the obtained mixture was stirred under nitrogen protection at −65° C. for 30 min. The reaction was terminated at −65° C. with a solution of HOAc (2 mL) in THF (1 mL). Then, the reaction mixture was poured into 50 mL of cold water. The obtained mixture was extracted with EtOAc (3×30 mL). Combined organic layers were washed with saline (2×15 mL) and dried with anhydrous sodium sulfate. After the organic layers were filtered, a filtrate was concentrated at a reduced pressure. A residue was purified by fast reversed-phase chromatography to obtain the yellow solid compound 67-1 (23 mg, 27.3%).

### Compound 67: (Z)-2-(4-((6-chloro-7-methyl-1H-indol-3-yl)methylene)-2,5-dioxoimidazolidin-1-yl)-2-(4-cyano-3-fluorophenyl)-N-(2-hydroxyethyl)acetamide

By using the method for the compound 41, the yellow solid compound 67 (3.7 mg, 13.0%) was prepared by using 67-1.

LC-MS m/z (ESI): 496.1 [M+H]⁺. ¹H NMR (300 MHz, DMSO-d₆) δ 12.01 (s, 1H), 10.59 (s, 1H), 8.29-8.19 (m, 2H), 7.98-7.87 (m, 1H), 7.67-7.57 (m, 2H), 7.53-7.40 (m, 1H), 7.20-7.05 (m, 1H), 6.85 (s, 1H), 5.88 (s, 1H), 3.48-3.40 (m, 2H), 3.30-3.06 (m, 2H), 2.50 (s, 3H).

### Example 68

### Compound 68: (Z)-2-(4-((6-chloro-7-methyl-1H-indol-3-yl)methylene)-2,5-dioxoimidazolidin-1-yl)-2-(3,4-difluorophenyl)-N-((3-hydroxyl bicyclo[1.1.1]pentan-1-yl)methyl)acetamide

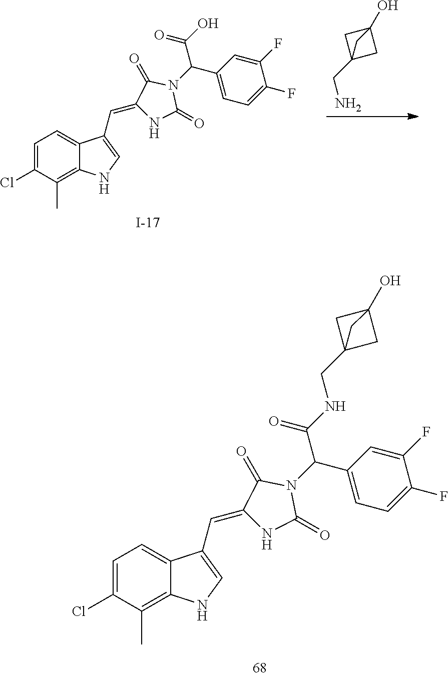

By using the method for the compound 41, the yellow solid compound 68 (5.1 mg, 24.44%) was prepared by using the I-17.

LC-MS m/z (ESI): 541.1 [M+H]⁺. ¹H NMR (400 MHz, DMSO-d6) δ 11.99 (s, 1H), 10.53 (s, 1H), 8.20 (s, 1H), 8.15-8.05 (m, 1H), 7.65 (d, J=8.5 Hz, 1H), 7.55-7.37 (m, 2H), 7.40-7.30 (m, 1H), 7.26 (s, 1H), 7.15 (d, J=8.5 Hz, 1H), 6.82 (s, 1H), 5.73 (s, 1H), 3.35-3.30 (m, 2H), 2.51 (s, 3H), 1.65 (s, 6H).

### Example 69

### Compound 69: (Z)-5-((6-chloro-7-fluoro-1H-indol-3-yl)methylene)-3-(1-(4-chlorophenyl)-2-hydroxyethyl)imidazolidine-2,4-dione

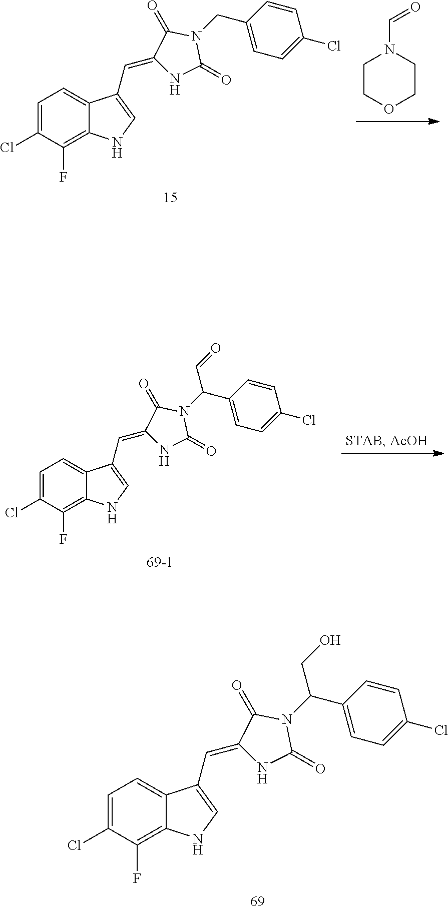

### 69-1: (Z)-2-(4-((6-chloro-7-fluoro-1H-indol-3-yl)methylene)-2,5-dioxoimidazolidin-1-yl)-2-(4-chlorophenyl)acetaldehyde

By using the method for the compound 33, the white solid compound 69-1 (80 mg, 53.3%) was prepared by using the compound 15.

LC-MS m/z (ESI): 430.0 [M−H]⁻

### Compound 69: (Z)-5-((6-chloro-7-fluoro-1H-indol-3-yl)methylene)-3-(1-(4-chlorophenyl)-2-hydroxyethyl)imidazolidine-2,4-dione

By using the method for the compound 37, the yellow solid compound 69 (12.3 mg, 20.4%) was prepared by using 69-1.

LC-MS m/z (ESI): 434.0 [M+H]⁺. ¹H NMR (400 MHz, DMSO-d₆) δ 12.53 (s, 1H), 10.55 (s, 1H), 8.25 (s, 1H), 7.66 (d, J=8.5 Hz, 1H), 7.49-7.39 (m, 4H), 7.26-7.16 (m, 1H), 6.81 (s, 1H), 5.25-5.15 (m, 2H), 4.40-4.31 (m, 1H), 3.98-3.92 (m, 1H).

### Example 70

### Compound 70: (Z)-5-((6-chloro-7-methyl-1H-indol-3-yl)methylene)-3-(1-(4-chlorophenyl)-2-hydroxyethyl)imidazolidine-2,4-dione

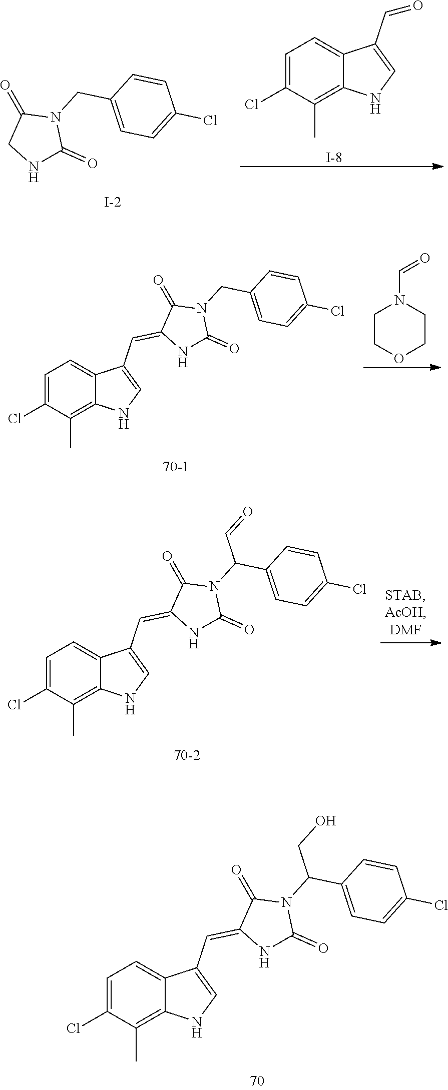

### 70-1: (Z)-5-((6-chloro-7-methyl-1H-indol-3-yl)methylene)-3-(4-chlorobenzyl)imidazolidine-2,4-dione

By using the method in Example 1, the yellow solid compound 70-1 (800 mg, 62%) was prepared by using the intermediates I-2 and I-8.

### 70-2: (Z)-2-(4-((6-chloro-7-methyl-1H-indol-3-yl)methylene)-2,5-dioxoimidazolidin-1-yl)-2-(4-chlorophenyl)acetaldehyde

By using the method for the compound 33, the white solid compound 70-2 (140 mg, 21.2%) was prepared by using the compound 70-1.

### Compound 70: (Z)-5-((6-chloro-7-methyl-1H-indol-3-yl)methylene)-3-(1-(4-chlorophenyl)-2-hydroxyethyl)imidazolidine-2,4-dione

By using the method for the compound 37, the yellow solid compound 70 (20 mg, 30.5%) was prepared by using the compound 70-2.

LC-MS m/z (ESI): 428.0 [M−H]⁻. ¹H NMR (400 MHz, DMSO-d₆) δ 11.99 (s, 1H), 10.50 (s, 1H), 8.20 (s, 1H), 7.64 (d, J=8.5 Hz, 1H), 7.46-7.41 (m, 4H), 7.15 (d, J=8.5 Hz, 1H), 6.82 (s, 1H), 5.26-5.14 (m, 2H), 4.41-4.30 (m, 1H), 4.00-3.90 (m, 1H), 2.52 (s, 3H).

### Example 71

### Compound 71: (Z)-(6-chloro-3-((1-(4-cyano-3-fluorobenzyl)-2,5-dioxoimidazolidin-4-ylene)methyl)-7-fluoro-1H-indol-1-yl)phosphonic acid

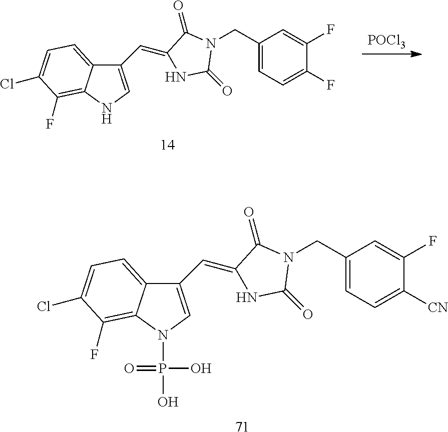

Under argon protection at 0° C., a solution of phosphorus oxychloride (186 mg, 1.21 mmol, 5.00 eq) in THF (1 mL) was added into a stirred solution of the compound 14 (100 mg, 0.24 mmol, 1.00 eq) in THF (1 mL). A THF solution (1 mL) of TEA (278 mg, 2.74 mmol, 11.32 eq) was dropwise added into the mixture at 0° C., and the obtained mixture was then stirred at 0° C. for 2 h. The mixture was alkalized with saturated sodium hydrogen carbonate till pH=8, and the obtained mixture was stirred at 0° C. for 30 min. The mixture was acidized with concentrated hydrochloric acid till pH=1. The obtained mixture was diluted with water (20 mL) and extracted with EtOAc (3×50 mL). Combined organic layers were washed with saline (50 mL) and dried with anhydrous sodium sulfate. After the organic layers were filtered, a filtrate was concentrated at a reduced pressure. The crude product was purified by Prep-HPLC to obtain the yellow solid compound 71 (13.1 mg, 0.018 mmol 7.4%).

LC-MS m/z (ESI): 491.0[M−H]⁻. ¹H NMR (300 MHz, DMSO-d₆) δ 8.15 (s, 1H), 7.91-7.79 (m, 1H), 7.47-7.36 (m, 1H), 7.32-7.19 (m, 2H), 7.07-6.97 (m, 1H), 6.27 (s, 1H), 4.70 (s, 2H).

### Example 72

### Compound 72: (Z)-2-(4-((6-chloro-7-fluoro-1H-indol-3-yl)methylene)-2,5-dioxoimidazolidin-1-yl)-2-(3,4-difluorophenyl)-N-(2-hydroxyethyl)acetamide

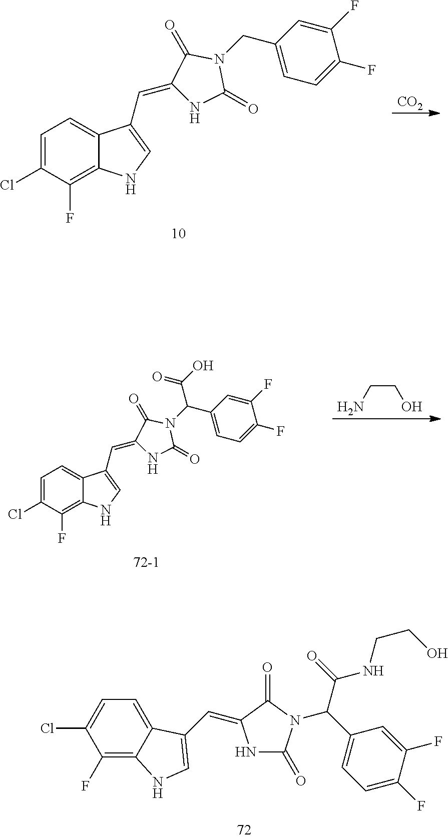

### 72-1: (Z)-2-(4-((6-chloro-7-fluoro-1H-indol-3-yl)methylene)-2,5-dioxoimidazolidin-1-yl)-2-(3,4-difluorophenyl)acetic acid

By using the method for the compound 67-1, the yellow solid compound 72-1 (23 mg, 47%) was prepared by using the compound 10.

LC-MS m/z (ESI): 448.01 [M−H]−. ¹H NMR (400 MHz, DMSO-d₆) δ

### Compound 72: (Z)-2-(4-((6-chloro-7-fluoro-1H-indol-3-yl)methylene)-2,5-dioxoimidazolidin-1-yl)-2-(3,4-difluorophenyl)-N-(2-hydroxyethyl)acetamide

By using the method for the compound 41, the yellow solid compound 72 (10.6 mg, 45.6%) was prepared by using the compound 72-1.

LC-MS m/z (ESI): 493.15 [M+H]⁺ 0.1H NMR (400 MHz, DMSO-d₆) δ 12.54 (d, J=2.8 Hz, 1H), 10.60 (s, 1H), 8.25 (d, J=2.8 Hz, 1H), 8.08 (t, J=5.7 Hz, 1H), 7.68 (d, J=8.6 Hz, 1H), 7.54-7.48 (m, 1H), 7.46-7.38 (m, 1H), 7.29-7.18 (m, 2H), 6.83 (s, 1H), 5.76 (s, 1H), 4.61 (s, 1H), 3.43-3.40 (m, 2H), 3.26-3.12 (m, 2H).

### Example 73

### Compound 73: (Z)-3-(2-(4-((6-chloro-7-methyl-1H-indol-3-yl)methylene)-2,5-dioxoimidazolidin-1-yl)-2-(3,4-difluorophenyl)acetamido)bicyclo[1.1.1]pentan-1-yl dihydrophosphate

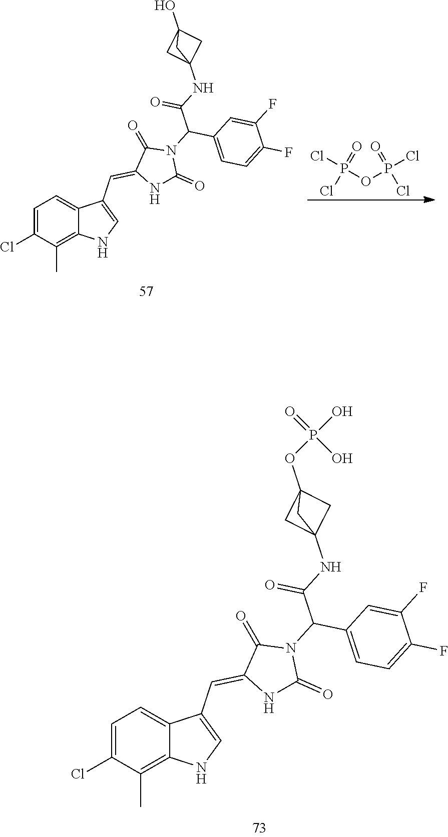

By using the method for the compound 42, the yellow solid compound 73 (6.1 mg, 16.69%) was prepared by using the compound 57.

LC-MS m/z (ESI): 607.01 [M+H]⁺. ¹H NMR (400 MHz, DMSO-d6) δ 12.04 (s, 1H), 8.72 (s, 1H), 8.12 (s, 1H), 7.65 (d, J=8.5 Hz, 1H), 7.48-7.34 (m, 2H), 7.26-7.18 (s, 1H), 7.15 (d, J=8.5 Hz, 1H), 6.82 (s, 1H), 5.69 (s, 1H), 3.60-3.50 (m, 5H), 2.49 (s, 3H), 2.27-2.16 (m, 6H).

### Example 74

### Compound 74: (Z)-2-(2-(4-((6-chloro-7-methyl-1H-indol-3-yl)methylene)-2,5-dioxoimidazolidin-1-yl)-2-(3,4-difluorophenyl)acetamido)propane-1,3-diylbis(dihydrophosphate)

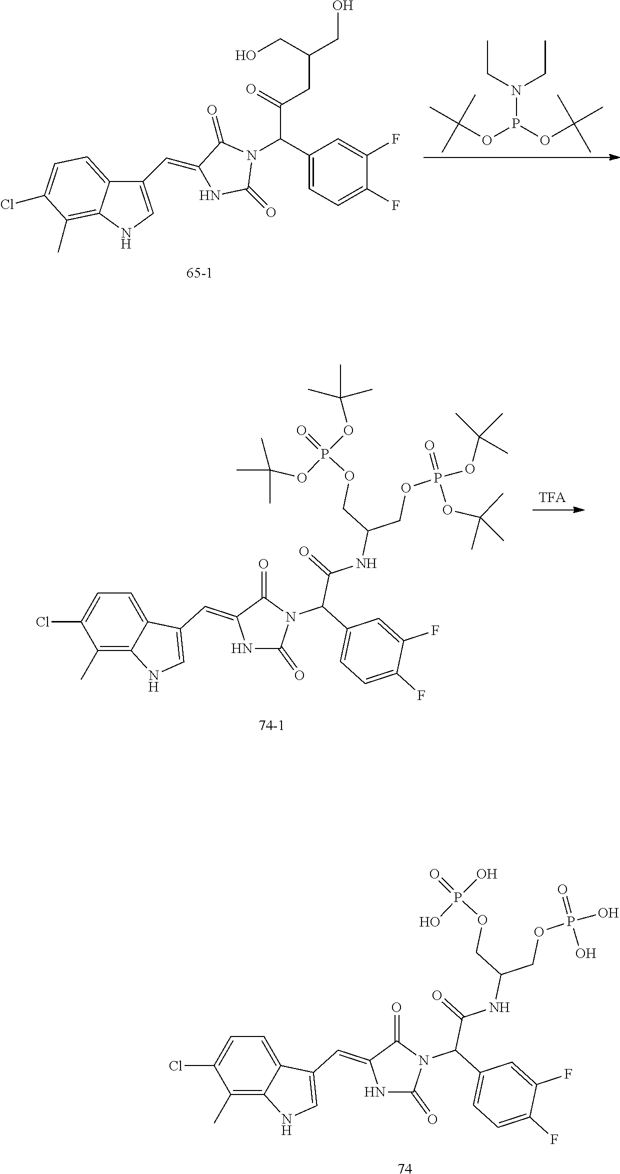

### 74-1: (Z)-tetra-tert-butyl (2-(2-(4-((6-chloro-7-methyl-1H-indol-3-yl)methylene)-2,5-dioxoimidazolidin-1-yl)-2-(3,4-difluorophenyl)acetamido)propane-1,3-diyl)bis(phosphate)

In an argon atmosphere, a MeCN (9.5 mL) of 1H-1,2,3,4-tetrazole (259 mg, 3.70 mmol, 8.00 eq) and [bis(tert-butoxyl)phosphono]diethylamine (464 mg, 1.86 mmol, 4.02 eq) were added into a solution of 65-1 (240 mg, 0.46 mmol, 1.00 eq) in THF (24 mL), and the obtained mixture was stirred under argon protection at room temperature for 12 h. A solution of metachloroperbenzoic acid (563 mg, 2.78 mmol, 6.00 eq, 85%) in DCM (24 mL) was added into the mixture at 0° C., and the obtained mixture was then stirred at 4° C. for 1.5 h. The obtained mixture was diluted with DCM (50 mL) and continuously washed 10% Na₂S₂O₅, sodium hydrogen carbonate, and saline respectively. Organic phases were concentrated. A residue was purified by fast reversed-phase chromatography to obtain the light brown syrupy compound 74-1 (120 mg, 26.5%).

LC-MS m/z (ESI): 901.3[M−H]⁻.

### Compound 74: (Z)-2-(2-(4-((6-chloro-7-methyl-1H-indol-3-yl)methylene)-2,5-dioxoimidazolidin-1-yl)-2-(3,4-difluorophenyl)acetamido)propane-1,3-diylbis(dihydrophosphate)

TFA (5 mL, 1283 eq) was dropwise added into a solution of 74-1 (50 mg, 0.05 mmol, 1.00 eq) in DCM (10 mL) at 0° C., and a reaction mixture was stirred at room temperature for 1 h. The obtained mixture was concentrated in vacuum. A crude product was purified by Prep-HPLC to obtain the yellow solid compound 74 (3.8 mg, 11.2%).

LC-MS m/z (ESI): 677.0[M−H]⁻. ¹H NMR (300 MHz, DMSO-d₆) δ 11.99 (s, 1H), 8.19 (s, 1H), 7.65 (d, J=8.7 Hz, 1H), 7.48-7.34 (m, 3H), 7.29-7.20 (m, 1H), 7.17-7.12 (m, 1H), 6.81 (s, 1H), 4.80 (s, 1H), 4.02-3.94 (m, 1H), 3.93-3.85 (m, 2H), 3.84-3.66 (m, 2H), 2.52 (s, 3H).

### Example 75

### Compound 75: (Z)-4-(2-(4-((6-chloro-7-methyl-1H-indol-3-yl)methylene)-2,5-dioxoimidazolidin-1-yl)-2-(3,4-difluorophenyl)acetamido)bicyclo[2.2.2]octan-1-yl dihydrophosphate

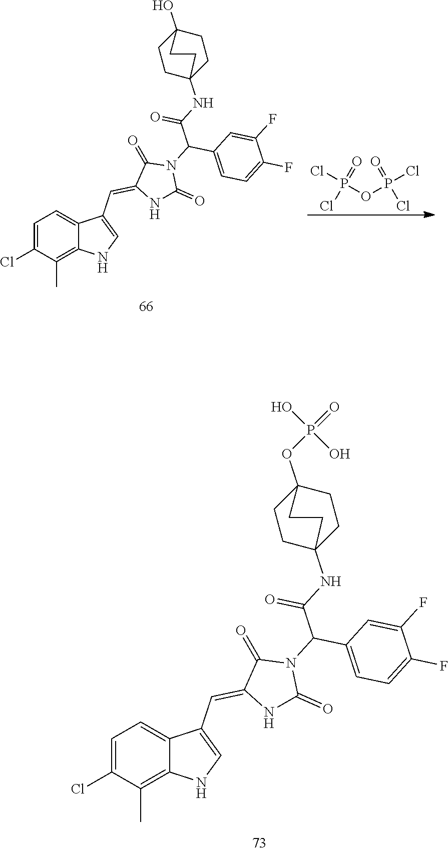

By using the method for the compound 42, the yellow solid compound 75 (15.2 mg, 33.7%) was prepared by using the compound 66.

LC-MS m/z (ESI): 647.05 [M−H]⁻. ¹H NMR (400 MHz, DMSO-d₆) δ 12.04 (s, 1H), 8.12 (s, 1H), 7.67-7.61 (m, 2H), 7.46-7.35 (m, 2H), 7.19-7.12 (m, 2H), 6.78 (s, 1H), 5.64 (s, 1H), 2.49 (s, 3H), 1.91 (s, 12H).

### Example 76

### Compound 76: (Z)-4-((2-(4-((6-chloro-7-methyl-1H-indol-3-yl)methylene)-2,5-dioxoimidazolidin-1-yl)-2-(3,4-difluorophenyl)acetamido)methyl)bicyclo[2.2.2]octan-1-yl dihydrophosphate

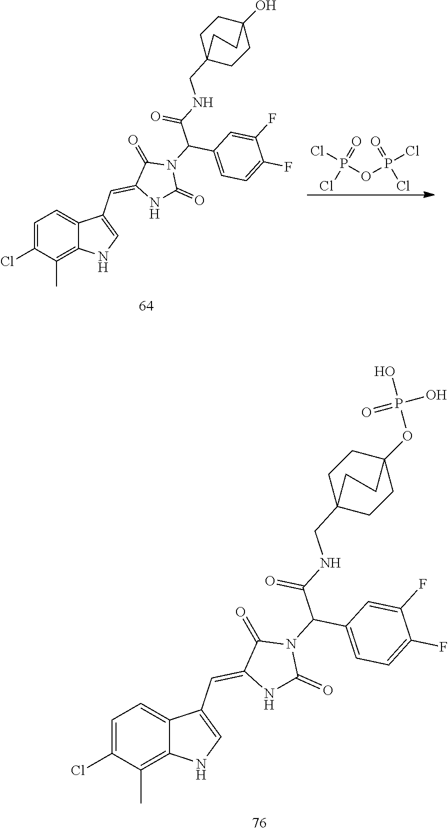 [SMILES: Cc1c(Cl)ccc2c(/C=C3\NC(=O)N(C(C(=O)NCC45CCC(O)(CC4)CC5)c4ccc(F)c(F)c4)C3=O)c[nH]c12 ; O=P(Cl)(Cl)OP(=O)(Cl)Cl ; Cc1c(Cl)ccc2c(/C=C3\NC(=O)N(C(C(=O)NCC45CCC(OP(=O)(O)O)(CC4)CC5)c4ccc(F)c(F)c4)C3=O)c[nH]c12]

By using the method for the compound 42, the yellow solid compound 76 (24.7 mg, 37.6%) was prepared by using the compound 64.

LC-MS m/z (ESI): 663.01 [M+H]⁺. ¹H NMR (400 MHz, DMSO-d₆) δ 11.93 (s, 1H), 8.13-8.11 (m, 1H), 7.53-7.45 (m, 1H), 7.57-7.59 (d, J=8.5 Hz, 1H), 7.42-7.29 (m, 2H), 7.22-7.14 (m, 1H), 7.07-7.05 (d, J=8.5 Hz, 1H), 6.75 (s, 1H), 5.67 (s, 1H), 2.82-2.60 (m, 2H), 2.44 (s, 3H), 1.79-1.69 (m, 6H), 1.41-1.31 (m, 6H).

### Example 77

### Compound 77: (Z)-3-(1-(4-chloro-3-fluorophenyl)-2-hydroxyethyl)-5-((6-chloro-7-fluoro-1H-indol-3-yl)methylene)imidazolidine-2,4-dione

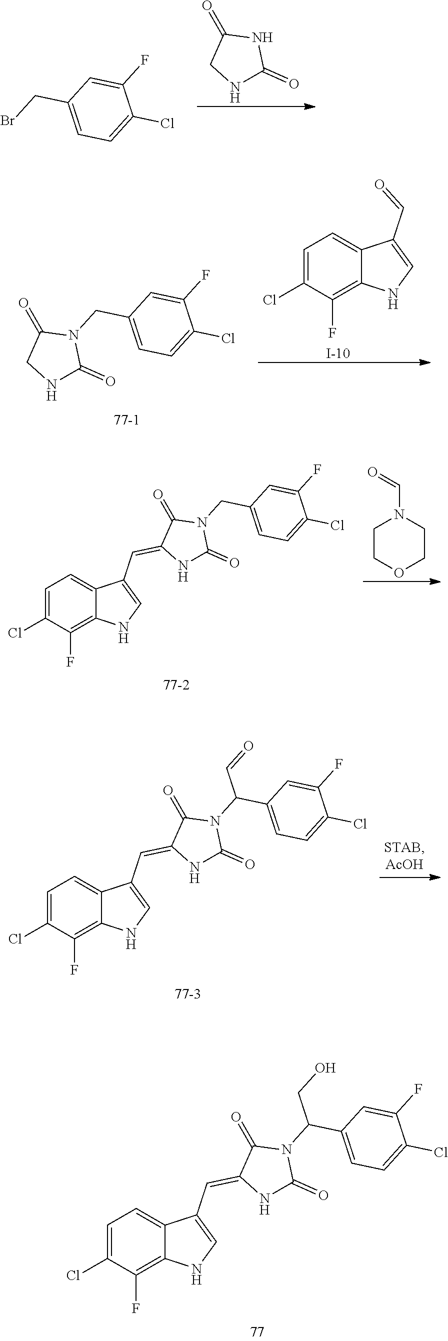

### 77-1: 3-(4-chloro-3-fluorobenzyl)imidazolidine-2,4-dione

By using the method for the compound 1-1, the offwhite solid compound 77-1 (10 g, 88.4%) was prepared by using hydantoin and 4-(bromomethyl)-1-chloro-2-fluorobenzene.

LC-MS m/z (ESI): 241.0[M−H]⁻.

### 77-2: (Z)-3-(4-chloro-3-fluorobenzyl)-5-((6-chloro-7-fluoro-1H-indol-3-yl)methylene)imidazolidine-2,4-dione

By using the method for the compound 1, the yellow solid compound 77-2 (2.0 g, 91.5%) was prepared by using the compounds 77-1 and I-10.

LC-MS m/z (ESI): 420.0[M−H]⁻.

### 77-3: (Z)-2-(4-chloro-3-fluorophenyl)-2-(4-((6-chloro-7-fluoro-1H-indol-3-yl)methylene)-2,5-dioxoimidazolidin-1-yl)acetaldehyde

By using the method for the compound 33, the yellow solid compound 77-3 (50 mg, 8.3%) was prepared by using the compound 77-2.

LC-MS m/z (ESI): 450.0[M+H]⁺.

### Compound 77: (Z)-3-(1-(4-chloro-3-fluorophenyl)-2-hydroxyethyl)-5-((6-chloro-7-fluoro-1H-indol-3-yl)methylene)imidazolidine-2,4-dione

By using the method for the compound 37, the yellow solid compound 77 (22.4 mg, 53.1%) was prepared by using the compound 77-3.

LC-MS m/z (ESI): 451.9[M+H]⁺. ¹H NMR (300 MHz, DMSO-d₆) δ 12.53 (s, 1H), 10.56 (s, 1H), 8.25 (s, 1H), 7.72-7.64 (m, 1H), 7.63-7.55 (m, 1H), 7.49 (d, J=10.6, 1H), 7.32-7.16 (m, 2H), 6.82 (s, 1H), 5.30-5.15 (m, 2H), 4.40-4.20 (m, 1H), 4.10-3.90 (m, 1H).

### Example 78

### Compound 78: (Z)-3-(1-(4-chloro-3-fluorophenyl)-2-hydroxyethyl)-5-((6-chloro-7-methyl-1H-indol-3-yl)methylene)imidazolidine-2,4-dione

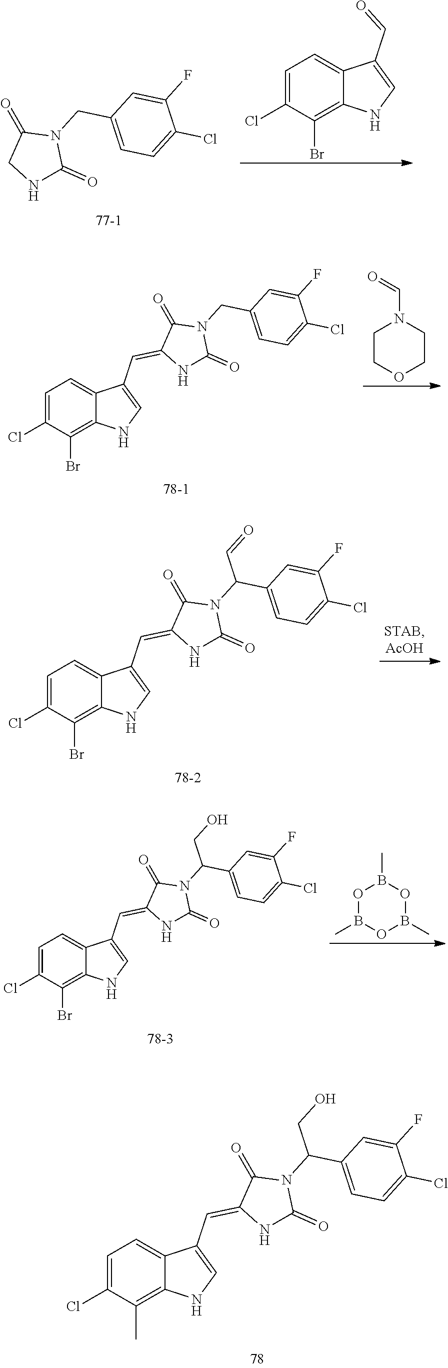

### 78-1: (Z)-5-((7-bromo-6-chloro-1H-indol-3-yl)methylene)-3-(4-chloro-3-fluorobenzyl)imidazolidine-2,4-dione

By using the method for the compound 1, the yellow solid compound 78-1 (1.9 g, 97.6%) was prepared by using the compound 77-1 and 7-bromo-6-chloro-1H-indole-3-formaldehyde.

LC-MS m/z (ESI): 481.9[M+H]⁺.

### 78-2: (Z)-2-(4-((7-bromo-6-chloro-1H-indol-3-yl)methylene)-2,5-dioxoimidazolidin-1-yl)-2-(4-chloro-3-fluorophenyl)acetaldehyde

By using the method for the compound 33, the yellow solid compound 78-2 (110 mg, 13.5%) was prepared by using the compound 78-1.

LC-MS m/z (ESI): 509.9[M+H]⁺.

### 78-3: (Z)-5-((7-bromo-6-chloro-1H-indol-3-yl)methylene)-3-(1-(4-chloro-3-fluorophenyl)-2-hydroxyethyl)imidazolidine-2,4-dione

By using the method for the compound 37, the yellow solid compound 78-3 (66 mg, 57.5%) was prepared by using the compound 78-2.

LC-MS m/z (ESI): 510.0[M−H]⁻. ¹H NMR (300 MHz, DMSO-d6) δ 12.18 (s, 1H), 10.64 (s, 1H), 8.26 (s, 1H), 7.86 (d, J=8.5 Hz, 1H), 7.60-7.58 (m, 1H), 7.48 (d, J=10.6, 1H), 7.34-7.20 (m, 2H), 6.81 (s, 1H), 5.30-5.10 (m, 2H), 4.40-4.20 (m, 1H), 4.05-3.90 (m, 1H).

### Compound 78: (Z)-3-(1-(4-chloro-3-fluorophenyl)-2-hydroxyethyl)-5-((6-chloro-7-methyl-1H-indol-3-yl)methylene)imidazolidine-2,4-dione

Trimethyl-1,3,5,2,4,6-trioxatripyridone (350 uL, 1.23 mmol, 19.64 eq) and potassium carbonate (51 mg, 0.5 mg) were added into a stirred mixture of the compound 78-3 (32 mg, 0.062 mmol, 1.00 eq) and dioxane (3 mL) of and H2O (0.3 mL) of Pd(dppf)Cl₂ (8 mg, 0.01 mmol, 0.18 eq), and the obtained mixture was stirred under argon protection at 120° C. for 12 h. The mixture was cooled to room temperature and diluted with EtOAc (50 mL). The obtained mixture was washed with saline (5×30 mL), dried with anhydrous Na₂SO₄, and filtered. A filtrate was concentrated at a reduced pressure. A crude product was purified by Prep-HPLC to obtain the yellow solid compound 78 (3.1 mg, 9.9%).

LC-MS m/z (ESI): 448.2[M+H]⁺ 0.1H NMR (300 MHz, DMSO-d₆) δ 12.00 (s, 1H), 10.53 (s, 1H), 8.21 (s, 1H), 7.72-7.40 (m, 3H), 7.27 (d, J=8.3, 1H), 7.16 (d, J=8.4 Hz, 1H), 6.84 (s, 1H), 5.30-5.16 (m, 2H), 4.48-4.23 (m, 1H), 4.10-3.90 (m, 1H), 2.45 (s, 3H).

### Example 79

### Compound 79: (Z)-5-((6-chloro-1H-indol-3-yl)methylene)-3-(1-(4-chloro-3-fluorophenyl)-2-hydroxyethyl)imidazolidine-2,4-dione

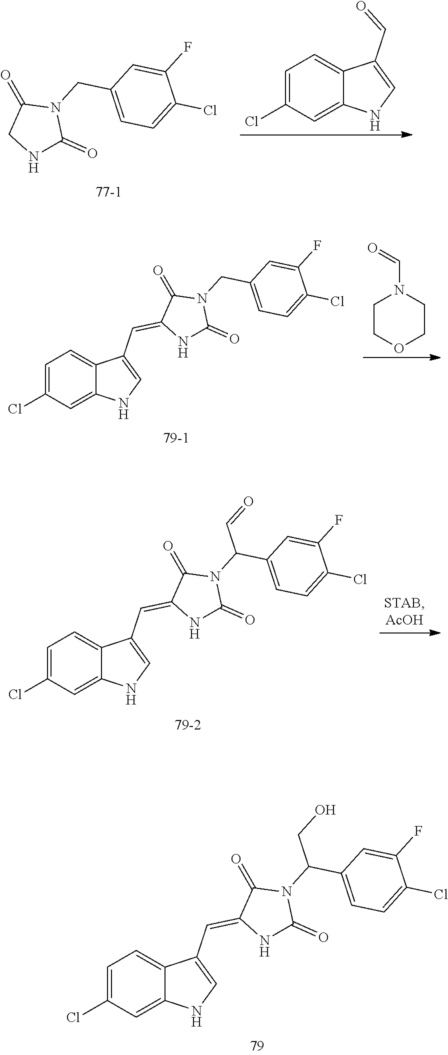

### 79-1: (Z)-5-((6-chloro-1H-indol-3-yl)methylene)-3-(4-chloro-3-fluorobenzyl)imidazolidine-2,4-dione

By using the method for the compound 1, the yellow solid compound 79-1 (2.1 g, 90.3%) was prepared by using the compound 77-1 and 6-chloro-1H-indole-3-formaldehyde.

LC-MS m/z (ESI): 402.0[M−H]⁻.

### 79-2: (Z)-2-(4-((6-chloro-1H-indol-3-yl)methylene)-2,5-dioxoimidazolidin-1-yl)-2-(4-chloro-3-fluorophenyl)acetaldehyde

By using the method for the compound 33, the yellow solid compound 79-2 (210 mg, 36.1%) was prepared by using the compound 79-1.

LC-MS m/z (ESI): 432.0[M+H]⁺.

### 79: (Z)-5-((6-chloro-1H-indol-3-yl)methylene)-3-(1-(4-chloro-3-fluorophenyl)-2-hydroxyethyl)imidazolidine-2,4-dione

By using the method for the compound 37, the yellow solid compound 79 (40.2 g, 39.2%) was prepared by using the compound 79-2.

LC-MS m/z (ESI): 431.8[M−H]⁻. ¹H NMR (400 MHz, DMSO-d₆) δ 11.97 (s, 1H), 10.50 (s, 1H), 8.19 (s, 1H), 7.82 (d, J=8.5 Hz, 1H), 7.65-7.55 (m, 1H), 7.54-7.45 (m, 2H), 7.27 (d, J=8.3, 1H), 7.14 (d, J=8.5, 1H), 6.85 (s, 1H), 5.30-5.15 (m, 2H), 4.40-4.18 (m, 1H), 4.10-3.90 (m, 1H).

### Example 84

### Compound 84: (Z)-5-((6-chloro-7-fluoro-1H-indol-3-yl)methylene)-3-(1-(4-fluorophenyl)-2-hydroxyethyl)imidazolidine-2,4-dione

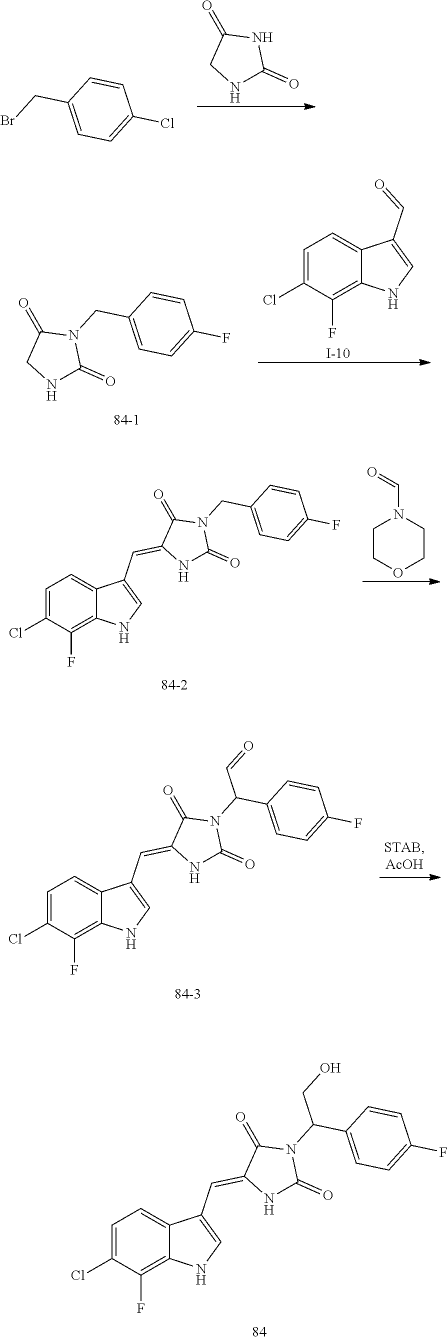

### 84-1: 3-(4-fluorobenzyl)imidazolidine-2,4-dione

By using the method for the compound I-1, the white solid compound 84-1 (4.12 g, 77.9%) was prepared by using the hydantoin and 4-(bromomethyl)-1,2-difluorbenzene.

LC-MS m/z (ESI): 207.0 [M−H]⁻.

### 84-2: (Z)-5-((6-chloro-7-fluoro-1H-indol-3-yl)methylene)-3-(4-fluorobenzyl)imidazolidine-2,4-dione

By using the method for the compound 1, the yellow solid compound 84-2 (1.1 g, 79.7%) was prepared by using the compounds 84-1 and I-10.

LC-MS m/z (ESI): 386.0 [M−H]⁻.

### 84-3: (Z)-2-(4-((6-chloro-7-fluoro-1H-indol-3-yl)methylene)-2,5-dioxoimidazolidin-1-yl)-2-(4-fluorophenyl)acetaldehyde

By using the method for the compound 33, the yellow solid compound 84-3 (340 mg, 61.45%) was prepared by using the compound 84-2.

LC-MS m/z (ESI): 414.0[M−H]⁻.

### Compound 84: (Z)-5-((6-chloro-7-fluoro-1H-indol-3-yl)methylene)-3-(1-(4-fluorophenyl)-2-hydroxyethyl)imidazolidine-2,4-dione

By using the method for the compound 37, the yellow solid compound 84 (28.0 mg, 28.2%) was prepared by using the compound 84-3.

LC-MS m/z (ESI): 418.1[M+H]⁺ 0.1H NMR (400 MHz, DMSO-d₆) δ 12.52 (s, 1H), 10.56 (s, 1H), 8.25 (s, 1H), 7.71-7.61 (m, 1H), 7.60-7.35 (m, 2H), 7.34-7.10 (m, 3H), 6.81 (s, 1H), 5.25-5.15 (m, 2H), 4.50-4.30 (m, 1H), 3.97-3.88 (m, 1H).

### Example 85

### Compound 85: (Z)-4-(1-(4-((6-chloro-7-fluoro-1H-indol-3-yl)methylene)-2,5-dioxoimidazolidin-1-yl)-2-hydroxyethyl)benzonitrile

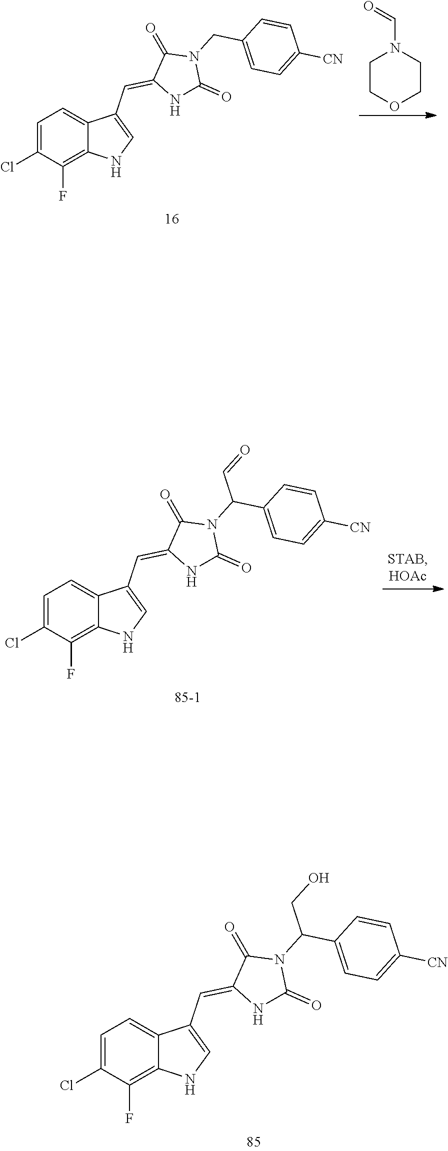

### 85-1: (Z)-4-(1-(4-((6-chloro-7-fluoro-1H-indol-3-yl)methylene)-2,5-dioxoimidazolidin-1-yl)-2-oxyethyl)benzonitrile

By using the method for the compound 33, the brown solid compound 85-1 (120 mg, 23.0%) was prepared by using the compound 16.

LC-MS m/z (ESI): 423.1[M+H]⁺.

### Compound 85: (Z)-4-(1-(4-((6-chloro-7-fluoro-1H-indol-3-yl)methylene)-2,5-dioxoimidazolidin-1-yl)-2-hydroxyethyl)benzonitrile

By using the method for the compound 37, the yellow solid compound 85 (38.1 mg, 31.1%) was prepared by using the compound 85-1.

LC-MS m/z (ESI): 423.1[M−H]⁻. ¹H NMR (300 MHz, DMSO-d₆) δ 12.53 (s, 1H), 10.59 (s, 1H), 8.26 (s, 1H), 7.89-7.79 (m, 2H), 7.71-7.61 (m, 3H), 7.24-7.19 (m, 1H), 6.83 (s, 1H), 5.31-5.24 (m, 2H), 4.37-4.29 (m, 1H), 4.06-3.99 (m, 1H).

### Example 86

### Compound 86: (Z)-5-((6-chloro-7-fluoro-1H-indol-3-yl)methylene)-3-(1-(3,4-difluorophenyl)-2-hydroxyethyl)imidazolidine-2,4-dione

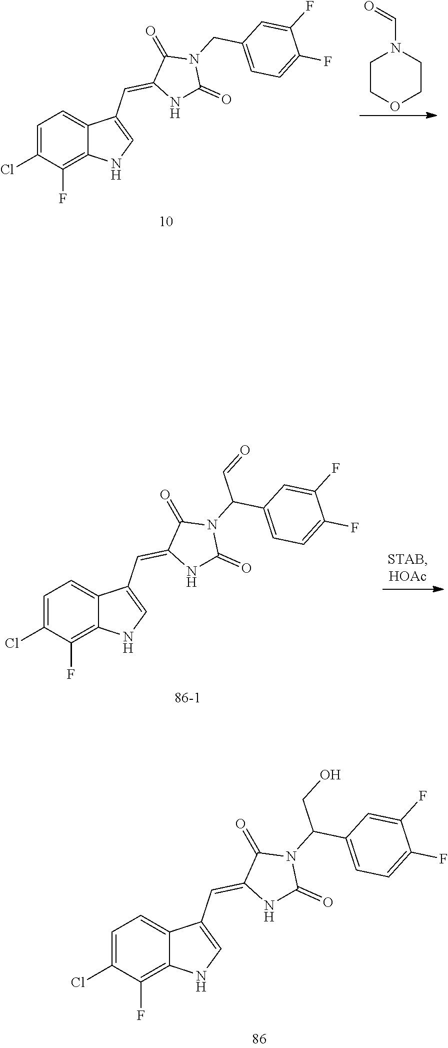

### 86-1: (Z)-2-(4-((6-chloro-7-fluoro-1H-indol-3-yl)methylene)-2,5-dioxoimidazolidin-1-yl)-2-(3,4-difluorophenyl)acetaldehyde

By using the method for the compound 33, the brown solid compound 86-1 (240 mg, 33.5%) was prepared by using the compound 10.

LC-MS m/z (ESI): 434.0[M+H]⁺.

### Compound 86: (Z)-5-((6-chloro-7-fluoro-1H-indol-3-yl)methylene)-3-(1-(3,4-difluorophenyl)-2-hydroxyethyl)imidazolidine-2,4-dione

By using the method for the compound 37, the yellow solid compound 86 (90 mg, 46.4%) was prepared by using the compound 86-1.

LC-MS m/z (ESI): 434.1[M−H]⁻. ¹H NMR (400 MHz, DMSO-d₆) δ 12.52 (s, 1H), 10.56 (s, 1H), 8.25 (s, 1H), 7.67 (d, J=8.6 Hz, 1H), 7.57-7.38 (m, 2H), 7.30-7.17 (m, 2H), 6.82 (s, 1H), 5.25-5.16 (m, 2H), 4.40-4.25 (m, 1H), 4.00-3.90 (m, 1H).

### Example 88

### Compound 88: (Z)-4-(1-(4-((6-chloro-7-methyl-1H-indol-3-yl)methylene)-2,5-dioxoimidazolidin-1-yl)-2-hydroxyethyl)benzonitrile

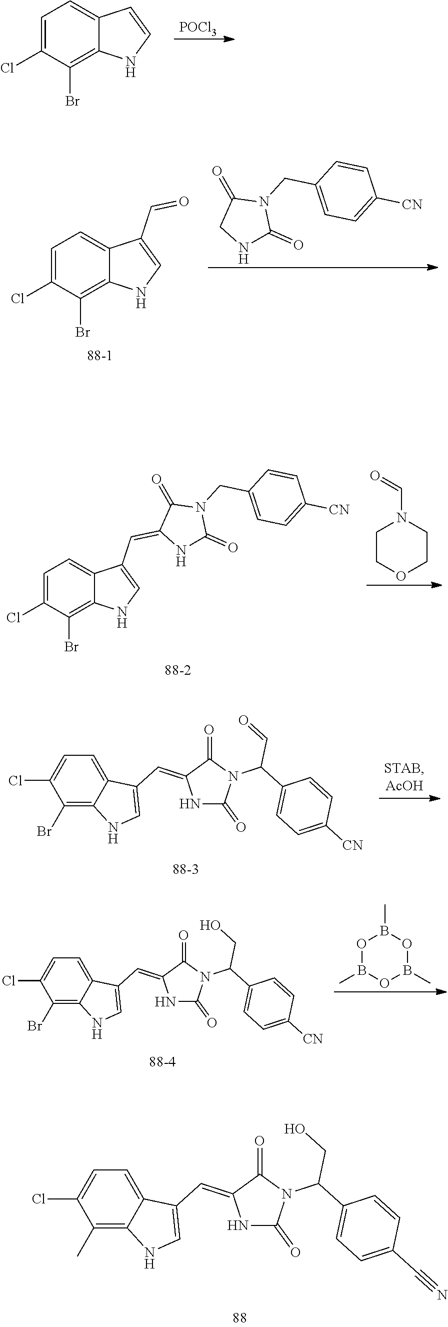

### 88-1: 7-bromo-6-chloro-1H-indole-3-formaldehyde

By using the method for the compound 1-10, the light brown solid compound 88-1 (4.45 g, 68.9%) was prepared by using 7-bromo-6-chloro-1H-indole.

LC-MS m/z (ESI): 257.9[M+H]⁺.

### 88-2: (Z)-4-((4-((7-bromo-6-chloro-1H-indol-3-yl)methylene)-2,5-dioxoimidazolidin-1-yl)methyl)benzonitrile

By using the method for the compound 1, the yellow solid compound 88-2 (5.0 g, 96.9%) was prepared by using the compound 88-1.

LC-MS m/z (ESI): 454.9[M+H]⁺.

### 88-3: (Z)-4-(1-(4-((7-bromo-6-chloro-1H-indol-3-yl)methylene)-2,5-dioxoimidazolidin-1-yl)-2-oxyethyl)benzonitrile

By using the method for the compound 33, the brown solid compound 88-3 (320 mg, 14.9%) was prepared by using the compound 88-2.

LC-MS m/z (ESI): 482.9[M+H]⁺.

### 88-4: (Z)-4-(1-(4-((7-bromo-6-chloro-1H-indol-3-yl)methylene)-2,5-dioxoimidazolidin-1-yl)-2-hydroxyethyl)benzonitrile

By using the method for the compound 37, the yellow solid compound 88-4 (120 mg, 46.7%) was prepared by using the compound 88-3.

LC-MS m/z (ESI): 485.0 [M+H]⁺.

### Compound 88: (Z)-4-(1-(4-((6-chloro-7-methyl-1H-indol-3-yl)methylene)-2,5-dioxoimidazolidin-1-yl)-2-hydroxyethyl)benzonitrile

Pd(dppf)Cl₂ (18.1 mg, 0.025 mmol, 0.10 eq) and K₂CO₃ (204 mg, 1.48 mmol, 6.00 eq) were added into a stirred mixture of the compound 88-4 (120 mg, 0.25 mmol, 1.00 eq) and trimethyl-1,3,5,2,4,6-trioxaterphenyl (186 mg, 1.48 mmol, 6.00 eq) in 1,4-dioxane (12 mL) and water (0.3 mL), and the obtained mixture was stirred under argon protection at 120° C. for 6 h. The mixture was cooled to room temperature and the reaction was terminated at room temperature with water. The obtained mixture was extracted with EtOAc (3×30 mL). Combined organic layers were washed with saline (3×30 mL), dried with anhydrous Na₂SO₄, and filtered. A filtrate was concentrated at a reduced pressure. A crude product was purified by Prep-HPLC to obtain the yellow solid compound 88 (19.3 mg, 18.2%).

LC-MS m/z (ESI): 421.1[M+H]⁺ 0.1H NMR (400 MHz, DMSO-d₆) δ 12.00 (s, 1H), 10.55 (s, 1H), 8.21 (s, 1H), 7.85-7.83 (m, 2H), 7.65-7.60 (m, 3H), 7.15 (d, J=8.5 Hz, 1H), 6.83 (s, 1H), 5.30-5.26 (m, 1H), 4.35-4.30 (m, 1H), 4.04-3.99 (m, 1H), 2.50 (s, 3H).

### Example 92

### Compound 92: (Z)-2-(4-((6-chloro-7-methyl-1H-indol-3-yl)methylene)-2,5-dioxoimidazolidin-1-yl)-2-(4-cyanophenyl)ethyl dihydrophosphate

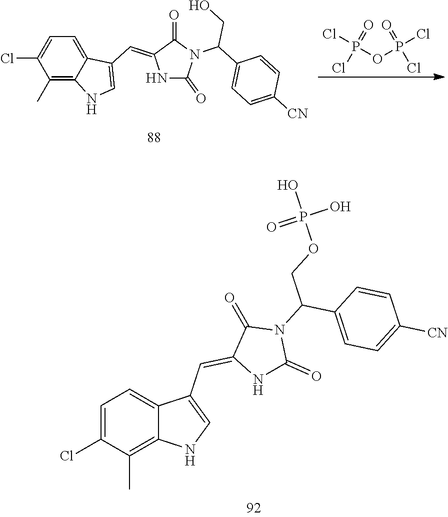 [SMILES: Cc1c(Cl)ccc2c(/C=C3\NC(=O)N(C(CO)c4ccc(C#N)cc4)C3=O)c[nH]c12 ; O=P(Cl)(Cl)OP(=O)(Cl)Cl ; Cc1c(Cl)ccc2c(/C=C3\NC(=O)N(C(COP(=O)(O)O)c4ccc(C#N)cc4)C3=O)c[nH]c12]

By using the method for the compound 42, the yellow solid compound 92 (44.7 mg, 24.6%) was prepared by using the compound 88.

LC-MS m/z (ESI): 499.1[M−H]⁻. ¹H NMR (300 MHz, DMSO-d₆) δ 12.04 (s, 1H), 8.23 (s, 1H), 7.85-7.75 (m, 2H), 7.69-7.56 (m, 3H), 7.13 (d, J=8.5 Hz, 1H), 6.83 (s, 1H), 5.55-5.39 (m, 1H), 4.54-4.36 (m, 2H), 2.52 (s, 3H).

### Example 93

### Compound 93: (Z)-2-(4-((6-chloro-7-fluoro-1H-indol-3-yl)methylene)-2,5-dioxoimidazolidin-1-yl)-2-(4-chlorophenyl)ethyl dihydrophosphate

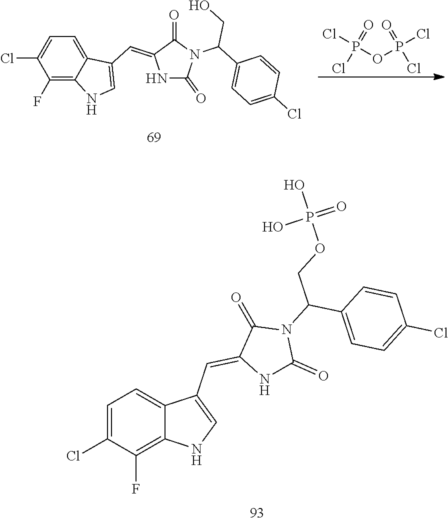 [SMILES: O=C1N/C(=C\c2c[nH]c3c(F)c(Cl)ccc23)C(=O)N1C(CO)c1ccc(Cl)cc1 ; O=P(Cl)(Cl)OP(=O)(Cl)Cl ; O=C1N/C(=C\c2c[nH]c3c(F)c(Cl)ccc23)C(=O)N1C(COP(=O)(O)O)c1ccc(Cl)cc1]

By using the method for the compound 42, the yellow solid compound 93 (232.5 mg, 45.0%) was prepared by using the compound 69.

LC-MS m/z (ESI): 511.9[M−H]⁻. ¹H NMR (400 MHz, DMSO-d₆) δ 12.55 (s, 1H), 10.63 (s, 1H), 8.26 (s, 1H), 7.67 (d, J=8.6 Hz, 1H), 7.52-7.38 (m, 4H), 7.30-7.15 (m, 1H), 6.84 (s, 1H), 5.50-5.32 (m, 1H), 4.90-4.67 (m, 1H), 4.55-4.36 (m, 1H).

### Example 94

### Compound 94: (S,Z)-2-(4-((6-chloro-1H-indol-3-yl)methylene)-2,5-dioxoimidazolidin-1-yl)-2-(3,4-difluorophenyl)dihydroethyl phosphate

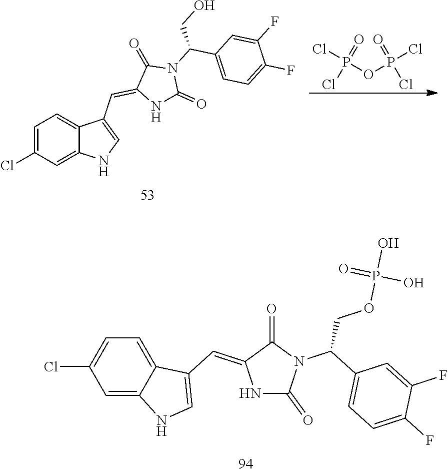 [SMILES: O=C1N/C(=C\c2c[nH]c3cc(Cl)ccc23)C(=O)N1[C@H](CO)c1ccc(F)c(F)c1 ; O=P(Cl)(Cl)OP(=O)(Cl)Cl ; O=C1N/C(=C\c2c[nH]c3cc(Cl)ccc23)C(=O)N1[C@H](COP(=O)(O)O)c1ccc(F)c(F)c1]

By using the method for the compound 42, the yellow solid compound 94 (16.4 mg, 0.032 mmol) was prepared by using the compound 53.

LC-MS m/z (ESI): 498.1 [M+H]⁺ 0.1H NMR (400 MHz, DMSO-d₆) δ 12.04 (s, 1H), 8.19 (s, 1H), 7.83-7.77 (d, J=8.4 Hz, 1H), 7.52-7.47 (m, 2H), 7.54-7.45 (m, 1H), 7.29-7.25 (m, 1H), 7.13 (d, J=8.4 Hz, 1H), 6.86 (s, 1H), 5.40-5.31 (m, 1H), 4.52-4.47 (m, 2H).

### Example 95

### Compound 95: (R,Z)-2-(4-((6-chloro-1H-indol-3-yl)methylene)-2,5-dioxoimidazolidin-1-yl)-2-(3,4-difluorophenyl)dihydroethyl phosphate

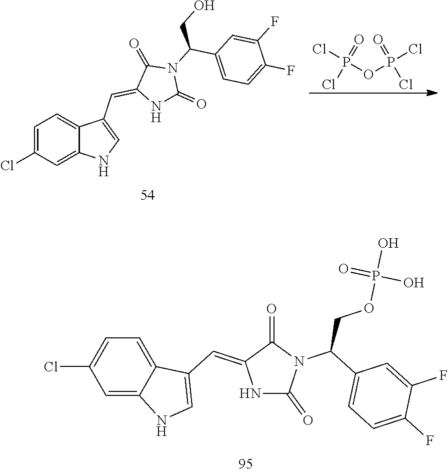 [SMILES: O=C1N/C(=C\c2c[nH]c3cc(Cl)ccc23)C(=O)N1[C@@H](CO)c1ccc(F)c(F)c1 ; O=P(Cl)(Cl)OP(=O)(Cl)Cl ; O=C1N/C(=C\c2c[nH]c3cc(Cl)ccc23)C(=O)N1[C@@H](COP(=O)(O)O)c1ccc(F)c(F)c1]

By using the method for the compound 42, the yellow solid compound 95 (21.2 mg, 0.042 mmol) was prepared by using the compound 54.

LC-MS m/z (ESI): 498.1 [M+H]⁺ 0.1H NMR (400 MHz, DMSO-d₆) δ 12.04 (s, 1H), 8.19 (s, 1H), 7.83-7.77 (d, J=8.4 Hz, 1H), 7.52-7.47 (m, 2H), 7.54-7.45 (m, 1H), 7.29-7.25 (m, 1H), 7.13 (d, J=8.4 Hz, 1H), 6.83 (s, 1H), 5.40-5.31 (m, 1H), 4.52-4.47 (m, 2H).

### Example 96

### Compound 96: (R,Z)-4-(1-(4-((6-chloro-7-methyl-1H-indol-3-yl)methylene)-2,5-dioxoimidazolidin-1-yl)-2-hydroxyethyl)benzonitrile

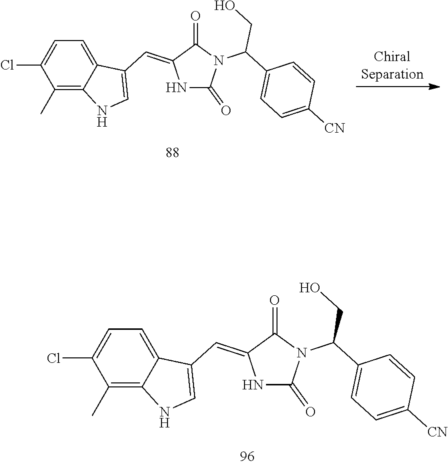 [SMILES: Cc1c(Cl)ccc2c(/C=C3\NC(=O)N(C(CO)c4ccc(C#N)cc4)C3=O)c[nH]c12 ; Cc1c(Cl)ccc2c(/C=C3\NC(=O)N([C@@H](CO)c4ccc(C#N)cc4)C3=O)c[nH]c12]

The compound 88 (60 mg, 0.14 mmol, 1.0 equiv) was separated by a chiral column: CHIRALPAKIG-34.6*50 mm 3 um; Mobile Phase A: MtBE (0.1% DEA): EtOH=93: 7; Flow rate: 1.0 mL/min mL/min; Gradient: isocratic; Injection Volume: 1.0 L mL to finally obtain the yellow solid compound 96 (17.0 mg, 29.1%).

LC-MS m/z (ESI): 419.1[M−H]−. ¹H NMR (400 MHz, DMSO-d₆) δ 12.00 (s, 1H), 10.55 (s, 1H), 8.24-8.19 (m, 1H), 7.88-7.81 (m, 2H), 7.67-7.58 (m, 3H), 7.19-7.12 (m, 1H), 6.84 (s, 1H), 5.32-5.22 (m, 2H), 4.39-4.28 (m, 1H), 4.07-3.97 (m, 1H), 2.52 (s, 3H).

### Example 97

### Compound 97: (S,Z)-4-(1-(4-((6-chloro-7-methyl-1H-indol-3-yl)methylene)-2,5-dioxoimidazolidin-1-yl)-2-hydroxyethyl)benzonitrile

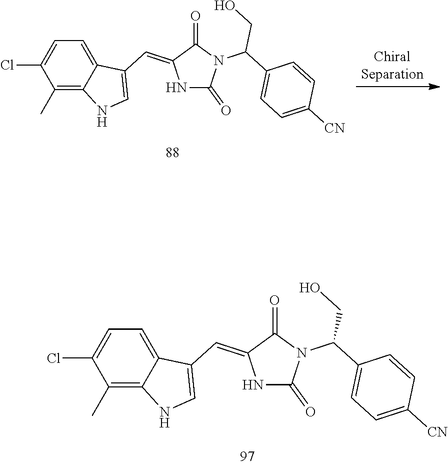 [SMILES: Cc1c(Cl)ccc2c(/C=C3\NC(=O)N(C(CO)c4ccc(C#N)cc4)C3=O)c[nH]c12 ; Cc1c(Cl)ccc2c(/C=C3\NC(=O)N([C@H](CO)c4ccc(C#N)cc4)C3=O)c[nH]c12]

The compound 88 (60 mg, 0.14 mmol, 1.0 equiv) was separated by a chiral column: CHIRALPAKIG-34.6*50 mm 3 um; Mobile Phase A: MtBE (0.1% DEA): EtOH=93: 7; Flow rate: 1.0 mL/min mL/min; Gradient: isocratic; Injection Volume: 1.0 L mL to finally obtain the yellow solid compound 97 (23.3 mg, 39.8%).

LC-MS m/z (ESI): 419.1[M−H]⁻. ¹H NMR (400 MHz, DMSO-d₆) δ 12.00 (s, 1H), 10.55 (s, 1H), 8.24-8.19 (m, 1H), 7.88-7.81 (m, 2H), 7.67-7.58 (m, 3H), 7.19-7.12 (m, 1H), 6.84 (s, 1H), 5.32-5.22 (m, 2H), 4.39-4.28 (m, 1H), 4.07-3.97 (m, 1H), 2.52 (s, 3H).

### Example 98: Compound 98: (Z)-3-(4-chloro-2-fluorobenzyl)-5-((6-chloro-7-fluoro-1H-indol-3-yl)methylene)imidazolidine-2,4-dione

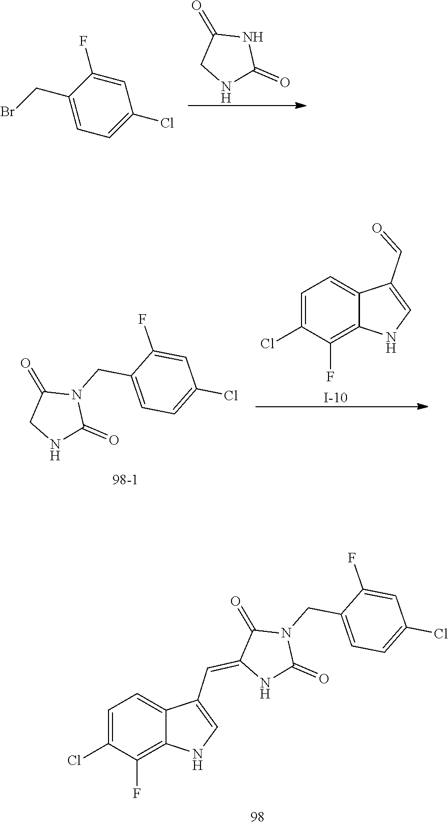

### 98-1: 3-(4-chloro-2-fluorobenzyl)imidazolidine-2,4-dione

By using the method for the compound I-1, the offwhite solid compound 98-1 (2.2 g, 66.1%) was prepared by using 1-(bromomethyl)-4-chloro-2-fluorobenzene and hydantoin.

LC-MS m/z (ESI): 241.0[M−H]⁻.

### Compound 98: (Z)-3-(4-chloro-2-fluorobenzyl)-5-((6-chloro-7-fluoro-1H-indol-3-yl)methylene)imidazolidine-2,4-dione

By using the method for the compound 1, the light yellow solid compound 98 (36.3 mg, 16.2%) was prepared by using 98-1 and I-10.

LC-MS m/z (ESI): 420.1[M−H]⁻. ¹H NMR (400 MHz, DMSO-d₆) δ 12.55 (s, 1H), 10.60 (s, 1H), 8.26 (s, 1H), 7.68 (d, J=8.6 Hz, 1H), 7.47-7.44 (m, 1H), 7.36-7.34 (m, 1H), 7.30-7.27 (m, 1H), 7.25-7.20 (m, 1H), 6.85 (s, 1H), 4.72 (s, 2H).

### Example 99: Compound 99: (Z)-3-(1-(4-chloro-2-fluorophenyl)-2-hydroxyethyl)-5-((6-chloro-7-fluoro-1H-indol-3-yl)methylene)imidazolidine-2,4-dione

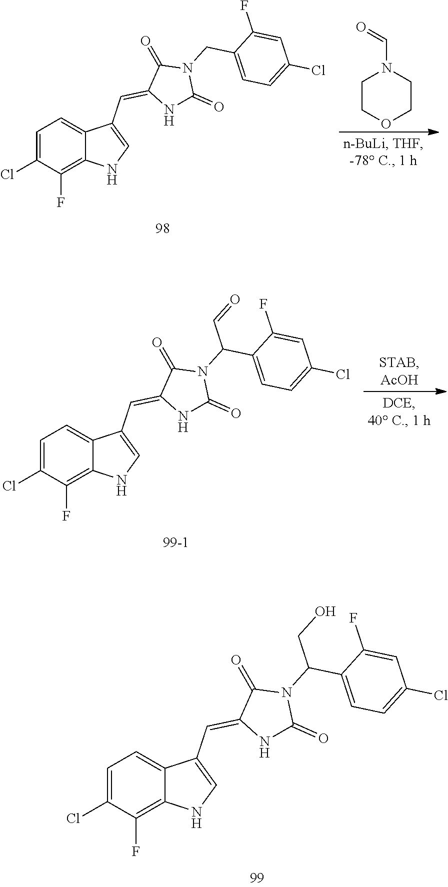 [SMILES: O=C1N/C(=C\c2c[nH]c3c(F)c(Cl)ccc23)C(=O)N1Cc1ccc(Cl)cc1F ; O=CN1CCOCC1 ; O=CC(c1ccc(Cl)cc1F)N1C(=O)N/C(=C\c2c[nH]c3c(F)c(Cl)ccc23)C1=O ; O=C1N/C(=C\c2c[nH]c3c(F)c(Cl)ccc23)C(=O)N1C(CO)c1ccc(Cl)cc1F]

### 99-1: (Z)-2-(4-chloro-2-fluorophenyl)-2-(4-((6-chloro-7-fluoro-1H-indol-3-yl)methylene)-2,5-dioxoimidazolidin-1-yl)acetaldehyde

By using the method for the compound 33, the brown solid compound 99-1 (120 mg, 33.1%) was prepared by using the compound 98.

LC-MS m/z (ESI): 450.0[M+H]⁺.

### Compound 99: (Z)-3-(1-(4-chloro-2-fluorophenyl)-2-hydroxyethyl)-5-((6-chloro-7-fluoro-1H-indol-3-yl)methylene)imidazolidine-2,4-dione

By using the method for the compound 37, the yellow solid compound 99 (32.5 mg, 30.8%) was prepared by using the compound 99-1.

LC-MS m/z (ESI): 450.1[M−H]⁻. ¹H NMR (400 MHz, DMSO-d₆) δ 12.53 (s, 1H), 10.55 (s, 1H), 8.24 (s, 1H), 7.67-7.62 (m, 2H), 7.46-7.44 (m, 1H), 7.34-7.32 (m, 1H), 7.23-7.21 (m, 1H), 6.80 (s, 1H), 5.45-5.41 (m, 1H), 5.30-5.27 (m, 1H), 4.40-4.20 (m, 1H), 3.99-3.81 (m, 1H).

### Example 100: Compound 100: (Z)-3-(4-chloro-2,5-difluorobenzyl)-5-((6-chloro-7-fluoro-1H-indol-3-yl)methylene)imidazolidine-2,4-dione

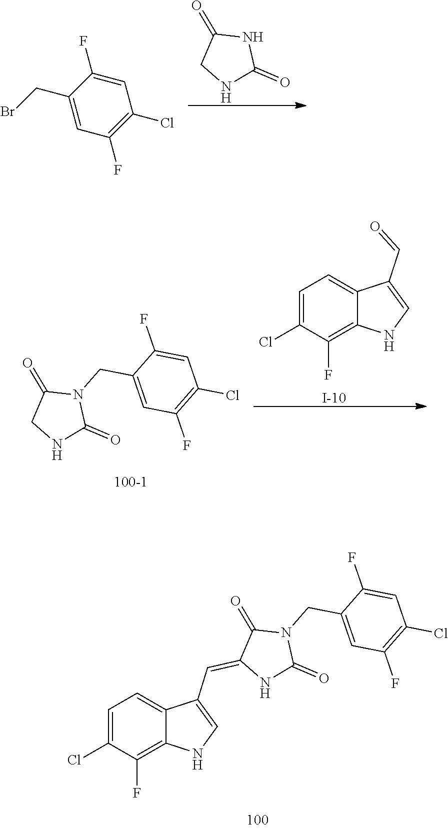

### 100-1: 3-(4-chloro-2,5-difluorobenzyl)imidazolidine-2,4-dione

By using the method for the compound 1-1, the light yellow solid compound 100-1 (1.8 g, 78.5%) was prepared by using hydantoin and 1-(bromomethyl)-4-chloro-2,5-difluorobenzene.

LC-MS m/z (ESI): 259.0[M−H]⁻.

### Compound 100: (Z)-3-(4-chloro-2,5-difluorobenzyl)-5-((6-chloro-7-fluoro-1H-indol-3-yl)methylene)imidazolidine-2,4-dione

By using the method for the compound 1, the light yellow solid compound 100 (45.6 mg, 19.5%) was prepared by using the compounds 100-1 and I-10.

LC-MS m/z (ESI): 438.0[M−H]⁻. ¹H NMR (400 MHz, DMSO-d₆) δ 12.55 (s, 1H), 10.60 (s, 1H), 8.26 (s, 1H), 7.69-7.66 (m, 2H), 7.50-7.42 (m, 1H), 7.28-7.17 (m, 1H), 6.85 (s, 1H), 4.72 (s, 2H).

### Example 101: Compound 101: (Z)-3-(1-(4-chloro-2,5-difluorophenyl)-2-hydroxyethyl)-5-((6-chloro-7-fluoro-1H-indol-3-yl)methylene)imidazolidine-2,4-dione

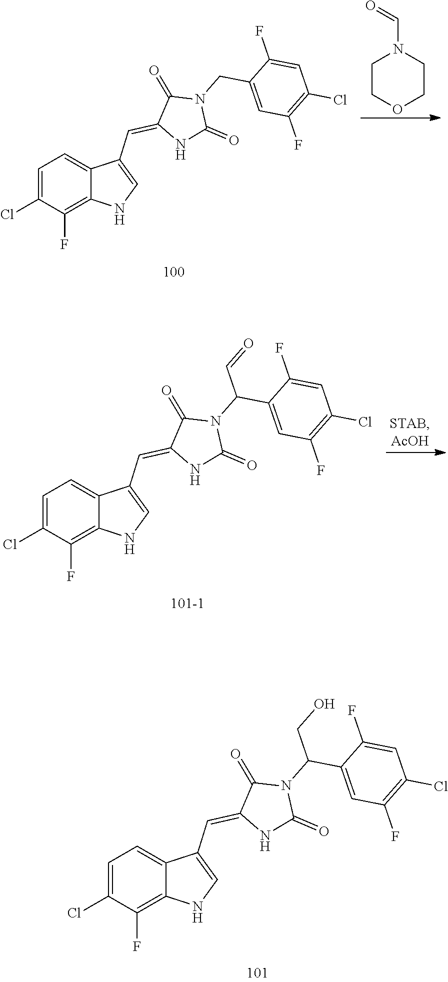

### 101-1: (Z)-2-(4-chloro-2,5-difluorophenyl)-2-(4-((6-chloro-7-fluoro-1H-indol-3-yl)methylene)-2,5-dioxoimidazolidin-1-yl)acetaldehyde

By using the method for the compound 33, the brown solid compound 101-1 (200 mg, 56.0%) was prepared by using the compound 100.

LC-MS m/z (ESI): 466.0[M−H]⁻.

### Compound 101: (Z)-3-(1-(4-chloro-2,5-difluorophenyl)-2-hydroxyethyl)-5-((6-chloro-7-fluoro-1H-indol-3-yl)methylene)imidazolidine-2,4-dione

By using the method for the compound 37, the yellow solid compound 101 (24.3 mg, 14.2%) was prepared by using the compound 101-1.

LC-MS m/z (ESI): 468.1[M−H]⁻. ¹H NMR (300 MHz, DMSO-d₆) δ 12.55 (s, 1H), 10.56 (s, 1H), δ 8.24 (s, 1H), 7.70-7.63 (m, 3H), 7.23-7.18 (m, 1H), 6.81 (s, 1H), 5.44-5.39 (m, 1H), 5.34-5.20 (m, 1H), 4.28-4.19 (m, 1H), 3.96-3.85 (m, 1H).

### Example 102: Compound 102: (Z)-3-(4-chloro-2,6-difluorobenzyl)-5-((6-chloro-7-fluoro-1H-indol-3-yl)methylene)imidazolidine-2,4-dione

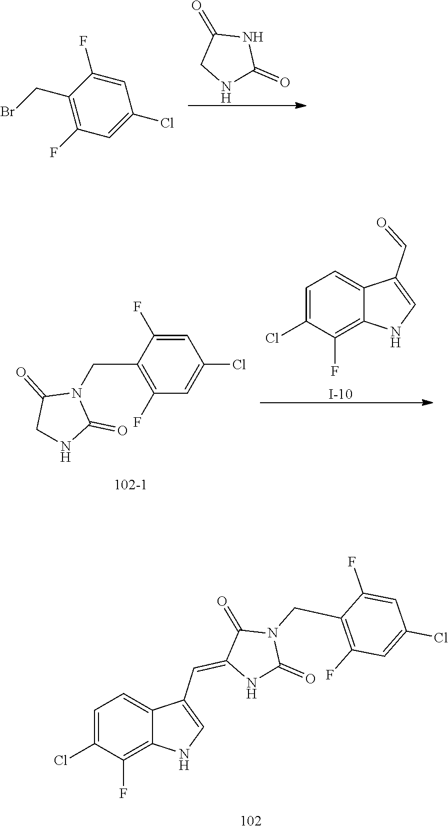

### 102-1: 3-(4-chloro-2,6-difluorobenzyl)imidazolidine-2,4-dione

By using the method for the compound I-1, the white solid compound 102-1 (1.3 g, 56.9%) was prepared by using hydantoin and 2-(bromomethyl)-5-1,3-difluorobenzene.

LC-MS m/z (ESI): 259.0[M−H]⁻.

### Compound 102: (Z)-3-(4-chloro-2,6-difluorobenzyl)-5-((6-chloro-7-fluoro-1H-indol-3-yl)methylene)imidazolidine-2,4-dione

By using the method for the compound 1, the light yellow solid compound 102 (41.4 mg, 18.3%) was prepared by using the compounds 102-1 and I-10.

LC-MS m/z (ESI): 438.0[M−H]⁻. ¹H NMR (400 MHz, DMSO-d₆) 12.52 (s, 1H), 10.50 (s, 1H), 8.23 (s, 1H), 7.65 (d, J=8.5 Hz, 1H), 7.42-7.32 (m, 2H), 7.22-7.18 (m, 1H), 6.80 (s, 1H), 4.76 (s, 2H).

### Example 103: Compound 103: (Z)-3-(1-(4-chloro-2,6-difluorophenyl)-2-hydroxyethyl)-5-((6-chloro-7-fluoro-1H-indol-3-yl)methylene)imidazolidine-2,4-dione

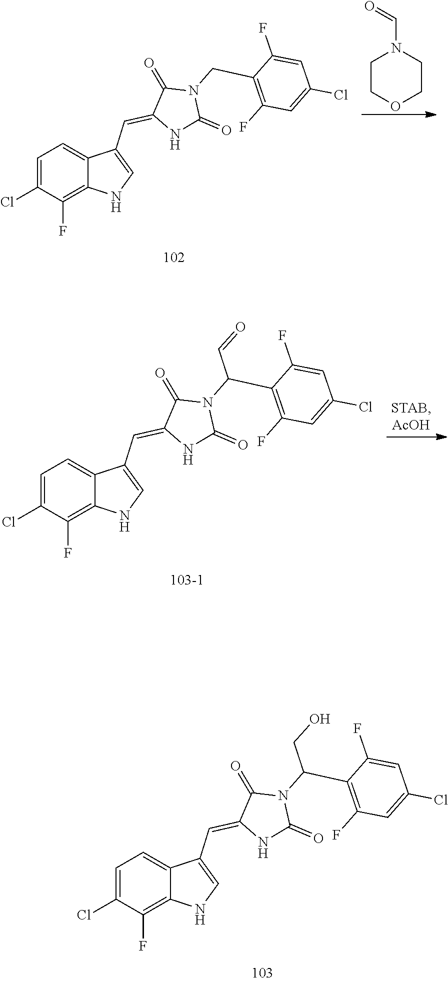

### 103-1: (Z)-2-(4-chloro-2,6 difluorophenyl)-2-(4-((6-chloro-7-fluoro-1H-indol-3-yl)methylene)-2,5-dioxoimidazolidin-1-yl)acetaldehyde

By using the method for the compound 33, the brown solid compound 103-1 (160 mg, 36.8%) was prepared by using the compound 102.

LC-MS m/z (ESI): 468.2[M+H]⁺.

### Compound 103: (Z)-3-(1-(4-chloro-2,6-difluorophenyl)-2-hydroxyethyl)-5-((6-chloro-7-fluoro-1H-indol-3-yl)methylene)imidazolidine-2,4-dione

By using the method for the compound 37, the light yellow solid compound 103 (23.3 mg, 12.48%) was prepared by using the compound 103-1.

LC-MS m/z (ESI): 468.1[M−H]⁻. ¹H NMR (300 MHz, DMSO-d₆) δ 12.51 (s, 1H), 10.48 (s, 1H), 8.22 (s, 1H), 7.64 (d, J=8.6 Hz, 1H), 7.42-7.29 (m, 2H), 7.23-7.10 (m, 1H), 6.77 (s, 1H), 5.50-5.30 (m, 1H), 5.28-5.10 (m, 1H), 4.50-4.15 (m, 1H), 4.10-3.90 (m, 1H)

### Example 104: Compound 104: (Z)-3-(4-chloro-2,3-difluorobenzyl)-5-((6-chloro-7-fluoro-1H-indol-3-yl)methylene)imidazolidine-2,4-dione

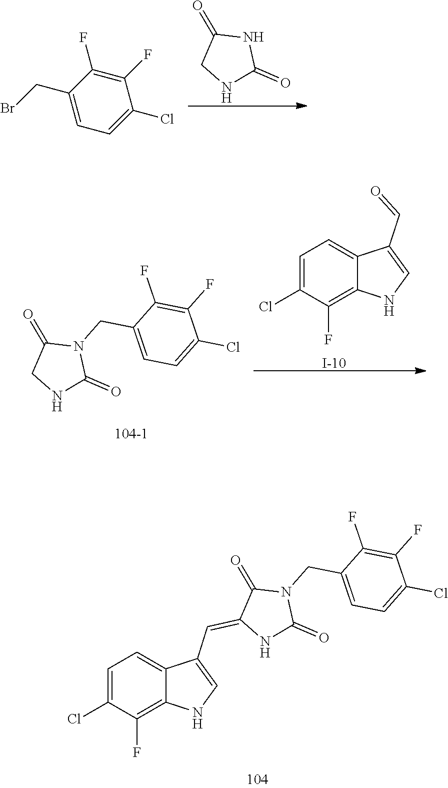

### 104-1: 3-(4-chloro-2,3-difluorobenzyl)imidazolidine-2,4-dione

By using the method for the compound I-1, the white solid compound 104-1 (0.9 g, 3.3 mmol, 79.0%) was prepared by using hydantoin and 1-(bromomethyl)-4-chloro-2,3-difluorobenzene.

LC-MS m/z (ESI): 259.0[M−H]⁻.

### Compound 104: (Z)-3-(4-chloro-2,3-difluorobenzyl)-5-((6-chloro-7-fluoro-1H-indol-3-yl)methylene)imidazolidine-2,4-dione

By using the method for the compound 1, the yellow solid compound 104 (41.6 mg, 16.2%) was prepared by using the compounds 104-1 and I-10.

LC-MS m/z (ESI): 438.0[M−H]⁻. ¹H NMR (400 MHz, DMSO-d₆) δ 12.55 (s, 1H), 10.59 (s, 1H), 8.25 (s, 1H), 7.69 (d, J=8.6 Hz, 1H), 7.52-7.38 (m, 1H), 7.28-7.16 (m, 2H), 6.86 (s, 1H), 4.77 (s, 2H)

### Example 105: Compound 105: (Z)-3-(1-(4-chloro-2,3-difluorophenyl)-2-hydroxyethyl)-5-((6-chloro-7-fluoro-1H-indol-3-yl)methylene)imidazolidine-2,4-dione

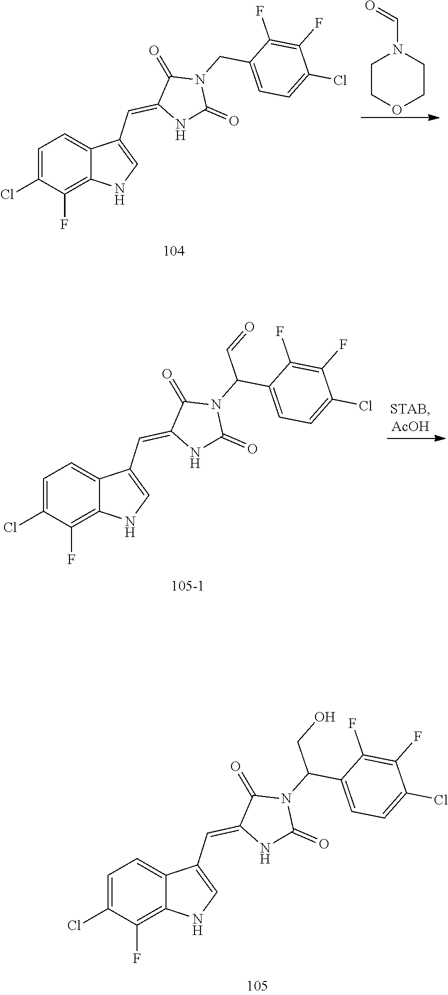

### 105-1: (Z)-2-(4-chloro-2,3-difluorophenyl)-2-(4-((6-chloro-7-fluoro-1H-indol-3-yl)methylene)-2,5-dioxoimidazolidin-1-yl)acetaldehyde

By using the method for the compound 33, the brown solid compound 105-1 (257 mg, 55.3%) was prepared by using the compound 104.

LC-MS m/z (ESI): 468.0[M+H]⁺.

### Compound 105: (Z)-3-(1-(4-chloro-2,3-difluorophenyl)-2-hydroxyethyl)-5-((6-chloro-7-fluoro-1H-indol-3-yl)methylene)imidazolidine-2,4-dione

By using the method for the compound 37, the light yellow solid compound 105 (20.5 mg, 22.8%) was prepared by using the compound 105-1.

LC-MS m/z (ESI): 468.1[M−H]⁻. ¹H NMR (400 MHz, DMSO-d₆) δ 12.54 (s, 1H), 10.58 (s, 1H), 8.24 (s, 1H), 7.66 (d, J=8.5 Hz, 1H), 7.55-7.36 (m, 2H), 7.34-7.11 (m, 1H), 6.81 (s, 1H), 5.61-5.40 (m, 1H), 5.39-5.20 (m, 1H), 4.50-4.20 (m, 1H), 4.05-3.85 (m, 1H).

### Example 106: Compound 106: (Z)-3-(4-chloro-3,5-difluorobenzyl)-5-((6-chloro-7-fluoro-1H-indol-3-yl)methylene)imidazolidine-2,4-dione

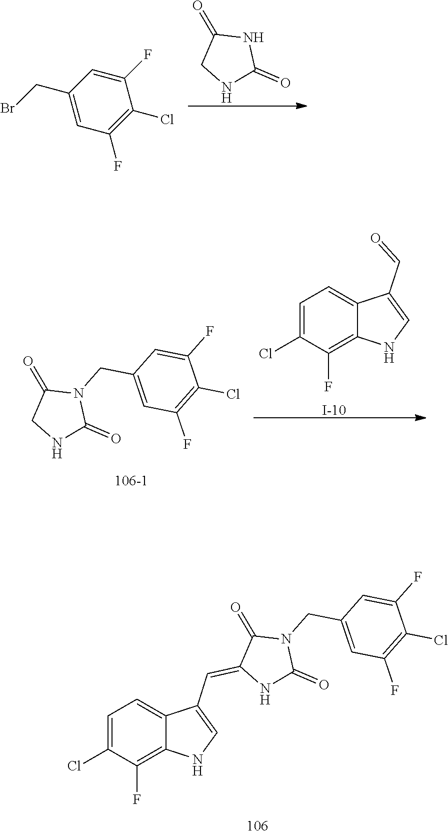

### 106-1: 3-(4-chloro-3,5-difluorobenzyl)imidazolidine-2,4-dione

By using the method for the compound I-1, the white solid compound 106-1 (1.82 g, 83.2%) was prepared by using the hydantoin and 5-(bromomethyl)-2-chloro-1,3-difluorbenzene.

LC-MS m/z (ESI): 259.0[M−H]⁻.

### Compound 106: (Z)-3-(4-chloro-3,5-difluorobenzyl)-5-((6-chloro-7-fluoro-1H-indol-3-yl)methylene)imidazolidine-2,4-dione

By using the method for the compound 1, the gray solid compound 106 (38.0 mg, 16.5%) was prepared by using the compounds 106-1 and I-10.

LC-MS m/z (ESI): 438.0[M−H]⁻. ¹H NMR (400 MHz, DMSO-d₆) δ 12.55 (s, 1H), 10.59 (s, 1H), 8.27 (s, 1H), 7.68 (d, J=8.6 Hz, 1H), 7.40-7.29 (m, 2H), 7.28-7.10 (m, 1H), 6.86 (s, 1H), 4.72 (s, 2H).

### Example 107: Compound 107: (Z)-3-(1-(4-chloro-3,5-difluorophenyl)-2-hydroxyethyl)-5-((6-chloro-7-fluoro-1H-indol-3-yl)methylene)imidazolidine-2,4-dione

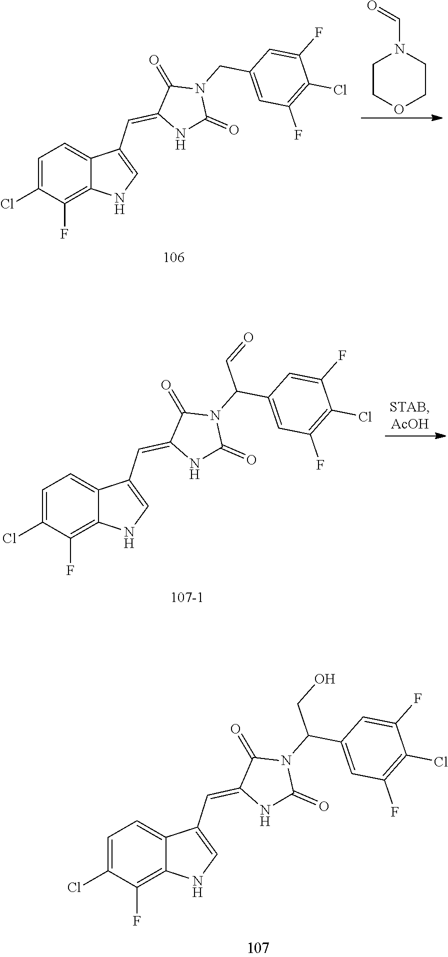

### 107-1: (Z)-2-(4-chloro-3,5-difluorophenyl)-2-(4-((6-chloro-7-fluoro-1H-indol-3-yl)methylene)-2,5-dioxoimidazolidin-1-yl)acetaldehyde

By using the method for the compound 33, the brown solid compound 107-1 (180 mg, 34.02%) was prepared by using the compound 106.

LC-MS m/z (ESI): 466.0[M−H]⁻.

### Compound 107: (Z)-3-(1-(4-chloro-3,5-difluorophenyl)-2-hydroxyethyl)-5-((6-chloro-7-fluoro-1H-indol-3-yl)methylene)imidazolidine-2,4-dione

By using the method for the compound 37, the yellow solid compound 107 (31.1 mg, 24.7%) was prepared by using the compound 107-1.

LC-MS m/z (ESI): 468.1[M−H]⁻. ¹H NMR (300 MHz, DMSO-d₆) δ 11.90-10.50 (br, 1H), 8.26 (s, 1H), 7.67 (d, J=8.6 Hz, 1H), 7.45-7.35 (m, 2H), 7.30-7.16 (m, 1H), 6.82 (s, 1H), 5.45-5.17 (m, 2H), 4.4-4.12 (m, 1H), 4.10-3.85 (m, 1H)

### Example 108: Compound 108: (Z)-5-((6-chloro-7-fluoro-1H-indol-3-yl)methylene)-3-(1-(4-chlorophenyl)-3-hydroxypropyl)imidazolidine-2,4-dione

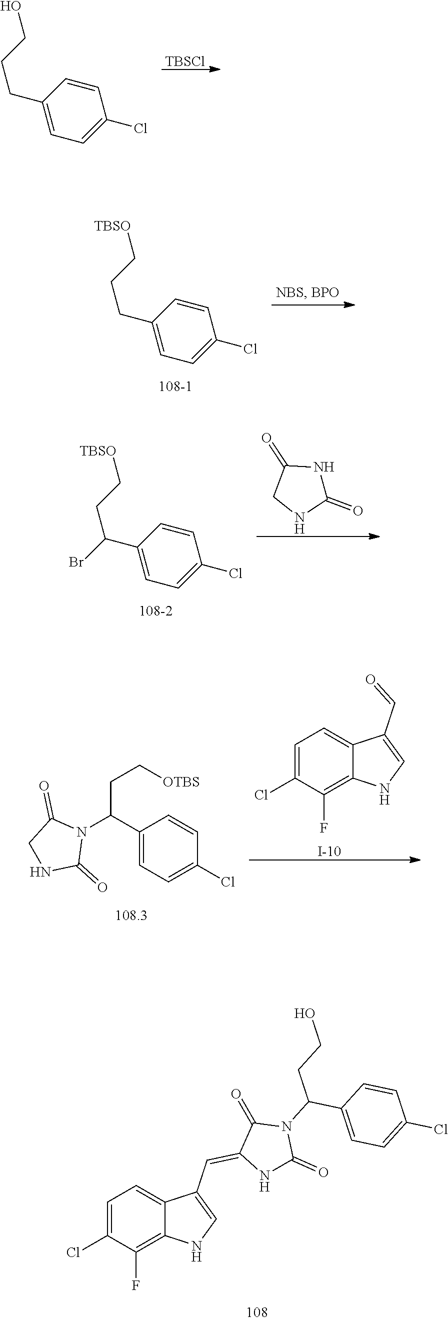

### 108-1: tert-butyl (3-(4-chlorophenyl)propoxyl)dimethylsilane

At room temperature and in a nitrogen atmosphere, TEA (8.9 g, 87.95 mmol, 3.00 eq) was added in batches into a stirred solution of 3-(4-chlorophenyl)-1-propanol (5 g, 29.30 mmol, 1.00 eq) and DMAP (0.36 g, 2.95 mmol, 0.10 eq) in DCM (50 mL), and the obtained mixture was stirred in the nitrogen atmosphere at room temperature for 16 h. The obtained mixture was concentrated at a reduced pressure. A residue was purified by silica gel column chromatography and eluted with PE/EA (10:1) to obtain the colorless liquid compound 108-1 (6.6 g, 78.5%).

### 108-2: (3-bromo-3-(4-chlorophenyl)propoxyl)(tert-butyl)dimethylsilane

At room temperature, a benzoylphenyl carbon peroxide (168 mg, 0.70 mmol, 0.03 eq) was added into a stirred mixture of the 108-1 (6.6 g, 23.17 mmol, 1.00 eq) and NBS (4.12 g, 23.15 mmol, 1.00 eq) in CCl₄ (120 mL), and the obtained mixture was stirred for 3 h. The mixture was cooled to room temperature. The obtained mixture was filtered. A filter cake was washed with CCl₄ (2×20 mL). A filtrate was concentrated at a reduced pressure. A residue was purified by silica gel column chromatography and eluted with PE/EA (15:1) to obtain the light brown liquid compound 108-2 (2.7 g, 25.8%).

### 108-3: 3-(3-((tert-butyldimethylsilyl)oxyl)-1-(4-chlorophenyl)propyl)imidazolidine-2,4-dione

At room temperature, the 108-2 (2.7 g, 7.42 mmol, 1.00 eq) was added into a mixture of hydantoin (2.23 g, 22.28 mmol, 3.00 eq) and K₂CO₃ (1.54 g, 11.13 mmol, 1.50 eq) in DMF (54 mL). In a nitrogen atmosphere, the obtained mixture was stirred at 60° C. for 16 h. The mixture was cooled to room temperature. A cold saturated ammonium chloride aqueous solution (200 mL) was added to terminate the reaction. The obtained mixture was extracted with ethyl acetate (3×150 mL). Combined organic layers were washed with saline (2×100 mL) and dried with anhydrous Na₂SO₄. After the organic layers were filtered, a filtrate was concentrated at a reduced pressure. A residue was purified by fast reversed-phase chromatography to obtain the light yellow syrupy compound 108-3 (1.5 g, 47.0%).

LC-MS m/z (ESI): 383.1[M+H]⁺.

### Compound 108: (Z)-5-((6-chloro-7-fluoro-1H-indol-3-yl)methylene)-3-(1-(4-chlorophenyl)-3-hydroxypropyl)imidazolidine-2,4-dione

At room temperature, HOAc (122 mg, 2.03 mmol, 5.00 eq) was dropwise added into a stirred solution of 108-3 (186 mg, 0.49 mmol, 1.20 eq) and 6-chloro-7-fluoro-1H-indole-3-formaldehyde (80 mg, 0.41 mmol, 1.00 eq) in ethanol (10 mL). In an argon atmosphere, the obtained mixture was stirred at 100° C. for 16 min and cooled to room temperature. The obtained mixture was concentrated at a reduced pressure. A residue was purified by silica gel column chromatography and eluted with PE/EA (3:1) to obtain a brown syrupy crude product. The residue was dissolved in methanol (2 mL). A methanol solution (0.51 mL, 2.04 mmol, 5.04 eq) was dropwise added into the mixture at room temperature. The obtained mixture was then stirred at room temperature for 2 h, and the obtained mixture was concentrated in vacuum. The crude product (50 mg) was purified by HPLC to obtain the yellow solid compound 108 (12.5 mg, 6.11%).

LC-MS m/z (ESI): 446.0[M−H]⁻. ¹H NMR (400 MHz, DMSO-d₆) δ 8.23 (s, 1H), 7.65 (d, J=8.6 Hz, 1H), 7.51-7.40 (m, 4H), 7.30-7.16 (m, 1H), 6.79 (s, 1H), 5.50-5.22 (m, J=9.4, 6.4 Hz, 1H), 4.64 (s, 1H), 3.46-3.42 (m, 2H), 2.71-2.56 (m, 1H), 2.34-2.22 (m, 1H).

### Example 109: compound 109: (Z)-5-((6-chloro-7-fluoro-1H-indol-3-yl)methylene)-3-(1-(4-chlorophenyl)-4-hydroxybutyl)imidazolidine-2,4-dione

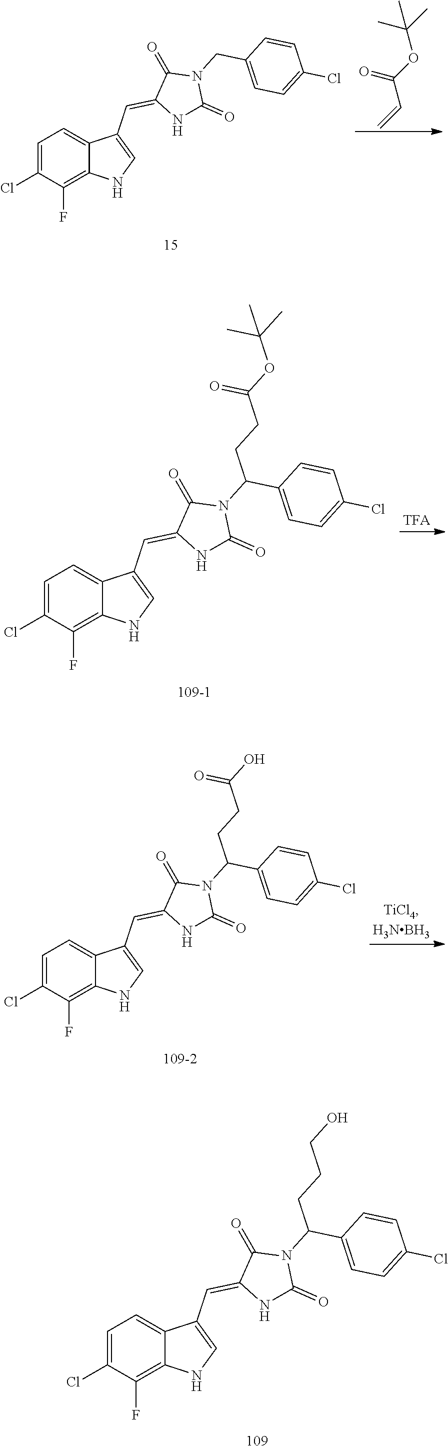

### 109-1: tert-butyl(Z)-4-(4-((6-chloro-7-fluoro-1H-indol-3-yl)methylene)-2,5-dioxoimidazolidin-1-yl)-4-(4-chlorophenyl)butyrate

At −78° C. and in an argon atmosphere, n-BuLi (4.2 mL, 10.50 mmol, 10.11 eq) was dropwise added into a stirred solution of the compound 15 (420 mg, 1.04 mmol, 1.00 eq) and tert-butyl 2-acrylate (2.66 g, 20.78 mmol, 20.00 eq) in THF (140 mL). In the argon atmosphere, the obtained mixture was stirred at −78° C. for 1 h. At −82° C., the reaction was quenched with a solution of THF in acetic acid. The mixture was heated to −30° C. and the obtained mixture was poured into 300 mL of water. The obtained mixture was extracted with ethyl acetate (1×300 mL). Combined organic layers was washed with saline (2×150 mL) and dried with anhydrous Na₂SO₄. After the organic layers were filtered, a filtrate was concentrated at a reduced pressure. A residue was purified by fast reversed-phase chromatography to obtain the light yellow solid compound 109-1 (305 mg, 53.2%).

LC-MS m/z (ESI): 530.2[M−H]⁻.

### 109-2: (Z)-4-(4-((6-chloro-7-fluoro-1H-indol-3-yl)methylene)-2,5-dioxoimidazolidin-1-yl)-4-(4-chlorophenyl)butyric acid

At room temperature, TFA (3 mL) was dropwise added into a stirred solution of 109-1 (300 mg, 0.56 mmol, 1.00 eq) in DCM (20 mL), and the obtained mixture was stirred at room temperature for 2 h. The obtained mixture was concentrated in vacuum. A residue was purified by fast reversed-phase chromatography to obtain the yellow solid compound 109-2 (135 mg, 49.8%).

LC-MS m/z (ESI): 476.1[M+H]⁺.

### Example 109: Compound 109: (Z)-5-((6-chloro-7-fluoro-1H-indol-3-yl)methylene)-3-(1-(4-chlorophenyl)-4-hydroxybutyl)imidazolidine-2,4-dione

At room temperature and in a nitrogen atmosphere, TiCl₄ (8.4 mg, 0.04 mmol, 0.20 eq) was dropwise added into a mixture of 109-2 (105 mg, 0.22 mmol, 1.00 eq) and ammonia borane (13.7 mg, 0.44 mmol, 2.01 eq) in ether (2.8 mL), and the obtained mixture was stirred in the nitrogen atmosphere at room temperature for 2 h. A saturated ammonium chloride (20 mL) solution was added at room temperature to terminate the reaction. The obtained mixture was extracted with ethyl acetate (4×50 mL). Combined organic layers were washed with saline (2×50 mL) and dried with anhydrous Na₂SO₄. After the organic layers were filtered, a filtrate was concentrated at a reduced pressure. A crude product was purified by HPLC to obtain the yellow solid compound 109 (50.6 mg, 48.9%).

LC-MS m/z (ESI): 460.0[M−H]⁻. ¹H NMR (400 MHz, DMSO-d₆) δ 12.04 br, 1H), 10.63 (br, 1H), 8.24 (s, 1H), 7.66 (d, J=8.6 Hz, 1H), 7.55-7.35 (m, 4H), 7.30-7.10 (m, 1H), 6.81 (s, 1H), 5.22-5.10 (m, 1H), 4.55-4.31 (m, 1H), 3.5-3.41 (m, 2H), 2.49-2.37 (m, 1H), 2.30-2.10 (m, 1H), 1.51-1.30 (m, 2H)

### Compound 110: (Z)-3-(4-((6-chloro-7-fluoro-1H-indol-3-yl)methylene)-2,5-dioxoimidazolidin-1-yl)-3-(4-chlorophenyl)propyl dihydrophosphate

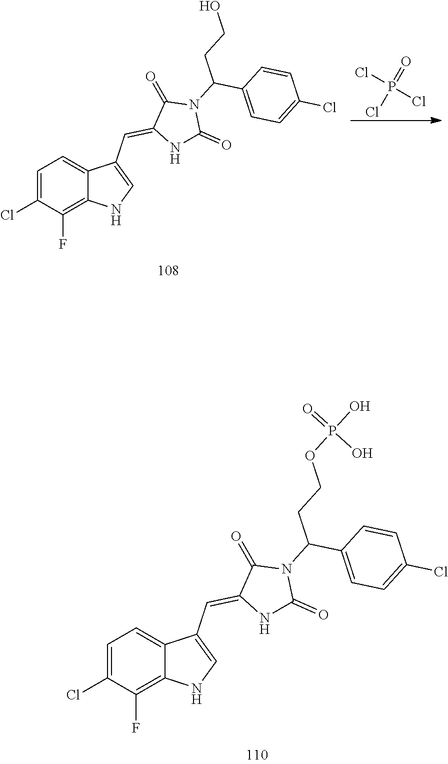 [SMILES: O=C1N/C(=C\c2c[nH]c3c(F)c(Cl)ccc23)C(=O)N1C(CCO)c1ccc(Cl)cc1 ; O=P(Cl)(Cl)Cl ; O=C1N/C(=C\c2c[nH]c3c(F)c(Cl)ccc23)C(=O)N1C(CCOP(=O)(O)O)c1ccc(Cl)cc1]

At 0° C. and in a nitrogen atmosphere, phosphoryl chloride (513 mg, 3.35 mmol, 10.00 eq) was added into a stirred solution of the compound 108 (150 mg, 0.34 mmol, 1.00 eq) in THF (9 mL). In the nitrogen atmosphere, the obtained mixture was stirred at room temperature for 7 h. The reaction was quenched with a saturated NaHCO₃ solution at room temperature, and the obtained mixture was stirred for 30 min. The mixture was acidized with HCl (aqueous solution) till pH=2 and extracted with EtOAc (3×50 mL). Combined organic layers were washed with saline (2×50 mL) and dried with anhydrous Na₂SO₄. After the organic layers were filtered, a filtrate was concentrated at a reduced pressure. A residue was purified by fast reversed-phase chromatography to obtain the yellow solid compound 110 (39.2 mg, 21.9%).

LC-MS m/z (ESI): 528.0[M+H]⁺ 0.1H NMR (300 MHz, DMSO-d₆)) δ 8.24 (s, 1H), 7.63 (d, J=8.6 Hz, 1H), 7.51-7.35 (m, 4H), 7.25-7.11 (m, 1H), 6.79 (s, 1H), 5.45-5.21 (m, 1H), 3.70-3.60 (m, 2H), 2.70-2.51 (m, 1H), 2.45-2.40 (m, 1H).

### Test Example 1: Activity Determination for Compounds

### Test of Candidate Compounds on Growth Inhibition of Cells Determined by CellTiter Glo

Candidate compounds were dissolved in DMSO, with an initial concentration of 50 mM, and were diluted to a 384-well storage plate at a triple gradient. The compounds in the storage plate were pumped into a 96-well cell culture plate (Corning 3610) with an Echo 520 instrument, with a volume of 100 nL/well, and were diluted at a triple gradient in duplicate. Blood cancer cells RS4; 11 were inoculated into the 96-well cell culture plate through Multidrop 3000 cells per well, with a volume of 100 μL. The final compound concentration ranged from 50 μM to 0.01 μM at 9 concentration gradients and the DMSO final concentration was 0.1%. The cells and compounds were co-incubated in a cell incubator at 37° C. for 72 h, a CellTiter-Glo Luminescent Cell Viability Assay reagent was added 100 μL/well, the cells and compound were uniformly mixed and incubated at room temperature for 10 min, and Envision reading was performed. A sample treated with 50 μM of a positive compound was taken as a positive control and a sample treated with 100 nL of DMSO was taken as a negative control. A calculation formula was as follows: (sample reading-positive control reading)/(negative control reading-positive control reading)*100%, and IC₅₀ was calculated by a four-parameter formula. The cell viability IC₅₀ intervals of the compounds were as follows: A≤1 μM, 1≤B≤10 μM, C>10 μM.

### HTRF Test: Test of Respectively Detecting Inhibition of Candidate Compounds on MDM2-p53 and MDMX-p53 Protein Binding

Candidate compounds were dissolved in DMSO, with an initial concentration of 50 mM, and were diluted to a 384-well storage plate at a triple gradient. The compounds in the storage plate were pumped into a 384-well cell culture plate (PE 6008280) with an Echo 520 instrument, with a volume of 100 nL/well, and were diluted at a triple gradient in duplicate. Proteins and/or polypeptides were respectively diluted with HTRT buffer solutions (1×PBS pH 7.4, 0.1% BSA and 1 mM DTT) as follows: An MDM2-p53 reaction system was purified 10 nM GST-MDM2 (2-138 aa) and 5 nM HIS-p53 (2-312 aa) proteins; an MDMX reaction system was 5 nM GST-MDMX (2-134 aa) proteins and 100 nM WI HIS-p53 peptide fragments (Rui Biotech). Proteins in a negative control group in the MDM2-p53 binding inhibition test were 10 nM GST proteins and 5 nM HIS-p53 (2-312 aa) proteins, proteins in a negative control group in the MDMX-p53 binding inhibition test were 10 nM GST proteins and 100 nM WT HIS-p53 peptide fragments, and positive control groups in the above two binding tests were DMSO treated groups. 10 μL of the above proteins and/or polypeptide reaction system were respectively added into the 384-well plate with the added compounds through multidrop. The final compound concentration ranged from 250 IIM to 0.05 nM, they were diluted at a triple gradient, with 15 concentrations in duplicate. The DMSO final concentration was 1%, and they were incubated at room temperature for 1 h. Anti 6HIS-Tb cryptate Gold (Cisbio 61HI2TLF) and Anti GST-XL665 (Cisbio 61GSTXLF) diluted at 400 times were added into the above buffer solution, and they were uniformly mixed. The antibody mixed solution was added into the 384-well plate through multidrop 10 μL/well, and was incubated at room temperature for 1 h. Data was read by Envision. The wavelength of laser-excited light was 340 nM, readings with the wavelengths of emitted light of 665 nM and 615 nM were respectively read, and a 665/615 ratio was calculated. A known MDM2-p53 binding inhibitor Siremadlin (NVP-HDM201; MCE HY-18658) was the positive control group. 100 nL of DMSO treated group was the negative control group. A calculation formula was as follows: (sample reading-positive control reading)/(negative control reading-positive control reading)*100%, and IC₅₀ was calculated by a four-parameter formula. The protein binding IC₅₀ intervals of the compounds were as follows: A≤10 nM; 10<B≤100 nM; C>100 nM.

| | HTRF test | | CellTiter Glo test |
|---|---|---|---|
| | MDM2 | MDMX | RS4; 11 |
| Examples | IC₅₀ (nM) | IC₅₀ (nM) | IC₅₀ (μM) |
| 1 | B | B | B |
| 2 | B | B | A |
| 3 | B | B | A |
| 4 | B | B | A |
| 5 | B | B | B |
| 6 | B | B | / |
| 7 | B | B | / |
| 8 | B | B | B |
| 9 | B | B | C |
| 10 | B | B | B |
| 11 | A | B | B |
| 12 | B | B | C |
| 13 | A | B | C |
| 14 | A | A | A |
| 15 | B | B | A |
| 16 | A | A | A |
| 17 | B | B | A |
| 18 | B | B | A |
| 19 | A | A | A |
| 20 | B | B | A |
| 21 | B | A | A |
| 22 | A | B | / |
| 23 | B | B | / |
| 24 | B | B | B |
| 25 | B | B | / |
| 26 | A | A | A |
| 27 | A | A | A |
| 28 | A | A | A |
| 29 | A | A | B |
| 30 | A | A | A |
| 31 | B | B | B |
| 32 | A | A | B |
| 33 | B | B | B |
| 34 | A | B | A |
| 35 | B | B | A |
| 36 | A | B | A |
| 37 | B | B | A |
| 38 | A | B | A |
| 39 | B | B | B |
| 40 | B | C | / |
| 41 | A | A | A |
| 43 | B | B | / |
| 44 | A | A | / |
| 45 | B | B | A |
| 48 | A | A | A |
| 49 | A | B | A |
| 50 | B | C | A |
| 53 | A | A | B |
| 54 | B | B | A |
| 56 | B | B | B |
| 57 | A | A | A |
| 58 | A | A | A |
| 59 | A | B | A |
| 60 | A | A | A |
| 61 | A | B | B |
| 62 | A | B | A |
| 64 | A | A | A |
| 66 | A | A | A |
| 67 | A | A | A |
| 68 | A | A | A |
| 69 | B | B | A |
| 70 | B | B | A |
| 72 | A | A | A |
| 74 | C | B | / |
| 77 | B | B | A |
| 78 | B | B | A |
| 79 | B | B | A |
| 84 | A | A | A |
| 85 | A | A | A |
| 86 | B | B | A |
| 88 | A | A | A |
| 92 | B | B | / |
| 94 | A | A | / |
| 95 | B | B | / |
| 96 | A | A | A |
| 97 | B | B | A |
| 98 | B | C | A |
| 99 | B | B | A |
| 100 | A | B | A |
| 101 | B | C | B |
| 102 | B | B | A |
| 103 | A | C | B |
| 104 | B | B | A |
| 105 | B | B | A |
| 106 | B | B | A |
| 107 | B | B | A |
| 108 | / | / | A |
| 109 | / | / | A |

### Test Example 2: Solubility Test

### 1) Preparation of a Stock Solution

A test substance and a control drug progesterone were prepared with DMSO into a 10 mM stock solution

### 2) Steps of Determining Solubility

30 μL 10 mM stock solution prepared from the test substance and the control drug progesterone was taken and added into bottles in corresponding positions of 96-well sample plate in a specified order. Then, 970 μL of phosphate buffer with pH 7.4 was added into corresponding bottles of the sample plate. The experiments were two parallel experiments. A stirring rod was added into each bottle, and each bottle was covered with a polytetrafluoroethylene/silicon organic resin bottle plug. Then, a sample tray was placed in Eppendorf Thermomixer Comfort and vibrated at a rotating speed of 1100/min at 25° C. for 2 h. 2 h later, the bottle plug was removed, the stirring rod was taken away with a large magnet, and then samples were transferred to a filter plate from the sample plate. A vacuum pump generated a negative pressure to filter the samples. 5 μL of a filtrate was transferred to a new sample plate. Then, 5 μL of DMSO and 490 μL of acetonitrile/water (1:1 in v/v) were added. The dilution ratio might be adjusted according to the magnitude of the solubility of the test substance or the intensity of a liquid response signal thereof.

### 3) Preparation of a Standard Product Solution

15 μL of the stock solution was transferred from 10 mM DMSO stock solution to an empty plate, and 485 μL of DMSO was added to prepare a 300 μM standard product solution. 5 μL of the standard product solution was transferred from a plate of the 300 μM standard product solution to another empty plate, and then 5 μL of a phosphate buffer with pH 7.4 and 490 μL of acetonitrile/water (1:1 in v/v) were added to obtain a standard product solution with the concentration of 3 μM. The concentration of the standard product solution might be adjusted according to the intensity of the liquid response signal thereof.

### 4) Sample Analysis Step

A sample injection plate was placed in a sample injection tray of an automatic sample injection, and samples were evaluated through liquid analysis.

### 5) Data Analysis

All calculations were performed through Microsoft Excel.

Analysis and quantification of the filter of the samples were completed by qualitatively and quantitatively analyzing the peak of a standard product with known concentration through liquid chromatography-mass spectrometry. A calculation formula for solubility values of the control drug and test substance was as follows:

$$[ Sample ] = \frac{{AREA}_{Sample} \times {DF}_{Sample} \times [ STD ]}{{AREA}_{Std}}$$

DF refers to the dilution ratio.

| | |
|---|---|
| | 40 |
| 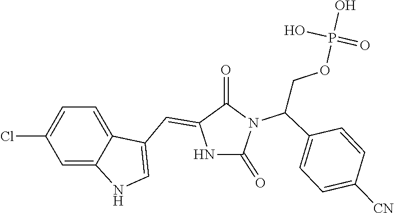 [SMILES: N#Cc1ccc(C(COP(=O)(O)O)N2C(=O)N/C(=C\c3c[nH]c4cc(Cl)ccc34)C2=O)cc1] | |
| | 38 |
| 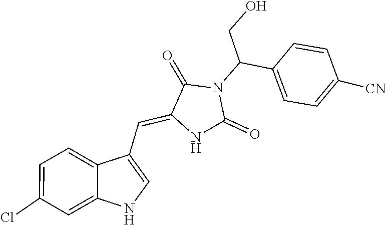 [SMILES: N#Cc1ccc(C(CO)N2C(=O)N/C(=C\c3c[nH]c4cc(Cl)ccc34)C2=O)cc1] | |

| Compound | Solubility (μg/mL) |
|---|---|
| Compound 38 | 0.075 |
| Compound 40 | 145.62 |

The embodiments of the technical solutions of the disclosure are exemplarily described above. It should be understood that the protection scope of the disclosure is not limited to the above embodiments. Any modification, equivalent replacement, improvement, etc. made by persons skilled in the art within the spirit and principle of this application should fall within the scope of claims of this application.

What is claimed is:

1. A compound shown in formula (I), and a raceme, a stereoisomer, a tautomer, an isotopic label, a solvate, a polymorph, a pharmaceutically acceptable salt or a prodrug compound thereof: 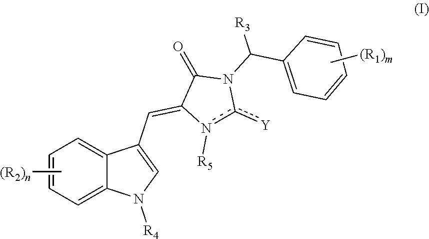
wherein
R₁ is selected from H, halogen, CN, C₁₋₆ alkyl, C₂₋₆ alkenyl, C₂₋₆ alkynyl, C₁₋₆ alkoxyl, halogenated C₁₋₆alkyl, and halogenated C₁₋₆ alkoxyl;
R₂ is selected from H, C₁₋₆ alkyl, halogen, C₁₋₆ alkoxyl, C₁₋₆ alkylthio, halogenated C₁₋₆ alkyl, and halogenated C₁₋₆ alkoxyl;
R₃ is selected from H, R₃ₐ—O—C₁₋₆ alkyl, —CH(═O), and —C(═O)—NH—R_{3b}; R₃ₐ is selected from H or —P(═O)(OR₃₁)(OR₃₂); R_{3b} is selected from the following radicals which are unsubstituted or optionally substituted with one, two, three or more R_{3c}: C₁₋₆ alkyl, C₃₋₈ cycloalkyl, and C₃₋₈cycloalkyl-C₁₋₆ alkyl; R_{3c} is selected from OH, C₁₋₆ alkyl-NH—, (C₁₋₆ alkyl)₂-N—, and —O—P(═O)(OR₃₁)(OR₃₂); and R₃₁ and R₃₂ are same or different, and are independently selected from H and C₁₋₆alkyl;
R₄ is selected from H and —P(═O)(OR₄₁)(OR₄₂); and R₄₁ and R₄₂ are same or different, and are independently selected from H and C₁₋₆ alkyl;
R₅ is selected from H and —P(═O)(OR₅₁)(OR₅₂); and R₅₁ and R₅₂ are same or different, and are independently selected from H and C₁₋₆ alkyl;
Y is O or OR₆; R₆ is selected from H and —P(═O)(OR₆₁)(OR₆₂); and R₆₁ and R₆₂ are same or different, and are independently selected from H and C₁₋₆ alkyl; [symbol]
is selected from a single bond or a double bond;
m is selected from 0, 1, 2, 3, 4 or 5; and
n is selected from 0, 1, 2, 3 or 4.

2. The compound, and the raceme, the stereoisomer, the tautomer, the isotopic label, the solvate, the polymorph, the pharmaceutically acceptable salt or the prodrug compound thereof according to claim 1, wherein R₁ is selected from H, halogen, CN, C₁₋₃ alkyl, C₂₋₃alkynyl, C₁₋₃ alkoxyl, halogenated C₁₋₃ alkyl, and halogenated C₁₋₃alkoxyl;
preferably, R₁ is selected from H, F, Cl, CN, acetenyl, methyl, ethyl, and propyl;
preferably, R₂ is selected from H, C₁₋₃ alkyl, halogen, C₁₋₃ alkoxyl, C₁₋₃ alkylthio, and halogenated C₁₋₃ alkoxyl; and
preferably, R₂ is selected from H, F, Cl, Br, methyl, methoxyl, fluoromethoxyl, and methylthio.

3. The compound, and the raceme, the stereoisomer, the tautomer, the isotopic label, the solvate, the polymorph, the pharmaceutically acceptable salt or the prodrug compound thereof according to claim 1, wherein R₃ is selected from H, R₃ₐ—O—C₁₋₃ alkyl, —CH(═O), and —C(═O)—NH—R_{3b}; R₃ₐ is selected from H or —P(═O)(OH)(OH); R_{3b} is selected from the following radicals which are unsubstituted or optionally substituted with one, two, three or more R_{3c}: C₁₋₃ alkyl, C₃₋₈ cycloalkyl, and C₃₋₈ cycloalkyl-C₁₋₃ alkyl; and R_{3c} is selected from OH, C₁₋₃ alkyl-NH—, (C₁₋₃ alkyl)₂-N—, and —O—P(═O)(OH)(OH);
preferably, R₃ is selected from H, methyl, 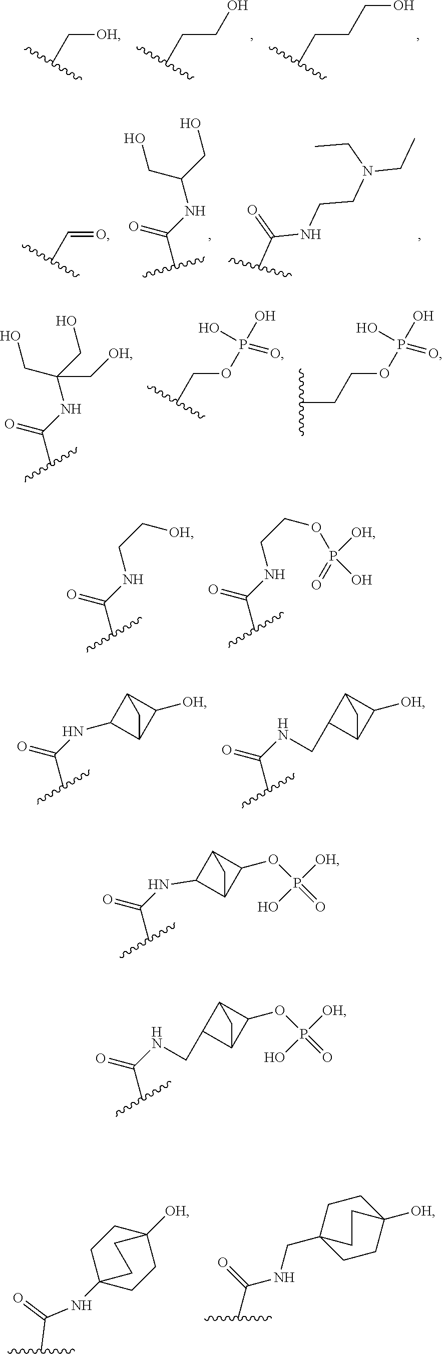 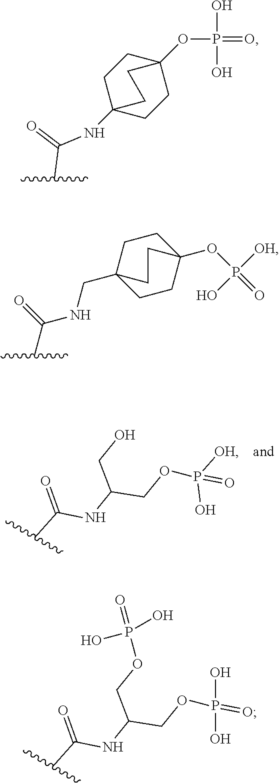 [SMILES: *C(=O)NC12CCC(OP(=O)(O)O)(CC1)CC2 ; *C(=O)NCC12CCC(OP(=O)(O)O)(CC1)CC2 ; *C(=O)NC(CO)COP(=O)(O)O ; *C(=O)NC(COP(=O)(O)O)COP(=O)(O)O]
for example, selected from

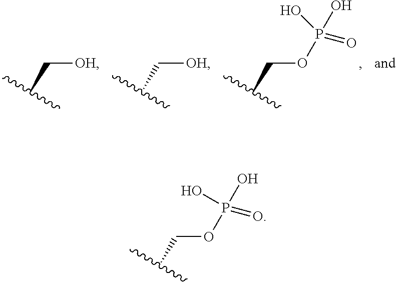

4. The compound, and the raceme, the stereoisomer, the tautomer, the isotopic label, the solvate, the polymorph, the pharmaceutically acceptable salt or the prodrug compound thereof according to any one of claim 1, wherein R₄ is selected from H and —P(═O)(OR₄₁)(OR₄₂); R₄₁ and R₄₂ are same or different, and are independently selected from H and C₁₋₃ alkyl;
preferably, R₄ is selected from H and —P(═O)(OH)(OH);
preferably, R₅ is selected from H and —P(═O)(OR₅₁)(OR₅₂); and R₅₁ and R₅₂ are same or different, and are independently selected from H and C₁₋₃ alkyl;
preferably, R₅ is selected from H and —P(═O)(OH)(OH);
preferably, Y is O or OR₆; R₆ is selected from H and —P(═O)(OR₆₁)(OR₆₂); and R₆₁ and R₆₂ are same or different, and are independently selected from H and C₁₋₃ alkyl;
preferably, R₆ is selected from H and —P(═O)(OH)(OH);
preferably, m is selected from 1, 2 or 3; and
preferably, n is selected from 1 or 2.

5. The compound, and the raceme, the stereoisomer, the tautomer, the isotopic label, the solvate, the polymorph, the pharmaceutically acceptable salt or the prodrug compound thereof according to any one of claim 1, wherein the prodrug compound has a structure shown in formula (II): 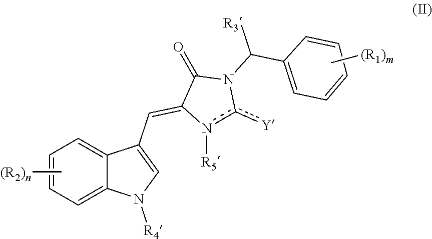
wherein R₁, R₂, m and n independently have definitions according to any one of claim 1;
R₃′ has a definition of R₃ according to any one of claim 1;
R₄′ is selected from H and —P(═O)(OR₄₁′)(OR₄₂′); R₄₁′ and R₄₂′ are same or different, and are independently selected from H and C₁₋₃ alkyl; preferably, R₄′ is selected from H and —P(═O)(OR₄₁′)(OR₄₂′); and R₄₁′ and R₄₂′ are same or different, and are independently selected from H and C₁₋₆alkyl;
R₅′ is selected from H and —P(═O)(OR₅₁′)(OR₅₂′); R₅₁′ and R₅₂′ are same or different, and are independently selected from H and C₁₋₃ alkyl; preferably, R₅′ is selected from H and —P(═O)(OR₅₁′)(OR₅₂′); and R₅₁′ and R₅₂′ are same or different, and are independently selected from H and C₁₋₆ alkyl;
Y′ is O or OR₆′; R₆′ is selected from H and —P(═O)(OR₆₁′)(OR₆₂′); and R₆₁′ and R₆₂′ are same or different, and are independently selected from H and C₁₋₃ alkyl; preferably, R6′ is selected from H and —P(═O)(OR₆₁′)(OR₆₂′); and R₆₁′ and R₆₂′ are same or different, and are independently selected from H and C₁₋₆ alkyl; [symbol]
is selected from a single bond or a double bond;
preferably, R₃′ is selected from H, R₃ₐ—O—C₁₋₆ alkyl, —CH(═O), and —C(═O)—NH—R_{3b}; R₃ₐ is selected from H or —P(═O)(OH)(OH); R_{3b} is selected from the following radicals which are unsubstituted or optionally substituted with one, two, three or more R_{3c}: C₁₋₆ alkyl, C₃₋₈ cycloalkyl, and C₃₋₈ cycloalkyl-C₁₋₆ alkyl; R_{3c} is selected from OH, C₁₋₆ alkyl-NH—, (C₁₋₆ alkyl)₂-N—, and —O—P(═O)(OH)(OH);
preferably, R₄′ is selected from H and —P(═O)(OH)(OH);
preferably, R₅′ is selected from H and —P(═O)(OH)(OH);
preferably, Y′ is O or OR₆′; and R₆′ is selected from H and —P(═O)(OH)(OH); [symbol]
is selected from a single bond or a double bond; and
at least one of R₄′, R₅′, R₆′, R₃ₐ, and R_{3c} is —P(═O)(OH)(OH).

6. The compound, and the raceme, the stereoisomer, the tautomer, the isotopic label, the solvate, the polymorph, the pharmaceutically acceptable salt or the prodrug compound thereof according to any one of claim 1, wherein the compound is selected from the following structures:

| Number | Structure |
|---|---|
| 1 | 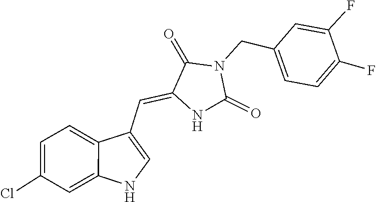 [SMILES: O=C1N/C(=C\c2c[nH]c3cc(Cl)ccc23)C(=O)N1Cc1ccc(F)c(F)c1] |
| 2 | 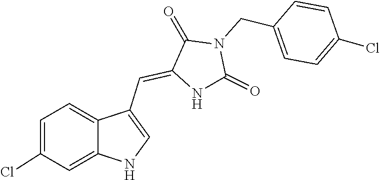 [SMILES: O=C1N/C(=C\c2c[nH]c3cc(Cl)ccc23)C(=O)N1Cc1ccc(Cl)cc1] |
| 3 | 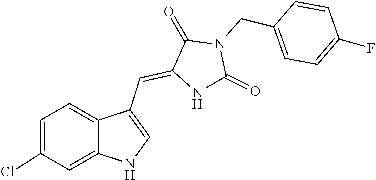 [SMILES: O=C1N/C(=C\c2c[nH]c3cc(Cl)ccc23)C(=O)N1Cc1ccc(F)cc1] |
| 4 | 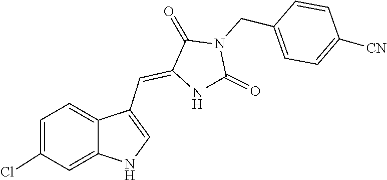 [SMILES: N#Cc1ccc(CN2C(=O)N/C(=C\c3c[nH]c4cc(Cl)ccc34)C2=O)cc1] |
| 5 | 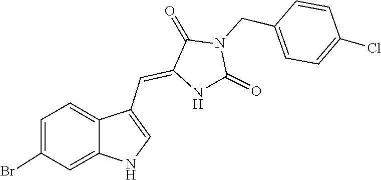 [SMILES: O=C1N/C(=C\c2c[nH]c3cc(Br)ccc23)C(=O)N1Cc1ccc(Cl)cc1] |
| 6 | 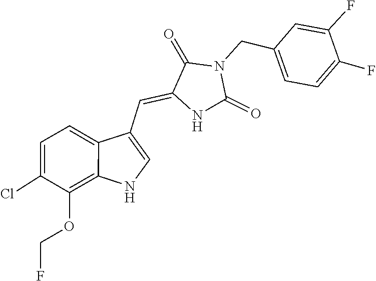 [SMILES: O=C1N/C(=C\c2c[nH]c3c(OCF)c(Cl)ccc23)C(=O)N1Cc1ccc(F)c(F)c1] |
| 7 | 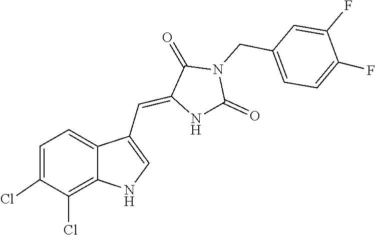 [SMILES: O=C1N/C(=C\c2c[nH]c3c(Cl)c(Cl)ccc23)C(=O)N1Cc1ccc(F)c(F)c1] |
| 8 | 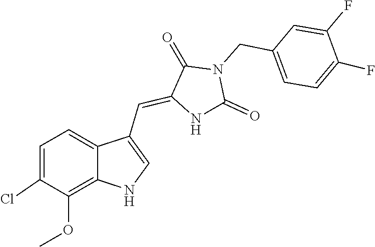 [SMILES: COc1c(Cl)ccc2c(/C=C3\NC(=O)N(Cc4ccc(F)c(F)c4)C3=O)c[nH]c12] |
| 9 | 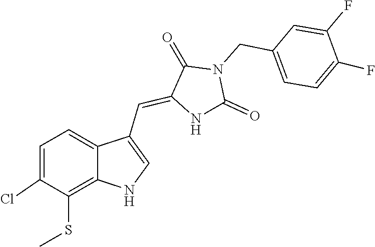 [SMILES: CSc1c(Cl)ccc2c(/C=C3\NC(=O)N(Cc4ccc(F)c(F)c4)C3=O)c[nH]c12] |
| 10 | 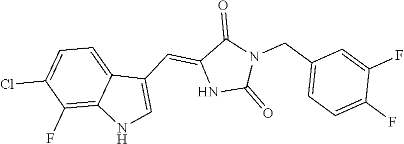 [SMILES: O=C1N/C(=C\c2c[nH]c3c(F)c(Cl)ccc23)C(=O)N1Cc1ccc(F)c(F)c1] |
| 11 | 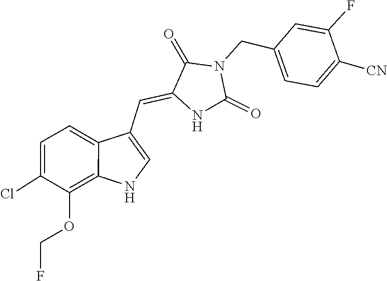 [SMILES: N#Cc1ccc(CN2C(=O)N/C(=C\c3c[nH]c4c(OCF)c(Cl)ccc34)C2=O)cc1F] |
| 12 | 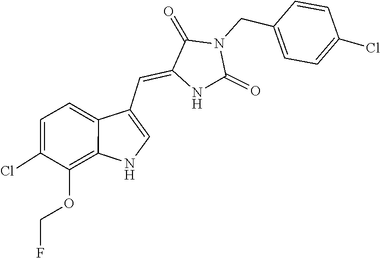 [SMILES: O=C1N/C(=C\c2c[nH]c3c(OCF)c(Cl)ccc23)C(=O)N1Cc1ccc(Cl)cc1] |
| 13 | 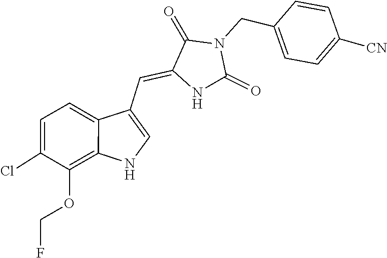 [SMILES: N#Cc1ccc(CN2C(=O)N/C(=C\c3c[nH]c4c(OCF)c(Cl)ccc34)C2=O)cc1] |
| 14 | 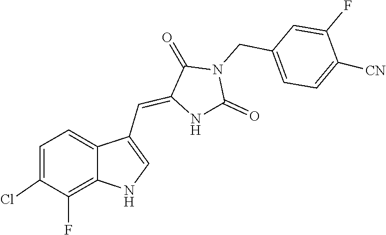 [SMILES: N#Cc1ccc(CN2C(=O)N/C(=C\c3c[nH]c4c(F)c(Cl)ccc34)C2=O)cc1F] |
| 15 | 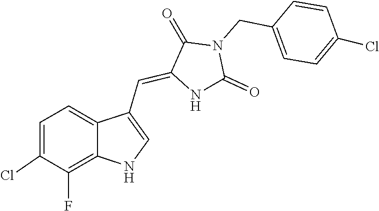 [SMILES: O=C1N/C(=C\c2c[nH]c3c(F)c(Cl)ccc23)C(=O)N1Cc1ccc(Cl)cc1] |
| 16 | 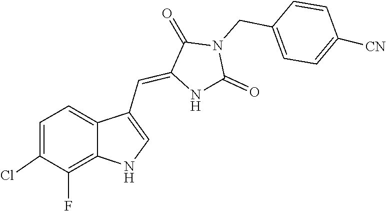 [SMILES: N#Cc1ccc(CN2C(=O)N/C(=C\c3c[nH]c4c(F)c(Cl)ccc34)C2=O)cc1] |
| 17 | 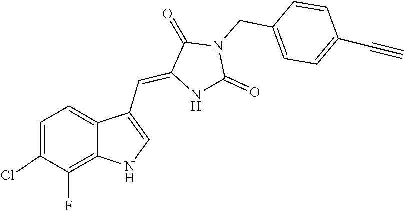 [SMILES: C#Cc1ccc(CN2C(=O)N/C(=C\c3c[nH]c4c(F)c(Cl)ccc34)C2=O)cc1] |
| 18 | 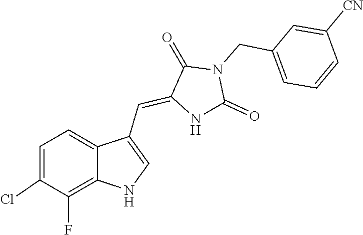 [SMILES: N#Cc1cccc(CN2C(=O)N/C(=C\c3c[nH]c4c(F)c(Cl)ccc34)C2=O)c1] |
| 19 | 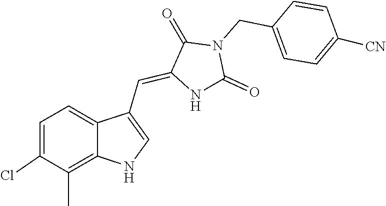 [SMILES: Cc1c(Cl)ccc2c(/C=C3\NC(=O)N(Cc4ccc(C#N)cc4)C3=O)c[nH]c12] |
| 20 | 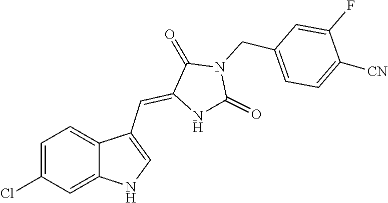 [SMILES: N#Cc1ccc(CN2C(=O)N/C(=C\c3c[nH]c4cc(Cl)ccc34)C2=O)cc1F] |
| 21 | 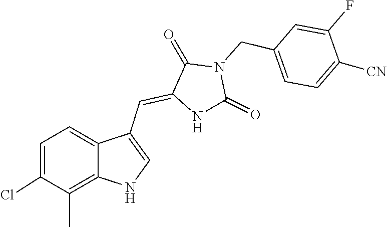 [SMILES: Cc1c(Cl)ccc2c(/C=C3\NC(=O)N(Cc4ccc(C#N)c(F)c4)C3=O)c[nH]c12] |
| 22 | 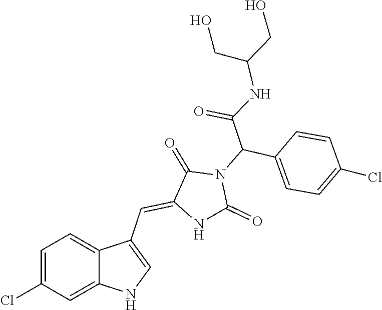 [SMILES: O=C(NC(CO)CO)C(c1ccc(Cl)cc1)N1C(=O)N/C(=C\c2c[nH]c3cc(Cl)ccc23)C1=O] |
| 23 | 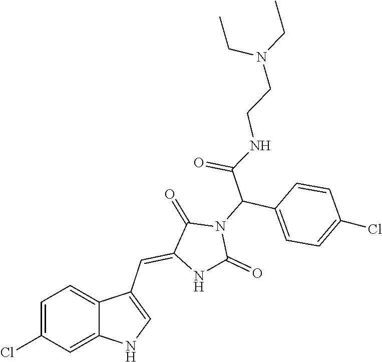 [SMILES: CCN(CC)CCNC(=O)C(c1ccc(Cl)cc1)N1C(=O)N/C(=C\c2c[nH]c3cc(Cl)ccc23)C1=O] |
| 24 | 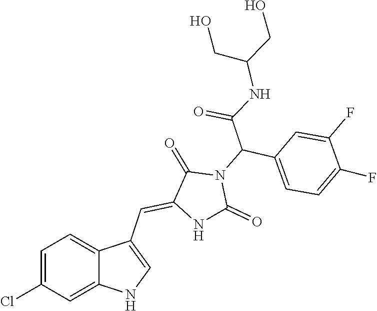 [SMILES: O=C(NC(CO)CO)C(c1ccc(F)c(F)c1)N1C(=O)N/C(=C\c2c[nH]c3cc(Cl)ccc23)C1=O] |
| 25 | 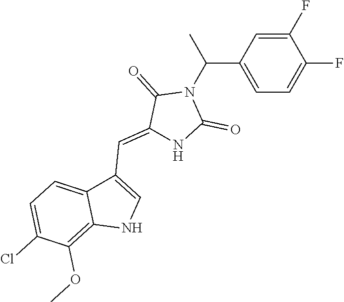 [SMILES: COc1c(Cl)ccc2c(/C=C3\NC(=O)N(C(C)c4ccc(F)c(F)c4)C3=O)c[nH]c12] |
| 26 | 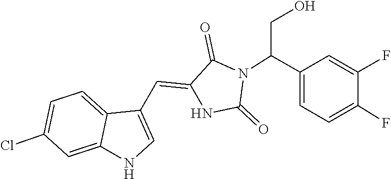 [SMILES: O=C1N/C(=C\c2c[nH]c3cc(Cl)ccc23)C(=O)N1C(CO)c1ccc(F)c(F)c1] |
| 27 | 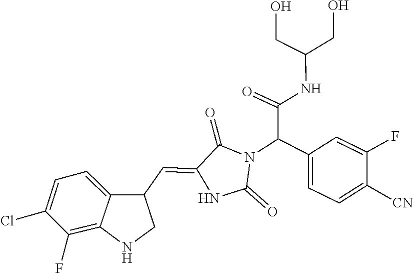 [SMILES: N#Cc1ccc(C(C(=O)NC(CO)CO)N2C(=O)N/C(=C\C3CNc4c3ccc(Cl)c4F)C2=O)cc1F] |
| 28 | 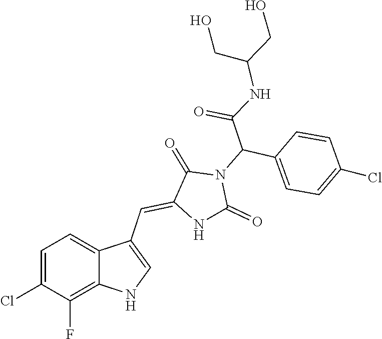 [SMILES: O=C(NC(CO)CO)C(c1ccc(Cl)cc1)N1C(=O)N/C(=C\c2c[nH]c3c(F)c(Cl)ccc23)C1=O] |
| 29 | 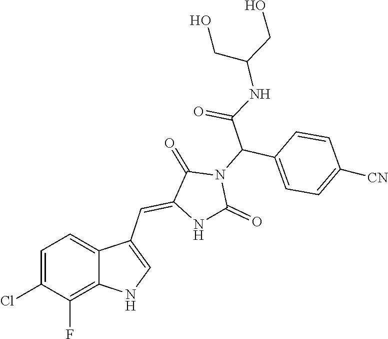 [SMILES: N#Cc1ccc(C(C(=O)NC(CO)CO)N2C(=O)N/C(=C\c3c[nH]c4c(F)c(Cl)ccc34)C2=O)cc1] |
| 30 | 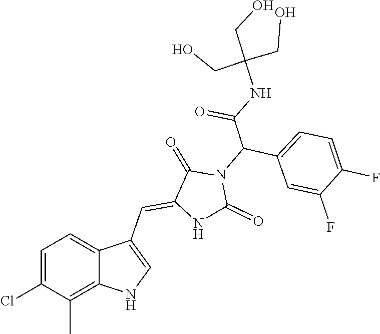 [SMILES: Cc1c(Cl)ccc2c(/C=C3\NC(=O)N(C(C(=O)NC(CO)(CO)CO)c4ccc(F)c(F)c4)C3=O)c[nH]c12] |
| 31 | 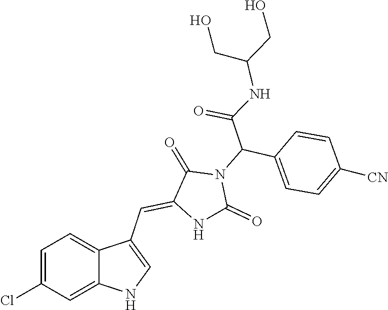 [SMILES: N#Cc1ccc(C(C(=O)NC(CO)CO)N2C(=O)N/C(=C\c3c[nH]c4cc(Cl)ccc34)C2=O)cc1] |
| 32 | 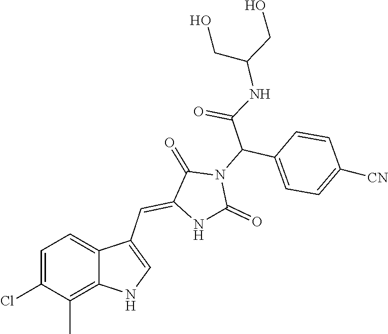 [SMILES: Cc1c(Cl)ccc2c(/C=C3\NC(=O)N(C(C(=O)NC(CO)CO)c4ccc(C#N)cc4)C3=O)c[nH]c12] |
| 33 | 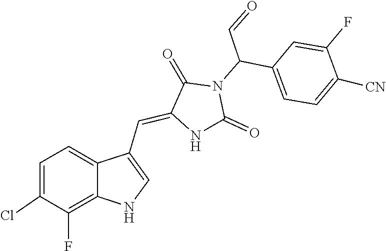 [SMILES: N#Cc1ccc(C(C=O)N2C(=O)N/C(=C\c3c[nH]c4c(F)c(Cl)ccc34)C2=O)cc1F] |
| 34 | 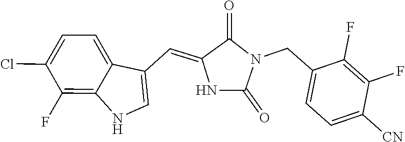 [SMILES: N#Cc1ccc(CN2C(=O)N/C(=C\c3c[nH]c4c(F)c(Cl)ccc34)C2=O)c(F)c1F] |
| 35 | 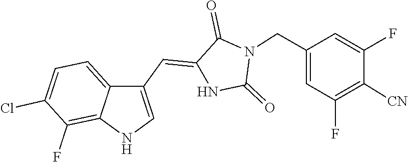 [SMILES: N#Cc1c(F)cc(CN2C(=O)N/C(=C\c3c[nH]c4c(F)c(Cl)ccc34)C2=O)cc1F] |
| 36 | 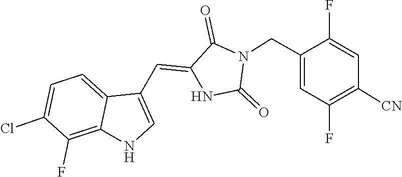 [SMILES: N#Cc1cc(F)c(CN2C(=O)N/C(=C\c3c[nH]c4c(F)c(Cl)ccc34)C2=O)cc1F] |
| 37 | 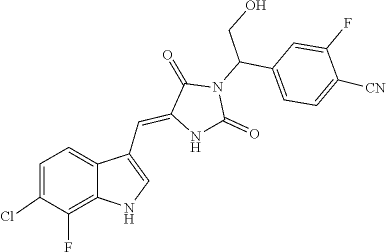 [SMILES: N#Cc1ccc(C(CO)N2C(=O)N/C(=C\c3c[nH]c4c(F)c(Cl)ccc34)C2=O)cc1F] |
| 38 | 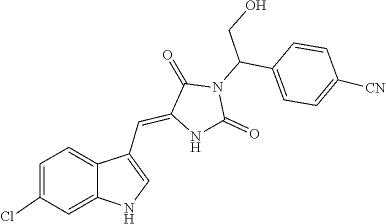 [SMILES: N#Cc1ccc(C(CO)N2C(=O)N/C(=C\c3c[nH]c4cc(Cl)ccc34)C2=O)cc1] |
| 39 | 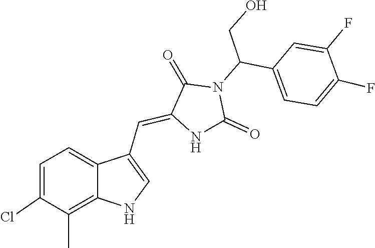 [SMILES: Cc1c(Cl)ccc2c(/C=C3\NC(=O)N(C(CO)c4ccc(F)c(F)c4)C3=O)c[nH]c12] |
| 40 | 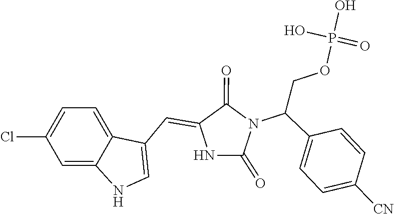 [SMILES: N#Cc1ccc(C(COP(=O)(O)O)N2C(=O)N/C(=C\c3c[nH]c4cc(Cl)ccc34)C2=O)cc1] |
| 41 | 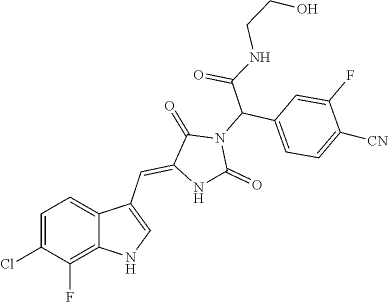 [SMILES: N#Cc1ccc(C(C(=O)NCCO)N2C(=O)N/C(=C\c3c[nH]c4c(F)c(Cl)ccc34)C2=O)cc1F] |
| 42 | 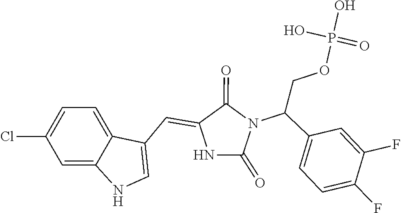 [SMILES: O=C1N/C(=C\c2c[nH]c3cc(Cl)ccc23)C(=O)N1C(COP(=O)(O)O)c1ccc(F)c(F)c1] |
| 43 | 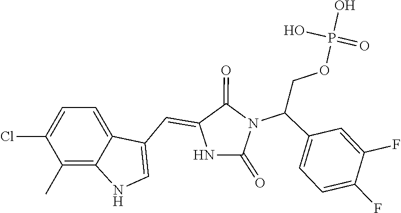 [SMILES: Cc1c(Cl)ccc2c(/C=C3\NC(=O)N(C(COP(=O)(O)O)c4ccc(F)c(F)c4)C3=O)c[nH]c12] |
| 44 | 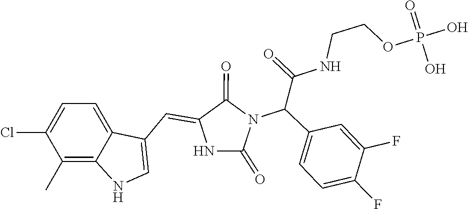 [SMILES: Cc1c(Cl)ccc2c(/C=C3\NC(=O)N(C(C(=O)NCCOP(=O)(O)O)c4ccc(F)c(F)c4)C3=O)c[nH]c12] |
| 45 | 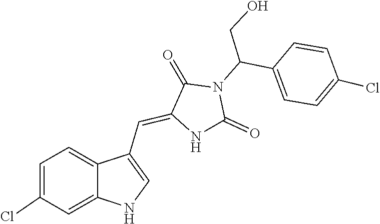 [SMILES: O=C1N/C(=C\c2c[nH]c3cc(Cl)ccc23)C(=O)N1C(CO)c1ccc(Cl)cc1] |
| 46 | 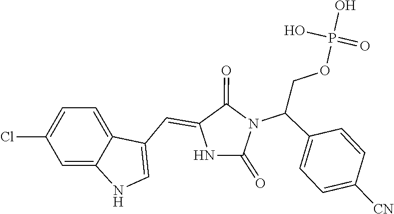 [SMILES: N#Cc1ccc(C(COP(=O)(O)O)N2C(=O)N/C(=C\c3c[nH]c4cc(Cl)ccc34)C2=O)cc1] |
| 47 | 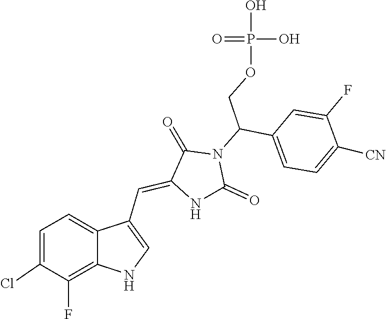 [SMILES: N#Cc1ccc(C(COP(=O)(O)O)N2C(=O)N/C(=C\c3c[nH]c4c(F)c(Cl)ccc34)C2=O)cc1F] |
| 48 | 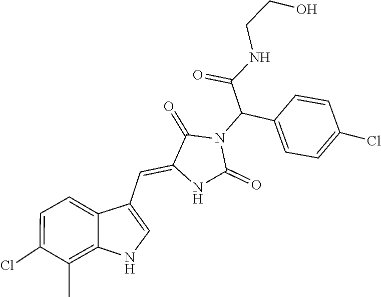 [SMILES: Cc1c(Cl)ccc2c(/C=C3\NC(=O)N(C(C(=O)NCCO)c4ccc(Cl)cc4)C3=O)c[nH]c12] |
| 49 | 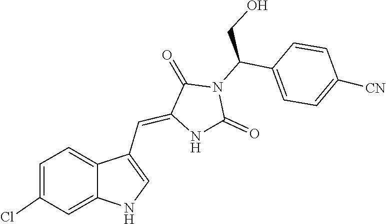 [SMILES: N#Cc1ccc([C@H](CO)N2C(=O)N/C(=C\c3c[nH]c4cc(Cl)ccc34)C2=O)cc1] |
| 50 | 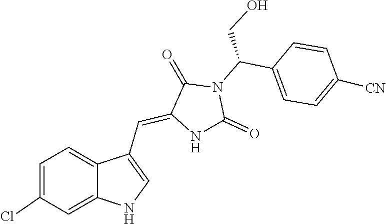 [SMILES: N#Cc1ccc([C@@H](CO)N2C(=O)N/C(=C\c3c[nH]c4cc(Cl)ccc34)C2=O)cc1] |
| 51 | 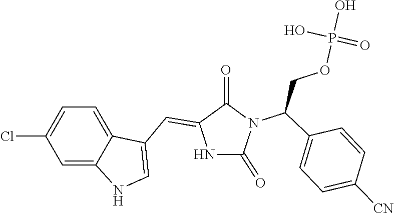 [SMILES: N#Cc1ccc([C@H](COP(=O)(O)O)N2C(=O)N/C(=C\c3c[nH]c4cc(Cl)ccc34)C2=O)cc1] |
| 52 | 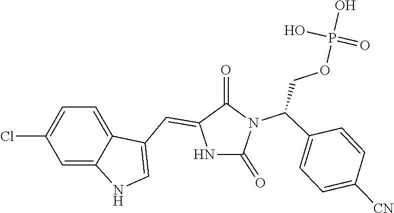 [SMILES: N#Cc1ccc([C@@H](COP(=O)(O)O)N2C(=O)N/C(=C\c3c[nH]c4cc(Cl)ccc34)C2=O)cc1] |
| 53 | 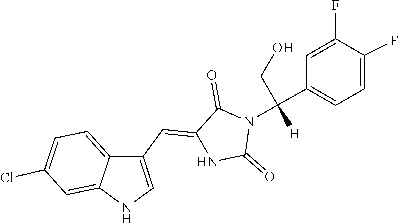 [SMILES: O=C1N/C(=C\c2c[nH]c3cc(Cl)ccc23)C(=O)N1[C@H](CO)c1ccc(F)c(F)c1] |
| 54 | 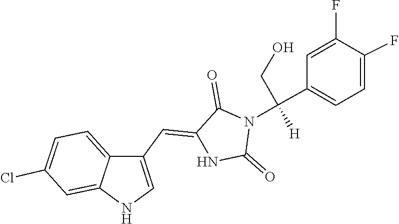 [SMILES: O=C1N/C(=C\c2c[nH]c3cc(Cl)ccc23)C(=O)N1[C@@H](CO)c1ccc(F)c(F)c1] |
| 55 | 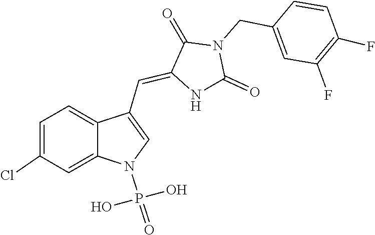 [SMILES: O=C1N/C(=C\c2cn(P(=O)(O)O)c3cc(Cl)ccc23)C(=O)N1Cc1ccc(F)c(F)c1] |
| 56 | 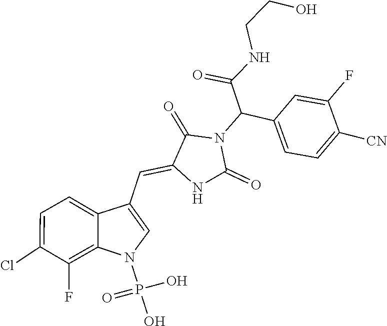 [SMILES: N#Cc1ccc(C(C(=O)NCCO)N2C(=O)N/C(=C\c3cn(P(=O)(O)O)c4c(F)c(Cl)ccc34)C2=O)cc1F] |
| 57 | 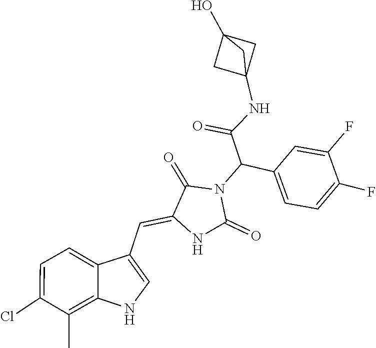 [SMILES: Cc1c(Cl)ccc2c(/C=C3\NC(=O)N(C(C(=O)NC45CC(O)(C4)C5)c4ccc(F)c(F)c4)C3=O)c[nH]c12] |
| 58 | 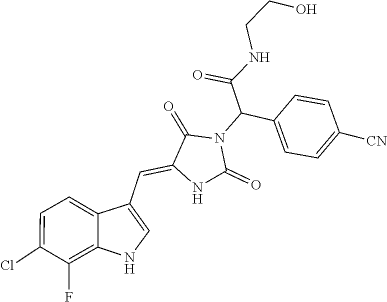 [SMILES: N#Cc1ccc(C(C(=O)NCCO)N2C(=O)N/C(=C\c3c[nH]c4c(F)c(Cl)ccc34)C2=O)cc1] |
| 59 | 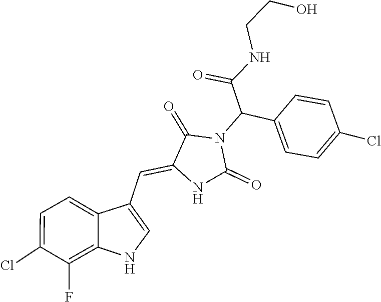 [SMILES: O=C(NCCO)C(c1ccc(Cl)cc1)N1C(=O)N/C(=C\c2c[nH]c3c(F)c(Cl)ccc23)C1=O] |
| 60 | 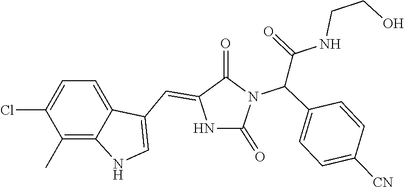 [SMILES: Cc1c(Cl)ccc2c(/C=C3\NC(=O)N(C(C(=O)NCCO)c4ccc(C#N)cc4)C3=O)c[nH]c12] |
| 61 | 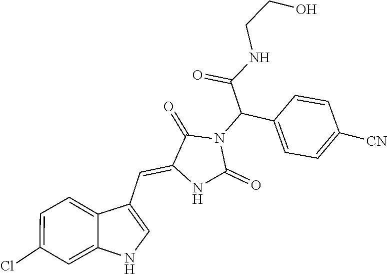 [SMILES: N#Cc1ccc(C(C(=O)NCCO)N2C(=O)N/C(=C\c3c[nH]c4cc(Cl)ccc34)C2=O)cc1] |
| 62 | 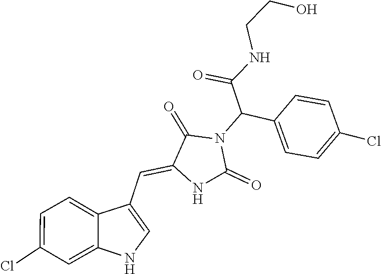 [SMILES: O=C(NCCO)C(c1ccc(Cl)cc1)N1C(=O)N/C(=C\c2c[nH]c3cc(Cl)ccc23)C1=O] |
| 63 | 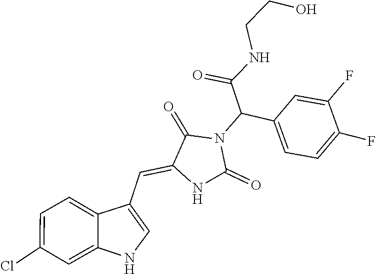 [SMILES: O=C(NCCO)C(c1ccc(F)c(F)c1)N1C(=O)N/C(=C\c2c[nH]c3cc(Cl)ccc23)C1=O] |
| 64 | 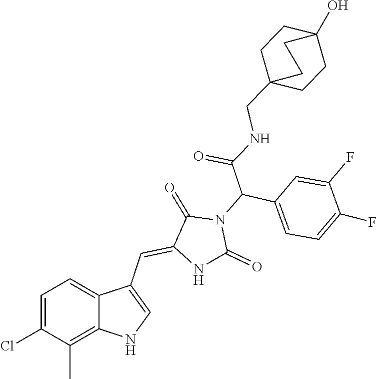 [SMILES: Cc1c(Cl)ccc2c(/C=C3\NC(=O)N(C(C(=O)NCC45CCC(O)(CC4)CC5)c4ccc(F)c(F)c4)C3=O)c[nH]c12] |
| 65 | 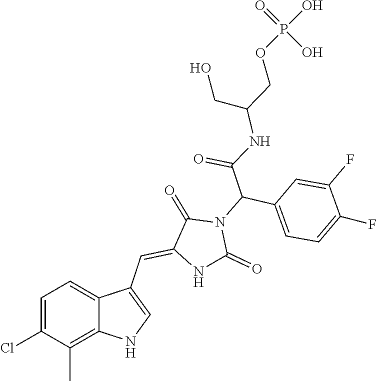 [SMILES: Cc1c(Cl)ccc2c(/C=C3\NC(=O)N(C(C(=O)NC(CO)COP(=O)(O)O)c4ccc(F)c(F)c4)C3=O)c[nH]c12] |
| 66 | 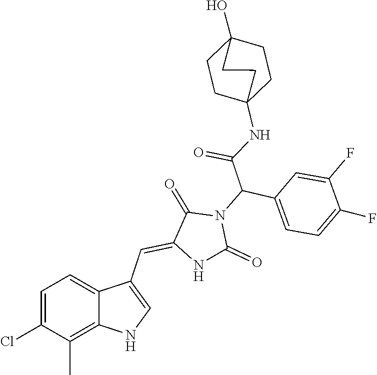 [SMILES: Cc1c(Cl)ccc2c(/C=C3\NC(=O)N(C(C(=O)NC45CCC(O)(CC4)CC5)c4ccc(F)c(F)c4)C3=O)c[nH]c12] |
| 67 | 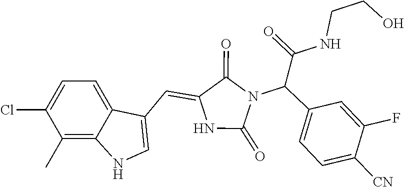 [SMILES: Cc1c(Cl)ccc2c(/C=C3\NC(=O)N(C(C(=O)NCCO)c4ccc(C#N)c(F)c4)C3=O)c[nH]c12] |
| 68 | 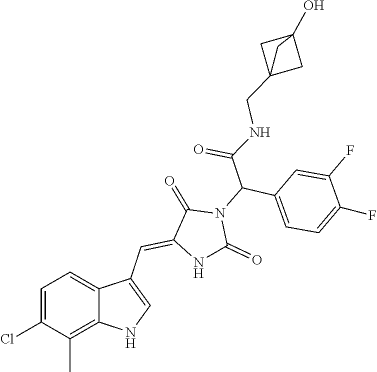 [SMILES: Cc1c(Cl)ccc2c(/C=C3\NC(=O)N(C(C(=O)NCC45CC(O)(C4)C5)c4ccc(F)c(F)c4)C3=O)c[nH]c12] |
| 69 | 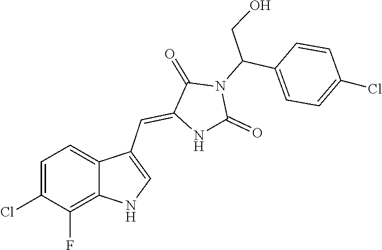 [SMILES: O=C1N/C(=C\c2c[nH]c3c(F)c(Cl)ccc23)C(=O)N1C(CO)c1ccc(Cl)cc1] |
| 70 | 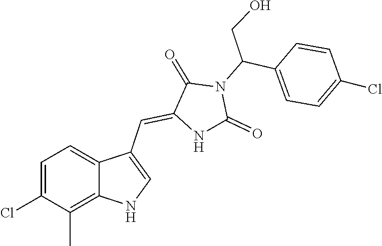 [SMILES: Cc1c(Cl)ccc2c(/C=C3\NC(=O)N(C(CO)c4ccc(Cl)cc4)C3=O)c[nH]c12] |
| 71 | 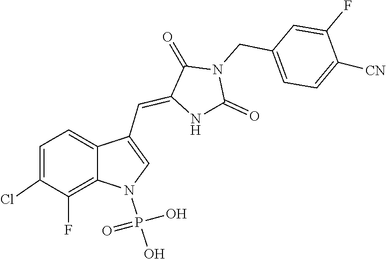 [SMILES: N#Cc1ccc(CN2C(=O)N/C(=C\c3cn(P(=O)(O)O)c4c(F)c(Cl)ccc34)C2=O)cc1F] |
| 72 | 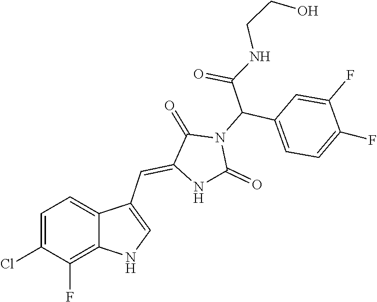 [SMILES: O=C(NCCO)C(c1ccc(F)c(F)c1)N1C(=O)N/C(=C\c2c[nH]c3c(F)c(Cl)ccc23)C1=O] |
| 73 | 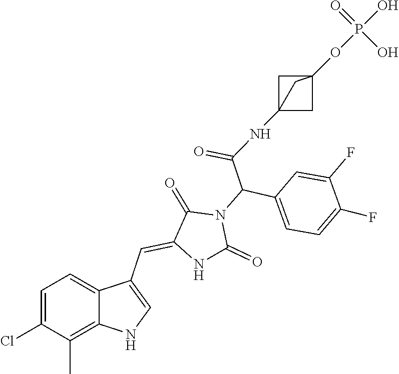 [SMILES: Cc1c(Cl)ccc2c(/C=C3\NC(=O)N(C(C(=O)NC45CC(OP(=O)(O)O)(C4)C5)c4ccc(F)c(F)c4)C3=O)c[nH]c12] |
| 74 | 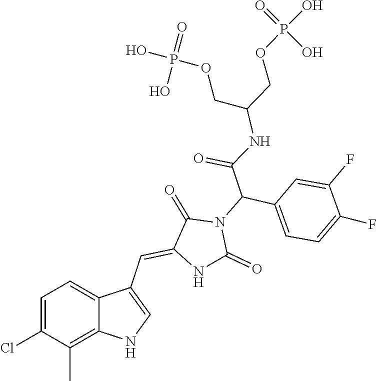 [SMILES: Cc1c(Cl)ccc2c(/C=C3\NC(=O)N(C(C(=O)NC(COP(=O)(O)O)COP(=O)(O)O)c4ccc(F)c(F)c4)C3=O)c[nH]c12] |
| 75 | 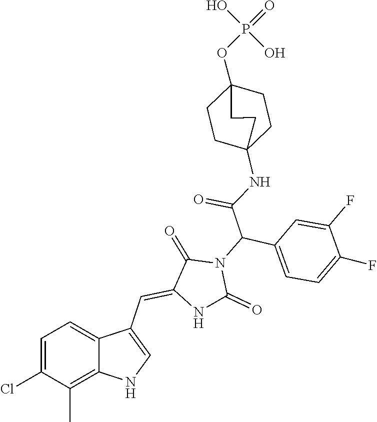 [SMILES: Cc1c(Cl)ccc2c(/C=C3\NC(=O)N(C(C(=O)NC45CCC(OP(=O)(O)O)(CC4)CC5)c4ccc(F)c(F)c4)C3=O)c[nH]c12] |
| 76 | 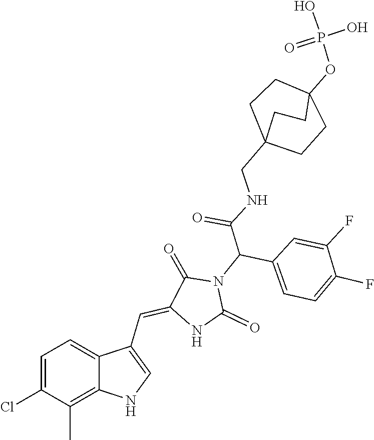 [SMILES: Cc1c(Cl)ccc2c(/C=C3\NC(=O)N(C(C(=O)NCC45CCC(OP(=O)(O)O)(CC4)CC5)c4ccc(F)c(F)c4)C3=O)c[nH]c12] |
| 77 | 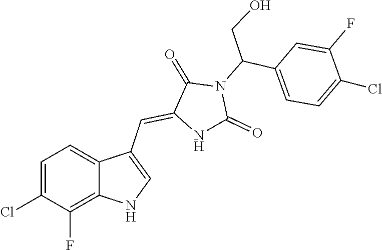 [SMILES: O=C1N/C(=C\c2c[nH]c3c(F)c(Cl)ccc23)C(=O)N1C(CO)c1ccc(Cl)c(F)c1] |
| 78 | 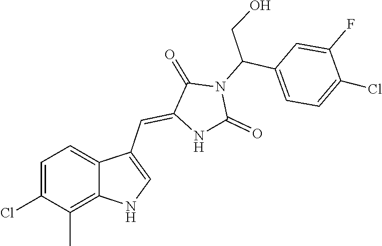 [SMILES: Cc1c(Cl)ccc2c(/C=C3\NC(=O)N(C(CO)c4ccc(Cl)c(F)c4)C3=O)c[nH]c12] |
| 79 | 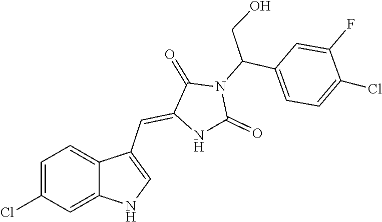 [SMILES: O=C1N/C(=C\c2c[nH]c3cc(Cl)ccc23)C(=O)N1C(CO)c1ccc(Cl)c(F)c1] |
| 80 | 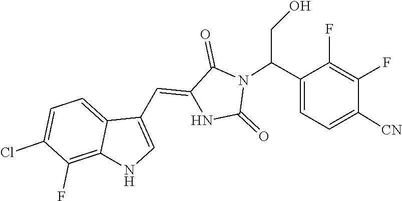 [SMILES: N#Cc1ccc(C(CO)N2C(=O)N/C(=C\c3c[nH]c4c(F)c(Cl)ccc34)C2=O)c(F)c1F] |
| 81 | 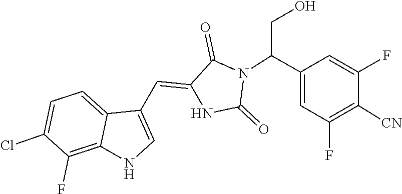 [SMILES: N#Cc1c(F)cc(C(CO)N2C(=O)N/C(=C\c3c[nH]c4c(F)c(Cl)ccc34)C2=O)cc1F] |
| 82 | 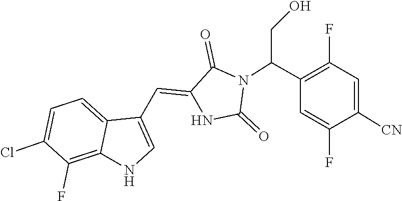 [SMILES: N#Cc1cc(F)c(C(CO)N2C(=O)N/C(=C\c3c[nH]c4c(F)c(Cl)ccc34)C2=O)cc1F] |
| 83 | 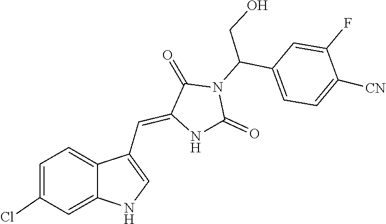 [SMILES: N#Cc1ccc(C(CO)N2C(=O)N/C(=C\c3c[nH]c4cc(Cl)ccc34)C2=O)cc1F] |
| 84 | 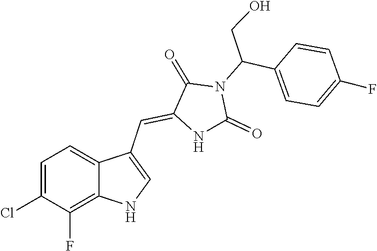 [SMILES: O=C1N/C(=C\c2c[nH]c3c(F)c(Cl)ccc23)C(=O)N1C(CO)c1ccc(F)cc1] |
| 85 | 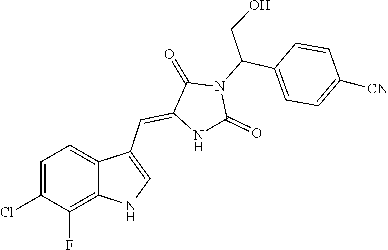 [SMILES: N#Cc1ccc(C(CO)N2C(=O)N/C(=C\c3c[nH]c4c(F)c(Cl)ccc34)C2=O)cc1] |
| 86 | 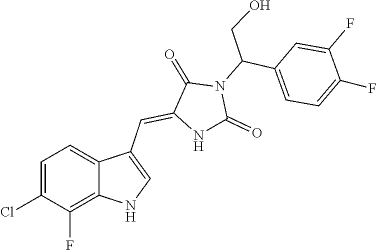 [SMILES: O=C1N/C(=C\c2c[nH]c3c(F)c(Cl)ccc23)C(=O)N1C(CO)c1ccc(F)c(F)c1] |
| 87 | 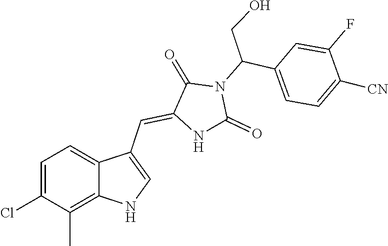 [SMILES: Cc1c(Cl)ccc2c(/C=C3\NC(=O)N(C(CO)c4ccc(C#N)c(F)c4)C3=O)c[nH]c12] |
| 88 | 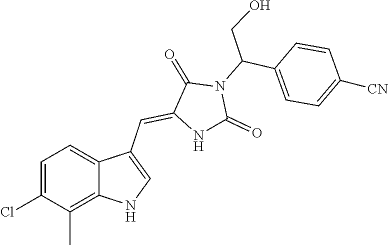 [SMILES: Cc1c(Cl)ccc2c(/C=C3\NC(=O)N(C(CO)c4ccc(C#N)cc4)C3=O)c[nH]c12] |
| 89 | 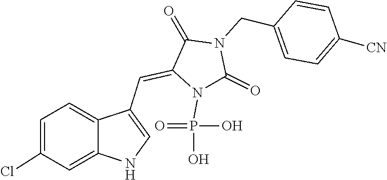 [SMILES: N#Cc1ccc(CN2C(=O)/C(=C/c3c[nH]c4cc(Cl)ccc34)N(P(=O)(O)O)C2=O)cc1] |
| 90 | 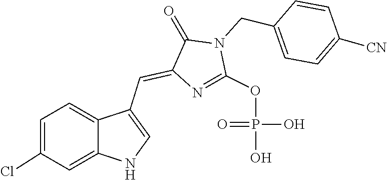 [SMILES: N#Cc1ccc(CN2C(=O)/C(=C/c3c[nH]c4cc(Cl)ccc34)N=C2OP(=O)(O)O)cc1] |
| 91 | 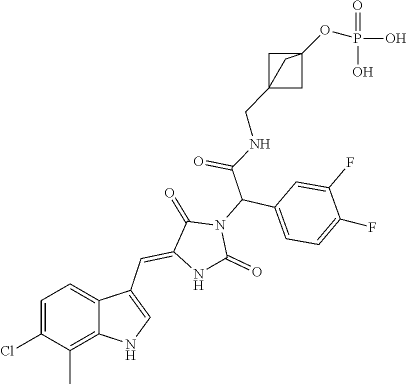 [SMILES: Cc1c(Cl)ccc2c(/C=C3\NC(=O)N(C(C(=O)NCC45CC(OP(=O)(O)O)(C4)C5)c4ccc(F)c(F)c4)C3=O)c[nH]c12] |
| 92 | 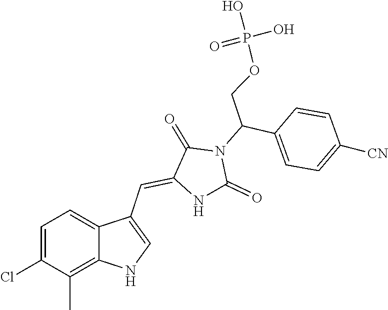 [SMILES: Cc1c(Cl)ccc2c(/C=C3\NC(=O)N(C(COP(=O)(O)O)c4ccc(C#N)cc4)C3=O)c[nH]c12] |
| 93 | 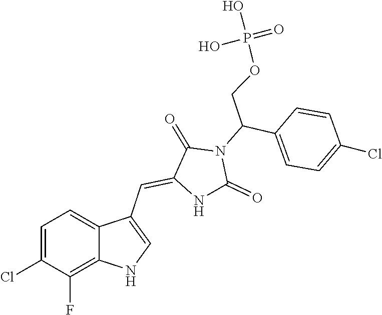 [SMILES: O=C1N/C(=C\c2c[nH]c3c(F)c(Cl)ccc23)C(=O)N1C(COP(=O)(O)O)c1ccc(Cl)cc1] |
| 94 | 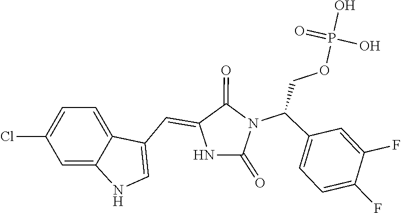 [SMILES: O=C1N/C(=C\c2c[nH]c3cc(Cl)ccc23)C(=O)N1[C@H](COP(=O)(O)O)c1ccc(F)c(F)c1] |
| 95 | 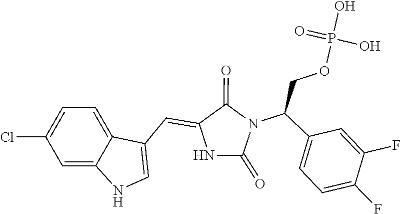 [SMILES: O=C1N/C(=C\c2c[nH]c3cc(Cl)ccc23)C(=O)N1[C@@H](COP(=O)(O)O)c1ccc(F)c(F)c1] |
| 96 | 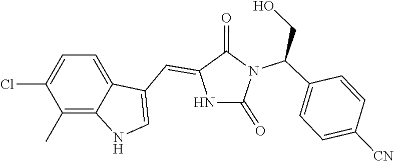 [SMILES: Cc1c(Cl)ccc2c(/C=C3\NC(=O)N([C@@H](CO)c4ccc(C#N)cc4)C3=O)c[nH]c12] |
| 97 | 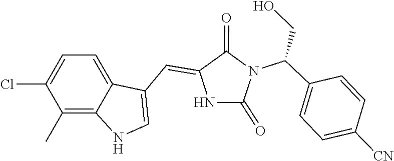 [SMILES: Cc1c(Cl)ccc2c(/C=C3\NC(=O)N([C@H](CO)c4ccc(C#N)cc4)C3=O)c[nH]c12] |
| 98 | 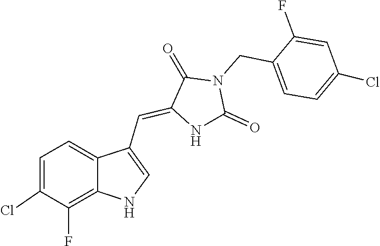 [SMILES: O=C1N/C(=C\c2c[nH]c3c(F)c(Cl)ccc23)C(=O)N1Cc1ccc(Cl)cc1F] |
| 99 | 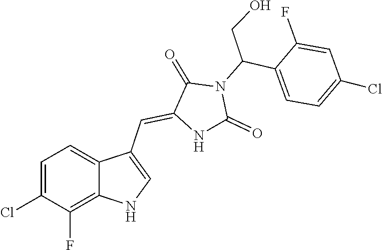 [SMILES: O=C1N/C(=C\c2c[nH]c3c(F)c(Cl)ccc23)C(=O)N1C(CO)c1ccc(Cl)cc1F] |
| 100 | 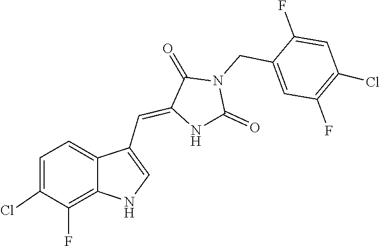 [SMILES: O=C1N/C(=C\c2c[nH]c3c(F)c(Cl)ccc23)C(=O)N1Cc1cc(F)c(Cl)cc1F] |
| 101 | 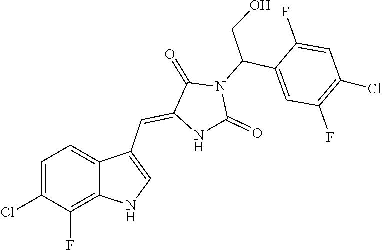 [SMILES: O=C1N/C(=C\c2c[nH]c3c(F)c(Cl)ccc23)C(=O)N1C(CO)c1cc(F)c(Cl)cc1F] |
| 102 | 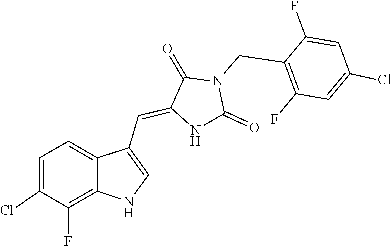 [SMILES: O=C1N/C(=C\c2c[nH]c3c(F)c(Cl)ccc23)C(=O)N1Cc1c(F)cc(Cl)cc1F] |
| 103 | 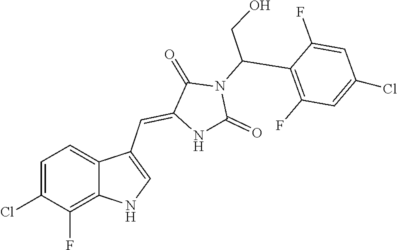 [SMILES: O=C1N/C(=C\c2c[nH]c3c(F)c(Cl)ccc23)C(=O)N1C(CO)c1c(F)cc(Cl)cc1F] |
| 104 | 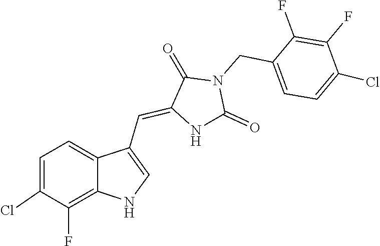 [SMILES: O=C1N/C(=C\c2c[nH]c3c(F)c(Cl)ccc23)C(=O)N1Cc1ccc(Cl)c(F)c1F] |
| 105 | 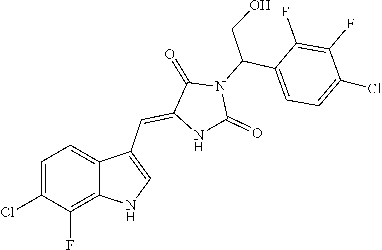 [SMILES: O=C1N/C(=C\c2c[nH]c3c(F)c(Cl)ccc23)C(=O)N1C(CO)c1ccc(Cl)c(F)c1F] |
| 106 | 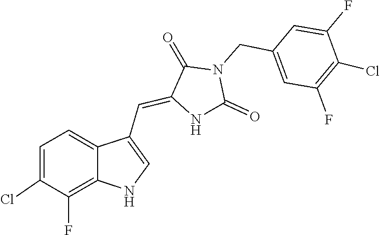 [SMILES: O=C1N/C(=C\c2c[nH]c3c(F)c(Cl)ccc23)C(=O)N1Cc1cc(F)c(Cl)c(F)c1] |
| 107 | 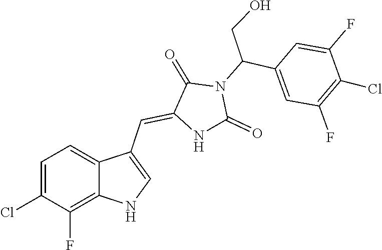 [SMILES: O=C1N/C(=C\c2c[nH]c3c(F)c(Cl)ccc23)C(=O)N1C(CO)c1cc(F)c(Cl)c(F)c1] |
| 108 | 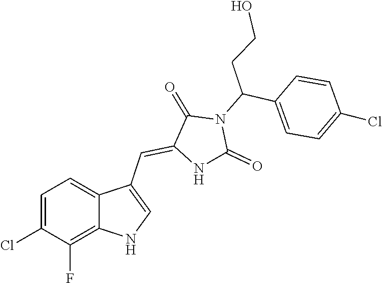 [SMILES: O=C1N/C(=C\c2c[nH]c3c(F)c(Cl)ccc23)C(=O)N1C(CCO)c1ccc(Cl)cc1] |
| 109 | 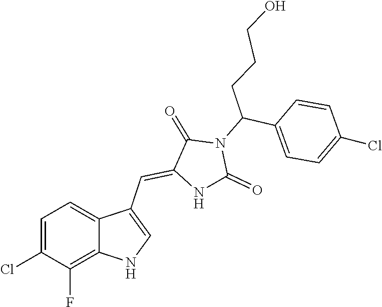 [SMILES: O=C1N/C(=C\c2c[nH]c3c(F)c(Cl)ccc23)C(=O)N1C(CCCO)c1ccc(Cl)cc1] |
| 110 | 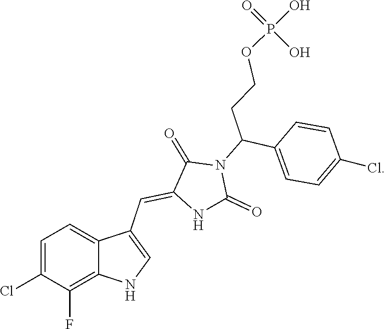 [SMILES: O=C1N/C(=C\c2c[nH]c3c(F)c(Cl)ccc23)C(=O)N1C(CCOP(=O)(O)O)c1ccc(Cl)cc1] |

7. A method for preparing the compound according to any one of claim 1, comprising a method I and/or a method II:
method I: performing a reaction on a compound a and a compound b to obtain the compound shown in formula (I); 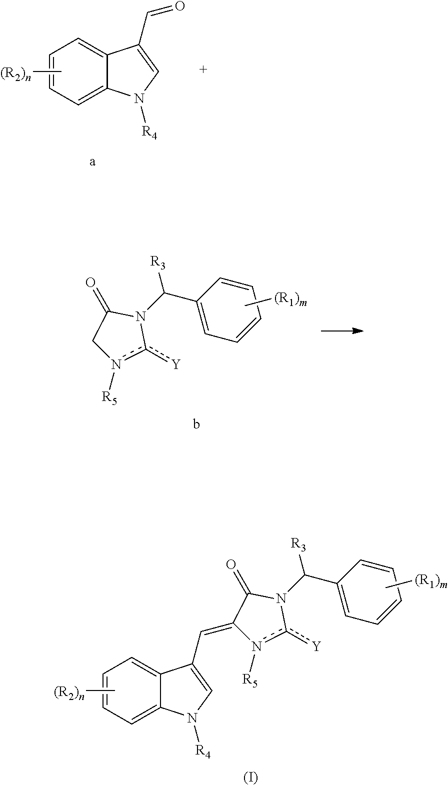
wherein R₁, R₂, R₃, R₄, R₅, Y, m, and n independently have definitions according to any one of claim 1;
method II: performing a reaction on a compound c and R_{3b}—NH₂ to obtain the compound shown in formula (I); 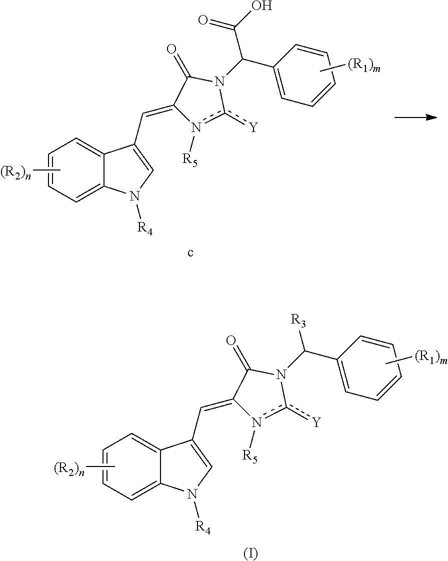
wherein R₁, R₂, R₃, R₄, R₅, R_{3b}, Y, m, and n independently have definitions according to any one of claim 1.

8. A pharmaceutical composition, comprising a therapeutically effective amount of at least one of the compound, and the raceme, the stereoisomer, the tautomer, the isotopic label, the solvate, the polymorph, the pharmaceutically acceptable salt or the prodrug compound thereof according to any one of claim 1.

9. Use of at least one of the compound, and the raceme, the stereoisomer, the tautomer, the isotopic label, the solvate, the polymorph, the pharmaceutically acceptable salt or the prodrug compound thereof according to any one of claim 1 in preparation of a drug, wherein
preferably, the use is use in preparation of a drug for treating an MDM2 and/or MDMX-mediated symptom and/or disease, for example, use in preparation of an MDM2 and/or MDMX inhibitor drug; and
preferably, the use is use in preparation of a drug for treating idiopathic pulmonary fibrosis.

10. The use according to claim 9, wherein the disease is a neoplastic disease, and the neoplastic disease comprises at least one of leukemia, chronic myeloid leukemia, acute myelocytic leukemia, lymphocytic leukemia, hairy cell leukemia, T lymphoblastic leukemia, lymphoma, B-cell lymphoma, burkitt lymphoma, hodgkin lymphoma, chondrosarcoma, fibrosarcoma, ewing sarcoma, rhabdomyosarcoma, small cell lung cancer, non-small cell lung cancer, myeloma, glioma, prostatic cancer, breast cancer, bone cancer, neuroblastoma, gastric cancer, ovarian cancer, intestinal cancer, renal cancer, medulloblastoma, pancreatic cancer, thyroid cancer, mesothelioma, cervical cancer, bladder cancer, liver cancer, skin cancer, and tumors in the central system or the peripheral nervous system.
